SEQUENCE LISTING (324 sequences)

SEQ ID NO: 1 - protein, 31 aa, Artificial Sequence
  Liraglutide backbone
  MOD_RES (20)..(20): Lys(Pal-Glu)
HAEGTFTSDVSSYLEGQAAKEFIAWLVRGRG

SEQ ID NO: 2 - protein, 31 aa, Artificial Sequence; identical to SEQ ID NO: 1
  Peptide fragment
  MOD_RES (1)..(1): His has N-terminal and side chain protecting groups
  MOD_RES (3)..(3): Glu has side chain protecting group
  MOD_RES (5)..(5): Thr has side chain protecting group
  MOD_RES (7)..(7): Thr has side chain protecting group
  MOD_RES (8)..(8): Ser has side chain protecting group
  MOD_RES (9)..(9): Asp has side chain protecting group
  MOD_RES (11)..(11): Ser has side chain protecting group
  MOD_RES (12)..(12): Ser has side chain protecting group
  MOD_RES (13)..(13): Tyr has side chain protecting group
  MOD_RES (15)..(15): Glu has side chain protecting group
  MOD_RES (17)..(17): Gln has side chain protecting group
  MOD_RES (20)..(20): Lys(Pal-Glu-side protecting group)
  MOD_RES (21)..(21): Glu has side chain protecting group
  MOD_RES (28)..(28): Arg has side chain protecting group
  MOD_RES (30)..(30): Arg has side chain protecting group
  MOD_RES (31)..(31): Gly-OH or Gly-O-solid support

SEQ ID NO: 3 - protein, 31 aa, Artificial Sequence; identical to SEQ ID NO: 1
  Peptide fragment
  MOD_RES (1)..(1): Boc-His(Trt)
  MOD_RES (3)..(3): Glu(OtBu)
  MOD_RES (5)..(5): Thr(tBu)
  MOD_RES (7)..(7): Thr(tBu)
  MOD_RES (8)..(8): Ser(tBu)
  MOD_RES (9)..(9): Asp(OtBu)
  MOD_RES (11)..(11): Ser(tBu)
  MOD_RES (12)..(12): Ser(PSI(Me,Me)Pro)
  MOD_RES (13)..(13): Tyr(tBu)
  MOD_RES (15)..(15): Glu(OtBu)
  MOD_RES (17)..(17): Gln(Trt)
  MOD_RES (20)..(20): Lys(Pal-Glu-OtBu)
  MOD_RES (21)..(21): Glu(OtBu)
  MOD_RES (28)..(28): Arg(Pbf)
  MOD_RES (30)..(30): Arg(Pbf)
  MOD_RES (31)..(31): Gly-O-Wang resin

SEQ ID NO: 4 - protein, 31 aa, Artificial Sequence; identical to SEQ ID NO: 1
  Peptide fragment
  MOD_RES (1)..(1): Boc-His(Trt)
  MOD_RES (3)..(3): Glu(OtBu)
  MOD_RES (5)..(5): Thr(tBu)
  MOD_RES (7)..(7): Thr(tBu)
  MOD_RES (8)..(8): Ser(tBu)
  MOD_RES (9)..(9): Asp(OtBu)
  MOD_RES (11)..(11): Ser(tBu)
  MOD_RES (12)..(12): Ser(PSI(Me,Me)Pro)
  MOD_RES (13)..(13): Tyr(tBu)
  MOD_RES (15)..(15): Glu(OtBu)
  MOD_RES (17)..(17): Gln(Trt)
  MOD_RES (20)..(20): Lys(Pal-Glu-OtBu)
  MOD_RES (21)..(21): Glu(OtBu)
  MOD_RES (28)..(28): Arg(Pbf)
  MOD_RES (30)..(30): Arg(Pbf)

SEQ ID NO: 5 - protein, 4 aa, Artificial Sequence
  Peptide fragment
HAEG

SEQ ID NO: 6 - protein, 4 aa, Artificial Sequence
  Peptide fragment
  MOD_RES (1)..(1): His has N-terminal and side chain protecting groups
  MOD_RES (3)..(3): Glu has side chain protecting group
  MOD_RES (4)..(4): Gly-OH or Gly-O-solid support or activated carboxylic ester of Gly
HAEG

SEQ ID NO: 7 - protein, 4 aa, Artificial Sequence
  Peptide fragment
  MOD_RES (1)..(1): Boc-His(Trt)
  MOD_RES (3)..(3): Glu(OtBu)
HAEG

SEQ ID NO: 8 - protein, 4 aa, Artificial Sequence
  Peptide fragment
  MOD_RES (1)..(1): Fmoc-His(Trt)
  MOD_RES (3)..(3): Glu(OtBu)
HAEG

SEQ ID NO: 9 - protein, 4 aa, Artificial Sequence
  Peptide fragment
  MOD_RES (1)..(1): Cbz-His(Trt)
  MOD_RES (3)..(3): Glu(OtBu)
HAEG

SEQ ID NO: 10 - protein, 4 aa, Artificial Sequence
  Peptide fragment
  MOD_RES (1)..(1): Boc-His(Trt)
  MOD_RES (3)..(3): Glu(OtBu)
  MOD_RES (4)..(4): Gly-OSu
HAEG

SEQ ID NO: 11 - protein, 4 aa, Artificial Sequence
  Peptide fragment
  MOD_RES (1)..(1): Fmoc-His(Trt)
  MOD_RES (3)..(3): Glu(OtBu)
  MOD_RES (4)..(4): Gly-OSu
HAEG

SEQ ID NO: 12 - protein, 4 aa, Artificial Sequence
  Peptide fragment
  MOD_RES (1)..(1): Cbz-His(Trt)
  MOD_RES (3)..(3): Glu(OtBu)
  MOD_RES (4)..(4): Gly-OSu
HAEG

SEQ ID NO: 13 - protein, 4 aa, Artificial Sequence
  Peptide fragment
  MOD_RES (1)..(1): Boc-His(Trt)
  MOD_RES (3)..(3): Glu(OtBu)
  MOD_RES (4)..(4): Gly-OBt
HAEG

SEQ ID NO: 14 - protein, 4 aa, Artificial Sequence
  Peptide fragment
  MOD_RES (1)..(1): Fmoc-His(Trt)
  MOD_RES (3)..(3): Glu(OtBu)
  MOD_RES (4)..(4): Gly-OBt
HAEG

SEQ ID NO: 15 - protein, 4 aa, Artificial Sequence
  Peptide fragment
  MOD_RES (1)..(1): Cbz-His(Trt)
  MOD_RES (3)..(3): Glu(OtBu)
  MOD_RES (4)..(4): Gly-OBt
HAEG

SEQ ID NO: 16 - protein, 4 aa, Artificial Sequence
  Peptide fragment
  MOD_RES (1)..(1): Boc-His(Trt)
  MOD_RES (3)..(3): Glu(OtBu)
  MOD_RES (4)..(4): Gly-O-CTC resin
HAEG

SEQ ID NO: 17 - protein, 4 aa, Artificial Sequence
  Peptide fragment
  MOD_RES (1)..(1): Fmoc-His(Trt)
  MOD_RES (3)..(3): Glu(OtBu)
  MOD_RES (4)..(4): Gly-O-CTC resin
HAEG

SEQ ID NO: 18 - protein, 4 aa, Artificial Sequence
  Peptide fragment
  MOD_RES (1)..(1): Cbz-His(Trt)
  MOD_RES (3)..(3): Glu(OtBu)
  MOD_RES (4)..(4): Gly-O-CTC resin
HAEG

SEQ ID NO: 19 - protein, 27 aa, Artificial Sequence
  Peptide fragment
TFTSDVSSYLEGQAAKEFIAWLVRGRG

SEQ ID NO: 20 - protein, 27 aa, Artificial Sequence
  Peptide fragment
  MOD_RES (1)..(1): Thr has H or N-terminal and side chain protecting groups
  MOD_RES (3)..(3): Thr has side chain protecting group
  MOD_RES (4)..(4): Ser has side chain protecting group
  MOD_RES (5)..(5): Asp has side chain protecting group
  MOD_RES (7)..(7): Ser has side chain protecting group
  MOD_RES (8)..(8): Ser has side chain protecting group
  MOD_RES (9)..(9): Tyr has side chain protecting group
  MOD_RES (11)..(11): Glu has side chain protecting group
  MOD_RES (13)..(13): Gln has side chain protecting group
  MOD_RES (16)..(16): Lys has side chain protecting group
  MOD_RES (17)..(17): Glu has side chain protecting group
  MOD_RES (24)..(24): Arg has side chain protecting group
  MOD_RES (26)..(26): Arg has side chain protecting group
  MOD_RES (27)..(27): Gly-OH or Gly-O-solid support
TFTSDVSSYLEGQAAKEFIAWLVRGRG

SEQ ID NO: 21 - protein, 27 aa, Artificial Sequence
  Peptide fragment
  MOD_RES (16)..(16): Lys(Glu)
TFTSDVSSYLEGQAAKEFIAWLVRGRG

SEQ ID NO: 22 - protein, 27 aa, Artificial Sequence
  Peptide fragment
  MOD_RES (1)..(1): Thr has H or N-terminal and side chain protecting groups
  MOD_RES (3)..(3): Thr has side chain protecting group
  MOD_RES (4)..(4): Ser has side chain protecting group
  MOD_RES (5)..(5): Asp has side chain protecting group
  MOD_RES (7)..(7): Ser has side chain protecting group
  MOD_RES (8)..(8): Ser has side chain protecting group
  MOD_RES (9)..(9): Tyr has side chain protecting group
  MOD_RES (11)..(11): Glu has side chain protecting group
  MOD_RES (13)..(13): Gln has side chain protecting group
  MOD_RES (16)..(16): Lys(Glu) has side chain protecting group
  MOD_RES (17)..(17): Glu has side chain protecting group
  MOD_RES (24)..(24): Arg has side chain protecting group
  MOD_RES (26)..(26): Arg has side chain protecting group
  MOD_RES (27)..(27): Gly-OH or Gly-O-solid support
TFTSDVSSYLEGQAAKEFIAWLVRGRG

SEQ ID NO: 23 - protein, 27 aa, Artificial Sequence
  Peptide fragment
  MOD_RES (1)..(1): Thr has H or N-terminal and side chain protecting groups
  MOD_RES (3)..(3): Thr has side chain protecting group
  MOD_RES (4)..(4): Ser has side chain protecting groups
  MOD_RES (5)..(5): Asp has side chain protecting groups
  MOD_RES (7)..(7): Ser has side chain protecting groups
  MOD_RES (8)..(8): Ser has side chain protecting groups
  MOD_RES (9)..(9): Tyr has side chain protecting groups
  MOD_RES (11)..(11): Glu has side chain protecting groups
  MOD_RES (13)..(13): Gln has side chain protecting groups
  MOD_RES (16)..(16): Lys(Side chain protecting group-Glu) has side chain protecting group
  MOD_RES (17)..(17): Glu has side chain protecting group
  MOD_RES (24)..(24): Arg has side chain protecting group
  MOD_RES (26)..(26): Arg has side chain protecting group
  MOD_RES (27)..(27): Gly-OH or Gly-O-solid support
TFTSDVSSYLEGQAAKEFIAWLVRGRG

SEQ ID NO: 24 - protein, 27 aa, Artificial Sequence
  Peptide fragment
  MOD_RES (16)..(16): Lys(Pal-Glu)
TFTSDVSSYLEGQAAKEFIAWLVRGRG

SEQ ID NO: 25 - protein, 27 aa, Artificial Sequence
  Peptide fragment
  MOD_RES (1)..(1): Thr has H or N-terminal and side chain protecting groups
  MOD_RES (3)..(3): Thr has side chain protecting group
  MOD_RES (4)..(4): Ser has side chain protecting group
  MOD_RES (5)..(5): Asp has side chain protecting group
  MOD_RES (7)..(7): Ser has side chain protecting group
  MOD_RES (8)..(8): Ser has side chain protecting group
  MOD_RES (9)..(9): Tyr has side chain protecting group
  MOD_RES (11)..(11): Glu has side chain protecting group
  MOD_RES (13)..(13): Gln has side chain protecting group
  MOD_RES (16)..(16): Lys(Pal-Glu) has side chain protecting group
  MOD_RES (17)..(17): Glu has side chain protecting group
  MOD_RES (24)..(24): Arg has side chain protecting group
  MOD_RES (26)..(26): Arg has side chain protecting group
  MOD_RES (27)..(27): Gly-OH or Gly-O-solid support
TFTSDVSSYLEGQAAKEFIAWLVRGRG

SEQ ID NO: 26 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mtt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 27 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mmt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 28 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 29 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 30 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Trt-Glu-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 31 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 32 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 33 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 34 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mtt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 35 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mmt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 36 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 37 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 38 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Trt-Glu-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC
TFTSDVSSYLEGQAAKEFIAWLVRGRG

SEQ ID NO: 39 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 40 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 41 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 42 - protein, 12 aa, Artificial Sequence
  Peptide fragment
TFTSDVSSYLEG

SEQ ID NO: 43 - protein, 12 aa, Artificial Sequence
  Peptide fragment
  MOD_RES (1)..(1): Thr has N-terminal and side chain protecting groups
  MOD_RES (3)..(3): Thr has side chain protecting group
  MOD_RES (4)..(4): Ser has side chain protecting group
  MOD_RES (5)..(5): Asp has side chain protecting group
  MOD_RES (7)..(7): Ser has side chain protecting group
  MOD_RES (8)..(8): Ser has side chain protecting group
  MOD_RES (9)..(9): Tyr has side chain protecting group
  MOD_RES (11)..(11): Glu has side chain protecting group
  MOD_RES (12)..(12): Gly-OH or Gly-O-solid support or activated carboxylic ester of Gly
TFTSDVSSYLEG

SEQ ID NO: 44 - protein, 12 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
TFTSDVSSYLEG

SEQ ID NO: 45 - protein, 12 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (12)..(12): Gly-OSu
TFTSDVSSYLEG

SEQ ID NO: 46 - protein, 12 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (12)..(12): Gly-O-CTC resin
TFTSDVSSYLEG

SEQ ID NO: 47 - protein, 12 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
TFTSDVSSYLEG

SEQ ID NO: 48 - protein, 12 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (12)..(12): Gly-OSu
TFTSDVSSYLEG

SEQ ID NO: 49 - protein, 12 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (12)..(12): Gly-O-CTC resin
TFTSDVSSYLEG

SEQ ID NO: 50 - protein, 15 aa, Artificial Sequence
  Peptide fragment
QAAKEFIAWLVRGRG

SEQ ID NO: 51 - protein, 15 aa, Artificial Sequence
  Peptide fragment
  MOD_RES (4)..(4): Lys(Glu)
QAAKEFIAWLVRGRG

SEQ ID NO: 52 - protein, 15 aa, Artificial Sequence
  Peptide fragment
  MOD_RES (4)..(4): Lys(Pal-Glu)
QAAKEFIAWLVRGRG

SEQ ID NO: 53 - protein, 15 aa, Artificial Sequence
  Peptide fragment
  MOD_RES (1)..(1): Gln has H or N-terminal and side chain protecting groups
  MOD_RES (4)..(4): Lys(side protecting group for Glu or Pal-Glu-side chain protecting group)
  MOD_RES (5)..(5): Glu has side chain protecting group
  MOD_RES (12)..(12): Arg has side chain protecting group
  MOD_RES (14)..(14): Arg has side chain protecting group
  MOD_RES (15)..(15): Gly-OH or Gly-O-solid support
QAAKEFIAWLVRGRG

SEQ ID NO: 54 - protein, 15 aa, Artificial Sequence
  Peptide fragment
  MOD_RES (1)..(1): Gln has H or N-terminal and side chain protecting groups
  MOD_RES (4)..(4): Lys(Mmt) or Lys(Mtt)
  MOD_RES (5)..(5): Glu has side chain protecting group
  MOD_RES (12)..(12): Arg has side chain protecting group
  MOD_RES (14)..(14): Arg has side chain protecting group
  MOD_RES (15)..(15): Gly-OH or Gly-O-solid support
QAAKEFIAWLVRGRG

SEQ ID NO: 55 - protein, 15 aa, Artificial Sequence
  Peptide fragment
  MOD_RES (1)..(1): Gln(Trt)
  MOD_RES (4)..(4): Lys(Pal-Glu-OtBu)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-Wang resin
QAAKEFIAWLVRGRG

SEQ ID NO: 56 - protein, 15 aa, Artificial Sequence
  Peptide fragment
  MOD_RES (1)..(1): Gln(Trt)
  MOD_RES (4)..(4): Lys(Pal-Glu-OtBu)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 57 - protein, 15 aa, Artificial Sequence
  Peptide fragment
  MOD_RES (1)..(1): Fmoc-Gln(Trt)
  MOD_RES (4)..(4): Lys(Pal-Glu-OtBu)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-Wang resin
QAAKEFIAWLVRGRG

SEQ ID NO: 58 - protein, 15 aa, Artificial Sequence
  Peptide fragment
  MOD_RES (1)..(1): Fmoc-Gln(Trt)
  MOD_RES (4)..(4): Lys(Trt-Glu-OtBu)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 59 - protein, 15 aa, Artificial Sequence
  Peptide fragment
  MOD_RES (1)..(1): Gln(Trt)
  MOD_RES (4)..(4): Lys(Trt-Glu-OtBu)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 60 - protein, 15 aa, Artificial Sequence
  Peptide fragment
  MOD_RES (1)..(1): Fmoc-Gln(Trt)
  MOD_RES (4)..(4): Lys(Glu-OtBu)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 61 - protein, 15 aa, Artificial Sequence
  Peptide fragment
  MOD_RES (1)..(1): Fmoc-Gln(Trt)
  MOD_RES (4)..(4): Lys(Pal-Glu-OtBu)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 62 - protein, 15 aa, Artificial Sequence
  Peptide fragment
  MOD_RES (1)..(1): Fmoc-Gln(Trt)
  MOD_RES (4)..(4): Lys(Mmt)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 63 - protein, 15 aa, Artificial Sequence
  Peptide fragment
  MOD_RES (1)..(1): Fmoc-Gln(Trt)
  MOD_RES (4)..(4): Lys(Mtt)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 64 - protein, 15 aa, Artificial Sequence
  Peptide fragment
  MOD_RES (1)..(1): Fmoc-Gln(Trt)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 65 - protein, 15 aa, Artificial Sequence
  Peptide fragment
  MOD_RES (1)..(1): Cbz-Gln(Trt)
  MOD_RES (4)..(4): Lys(Pal-Glu-OtBu)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-Wang resin
QAAKEFIAWLVRGRG

SEQ ID NO: 66 - protein, 15 aa, Artificial Sequence
  Peptide fragment
  MOD_RES (1)..(1): Cbz-Gln(Trt)
  MOD_RES (4)..(4): Lys(Trt-Glu-OtBu)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 67 - protein, 15 aa, Artificial Sequence
  Peptide fragment
  MOD_RES (1)..(1): Cbz-Gln(Trt)
  MOD_RES (4)..(4): Lys(Glu-OtBu)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 68 - protein, 15 aa, Artificial Sequence
  Peptide fragment
  MOD_RES (1)..(1): Cbz-Gln(Trt)
  MOD_RES (4)..(4): Lys(Pal-Glu-OtBu)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 69 - protein, 15 aa, Artificial Sequence
  Peptide fragment
  MOD_RES (1)..(1): Cbz-Gln(Trt)
  MOD_RES (4)..(4): Lys(Mmt)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 70 - protein, 15 aa, Artificial Sequence
  Peptide fragment
  MOD_RES (1)..(1): Cbz-Gln(Trt)
  MOD_RES (4)..(4): Lys(Mtt)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 71 - protein, 15 aa, Artificial Sequence
  Peptide fragment
  MOD_RES (1)..(1): Cbz-Gln(Trt)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 72 - protein, 15 aa, Artificial Sequence
  Peptide fragment
  MOD_RES (1)..(1): Fmoc-Gln(Trt)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-Wang resin
QAAKEFIAWLVRGRG

SEQ ID NO: 73 - protein, 15 aa, Artificial Sequence
  Peptide fragment
  MOD_RES (1)..(1): Fmoc-Gln(Trt)
  MOD_RES (4)..(4): Lys(Glu-OtBu)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-Wang resin
QAAKEFIAWLVRGRG

SEQ ID NO: 74 - protein, 15 aa, Artificial Sequence
  Peptide fragment
  MOD_RES (1)..(1): Gln(Trt)
  MOD_RES (4)..(4): Lys(Mmt)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 75 - protein, 15 aa, Artificial Sequence
  Peptide fragment
  MOD_RES (1)..(1): Gln(Trt)
  MOD_RES (4)..(4): Lys(Mmt)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-Wang resin
QAAKEFIAWLVRGRG

SEQ ID NO: 76 - protein, 15 aa, Artificial Sequence
  Peptide fragment
  MOD_RES (1)..(1): Gln(Trt)
  MOD_RES (4)..(4): Lys(Mtt)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 77 - protein, 15 aa, Artificial Sequence
  Peptide fragment
  MOD_RES (1)..(1): Gln(Trt)
  MOD_RES (4)..(4): Lys(Mtt)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-Wang resin
QAAKEFIAWLVRGRG

SEQ ID NO: 78 - protein, 8 aa, Artificial Sequence
  Peptide fragment
TFTSDVSS

SEQ ID NO: 79 - protein, 8 aa, Artificial Sequence
  Peptide fragment
  MOD_RES (1)..(1): Thr has N-terminal and side chain protecting groups
  MOD_RES (3)..(3): Thr has side chain protecting group
  MOD_RES (4)..(4): Ser has side chain protecting group
  MOD_RES (5)..(5): Asp has side chain protecting group
  MOD_RES (7)..(7): Ser has side chain protecting group
  MOD_RES (8)..(8): Ser has side chain protecting group and is Ser-OH or Ser-O-solid support
TFTSDVSS

SEQ ID NO: 80 - protein, 8 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)-O-CTC resin
TFTSDVSS

SEQ ID NO: 81 - protein, 8 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)-O-CTC resin
TFTSDVSS

SEQ ID NO: 82 - protein, 8 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
TFTSDVSS

SEQ ID NO: 83 - protein, 8 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
TFTSDVSS

SEQ ID NO: 84 - protein, 4 aa, Artificial Sequence
  Peptide fragment
YLEG

SEQ ID NO: 85 - protein, 4 aa, Artificial Sequence
  Peptide fragment
  MOD_RES (1)..(1): Tyr has N-terminal and side chain protecting groups
  MOD_RES (3)..(3): Glu has side chain protecting group
  MOD_RES (4)..(4): Gly-O-solid support
YLEG

SEQ ID NO: 86 - protein, 4 aa, Artificial Sequence
  Peptide fragment
  MOD_RES (1)..(1): Fmoc-Tyr(tBu)
  MOD_RES (3)..(3): Glu(OtBu)
  MOD_RES (4)..(4): Gly-O-CTC resin
YLEG

SEQ ID NO: 87 - protein, 4 aa, Artificial Sequence
  Peptide fragment
  MOD_RES (1)..(1): Cbz-Tyr(tBu)
  MOD_RES (3)..(3): Glu(OtBu)
  MOD_RES (4)..(4): Gly-O-CTC resin
YLEG

SEQ ID NO: 88 - protein, 4 aa, Artificial Sequence
  Peptide fragment
  MOD_RES (1)..(1): Tyr(tBu)
  MOD_RES (3)..(3): Glu(OtBu)
  MOD_RES (4)..(4): Gly-O-CTC resin
YLEG

SEQ ID NO: 89 - protein, 12 aa, Artificial Sequence
  Peptide fragment
KEFIAWLVRGRG

SEQ ID NO: 90 - protein, 12 aa, Artificial Sequence
  Peptide fragment
  MOD_RES (1)..(1): Lys has N-terminal and side chain protecting groups
  MOD_RES (2)..(2): Glu has side chain protecting group
  MOD_RES (9)..(9): Arg has side chain protecting group
  MOD_RES (11)..(11): Arg has side chain protecting group
  MOD_RES (12)..(12): Gly-O-solid support
KEFIAWLVRGRG

SEQ ID NO: 91 - protein, 12 aa, Artificial Sequence
  Peptide fragment
  MOD_RES (1)..(1): Fmoc-Lys(Mtt)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 92 - protein, 12 aa, Artificial Sequence
  Peptide fragment
  MOD_RES (1)..(1): Fmoc-Lys(Mmt)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 93 - protein, 12 aa, Artificial Sequence
  Peptide fragment
  MOD_RES (1)..(1): Fmoc-Lys
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 94 - protein, 12 aa, Artificial Sequence
  Peptide fragment
  MOD_RES (1)..(1): Fmoc-Lys(Pal-Glu-OtBu)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 95 - protein, 12 aa, Artificial Sequence
  Peptide fragment
  MOD_RES (1)..(1): Cbz-Lys(Mtt)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 96 - protein, 12 aa, Artificial Sequence
  Peptide fragment
  MOD_RES (1)..(1): Cbz-Lys(Mmt)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 97 - protein, 12 aa, Artificial Sequence
  Peptide fragment
  MOD_RES (1)..(1): Cbz-Lys
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 98 - protein, 12 aa, Artificial Sequence
  Peptide fragment
  MOD_RES (1)..(1): Cbz-Lys(Pal-Glu-OtBu)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 99 - protein, 12 aa, Artificial Sequence
  Peptide fragment
  MOD_RES (1)..(1): Fmoc-Lys(Trt-Glu-OtBu)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 100 - protein, 12 aa, Artificial Sequence
  Peptide fragment
  MOD_RES (1)..(1): Fmoc-Lys(Glu-OtBu)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 101 - protein, 13 aa, Artificial Sequence
  Peptide fragment
AKEFIAWLVRGRG

SEQ ID NO: 102 - protein, 13 aa, Artificial Sequence
  Peptide fragment
  MOD_RES (2)..(2): Lys(Pal-Glu) has side chain protecting group
  MOD_RES (3)..(3): Glu has side chain protecting group
  MOD_RES (10)..(10): Arg has side chain protecting group
  MOD_RES (12)..(12): Arg has side chain protecting group
  MOD_RES (13)..(13): Gly-O-solid support
AKEFIAWLVRGRG

SEQ ID NO: 103 - protein, 13 aa, Artificial Sequence
  Peptide fragment
  MOD_RES (2)..(2): Lys(Pal-Glu-OtBu)
  MOD_RES (3)..(3): Glu(OtBu)
  MOD_RES (10)..(10): Arg(Pbf)
  MOD_RES (12)..(12): Arg(Pbf)
  MOD_RES (13)..(13): Gly-O-Wang resin
AKEFIAWLVRGRG

SEQ ID NO: 104 - protein, 14 aa, Artificial Sequence
  Peptide fragment
AAKEFIAWLVRGRG

SEQ ID NO: 105 - protein, 14 aa, Artificial Sequence
  Peptide fragment
  MOD_RES (3)..(3): Lys(Pal-Glu) has side chain protecting group
  MOD_RES (4)..(4): Glu has side chain protecting group
  MOD_RES (11)..(11): Arg has side chain protecting group
  MOD_RES (13)..(13): Arg has side chain protecting group
  MOD_RES (14)..(14): Gly-O-solid support
AAKEFIAWLVRGRG

SEQ ID NO: 106 - protein, 12 aa, Artificial Sequence
  Peptide fragment
  MOD_RES (1)..(1): Fmoc-Lys(Pal-Glu-OtBu)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 107 - protein, 13 aa, Artificial Sequence
  Peptide fragment
  MOD_RES (1)..(1): Fmoc-Ala
  MOD_RES (2)..(2): Lys(Pal-Glu-OtBu)
  MOD_RES (3)..(3): Glu(OtBu)
  MOD_RES (10)..(10): Arg(Pbf)
  MOD_RES (12)..(12): Arg(Pbf)
  MOD_RES (13)..(13): Gly-O-Wang resin
AKEFIAWLVRGRG

SEQ ID NO: 108 - protein, 14 aa, Artificial Sequence
  Peptide fragment
  MOD_RES (1)..(1): Fmoc-Ala
  MOD_RES (3)..(3): Lys(Pal-Glu-OtBu)
  MOD_RES (4)..(4): Glu(OtBu)
  MOD_RES (11)..(11): Arg(Pbf)
  MOD_RES (13)..(13): Arg(Pbf)
  MOD_RES (14)..(14): Gly-O-Wang resin
AAKEFIAWLVRGRG

SEQ ID NO: 109 - protein, 12 aa, Artificial Sequence
  Peptide fragment
  MOD_RES (1)..(1): Fmoc-Lys(Glu-OtBu)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 110 - protein, 13 aa, Artificial Sequence
  Peptide fragment
  MOD_RES (1)..(1): Fmoc-Ala
  MOD_RES (2)..(2): Lys(Glu-OtBu)
  MOD_RES (3)..(3): Glu(OtBu)
  MOD_RES (10)..(10): Arg(Pbf)
  MOD_RES (12)..(12): Arg(Pbf)
  MOD_RES (13)..(13): Gly-O-Wang resin
AKEFIAWLVRGRG

SEQ ID NO: 111 - protein, 14 aa, Artificial Sequence
  Peptide fragment
  MOD_RES (1)..(1): Fmoc-Ala
  MOD_RES (3)..(3): Lys(Glu-OtBu)
  MOD_RES (4)..(4): Glu(OtBu)
  MOD_RES (11)..(11): Arg(Pbf)
  MOD_RES (13)..(13): Arg(Pbf)
  MOD_RES (14)..(14): Gly-O-Wang resin
AAKEFIAWLVRGRG

SEQ ID NO: 112 - protein, 14 aa, Artificial Sequence
  Peptide fragment
  MOD_RES (3)..(3): Lys(Pal-Glu-OtBu)
  MOD_RES (4)..(4): Glu(OtBu)
  MOD_RES (11)..(11): Arg(Pbf)
  MOD_RES (13)..(13): Arg(Pbf)
  MOD_RES (14)..(14): Gly-O-Wang resin
AAKEFIAWLVRGRG

SEQ ID NO: 113 - protein, 31 aa, Artificial Sequence; identical to SEQ ID NO: 1
  Peptide fragment
  MOD_RES (1)..(1): D-His
  MOD_RES (20)..(20): Lys(Pal-Glu-OH)

SEQ ID NO: 114 - protein, 32 aa, Artificial Sequence
  Peptide fragment
  MOD_RES (21)..(21): Lys(Pal-Glu-OH)
HAEGGTFTSDVSSYLEGQAAKEFIAWLVRGRG

SEQ ID NO: 115 - protein, 5 aa, Artificial Sequence
  Peptide fragment
HAEGG

SEQ ID NO: 116 - protein, 32 aa, Artificial Sequence
  Peptide fragment
  MOD_RES (21)..(21): Lys(Pal-Glu-OH)
HAEGTFTSDVSSYLEGGQAAKEFIAWLVRGRG

SEQ ID NO: 117 - protein, 32 aa, Artificial Sequence
  Peptide fragment
  MOD_RES (20)..(20): Lys(Pal-Glu-OH)
HAEGTFTSDVSSYLEGQAAKEFIAWLVRGRGG

SEQ ID NO: 118 - protein, 4 aa, Artificial Sequence
  Peptide fragment
  MOD_RES (1)..(1): His(Trt)
  MOD_RES (3)..(3): Glu(OtBu)
HAEG

SEQ ID NO: 119 - protein, 4 aa, Artificial Sequence
  Peptide fragment
  MOD_RES (1)..(1): His(Trt)
  MOD_RES (3)..(3): Glu(OtBu)
  MOD_RES (4)..(4): Gly-OSu
HAEG

SEQ ID NO: 120 - protein, 4 aa, Artificial Sequence
  Peptide fragment
  MOD_RES (1)..(1): His(Trt)
  MOD_RES (3)..(3): Glu(OtBu)
  MOD_RES (4)..(4): Gly-OBt
HAEG

SEQ ID NO: 121 - protein, 4 aa, Artificial Sequence
  Peptide fragment
  MOD_RES (1)..(1): His(Trt)
  MOD_RES (3)..(3): Glu(OtBu)
  MOD_RES (4)..(4): Gly(OPfp)
HAEG

SEQ ID NO: 122 - protein, 4 aa, Artificial Sequence
  Peptide fragment
  MOD_RES (1)..(1): His(Trt)
  MOD_RES (3)..(3): Glu(OtBu)
  MOD_RES (4)..(4): Gly-O-CTC resin
HAEG

SEQ ID NO: 123 - protein, 12 aa, Artificial Sequence
  Peptide fragment
  MOD_RES (1)..(1): Lys(Mtt)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 124 - protein, 12 aa, Artificial Sequence
  Peptide fragment
  MOD_RES (1)..(1): Lys(Mmt)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 125 - protein, 12 aa, Artificial Sequence
  Peptide fragment
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 126 - protein, 12 aa, Artificial Sequence
  Peptide fragment
  MOD_RES (1)..(1): Lys(Glu-OtBu)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 127 - protein, 12 aa, Artificial Sequence
  Peptide fragment
  MOD_RES (1)..(1): Lys(Pal-Glu-OtBu)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 128 - protein, 15 aa, Artificial Sequence
  Peptide fragment
  MOD_RES (1)..(1): Gln(Trt)
  MOD_RES (4)..(4): Lys(Pal-Glu-OtBu)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-Wang resin
QAAKEFIAWLVRGRG

SEQ ID NO: 129 - protein, 13 aa, Artificial Sequence
  Peptide fragment
  MOD_RES (2)..(2): Lys(Mtt)
  MOD_RES (3)..(3): Glu(OtBu)
  MOD_RES (10)..(10): Arg(Pbf)
  MOD_RES (12)..(12): Arg(Pbf)
  MOD_RES (13)..(13): Gly-O-Wang resin
AKEFIAWLVRGRG

SEQ ID NO: 130 - protein, 13 aa, Artificial Sequence
  Peptide fragment
  MOD_RES (2)..(2): Lys(Mmt)
  MOD_RES (3)..(3): Glu(OtBu)
  MOD_RES (10)..(10): Arg(Pbf)
  MOD_RES (12)..(12): Arg(Pbf)
  MOD_RES (13)..(13): Gly-O-Wang resin
AKEFIAWLVRGRG

SEQ ID NO: 131 - protein, 13 aa, Artificial Sequence
  Peptide fragment
  MOD_RES (2)..(2): Lys(Trt-Glu-OtBu)
  MOD_RES (3)..(3): Glu(OtBu)
  MOD_RES (10)..(10): Arg(Pbf)
  MOD_RES (12)..(12): Arg(Pbf)
  MOD_RES (13)..(13): Gly-O-Wang resin
AKEFIAWLVRGRG

SEQ ID NO: 132 - protein, 13 aa, Artificial Sequence
  Peptide fragment
  MOD_RES (2)..(2): Lys(Glu-OtBu)
  MOD_RES (3)..(3): Glu(OtBu)
  MOD_RES (10)..(10): Arg(Pbf)
  MOD_RES (12)..(12): Arg(Pbf)
  MOD_RES (13)..(13): Gly-O-Wang resin
AKEFIAWLVRGRG

SEQ ID NO: 133 - protein, 13 aa, Artificial Sequence
  Peptide fragment
  MOD_RES (3)..(3): Glu(OtBu)
  MOD_RES (10)..(10): Arg(Pbf)
  MOD_RES (12)..(12): Arg(Pbf)
  MOD_RES (13)..(13): Gly-O-Wang resin
AKEFIAWLVRGRG

SEQ ID NO: 134 - protein, 13 aa, Artificial Sequence
  Peptide fragment
  MOD_RES (2)..(2): Lys(Pal-Glu-OtBu)
  MOD_RES (3)..(3): Glu(OtBu)
  MOD_RES (10)..(10): Arg(Pbf)
  MOD_RES (12)..(12): Arg(Pbf)
  MOD_RES (13)..(13): Gly-O-Wang resin
AKEFIAWLVRGRG

SEQ ID NO: 135 - protein, 14 aa, Artificial Sequence
  Peptide fragment
  MOD_RES (3)..(3): Lys(Mtt)
  MOD_RES (4)..(4): Glu(OtBu)
  MOD_RES (11)..(11): Arg(Pbf)
  MOD_RES (13)..(13): Arg(Pbf)
  MOD_RES (14)..(14): Gly-O-Wang resin
AAKEFIAWLVRGRG

SEQ ID NO: 136 - protein, 14 aa, Artificial Sequence
  Peptide fragment
  MOD_RES (3)..(3): Lys(Mmt)
  MOD_RES (4)..(4): Glu(OtBu)
  MOD_RES (11)..(11): Arg(Pbf)
  MOD_RES (13)..(13): Arg(Pbf)
  MOD_RES (14)..(14): Gly-O-Wang resin
AAKEFIAWLVRGRG

SEQ ID NO: 137 - protein, 14 aa, Artificial Sequence
  Peptide fragment
  MOD_RES (3)..(3): Lys(Trt-Glu-OtBu)
  MOD_RES (4)..(4): Glu(OtBu)
  MOD_RES (11)..(11): Arg(Pbf)
  MOD_RES (13)..(13): Arg(Pbf)
  MOD_RES (14)..(14): Gly-O-Wang resin
AAKEFIAWLVRGRG

SEQ ID NO: 138 - protein, 14 aa, Artificial Sequence
  Peptide fragment
  MOD_RES (3)..(3): Lys(Glu-OtBu)
  MOD_RES (4)..(4): Glu(OtBu)
  MOD_RES (11)..(11): Arg(Pbf)
  MOD_RES (13)..(13): Arg(Pbf)
  MOD_RES (14)..(14): Gly-O-Wang resin
AAKEFIAWLVRGRG

SEQ ID NO: 139 - protein, 14 aa, Artificial Sequence
  Peptide fragment
  MOD_RES (4)..(4): Glu(OtBu)
  MOD_RES (11)..(11): Arg(Pbf)
  MOD_RES (13)..(13): Arg(Pbf)
  MOD_RES (14)..(14): Gly-O-Wang resin
AAKEFIAWLVRGRG

SEQ ID NO: 140 - protein, 14 aa, Artificial Sequence
  Peptide fragment
  MOD_RES (3)..(3): Lys(Pal-Glu-OtBu)
  MOD_RES (4)..(4): Glu(OtBu)
  MOD_RES (11)..(11): Arg(Pbf)
  MOD_RES (13)..(13): Arg(Pbf)
  MOD_RES (14)..(14): Gly-O-Wang resin
AAKEFIAWLVRGRG

SEQ ID NO: 141 - protein, 12 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
TFTSDVSSYLEG

SEQ ID NO: 142 - protein, 12 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (12)..(12): Gly-OSu
TFTSDVSSYLEG

SEQ ID NO: 143 - protein, 15 aa, Artificial Sequence
  Peptide fragment
  MOD_RES (1)..(1): Gln(Trt)
  MOD_RES (4)..(4): Lys(Trt-Glu-OtBu)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 144 - protein, 15 aa, Artificial Sequence
  Peptide fragment
  MOD_RES (1)..(1): Gln(Trt)
  MOD_RES (4)..(4): Lys(Mmt)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 145 - protein, 15 aa, Artificial Sequence
  Peptide fragment
  MOD_RES (1)..(1): Gln(Trt)
  MOD_RES (4)..(4): Lys(Mtt)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 146 - protein, 15 aa, Artificial Sequence
  Peptide fragment
  MOD_RES (1)..(1): Gln(Trt)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 147 - protein, 15 aa, Artificial Sequence
  Peptide fragment
  MOD_RES (1)..(1): Gln(Trt)
  MOD_RES (4)..(4): Lys(Glu-OtBu)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 148 - protein, 15 aa, Artificial Sequence
  Peptide fragment
  MOD_RES (1)..(1): Gln(Trt)
  MOD_RES (4)..(4): Lys(Pal-Glu-OtBu)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 149 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 150 - protein, 15 aa, Artificial Sequence
  Peptide fragment
  MOD_RES (1)..(1): Gln(Trt)
  MOD_RES (4)..(4): Lys(Trt-Glu-OtBu)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-Wang resin
QAAKEFIAWLVRGRG

SEQ ID NO: 151 - protein, 15 aa, Artificial Sequence
  Peptide fragment
  MOD_RES (1)..(1): Gln(Trt)
  MOD_RES (4)..(4): Lys(Glu-OtBu)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-Wang resin
QAAKEFIAWLVRGRG

SEQ ID NO: 152 - protein, 15 aa, Artificial Sequence
  Peptide fragment
  MOD_RES (1)..(1): Gln(Trt)
  MOD_RES (4)..(4): Lys(Mtt)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 153 - protein, 15 aa, Artificial Sequence
  Peptide fragment
  MOD_RES (1)..(1): Gln(Trt)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 154 - protein, 15 aa, Artificial Sequence
  Peptide fragment
  MOD_RES (1)..(1): Gln(Trt)
  MOD_RES (4)..(4): Lys(Mmt)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-CTC resin
QAAKEFIAWLVRGRG

SEQ ID NO: 155 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mtt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 156 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 157 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mtt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 158 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mmt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 159 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 160 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Trt-Gly-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 161 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 162 - protein, 26 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Glu-OtBu)
  MOD_RES (23)..(23): Arg(Pbf)
  MOD_RES (25)..(25): Arg(Pbf)
TFTSDVSSYLEGQAAKFIAWLVRGRG

SEQ ID NO: 163 - protein, 4 aa, Artificial Sequence
  Peptide fragment
  MOD_RES (1)..(1): Tyr(tBu)
  MOD_RES (3)..(3): Glu(OtBu)
  MOD_RES (4)..(4): Gly-O-CTC resin
YLEG

SEQ ID NO: 164 - protein, 8 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)-O-CTC resin
TFTSDVSS

SEQ ID NO: 165 - protein, 12 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (12)..(12): Gly-O-CTC resin
TFTSDVSSYLEG

SEQ ID NO: 166 - protein, 12 aa, Artificial Sequence
  Peptide fragment
  MOD_RES (1)..(1): Lys(Trt-Glu-OtBu)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 167 - protein, 12 aa, Artificial Sequence
  Peptide fragment
  MOD_RES (1)..(1): Lys(Glu-OtBu)
  MOD_RES (2)..(2): Glu(OtBu)
  MOD_RES (9)..(9): Arg(Pbf)
  MOD_RES (11)..(11): Arg(Pbf)
  MOD_RES (12)..(12): Gly-O-Wang resin
KEFIAWLVRGRG

SEQ ID NO: 168 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mmt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 169 - protein, 26 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Trt-Glu-OtBu)
  MOD_RES (23)..(23): Arg(Pbf)
  MOD_RES (25)..(25): Arg(Pbf)
  MOD_RES (26)..(26): Gly-O-CTC resin
TFTSDVSSYLEGQAAKFIAWLVRGRG

SEQ ID NO: 170 - protein, 30 aa, Artificial Sequence
  Peptide fragment
  MOD_RES (1)..(1): His(Trt)
  MOD_RES (3)..(3): Glu(OtBu)
  MOD_RES (5)..(5): Thr(tBu)
  MOD_RES (7)..(7): Thr(tBu)
  MOD_RES (8)..(8): Ser(tBu)
  MOD_RES (9)..(9): Asp(OtBu)
  MOD_RES (11)..(11): Ser(tBu)
  MOD_RES (12)..(12): Ser(PSI(Me,Me)Pro)
  MOD_RES (13)..(13): Tyr(tBu)
  MOD_RES (15)..(15): Glu(OtBu)
  MOD_RES (17)..(17): Gln(Trt)
  MOD_RES (20)..(20): Lys(Glu-OtBu)
  MOD_RES (27)..(27): Arg(Pbf)
  MOD_RES (29)..(29): Arg(Pbf)
  MOD_RES (30)..(30): Gly-O-Wang resin
HAEGTFTSDVSSYLEGQAAKFIAWLVRGRG

SEQ ID NO: 171-172 - skipped (2 entries)

SEQ ID NO: 173 - protein, 4 aa, Artificial Sequence
  Peptide fragment
  MOD_RES (1)..(1): Boc-D-His(Trt)
  MOD_RES (3)..(3): Glu(OtBu)
HAEG

SEQ ID NO: 174 - protein, 31 aa, Artificial Sequence
  Semaglutide backbone
  MOD_RES (2)..(2): Aib
  MOD_RES (20)..(20): Lys is derivatized with N-(17-carboxy-1-oxoheptadecyl)-L-gamma-glutamyl-2-[2-(2-aminoetho xy)ethoxy]acetyl-2-[2-(2-aminoethoxy)ethoxy]acetyl
HXEGTFTSDVSSYLEGQAAKEFIAWLVRGRG

SEQ ID NO: 175 - protein, 4 aa, Artificial Sequence
  Peptide fragment
  MOD_RES (2)..(2): Aib
HXEG

SEQ ID NO: 176 - protein, 27 aa, Artificial Sequence
  Peptide fragment
  MOD_RES (16)..(16): Lys(N-(17-carboxy-1-oxoheptadecyl)-L-gamma- glutamyl-2-[2-(2-amino ethoxy)ethoxy]acetyl-2-[2-(2-aminoethoxy) ethoxy]acetyl)
TFTSDVSSYLEGQAAKEFIAWLVRGRG

SEQ ID NO: 177 - protein, 4 aa, Artificial Sequence
  Peptide fragment
  MOD_RES (1)..(1): His has N-terminal and side chain protecting groups
  MOD_RES (2)..(2): Aib
  MOD_RES (3)..(3): Glu has side chain protecting group
  MOD_RES (4)..(4): Gly-OH or Gly-O-solid support or activated carboxylic ester of Gly
HXEG

SEQ ID NO: 178 - protein, 4 aa, Artificial Sequence
  Peptide fragment
  MOD_RES (1)..(1): Boc-His(Trt)
  MOD_RES (2)..(2): Aib
  MOD_RES (3)..(3): Glu(OtBu)
HXEG

SEQ ID NO: 179 - protein, 4 aa, Artificial Sequence
  Peptide fragment
  MOD_RES (1)..(1): Fmoc-His(Trt)
  MOD_RES (2)..(2): Aib
  MOD_RES (3)..(3): Glu(OtBu)
HXEG

SEQ ID NO: 180 - protein, 4 aa, Artificial Sequence
  Peptide fragment
  MOD_RES (1)..(1): Cbz-His(Trt)
  MOD_RES (2)..(2): Aib
  MOD_RES (3)..(3): Glu(OtBu)
HXEG

SEQ ID NO: 181 - protein, 4 aa, Artificial Sequence
  Peptide fragment
  MOD_RES (1)..(1): Boc-His(Trt)
  MOD_RES (2)..(2): Aib
  MOD_RES (3)..(3): Glu(OtBu)
  MOD_RES (4)..(4): Gly-OSu
HXEG

SEQ ID NO: 182 - protein, 4 aa, Artificial Sequence
  Peptide fragment
  MOD_RES (1)..(1): Fmoc-His(Trt)
  MOD_RES (2)..(2): Aib
  MOD_RES (3)..(3): Glu(OtBu)
  MOD_RES (4)..(4): Gly-OSu
HXEG

SEQ ID NO: 183 - protein, 4 aa, Artificial Sequence
  Peptide fragment
  MOD_RES (1)..(1): Cbz-His(Trt)
  MOD_RES (2)..(2): Aib
  MOD_RES (3)..(3): Glu(OtBu)
  MOD_RES (4)..(4): Gly-OSu
HXEG

SEQ ID NO: 184 - protein, 4 aa, Artificial Sequence
  Peptide fragment
  MOD_RES (1)..(1): Boc-His(Trt)
  MOD_RES (2)..(2): Aib
  MOD_RES (3)..(3): Glu(OtBu)
  MOD_RES (4)..(4): Gly-OBt
HXEG

SEQ ID NO: 185 - protein, 4 aa, Artificial Sequence
  Peptide fragment
  MOD_RES (1)..(1): Fmoc-His(Trt)
  MOD_RES (2)..(2): Aib
  MOD_RES (3)..(3): Glu(OtBu)
  MOD_RES (4)..(4): Gly-OBt
HXEG

SEQ ID NO: 186 - protein, 4 aa, Artificial Sequence
  Peptide fragment
  MOD_RES (1)..(1): Cbz-His(Trt)
  MOD_RES (2)..(2): Aib
  MOD_RES (3)..(3): Glu(OtBu)
  MOD_RES (4)..(4): Gly-OBt
HXEG

SEQ ID NO: 187 - protein, 4 aa, Artificial Sequence
  Peptide fragment
  MOD_RES (1)..(1): Boc-His(Trt)
  MOD_RES (2)..(2): Aib
  MOD_RES (3)..(3): Glu(OtBu)
  MOD_RES (4)..(4): Gly(OPfp)
HXEG

SEQ ID NO: 188 - protein, 4 aa, Artificial Sequence
  Peptide fragment
  MOD_RES (1)..(1): Fmoc-His(Trt)
  MOD_RES (2)..(2): Aib
  MOD_RES (3)..(3): Glu(OtBu)
  MOD_RES (4)..(4): Gly(OPfp)
HXEG

SEQ ID NO: 189 - protein, 4 aa, Artificial Sequence
  Peptide fragment
  MOD_RES (1)..(1): Cbz-His(Trt)
  MOD_RES (2)..(2): Aib
  MOD_RES (3)..(3): Glu(OtBu)
  MOD_RES (4)..(4): Gly(OPfp)
HXEG

SEQ ID NO: 190 - protein, 27 aa, Artificial Sequence
  Peptide fragment
  MOD_RES (1)..(1): Thr has H or N-terminal and side chain protecting groups
  MOD_RES (3)..(3): Thr has side chain protecting group
  MOD_RES (4)..(4): Ser has side chain protecting group
  MOD_RES (5)..(5): Asp has side chain protecting group
  MOD_RES (7)..(7): Ser has side chain protecting group
  MOD_RES (8)..(8): Ser has side chain protecting group
  MOD_RES (9)..(9): Tyr has side chain protecting group
  MOD_RES (11)..(11): Glu has side chain protecting group
  MOD_RES (13)..(13): Gln has side chain protecting group
  MOD_RES (16)..(16): Lys(N-(17-carboxy(P)-1-oxoheptadecyl)-L-gamma- glutamyl(P)-2-[2-(2-aminoethoxy)ethoxy]acetyl-2-[2-(2- aminoethoxy)ethoxy]acetyl)
  MOD_RES (17)..(17): Glu has side chain protecting group
  MOD_RES (24)..(24): Arg has side chain protecting group
  MOD_RES (26)..(26): Arg has side chain protecting group
  MOD_RES (27)..(27): Gly-OH or Gly-O-solid support
TFTSDVSSYLEGQAAKEFIAWLVRGRG

SEQ ID NO: 191 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(N-(17-carboxy(OtBu)-1-oxoheptadecyl)-L- gamma-glutamyl(OtBu)-2-[2-(2-aminoethoxy)ethoxy]acetyl-2-[2-(2- aminoethoxy)ethoxy]acetyl)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 192 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(N-(17-carboxy(OtBu)-1-oxoheptadecyl)-L- gamma-glutamyl(OtBu)-2-[2-(2-aminoethoxy)ethoxy]acetyl-2-[2-(2- aminoethoxy)ethoxy]acetyl)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 193 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(N-(17-carboxy(OtBu)-1-oxoheptadecyl)-L- gamma-glutamyl(OtBu)-2-[2-(2-aminoethoxy)ethoxy]acetyl-2-[2-(2- aminoethoxy)ethoxy]acetyl)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 194 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(N-(17-carboxy(OtBu)-1-oxoheptadecyl)-L- gamma-glutamyl(OtBu)-2-[2-(2-aminoethoxy)ethoxy]acetyl-2-[2-(2- aminoethoxy)ethoxy]acetyl)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 195 - protein, 15 aa, Artificial Sequence
  Peptide fragment
  MOD_RES (4)..(4): Lys(N-(17-carboxy-1-oxoheptadecyl)-L-gamma- glutamyl-2-[2-(2-amino ethoxy)ethoxy]acetyl-2-[2-(2-aminoethoxy) ethoxy]acetyl)
QAAKEFIAWLVRGRG

SEQ ID NO: 196 - protein, 15 aa, Artificial Sequence
  Peptide fragment
  MOD_RES (1)..(1): Gln has H or N-terminal and side chain protecting groups
  MOD_RES (4)..(4): Lys(N-(17-carboxy(P)-1-oxoheptadecyl)-L-gamma- glutamyl(P)-2-[2-(2-aminoethoxy)ethoxy]acetyl-2-[2-(2-aminoethoxy) ethoxy]acetyl)
  MOD_RES (5)..(5): Glu has side chain protecting group
  MOD_RES (12)..(12): Arg has side chain protecting group
  MOD_RES (14)..(14): Arg has side chain protecting group
  MOD_RES (15)..(15): Gly-OH or Gly-O-solid support
QAAKEFIAWLVRGRG

SEQ ID NO: 197 - protein, 15 aa, Artificial Sequence
  Peptide fragment
  MOD_RES (1)..(1): Gln(Trt)
  MOD_RES (4)..(4): Lys(N-(17-carboxy(OtBu)-1-oxoheptadecyl)-L- gamma-glutamyl(OtBu)-2-[2-(2-aminoethoxy)ethoxy]acetyl-2-[2-(2- aminoethoxy)ethoxy]acetyl)
  MOD_RES (5)..(5): Glu(OtBu)
  MOD_RES (12)..(12): Arg(Pbf)
  MOD_RES (14)..(14): Arg(Pbf)
  MOD_RES (15)..(15): Gly-O-Wang resin
QAAKEFIAWLVRGRG

SEQ ID NO: 198 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(N-(17-carboxy(OtBu)-1-oxoheptadecyl)-L- gamma-glutamyl(OtBu)-2-[2-(2-aminoethoxy)ethoxy]acetyl-2-[2-(2- aminoethoxy)ethoxy]acetyl)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 199 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(N-(17-carboxy(OtBu)-1-oxoheptadecyl)-L- gamma-glutamyl(OtBu)-2-[2-(2-aminoethoxy)ethoxy]acetyl-2-[2-(2- aminoethoxy)ethoxy]acetyl)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 200 - protein, 15 aa, Artificial Sequence
  Peptide fragment
  MOD_RES (1)..(1): Gln(Trt)
  MOD_RES (4)..(4): Lys(N-(17-carboxy(OtBu)-1-oxoheptadecyl)-L- gamma-glutamyl(OtBu)-2-[2-(2-aminoethoxy)ethoxy]acetyl-2-[2-(2- aminoethoxy)ethoxy]acetyl)
  MOD_RES (5)..(5): Glu(OtBu)
  MOD_RES (12)..(12): Arg(Pbf)
  MOD_RES (14)..(14): Arg(Pbf)
QAAKEFIAWLVRGRG

SEQ ID NO: 201 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(N-(17-carboxy(OtBu)-1-oxoheptadecyl)-L- gamma-glutamyl(OtBu)-2-[2-(2-aminoethoxy)ethoxy]acetyl-2-[2-(2- aminoethoxy)ethoxy]acetyl)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 202 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(N-(17-carboxy(OtBu)-1-oxoheptadecyl)-L- gamma-glutamyl(OtBu)-2-[2-(2-aminoethoxy)ethoxy]acetyl-2-[2-(2- aminoethoxy)ethoxy]acetyl)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 203 - protein, 31 aa, Artificial Sequence; identical to SEQ ID NO: 174
  Peptide fragment
  MOD_RES (1)..(1): D-His
  MOD_RES (2)..(2): Aib
  MOD_RES (20)..(20): Lys(N-(17-carboxy-1-oxoheptadecyl)-L-gamma- glutamyl-2-[2-(2-aminoethoxy)ethoxy]acetyl-2-[2-(2-aminoethoxy) ethoxy]acetyl)

SEQ ID NO: 204 - protein, 5 aa, Artificial Sequence
  Peptide fragment
  MOD_RES (2)..(2): Aib
HXEGG

SEQ ID NO: 205 - protein, 32 aa, Artificial Sequence
  Peptide fragment
  MOD_RES (2)..(2): Aib
  MOD_RES (21)..(21): Lys(N-(17-carboxy-1-oxoheptadecyl)-L-gamma- glutamyl-2-[2-(2-aminoethoxy)ethoxy]acetyl-2-[2-(2-aminoethoxy) ethoxy]acetyl)
HXEGGTFTSDVSSYLEGQAAKEFIAWLVRGRG

SEQ ID NO: 206 - protein, 32 aa, Artificial Sequence
  Peptide fragment
  MOD_RES (2)..(2): Aib
  MOD_RES (21)..(21): Lys(N-(17-carboxy-1-oxoheptadecyl)-L-gamma- glutamyl-2-[2-(2-amino ethoxy)ethoxy]acetyl-2-[2-(2-aminoethoxy) ethoxy]acetyl)
HXEGTFTSDVSSYLEGGQAAKEFIAWLVRGRG

SEQ ID NO: 207 - protein, 32 aa, Artificial Sequence
  Peptide fragment
  MOD_RES (2)..(2): Aib
  MOD_RES (20)..(20): Lys(N-(17-carboxy-1-oxoheptadecyl)-L-gamma- glutamyl-2-[2-(2-amino ethoxy)ethoxy]acetyl-2-[2-(2-aminoethoxy) ethoxy]acetyl)
HXEGTFTSDVSSYLEGQAAKEFIAWLVRGRGG

SEQ ID NO: 208-265 - skipped (58 entries)

SEQ ID NO: 266 - protein, 31 aa, Artificial Sequence; identical to SEQ ID NO: 1
  Peptide fragment
  MOD_RES (1)..(1): Boc-His(Trt)
  MOD_RES (3)..(3): Glu(OtBu)
  MOD_RES (5)..(5): Thr(tBu)
  MOD_RES (7)..(7): Thr(tBu)
  MOD_RES (8)..(8): Ser(tBu)
  MOD_RES (9)..(9): Asp(OtBu)
  MOD_RES (11)..(11): Ser(Trt)
  MOD_RES (12)..(12): Ser(Trt)
  MOD_RES (13)..(13): Tyr(tBu)
  MOD_RES (15)..(15): Glu(OtBu)
  MOD_RES (17)..(17): Gln(Trt)
  MOD_RES (20)..(20): Lys(Pal-Glu-OtBu)
  MOD_RES (21)..(21): Glu(OtBu)
  MOD_RES (28)..(28): Arg(Pbf)
  MOD_RES (30)..(30): Arg(Pbf)
  MOD_RES (31)..(31): Gly-O-Wang resin

SEQ ID NO: 267 - protein, 31 aa, Artificial Sequence; identical to SEQ ID NO: 1
  Peptide fragment
  MOD_RES (1)..(1): Boc-His(Trt)
  MOD_RES (3)..(3): Glu(OtBu)
  MOD_RES (5)..(5): Thr(tBu)
  MOD_RES (7)..(7): Thr(tBu)
  MOD_RES (8)..(8): Ser(tBu)
  MOD_RES (9)..(9): Asp(OtBu)
  MOD_RES (11)..(11): Ser(Trt)
  MOD_RES (12)..(12): Ser(Trt)
  MOD_RES (13)..(13): Tyr(tBu)
  MOD_RES (15)..(15): Glu(OtBu)
  MOD_RES (17)..(17): Gln(Trt)
  MOD_RES (20)..(20): Lys(Pal-Glu-OtBu)
  MOD_RES (21)..(21): Glu(OtBu)
  MOD_RES (28)..(28): Arg(Pbf)
  MOD_RES (30)..(30): Arg(Pbf)

SEQ ID NO: 268 - protein, 4 aa, Artificial Sequence
  Peptide fragment
  MOD_RES (1)..(1): Boc-His(Trt)
  MOD_RES (3)..(3): Glu(OtBu)
  MOD_RES (4)..(4): Gly(OPfp)
HAEG

SEQ ID NO: 269 - protein, 4 aa, Artificial Sequence
  Peptide fragment
  MOD_RES (1)..(1): Fmoc-His(Trt)
  MOD_RES (3)..(3): Glu(OtBu)
  MOD_RES (4)..(4): Gly(OPfp)
HAEG

SEQ ID NO: 270 - protein, 4 aa, Artificial Sequence
  Peptide fragment
  MOD_RES (1)..(1): Cbz-His(Trt)
  MOD_RES (3)..(3): Glu(OtBu)
  MOD_RES (4)..(4): Gly(OPfp)
HAEG

SEQ ID NO: 271 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mtt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 272 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mmt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 273 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 274 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 275 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Trt-Glu-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 276 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 277 - protein, 27 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 278 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 279 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mtt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 280 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mmt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 281 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 282 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Glu-OtBu)
  MOD_RES (17)..(17): Glu(tBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 283 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Trt-Glu-OtBu)
  MOD_RES (17)..(17): Glu(tBu)
  MOD_RES (24)..(24): Arg(Pbf)
  MOD_RES (26)..(26): Arg(Pbf)
  MOD_RES (27)..(27): Gly-O-CTC
TFTSDVSSYLEGQAAKEFIAWLVRGRG

SEQ ID NO: 284 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 285 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 286 - protein, 27 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 287 - protein, 12 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
TFTSDVSSYLEG

SEQ ID NO: 288 - protein, 12 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (12)..(12): Gly-OSu
TFTSDVSSYLEG

SEQ ID NO: 289 - protein, 12 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (12)..(12): Gly(OPfp)
TFTSDVSSYLEG

SEQ ID NO: 290 - protein, 12 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (12)..(12): Gly(OPfp)
TFTSDVSSYLEG

SEQ ID NO: 291 - protein, 12 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (12)..(12): Gly-O-CTC resin
TFTSDVSSYLEG

SEQ ID NO: 292 - protein, 12 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
TFTSDVSSYLEG

SEQ ID NO: 293 - protein, 12 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (12)..(12): Gly-OSu
TFTSDVSSYLEG

SEQ ID NO: 294 - protein, 12 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (12)..(12): Gly(OPfp)
TFTSDVSSYLEG

SEQ ID NO: 295 - protein, 12 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (12)..(12): Gly(OPfp)
TFTSDVSSYLEG

SEQ ID NO: 296 - protein, 12 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (12)..(12): Gly-O-CTC resin
TFTSDVSSYLEG

SEQ ID NO: 297 - protein, 8 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
TFTSDVSS

SEQ ID NO: 298 - protein, 8 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)-O-CTC resin
TFTSDVSS

SEQ ID NO: 299 - protein, 8 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)-O-CTC resin
TFTSDVSS

SEQ ID NO: 300 - protein, 8 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
TFTSDVSS

SEQ ID NO: 301 - protein, 8 aa, Artificial Sequence
  Peptide fragment
  MOD_RES (1)..(1): Cbz-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
TFTSDVSS

SEQ ID NO: 302 - protein, 4 aa, Artificial Sequence
  Peptide fragment
  MOD_RES (1)..(1): Fmoc-Tyr(tBu)
  MOD_RES (3)..(3): Glu(OtBu)
YLEG

SEQ ID NO: 303 - protein, 4 aa, Artificial Sequence
  Peptide fragment
  MOD_RES (1)..(1): Cbz-Tyr(tBu)
  MOD_RES (3)..(3): Glu(OtBu)
YLEG

SEQ ID NO: 304 - protein, 4 aa, Artificial Sequence
  Peptide fragment
  MOD_RES (1)..(1): Tyr(tBu)
  MOD_RES (3)..(3): Glu(OtBu)
YLEG

SEQ ID NO: 305 - protein, 12 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
TFTSDVSSYLEG

SEQ ID NO: 306 - protein, 12 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (12)..(12): Gly-OSu
TFTSDVSSYLEG

SEQ ID NO: 307 - protein, 12 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
  MOD_RES (9)..(9): Tyr(tBu)
  MOD_RES (11)..(11): Glu(OtBu)
  MOD_RES (12)..(12): Gly(OPfp)
TFTSDVSSYLEG

SEQ ID NO: 308 - protein, 12 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (12)..(12): Gly(OPfp)
TFTSDVSSYLEG

SEQ ID NO: 309 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 310 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mtt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 311 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (17)..(17): Glu(OtBu)
  MOD_RES (24)..(24): Arg(Pbf)
  MOD_RES (26)..(26): Arg(Pbf)
TFTSDVSSYLEGQAAKEFIAWLVRGRG

SEQ ID NO: 312 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mtt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 313 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mmt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 314 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 315 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Trt-Gly-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 316 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Pal-Glu-OtBu)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-Wang resin
TFTSDVSSYLEGQAAKEFIAWLVRGRG

SEQ ID NO: 317 - protein, 26 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Glu-OtBu)
  MOD_RES (23)..(23): Arg(Pbf)
  MOD_RES (25)..(25): Arg(Pbf)
TFTSDVSSYLEGQAAKFIAWLVRGRG

SEQ ID NO: 318 - protein, 8 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)-O-CTC resin
TFTSDVSS

SEQ ID NO: 319 - protein, 12 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (12)..(12): Gly-O-CTC resin
TFTSDVSSYLEG

SEQ ID NO: 320 - protein, 27 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Mmt)
  MOD_RES (17)..(17): Glu(OtBu)
  MOD_RES (24)..(24): Arg(Pbf)
  MOD_RES (26)..(26): Arg(Pbf)
  MOD_RES (27)..(27): Gly-O-CTC resin
TFTSDVSSYLEGQAAKEFIAWLVRGRG

SEQ ID NO: 321 - protein, 26 aa, Artificial Sequence
  Peptide fragment
  MOD_RES (1)..(1): Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(Trt)
  MOD_RES (8)..(8): Ser(Trt)
  MOD_RES (9)..(9): Tyr(tBu)
  MOD_RES (11)..(11): Glu(OtBu)
  MOD_RES (13)..(13): Gln(Trt)
  MOD_RES (16)..(16): Lys(Trt-Glu-OtBu)
  MOD_RES (23)..(23): Arg(Pbf)
  MOD_RES (25)..(25): Arg(Pbf)
  MOD_RES (26)..(26): Gly-O-CTC resin
TFTSDVSSYLEGQAAKFIAWLVRGRG

SEQ ID NO: 322 - protein, 30 aa, Artificial Sequence; identical to SEQ ID NO: 170
  Peptide fragment
  MOD_RES (1)..(1): His(Trt)
  MOD_RES (3)..(3): Glu(OtBu)
  MOD_RES (5)..(5): Thr(tBu)
  MOD_RES (7)..(7): Thr(tBu)
  MOD_RES (8)..(8): Ser(tBu)
  MOD_RES (9)..(9): Asp(OtBu)
  MOD_RES (11)..(11): Ser(Trt)
  MOD_RES (12)..(12): Ser(Trt)
  MOD_RES (13)..(13): Tyr(tBu)
  MOD_RES (15)..(15): Glu(OtBu)
  MOD_RES (17)..(17): Gln(Trt)
  MOD_RES (20)..(20): Lys(Glu-OtBu)
  MOD_RES (27)..(27): Arg(Pbf)
  MOD_RES (29)..(29): Arg(Pbf)
  MOD_RES (30)..(30): Gly-O-Wang resin

SEQ ID NO: 323 - protein, 8 aa, Artificial Sequence
  Peptide fragment
  MOD_RES (1)..(1): Fmoc-Thr(tBu)
  MOD_RES (3)..(3): Thr(tBu)
  MOD_RES (4)..(4): Ser(tBu)
  MOD_RES (5)..(5): Asp(OtBu)
  MOD_RES (7)..(7): Ser(tBu)
  MOD_RES (8)..(8): Ser(PSI(Me,Me)Pro)
TFTSDVSS

SEQ ID NO: 324 - protein, 31 aa, Artificial Sequence
  GLP-1 peptide agonist
  MISC_FEATURE (1)..(1): Xaa is His, D-His, desamino-histidine, 2-amino-histidine, beta-hydroxy-histidine, homohistidine, alpha-fluoromethyl-histidine or alpha-methyl-histidine
  MISC_FEATURE (1)..(1): Xaa has side chain H-, Ac-, Boc-, or Fmoc-
  MISC_FEATURE (1)..(1): Xaa may occur either one or zero times
  MISC_FEATURE (2)..(2): Xaa is -Ala, D-Ala, -Val, D-Val, Gly, Aib (alpha-aminoisobutyric acid)
  MISC_FEATURE (2)..(2): Xaa may occur one or zero times, but will always occur once if the first residue occurs once
  MISC_FEATURE (3)..(3): Xaa can be Glu, Asp, Met, Leu, but is preferably Glu or Asp, and is most preferably Asp
  MISC_FEATURE (16)..(16): Xaa is Gly or Aib
  MISC_FEATURE (28)..(28): Xaa is Lys or Arg
  MISC_FEATURE (29)..(29): Xaa is Gly, Aib, Ala, D-Ala
  MISC_FEATURE (30)..(30): Xaa is Arg, Lys or Gly
  MISC_FEATURE (30)..(30): Xaa may occur one or zero times, but will always occur once if the 31st residue occurs once
  MISC_FEATURE (31)..(31): Xaa can be Arg, Lys or Gly but is preferably Lys or Gly
  MISC_FEATURE (31)..(31): Xaa may occur either one or zero times
  MISC_FEATURE (31)..(31): Xaa has side chain -OH or -NH2
XXXGTFTSDVSSYLEXQAAKEFIAWLVXXXX

(19) United States
(12) Patent Application Publication
(10) Pub. No.: US 2018/0057558 A1
(43) Pub. Date: Mar. 1, 2018

(54) Synthesis of GLP-1 Peptides

(71) Applicant: Novetide, Ltd., Keryat Ata (IL)

(72) Inventors: Sharon Penias Navon, Kyriat Ata (IL); Shirly Naveh, Haifa (IL); Zoi Vasileiou, Patra (GR); Konstantinos Barlos, Patra (GR)

(21) Appl. No.: 15/513,488

(22) PCT Filed: Sep. 22, 2015
(86) PCT No.: PCT/IB2015/057307

§ 371 Date: Mar. 22, 2017

(30) Foreign Application Priority Data
Sep. 23, 2014 (GR) 20140100479

(51) Int. Cl. C07K 14/605

(52) U.S. Cl. CPC C07K 14/605

(57) ABSTRACT

Disclosed are processes for the synthesis of GLP-1 peptides, such as liraglutide and semaglutide, and a process for purifying liraglutide.

### CROSS-REFERENCE TO RELATED APPLICATIONS

This application is a national phase entry of PCT/IB2015/057307, filed Sep. 22, 2015, which claims foreign priority to Greece Patent Application No. 20140100479, filed Sep. 23, 2014.

### REFERENCE TO A SEQUENCE LISTING SUBMITTED ELECTRONICALLY VIA EFS-WEB

The content of the electronically submitted sequence listing (Name: 2873_2680001_SeqListing.txt; Size: 193,940 bytes; and Date of Creation: Jul. 24, 2017) is herein incorporated by reference in its entirety.

### FIELD OF THE INVENTION

The present invention encompasses a method for the synthesis of GLP-1 peptides, including Liraglutide and Semaglutide. The methods for preparing Liraglutide and Semaglutide involve a convergent synthetic strategy, wherein the coupling of the palmitoyl derivative on the side chain is carried out on a fragment of a Liraglutide sequence. The present invention also encompasses a linear synthesis of Semaglutide as well as a process for purifying liraglutide.

### BACKGROUND OF THE INVENTION

Liraglutide, Glycine, L-histidyl-L-alanyl-L-α-glutamylglycyl-L-threonyl-L-phenylalanyl-L-threonyl-L-seryl-L-α-aspartyl-L-valyl-L-seryl-L-seryl-L-tyrosyl-L-leucyl-L-α-glutamylglycyl-L-glutaminyl-L-alanyl-L-alanyl-N⁶—[N-(1-oxohexadecyl)-L-γ-glutamyl]-L-lysyl-L-α-glutamyl-L-phenylalanyl-L-isoleucyl-L-alanyl-L-tryptophyl-L-leucyl-L-valyl-L-arginylglycyl-L-arginyl-, [SEQ ID NO: 1] is also described as Nε26-(N-hexadecanoyl-L-γ-glutamyl)-[34-L-arginine]glucagon-like peptide 1-(7-37)-peptide. Liraglutide is a once-daily human GLP-1 analog, classified as a GLP-1 receptor agonist. Liraglutide is a slightly modified analog of the native human Glucagon-Like-Peptide-1 (GLP-1). Liraglutide is an Arg³⁴-GLP-1 analog substituted on the ε-amino group of the lysine in position 26 with a Glu-spaced palmitic acid, having the following formula [SEQ ID NO: 1]:

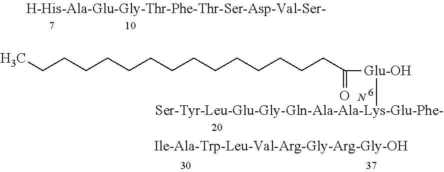

GLP-1 is a naturally occurring peptide, which stimulates insulin release and decreases the level of the anti-insulin hormone glucagon in response to increases in blood sugar levels. GLP-1 is typically produced by yeast through recombinant gene technology.

Liraglutide is thus a peptide containing a backbone of 31 amino acids, wherein the Lys is condensed with a Glu-Pal group. Liraglutide is produced by covalently linking GLP-1 to a fatty acid. It has the effects of lowering blood sugar level, reducing body weight, promoting islet cell regeneration, as well as protecting cardiovascular system.

Semaglutide, Glycine, L-histidyl-2-methylalanyl-L-α-glutamylglycyl-L-threonyl-L-phenylalanyl-L-threonyl-L-seryl-L-α-aspartyl-L-valyl-L-seryl-L-seryl-L-tyrosyl-L-leucyl-L-α-glutamylglycyl-L-glutaminyl-L-alanyl-L-alanyl-N6-[N-(17-carboxy-1-oxoheptadecyl)-L-γ-glutamyl[2-(2-aminoethoxy)ethoxy]acetyl[2-(2-aminoethoxy)ethoxy]acetyl]-L-lysyl-L-α-glutamyl-L-phenylalanyl-L-isoleucyl-L-alanyl-L-tryptophyl-L-leucyl-L-valyl-L-arginylglycyl-L-arginyl- [SEQ ID NO: 174], is another GLP-1 peptide, and has the following formula:

```
  1 2 3 4 5 6 7 8 9 10 11 12
His-Aib-Glu-Gly-Thr-Phe-Thr-Ser-Asp-Val-Ser-Ser-
  13 14 15 16 17 18 19 20 21
Tyr-Leu-Glu-Gly-Gln-Ala-Ala-Lys(W)-Glu-Phe-
    22 23 24 25 26 27 28 29 30 31
Ile-Ala-Trp-Leu-Val-Arg-Gly-Arg-Gly-OH
```

wherein W=N-(17-carboxy-1-oxoheptadecyl)-L-γ-glutamyl-2-[2-(2-aminoethoxy)ethoxy]acetyl-2-[2-(2-aminoethoxy)ethoxy]acetyl.

Semaglutide shares a similar backbone to liraglutide, with the Ala² being substituted for Aib, and wherein the Lys²⁰ is derivatized with N-(17-carboxy-1-oxoheptadecyl)-L-γ-glutamyl-2-[2-(2-aminoethoxy)ethoxy]acetyl-2-[2-(2-aminoethoxy)ethoxy]acetyl. Semaglutide is currently undergoing clinical trials for once-weekly management of Type-2 diabetes.

Liraglutide, as well as its synthesis and purification, are described in U.S. Pat. No. 6,268,343B1, U.S. Pat. No. 6,458,924B2 and U.S. Pat. No. 6,451,974B1. Recombinant synthesis provided the peptide intermediate (1-31) which is obtained in an unprotected form containing two free amino groups (at N-terminus and on Lys side chain). The Pal-Glu unit is then coupled to the Lys in the peptide intermediate (1-31). However, the Pal-Glu unit is not added only to the Lys to form the side chain but also to the N-terminus resulting formation of impurity of Liraglutide that reduces the yield of the synthesis and also results in the formation of another closely related impurity that must be separated from the final product.

U.S. Pat. No. 8,445,433B2 describes a method of synthesizing GLP-1 analogs by linear (i.e. sequential) synthesis of the peptide on solid support, wherein an Fmoc-pseudoproline dipeptide unit is employed instead of only single Fmoc-amino acids, during solid phase synthesis. This method is said to improve the synthesis of the peptide; however the final peptide is obtained as a mixture which is difficult to purify.

CN102286092A describes a linear solid state synthesis of Liraglutide on a resin, in which the Liraglutide backbone is prepared by sequential coupling of single Fmoc protected amino acids. The Lys group chain is introduced using Fmoc-Lys(Alloc)-OH. At the end of the production of the Liraglutide sequence, the Pal-Glu side chain is coupled onto the Lys residue by firstly removing the Alloc protecting group using Pd(PPh₃)₄ and then coupling with Pal-Glu-OtBu before deprotecting and resin removal. The use of Fmoc-Lys (Alloc)-OH has the following drawbacks: the use of Pd(PPh₃)₄ reagent in the removal of the Alloc protecting group is not particularly suitable for industrial scale synthesis as the reagent is very sensitive to air, light and heat, thus, the reaction can only be effectively performed in the absence of air and light. Also, Pd(PPh₃)₄ is very expensive and its reactions preferably should be conducted in an argon atmosphere. Accordingly, the use of this reagent is not applicable for large scale industrial production. Moreover, Pd is defined as a highly toxic impurity and as such its presence in a drug product must be minimized. Therefore, the use of Pd reagents in the pharmaceutical industry should be avoided. Also the peptide is synthesized by a linear, i.e. sequential synthesis, which, as mentioned above, results in a lower purity of the final peptide. Moreover, impurities in the final peptide are typically difficult to remove.

CN 103145828 describes a similar method for preparing Liraglutide as CN102286092A, which involves sequential coupling of single amino acids to form the Liraglutide backbone sequence. The Lys residue is introduced using Fmoc-Lys(ivDde). The ivDde protecting group is removed at the end of the production of the Liraglutide sequence and Pal-Glu-OtBu is then coupled to the Lys residue of the liraglutide backbone, before deprotection and resin removal. However, according to this publication, the use of Fmoc-Lys(ivDde) requires the removal of the ivDde group using hydrazine. Hydrazine is an extremely toxic and flammable compound, and its use on an industrial scale should be avoided.

CN 103864918 discloses a solid phase synthesis of liraglutide involving coupling a peptide sequence containing amino acid residues (1-10) to a sequence containing amino acid residues (11-31), and removing the resin and protecting groups, before purifying and freeze drying the liraglutide.

CN 104004083 discloses solid phase synthesis of liraglutide involving the preparation of peptide sequences containing amino acid residues (1-4), (15-16) and (17-31), coupling the peptides containing amino acid residues (15-16) with (17-31) and sequential addition of amino acids before coupling with the peptide containing amino acid sequence (1-4), removing the resin and protecting groups, and purifying.

WO2007090496 discloses a method of synthesizing other GLP-1 peptide agonists, e.g. of formula:

A-(R1)x-(R2)y-R3-Gly-Thr-Phe-Thr-Ser-Asp-Val-Ser-Ser-Tyr-Leu-Glu-R8-Gln-Ala-Ala-Lys-Glu-Phe-Ile-Ala-Trp-Leu-Val-R4-R5-(R6)w-(R7)z-B. [SEQ ID NO: 324]

by linear sequential synthesis, using an Fmoc-pseudoproline dipeptide unit at the relevant position in order to prepare the Val-Ser or Ser-Ser segment of the peptide chain. The remaining sequence is then prepared by stepwise sequential synthesis.

Discovering new methods for the synthesis of GLP-1 proteins such as Liraglutide or Semaglutide, can provide a better and more efficient processes, and further can provide a product which can be more readily purified in order to achieve a product with improved yield and purity. In particular, there is a need to provide a methods for preparing GLP-1 proteins such as Liraglutide or Semaglutide, especially on an industrial scale, which should not require the use of toxic or otherwise undesirable reagents. Preferably the methods should be capable of preparing GLP-1 proteins such as Liraglutide or Semaglutide in good yields and which can be readily purified to obtain a product having high purity. For at least these reasons, there is a need for additional synthetic processes that can be used for preparing GLP-1 proteins such as Liraglutide or Semaglutide, especially on an industrial scale.

### SUMMARY OF THE INVENTION

In a first aspect, the present invention provides a convergent process for preparing a GLP-1 peptide comprising liquid or solid phase peptide synthesis or a combination thereof, wherein the process comprises a final coupling step in which at least two fragments are coupled at a terminal Gly residue, and wherein at least one of the fragments is prepared by coupling of at least two sub-fragments. The GLP-1 peptide therefore comprises at least one non-terminal Gly residue.

Preferably, the GLP-1 peptide can contain at least two non-terminal Gly residues, such as two, three or four non-terminal Gly residues. By non-terminal Gly residues, it is meant that the GLP-1 peptide contains at least one Gly residue that is not at the N- or C-terminus of the peptide. Nevertheless, the GLP-1 peptide may, in addition to the non-terminal Gly residue, contain a Gly residue at the N- and/or C-terminus. The process is especially applicable to any GLP-1 peptide containing at least one-non-terminal Gly residue, wherein the non-terminal Gly residue is at least the third (i.e. Gly³), and preferably at least the fourth (i.e. Gly⁴) amino acid from the N-terminus. For example, the GLP-1 peptide may be Liraglutide or Semaglutide, each of which contains a Gly residue (i.e. Gly⁴) which is the fourth amino acid from the N-terminus. Liraglutide and Semaglutide each also contains a Gly residue as the 16^{th} amino acid from the N-terminus, i.e. Gly¹⁶.

In accordance with the processes of the present invention, these Gly groups in the GLP-1 peptides, such as the Gly⁴ and Gly¹⁶ residues in Liraglutide and Semaglutide enable convenient chemical ligation to form the peptide, and optionally peptide fragments and/or peptide sub-fragments. In particular, such ligation to form the final peptide and peptide fragments and/or sub-fragments at Gly residues is particularly advantageous where the final peptide or peptide fragments/subfragments contain a terminal His residue, since coupling reactions with His to form the final peptide, which have a tendency to result in racemization to produce D-His isomer impurities in the final peptide, can be reduced or avoided. The D-His isomers are typically difficult to separate from the final peptide. The convergent processes of the present invention in particular avoid final coupling reactions involving His.

In one aspect, the present invention provides methods for preparing GLP-1 peptides such as Liraglutide or Semaglutide, which do not involve the use of unusual or toxic reagents, and also does not require the use of special building units. The processes disclosed herein can provide GLP-1 peptides such as Liraglutide or Semaglutide in high yield. Moreover, the GLP-1 peptides such as Liraglutide or Semaglutide can be prepared in high purity using the processes of the present invention. Thus, the methods are highly suitable for the preparation of GLP-1 proteins such as Liraglutide or Semaglutide on an industrial scale.

As used herein, the amino acid forming the liraglutide backbone are numbered consecutively from 1 to 31, starting from the terminal His residue as follows:

[SEQ ID NO: 1]

```
  1 2 3 4 5 6 7 8 9 10 11 12
His-Ala-Glu-Gly-Thr-Phe-Thr-Ser-Asp-Val-Ser-Ser-
  13 14 15 16 17 18 19 20 21
Tyr-Leu-Glu-Gly-Gln-Ala-Ala-Lys(Pal-Glu)-Glu-Phe-
  22 23 24 25 26 27 28 29 30 31
Ile-Ala-Trp-Leu-Val-Arg-Gly-Arg-Gly-OH
```

Thus, according to this numbering, the Lys at position 20 is substituted with the Glu-spaced palmitic acid group. Unless otherwise indicated, the same numbering system for the amino acids is applied throughout, both when referring to the complete amino acid sequence forming liraglutide or the backbone of liraglutide, or to the individual amino acids or amino acid sequences which form the peptide fragments that make up liraglutide or the liraglutide backbone.

Similarly, the amino acid forming the semaglutide backbone are numbered consecutively from 1-31, starting from the terminal His residue as follows:

```
  1 2 3 4 5 6 7 8 9 10 11 12
His-Aib-Glu-Gly-Thr-Phe-Thr-Ser-Asp-Val-Ser-Ser-
  13 14 15 16 17 18 19 20 21
Tyr-Leu-Glu-Gly-Gln-Ala-Ala-Lys(W)-Glu-Phe-
    22 23 24 25 26 27 28 29 30 31
Ile-Ala-Trp-Leu-Val-Arg-Gly-Arg-Gly-OH
```

wherein W=N-(17-carboxy-1-oxoheptadecyl)-L-γ-glutamyl-2-[2-(2-aminoethoxy)ethoxy]acetyl-2-[2-(2-aminoethoxy)ethoxy]acetyl [SEQ ID NO: 174]

The present invention encompasses a method for the synthesis of GLP-1 peptides such as liraglutide or semaglutide using a convergent synthetic strategy. In particular, the present invention encompasses a method for the synthesis of GLP-1 peptides such as liraglutide or semaglutide, using a two, three or four fragment convergent strategy. The present process provides synthetic procedures that can be carried out as a solid state peptide synthesis, or may be conveniently conducted as a liquid phase synthesis.

In particular, the present invention provides a process for preparing liraglutide which involves coupling a peptide fragment containing amino acids (1-4) with a peptide fragment containing amino acids (5-31) which carries the Lys(Pal-Glu) residue, to form, after any deprotection and resin removal, liraglutide. In particular, the present invention encompasses a process for preparing liraglutide [SEQ ID NO: 1] of formula:

```
  1 2 3 4 5 6 7 8 9 10 11 12
His-Ala-Glu-Gly-Thr-Phe-Thr-Ser-Asp-Val-Ser-Ser-
  13 14 15 16 17 18 19 20 21
Tyr-Leu-Glu-Gly-Gln-Ala-Ala-Lys(Pal-Glu)-Glu-
  22 23 24 25 26 27 28 29 30 31
Phe-Ile-Ala-Trp-Leu-Val-Arg-Gly-Arg-Gly-OH
```

(i) coupling a Peptide 1 having the sequence:

[SEQ ID NO: 5]

His-Ala-Glu-Gly

wherein:
   the N-terminal of His is optionally protected with a protecting group, preferably selected from the group consisting of Boc, Cbz or Fmoc, and
   the Gly carboxylic acid group in Peptide 1 may be in the form of an activated carboxylic acid derivative;
with a Peptide 2 having the sequence:

[SEQ ID NO: 24]

Thr-Phe-Thr-Ser-Asp-Val-Ser-Ser-Tyr-Leu-Glu-Gly-
Gln-Ala-Ala-Lys(Pal-Glu-OX)-Glu-Phe-Ile-Ala-Trp-
Leu-Val-Arg-Gly-Arg-Gly-OH

wherein:
   Peptide 2 is optionally conjugated to a solid support such as a resin;
   X represents H or a protecting group for the Glu carboxylic acid group, and
   one or more of the amino acid residues in Peptide 1 and Peptide 2 may be protected at the side chains or unprotected (preferably protected).

Preferably, the Peptide 2 is either conjugated to a Wang resin, or Peptide 2 is not present on a resin. When the Peptide 2 is not present on a resin, the coupling of Peptide 1 with Peptide 2 is conducted as a liquid phase synthesis. In one preferred embodiment, Peptide 2 is not on a resin, and its coupling with Peptide 1 is carried out by liquid phase synthesis.

The coupling of Peptide 1 with Peptide 2 forms an optionally protected liraglutide sequence which (when Peptide 2 is attached to a solid support) is optionally attached to a resin at the Gly³¹ residue. Subsequent removal of any protecting groups and resin, and optional purification enables the Liraglutide to be obtained in high yield and high purity.

In preferred embodiments, Peptide 2 is prepared by a convergent process, which preferably involves a two-fragment convergent synthesis. Preferably, Peptide 2 is prepared by coupling a peptide fragment containing amino acids (5-16) with a peptide containing amino acids (17-31) of liraglutide. Thus, in this preferred embodiment, liraglutide may be conveniently prepared by a three fragment convergent synthesis, wherein a peptide fragment containing amino acids (5-16) of liraglutide is coupled to a peptide fragment containing amino acids (17-31) of liraglutide to form a peptide fragment containing amino acids (5-31) of liraglutide, and coupling a peptide fragment containing amino acids (1-4) to the peptide containing amino acids (5-31) to form, after any deprotection and resin removal, liraglutide.

In a further preferred embodiment, the peptide containing amino acids (5-16) can also be prepared by a convergent synthesis coupling a peptide fragment containing amino acids (5-12) with a peptide fragment containing amino acids (13-16). In this further preferred embodiment, liraglutide may be conveniently prepared by a four fragment convergent synthesis wherein the fragments are (1-4)+(5-12)+(13-16)+(17-31), i.e. by coupling (5-12) with (13-16) to prepare (5-16), then coupling this with (17-31) to prepare (5-31), and finally coupling (1-4) to (5-31). Thus, in this embodiment, liraglutide may be conveniently prepared by a four fragment convergent synthesis, wherein a peptide fragment containing amino acids (5-12) is coupled with a peptide fragment containing amino acids (13-16) to form a peptide fragment containing amino acids (5-16) of liraglutide, coupling this peptide fragment to a peptide fragment containing amino acids (17-31) of liraglutide to form a peptide fragment containing amino acids (5-31) of liraglutide, and coupling a peptide fragment containing amino acids (1-4) to the peptide fragment containing amino acids (5-31) to form, after any deprotection and resin removal, liraglutide. In any of the processes of the present invention described herein, the Pal-Glu residue is preferably present (optionally protected at the Glu carboxylic acid) on the Lys residue at position 20 in the peptide fragment containing amino acids (5-31) of liraglutide during the coupling with peptide fragment (1-4).

In a further aspect, the present invention further provides peptide fragments and intermediates, which may be useful in the synthesis of liraglutide [SEQ ID NO: 1]. Particularly useful intermediates include:

A.

[SEQ ID NO: 2]

P1-His(P)-Ala-Glu(P)-Gly-Thr(P)-Phe-Thr(P)-Ser(P)-
Asp(P)-Val-Ser(P)-Ser(P)-Tyr(P)-Leu-Glu(P)-Gly-
Gln(P)-Ala-Ala-Lys(Pal-Glu-P)-Glu(P)-Phe-Ile-Ala-
Trp-Leu-Val-Arg(P)-Gly-Arg(P)-Gly-O-P2

wherein P1 represents a protecting group for the N-terminal of His (preferably Boc), each P represents side chain protecting groups which may be the same or different, and P2 is H (i.e. the carboxylic acid of the Gly³¹ residue is unsubstituted, and thus contains a free —OH group), or P2 represents a solid support, preferably a Wang resin.

Preferred intermediates include the following [SEQ ID NO: 3] and [SEQ ID NO: 4]:

[SEQ ID NO: 3]

Boc-His(Trt)-Ala-Glu(OtBu)-Gly-Thr(tBu)-Phe-
Thr(tBu)-Ser(tBu)-Asp(OtBu)-Val-Ser(tBu)-
Ser(Ψ^{Me,Me}pro)-Tyr(tBu)-Leu-Glu(OtBu)-Gly-
Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-O-
Wang resin

[SEQ ID NO: 266]

Boc-His(Trt)-Ala-Glu(OtBu)-Gly-Thr(tBu)-Phe-
Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-
Lys(Pal-Glu-OtBu)-Glu(OtBu)-Phe-Ile-Ala-Trp-Leu-
Val-Arg(Pbf)-Gly-Arg(Pbf)-Gly-O-Wang resin

[SEQ ID NO: 4]

Boc-His(Trt)-Ala-Glu(OtBu)-Gly-Thr(tBu)-Phe-
Thr(tBu)-Ser(tBu)-Asp(OtBu)-Val-Ser(tBu)-
Ser(Ψ^{Me,Me}pro)-Tyr(tBu)-Leu-Glu(OtBu)-Gly-
Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-OH

[SEQ ID NO: 267]

Boc-His(Trt)-Ala-Glu(OtBu)-Gly-Thr(tBu)-Phe-
Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-
Lys(Pal-Glu-OtBu)-Glu(OtBu)-Phe-Ile-Ala-Trp-Leu-
Val-Arg(Pbf)-Gly-Arg(Pbf)-Gly-OH

Particularly useful peptide fragments for use in the synthesis of liraglutide in accordance with the present invention include peptide fragments containing amino acid sequences (1-4) of liraglutide, i.e. His-Ala-Glu-Gly [SEQ ID NO: 5], such as:

B.

[SEQ ID NO: 6]

P1-His(P)-Ala-Glu(P)-Gly-O-P2

wherein P1 represents a protecting group for the N-terminal of His (preferably Boc, Fmoc or Cbz), each P represents side chain protecting groups which may be the same or different, and P2 is selected from: H (i.e. the carboxylic acid of the Gly⁴ residue is unsubstituted, and thus contains a free —OH group), or a solid support (preferably a CTC resin), or P2 represents an activated carboxylic ester of the Gly⁴ residue (preferably Su, Bt or Pfp). Preferably P1 represents Boc, Fmoc or CBz and P2 represents H, Su, Bt, Pfp or a CTC resin.

Preferred (1-4) peptide fragments are as follows:

[SEQ ID NO: 7]

Boc-His(Trt)-Ala-Glu(OtBu)-Gly-OH

[SEQ ID NO: 8]

Fmoc-His(Trt)-Ala-Glu(OtBu)-Gly-OH

[SEQ ID NO: 9]

Cbz-His(Trt)-Ala-Glu(OtBu)-Gly-OH

[SEQ ID NO: 10]

Boc-His(Trt)-Ala-Glu(OtBu)-Gly-OSu

[SEQ ID NO: 11]

Fmoc-His(Trt)-Ala-Glu(OtBu)-Gly-OSu

[SEQ ID NO: 12]

Cbz-His(Trt)-Ala-Glu(OtBu)-Gly-OSu

[SEQ ID NO: 13]

Boc-His(Trt)-Ala-Glu(OtBu)-Gly-OBt

[SEQ ID NO: 14]

Fmoc-His(Trt)-Ala-Glu(OtBu)-Gly-OBt

[SEQ ID NO: 15]

Cbz-His(Trt)-Ala-Glu(OtBu)-Gly-OBt

[SEQ ID NO: 16]

Boc-His(Trt)-Ala-Glu(OtBu)-Gly-O-CTC resin

[SEQ ID NO: 17]

Fmoc-His(Trt)-Ala-Glu(OtBu)-Gly-O-CTC resin

[SEQ ID NO: 18]

Cbz-His(Trt)-Ala-Glu(OtBu)-Gly-O-CTC resin

or

[SEQ ID NO: 268]

Boc-His(Trt)-Ala-Glu(OtBu)-Gly-OPfp

[SEQ ID NO: 269]

Fmoc-His(Trt)-Ala-Glu(OtBu)-Gly-OPfp

[SEQ ID NO: 270]

Cbz-His(Trt)-Ala-Glu(OtBu)-Gly-OPfp.

These peptide fragments {especially Boc-His(Trt)-Ala-Glu(OtBu)-Gly-OH—[SEQ ID NO: 7], Fmoc-His(Trt)-Ala-Glu(OtBu)-Gly-OH—[SEQ ID NO: 8], and Cbz-His(Trt)-Ala-Glu(OtBu)-Gly-OH—[SEQ ID NO: 9]} can be readily purified by simple procedures, and enable the efficient production of liraglutide in high yield and purity.

An additional aspect of the present invention provides the following peptide fragments useful as intermediates in the process of the present invention:
(i) Peptide fragments containing amino acid sequences (5-31) of liraglutide, including:
   (i) Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly [SEQ ID NO: 19], including:

C1.

[SEQ ID NO: 20]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(P)-Glu(P)-Phe-Ile-Ala-Trp-Leu-Val-Arg(P)-Gly-
Arg(P)-Gly-OP2

wherein P1 represents H, or a protecting group for the N-terminal of Thr (preferably Fmoc or Cbz and more preferably Fmoc), each P represents side chain protecting groups which may be the same or different, and P2 is selected from H (i.e. the carboxylic acid of the Gly³¹ residue is unsubstituted, and thus contains a free —OH group), or a solid support, preferably a CTC or Wang resin;
(ii) Thr-Phe-Thr-Ser-Asp-Val-Ser-Ser-Tyr-Leu-Glu-Gly-Gln-Ala-Ala-Lys(Glu)-Glu-Phe-Ile-Ala-Trp-Leu-Val-Arg-Gly-Arg-Gly [SEQ ID NO: 21], including:

C2.

[SEQ ID NO: 22]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(Glu-P)-Glu(P)-Phe-Ile-Ala-Trp-Leu-Val-Arg(P)-
Gly-Arg(P)-Gly-OP2

wherein P1 represents H, or a protecting group for the N-terminal of Thr (preferably Fmoc or Cbz and more preferably Fmoc), each P represents side chain protecting groups which may be the same or different, and P2 is selected from H (i.e. the carboxylic acid of the Gly³¹ residue is unsubstituted, and thus contains a free —OH group), or a solid support, preferably a CTC or Wang resin;
or

C3.

[SEQ ID NO: 23]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(P1-Glu-P)-Glu(P)-Phe-Ile-Ala-Trp-Leu-Val-
Arg(P)-Gly-Arg(P)-Gly-OP2

wherein P1 represents H, or N-terminal protecting groups for the N-Thr and/or Glu wherein P1 may be the same or different (preferably for Thr, P1 is Fmoc or Cbz and more preferably Fmoc, and preferably for Glu, P1 is Trt), each P represents side chain protecting groups which may be the same or different, and P2 is selected from H (i.e. the carboxylic acid of the Gly³¹ residue is unsubstituted, and thus contains a free —OH group), or a solid support, preferably a CTC or Wang resin;
and
(iii) Thr-Phe-Thr-Ser-Asp-Val-Ser-Ser-Tyr-Leu-Glu-Gly-Gln-Ala-Ala-Lys(Pal-Glu)-Glu-Phe-Ile-Ala-Trp-Leu-Val-Arg-Gly-Arg-Gly [SEQ ID NO: 24], including:

C4.

[SEQ ID NO: 25]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(Pal-Glu-P)-Glu(P)-Phe-Ile-Ala-Trp-Leu-Val-
Arg(P)-Gly-Arg(P)-Gly-OP2

wherein P1 represents H, or a protecting group for the N-terminal of Thr (preferably Fmoc or Cbz and more preferably Fmoc), each P represents side chain protecting groups which may be the same or different, and P2 is selected from H (i.e. the carboxylic acid of the Gly³¹ residue is unsubstituted, and thus contains a free —OH group), or a solid support, preferably a CTC or Wang resin.
Preferred (5-31) peptides are as follows:

[SEQ ID NO: 26]
Fmoc-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mtt)-Glu(OtBu)-Phe-Ile-Ala-Trp-Leu-Val-Arg(Pbf)-Gly-Arg(Pbf)-
Gly-O-CTC resin
[SEQ ID NO: 271]
Fmoc-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mtt)-Glu(OtBu)-
Phe-Ile-Ala-Trp-Leu-Val-Arg(Pbf)-Gly-Arg(Pbf)-Gly-O-CTC resin
[SEQ ID NO: 27]
Fmoc-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mmt)-Glu(OtBu)-Phe-Ile-Ala-Trp-Leu-Val-Arg(Pbf)-Gly-Arg(Pbf)-
Gly-O-CTC resin
[SEQ ID NO: 272]
Fmoc-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mmt)-Glu(OtBu)-
Phe-Ile-Ala-Trp-Leu-Val-Arg(Pbf)-Gly-Arg(Pbf)-Gly-O-CTC resin
[SEQ ID NO: 28]
Fmoc-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Glu-
OtBu)-Glu(OtBu)-Phe-Ile-Ala-Trp-Leu-Val-Arg(Pbf)-Gly-Arg(Pbf)-Gly-
OH
[SEQ ID NO: 273]
Fmoc-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Glu-OtBu)-
Glu(OtBu)-Phe-Ile-Ala-Trp-Leu-Val-Arg(Pbf)-Gly-Arg(Pbf)-Gly-OH
[SEQ ID NO: 29]
Fmoc-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Pal-
Glu-OtBu)-Glu(OtBu)-Phe-Ile-Ala-Trp-Leu-Val-Arg(Pbf)-Gly-Arg(Pbf)-
Gly-OH
[SEQ ID NO: 274]
Fmoc-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Pal-Glu-OtBu)-
Glu(OtBu)-Phe-Ile-Ala-Trp-Leu-Val-Arg(Pbf)-Gly-Arg(Pbf)-Gly-OH
[SEQ ID NO: 30]
Fmoc-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Trt-
Glu-OtBu)-Glu(OtBu)-Phe-Ile-Ala-Trp-Leu-Val-Arg(Pbf)-Gly-Arg(Pbf)-
Gly-O-CTC resin
[SEQ ID NO: 275]
Fmoc-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Trt-Glu-OtBu)-
Glu(OtBu)-Phe-Ile-Ala-Trp-Leu-Val-Arg(Pbf)-Gly-Arg(Pbf)-Gly-O-CTC
resin
[SEQ ID NO: 31]
H-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Pal-
Glu-OtBu)-Glu(OtBu)-Phe-Ile-Ala-Trp-Leu-Val-Arg(Pbf)-Gly-Arg(Pbf)-
Gly-OH
[SEQ ID NO: 276 or SEQ ID NO: 309]
H-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Pal-Glu-OtBu)-
Glu(OtBu)-Phe-Ile-Ala-Trp-Leu-Val-Arg(Pbf)-Gly-Arg(Pbf)-Gly-OH
[SEQ ID NO: 32]
Fmoc-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Pal-
Glu-OtBu)-Glu(OtBu)-Phe-Ile-Ala-Trp-Leu-Val-Arg(Pbf)-Gly-Arg(Pbf)-
Gly-O-Wang resin
[SEQ ID NO: 277]
Fmoc-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Pal-Glu-OtBu)-
Glu(OtBu)-Phe-Ile-Ala-Trp-Leu-Val-Arg(Pbf)-Gly-Arg(Pbf)-Gly-O-
Wang resin
[SEQ ID NO: 33]
H-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Pal-
Glu-OtBu)-Glu(OtBu)-Phe-Ile-Ala-Trp-Leu-Val-Arg(Pbf)-Gly-Arg(Pbf)-
Gly-O-Wang resin
[SEQ ID NO: 278 or SEQ ID NO: 316]
H-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Pal-Glu-OtBu)-
Glu(OtBu)-Phe-Ile-Ala-Trp-Leu-Val-Arg(Pbf)-Gly-Arg(Pbf)-Gly-O-
Wang resin
[SEQ ID NO: 34]
Cbz-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mtt)-Glu(OtBu)-Phe-Ile-Ala-Trp-Leu-Val-Arg(Pbf)-Gly-Arg(Pbf)-
Gly-O-CTC resin
[SEQ ID NO: 279]
Cbz-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mtt)-Glu(OtBu)-
Phe-Ile-Ala-Trp-Leu-Val-Arg(Pbf)-Gly-Arg(Pbf)-Gly-O-CTC resin
[SEQ ID NO: 35]
Cbz-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mmt)-Glu(OtBu)-Phe-Ile-Ala-Trp-Leu-Val-Arg(Pbf)-Gly-Arg(Pbf)-
Gly-O-CTC resin
[SEQ ID NO: 280]
Cbz-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mmt)-Glu(OtBu)-
Phe-Ile-Ala-Trp-Leu-Val-Arg(Pbf)-Gly-Arg(Pbf)-Gly-O-CTC resin
[SEQ ID NO: 36 or SEQ ID NO: 37]
Cbz-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Glu-
OtBu)-Glu(OtBu)-Phe-Ile-Ala-Trp-Leu-Val-Arg(Pbf)-Gly-Arg(Pbf)-Gly-
OH
[SEQ ID NO: 281 or SEQ ID NO: 282]
Cbz-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Glu-OtBu)-
Glu(OtBu)-Phe-Ile-Ala-Trp-Leu-Val-Arg(Pbf)-Gly-Arg(Pbf)-Gly-OH
[SEQ ID NO: 37 or SEQ ID NO: 36]
Cbz-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Glu-
OtBu)-Glu(OtBu)-Phe-Ile-Ala-Trp-Leu-Val-Arg(Pbf)-Gly-Arg(Pbf)-Gly-
OH
[SEQ ID NO: 282 or SEQ ID NO: 281]
Cbz-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Glu-OtBu)-
Glu(OtBu)-Phe-Ile-Ala-Trp-Leu-Val-Arg(Pbf)-Gly-Arg(Pbf)-Gly-OH
[SEQ ID NO: 38]
Cbz-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Trt-
Glu-OtBu)-Glu(OtBu)-Phe-Ile-Ala-Trp-Leu-Val-Arg(Pbf)-Gly-Arg(Pbf)-
Gly-O-CTC
[SEQ ID NO: 283]
Cbz-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Trt-Glu-OtBu)-
Glu(OtBu)-Phe-Ile-Ala-Trp-Leu-Val-Arg(Pbf)-Gly-Arg(Pbf)-Gly-O-CTC
[SEQ ID NO: 39]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Glu-
OtBu)-Glu(OtBu)-Phe-Ile-Ala-Trp-Leu-Val-Arg(Pbf)-Gly-Arg(Pbf)-Gly-
OH
[SEQ ID NO: 284]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Glu-OtBu)-
Glu(OtBu)-Phe-Ile-Ala-Trp-Leu-Val-Arg(Pbf)-Gly-Arg(Pbf)-Gly-OH
[SEQ ID NO: 40]
Cbz-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Pal-
Glu-OtBu)-Glu(OtBu)-Phe-Ile-Ala-Trp-Leu-Val-Arg(Pbf)-Gly-Arg(Pbf)-
Gly-OH
[SEQ ID NO: 285]
Cbz-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Pal-Glu-OtBu)-
Glu(OtBu)-Phe-Ile-Ala-Trp-Leu-Val-Arg(Pbf)-Gly-Arg(Pbf)-Gly-OH
[SEQ ID NO: 41]
Cbz-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-Lys(Pal-
Glu-OtBu)-Glu(OtBu)-Phe-Ile-Ala-Trp-Leu-Val-Arg(Pbf)-Gly-Arg(Pbf)-
Gly-O-Wang resin
[SEQ ID NO: 286]
Cbz-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Pal-Glu-OtBu)-
Glu(OtBu)-Phe-Ile-Ala-Trp-Leu-Val-Arg(Pbf)-Gly-Arg(Pbf)-Gly-O-
Wang resin

(ii) Peptide fragments containing amino acid sequence (5-16) of liraglutide, i.e. Thr-Phe-Thr-Ser-Asp-Val-Ser-Ser-Tyr-Leu-Glu-Gly [SEQ ID NO: 42], including:

D.

[SEQ ID NO: 43]

P1-Thr(P)-Phe-Thr(P)-Ser(P)-Asp(P)-Val-Ser(P)-
Ser(P)-Tyr(P)-Leu-Glu(P)-Gly-OP2

wherein P1 represents a protecting group for the N-terminal of Thr (preferably Fmoc or Cbz and more preferably Fmoc), each P represents side chain protecting groups which may be the same or different, and P2 is selected from H (i.e. the carboxylic acid of the Gly¹⁶ residue is unsubstituted, and thus contains a free —OH group), or P2 represents an activated carboxylic ester of the Gly¹⁶ residue (preferably Su, Bt or Pfp, more preferably Su or Pfp, and most preferably Pfp), or P2 represents a solid support, preferably a CTC resin.
Preferred (5-16) peptides are as follows:

[SEQ ID NO: 44]
Fmoc-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-OH
[SEQ ID NO: 287]
Fmoc-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OH
[SEQ ID NO: 45]
Fmoc-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-OSu
[SEQ ID NO: 288]
Fmoc-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OSu
[SEQ ID NO: 289]
Fmoc-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OPfp
[SEQ ID NO: 290]
Fmoc-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-OPfp
[SEQ ID NO: 46]
Fmoc-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-O-CTC resin
[SEQ ID NO: 291]
Fmoc-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-O-CTC resin
[SEQ ID NO: 47]
Cbz-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-OH
[SEQ ID NO: 292]
Cbz-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OH
[SEQ ID NO: 48]
Cbz-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-OSu
[SEQ ID NO: 293]
Cbz-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OSu
[SEQ ID NO: 294]
Cbz-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-OPfp
[SEQ ID NO: 295]
Cbz-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OPfp
[SEQ ID NO: 49]
Cbz-Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-O-CTC resin
[SEQ ID NO: 296]
Cbz-Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-O-CTC resin

(iii) Peptide fragments containing amino acid sequence (17-31) of liraglutide, i.e. Gln-Ala-Ala-Lys-Glu-Phe-Ile-Ala-Trp-Leu-Val-Arg-Gly-Arg-Gly [SEQ ID NO: 50], Gln-Ala-Ala-Lys(Glu)-Glu-Phe-Ile-Ala-Trp-Leu-Val-Arg-Gly-Arg-Gly-OH [SEQ ID NO: 51], or Gln-Ala-Ala-Lys(Pal-Glu)-Glu-Phe-Ile-Ala-Trp-Leu-Val-Arg-Gly-Arg-Gly-OH [SEQ ID NO: 52] including:

E1.

[SEQ ID NO: 53]

P1-Gln(P)-Ala-Ala-Lys(P3-Glu-P)-Glu(P)-Phe-Ile-
Ala-Trp-Leu-Val-Arg(P)-Gly-Arg(P)-Gly-O-P2

wherein P1 represents H or a protecting group for the N-terminal of Gin (preferably Fmoc or Cbz and more preferably Fmoc), each P represents side chain protecting groups which may be the same or different, P2 is selected from H (i.e. the carboxylic acid of the Gly³¹ residue is unsubstituted, and thus contains a free —OH group), or P2 represents a solid support, preferably a CTC or Wang resin, and P3 represents a protecting group for the Glu nitrogen atom or Pal
and

E2.

[SEQ ID NO: 54]

P1-Gln(P)-Ala-Ala-Lys(Y)-Glu(P)-Phe-Ile-Ala-Trp-
Leu-Val-Arg(P)-Gly-Arg(P)-Gly-O-P2

wherein P1 represents H or a protecting group for the N-terminal of Gln (preferably Fmoc or Cbz and more preferably Fmoc), each P represents side chain protecting groups which may be the same or different, P2 is selected from H (i.e. the carboxylic acid of the Gly³¹ residue is unsubstituted, and thus contains a free —OH group), or P2 represents a solid support, preferably a CTC or Wang resin, and Y represents Mmt or Mtt.
Preferred (17-31) peptides are as follows:

[SEQ ID NO: 55 or SEQ ID NO: 128]
H-Gln(Trt)-Ala-Ala-Lys(Pal-Glu-OtBu)-Glu(OtBu)-Phe-Ile-Ala-Trp-Leu-
Val-Arg(Pbf)-Gly-Arg(Pbf)-Gly-O-Wang resin
[SEQ ID NO: 56 or SEQ ID NO: 148]
H-Gln(Trt)-Ala-Ala-Lys(Pal-Glu-OtBu)-Glu(OtBu)-Phe-Ile-Ala-Trp-Leu-
Val-Arg(Pbf)-Gly-Arg(Pbf)-Gly-OH
[SEQ ID NO: 57]
Fmoc-Gln(Trt)-Ala-Ala-Lys(Pal-Glu-OtBu)-Glu(OtBu)-Phe-Ile-Ala-Trp-
Leu-Val-Arg(Pbf)-Gly-Arg(Pbf)-Gly-O-Wang resin
[SEQ ID NO: 58]
Fmoc-Gln(Trt)-Ala-Ala-Lys(Trt-Glu-OtBu)-Glu(OtBu)-Phe-Ile-Ala-Trp-
Leu-Val-Arg(Pbf)-Gly-Arg(Pbf)-Gly-O-CTC resin
[SEQ ID NO: 59 or SEQ ID NO: 143]
H-Gln(Trt)-Ala-Ala-Lys(Trt-Glu-OtBu)-Glu(OtBu)-Phe-Ile-Ala-Trp-Leu-
Val-Arg(Pbf)-Gly-Arg(Pbf)-Gly-O-CTC resin
[SEQ ID NO: 60]
Fmoc-Gln(Trt)-Ala-Ala-Lys(Glu-OtBu)-Glu(OtBu)-Phe-Ile-Ala-Trp-Leu-
Val-Arg(Pbf)-Gly-Arg(Pbf)-Gly-OH
[SEQ ID NO: 61]
Fmoc-Gln(Trt)-Ala-Ala-Lys(Pal-Glu-OtBu)-Glu(OtBu)-Phe-Ile-Ala-Trp-
Leu-Val-Arg(Pbf)-Gly-Arg(Pbf)-Gly-OH
[SEQ ID NO: 62]
Fmoc-Gln(Trt)-Ala-Ala-Lys(Mmt)-Glu(OtBu)-Phe-Ile-Ala-Trp-Leu-Val-
Arg(Pbf)-Gly-Arg(Pbf)-Gly-O-CTC resin
[SEQ ID NO: 63]
Fmoc-Gln(Trt)-Ala-Ala-Lys(Mtt)-Glu(OtBu)-Phe-Ile-Ala-Trp-Leu-Val-
Arg(Pbf)-Gly-Arg(Pbf)-Gly-O-CTC resin
[SEQ ID NO: 64]
Fmoc-Gln(Trt)-Ala-Ala-Lys-Glu(OtBu)-Phe-Ile-Ala-Trp-Leu-Val-
Arg(Pbf)-Gly-Arg(Pbf)-Gly-OH
[SEQ ID NO: 65]
Cbz-Gln(Trt)-Ala-Ala-Lys(Pal-Glu-OtBu)-Glu(OtBu)-Phe-Ile-Ala-Trp-
Leu-Val-Arg(Pbf)-Gly-Arg(Pbf)-Gly-O-Wang resin
[SEQ ID NO: 66]
Cbz-Gln(Trt)-Ala-Ala-Lys(Trt-Glu-OtBu)-Glu(OtBu)-Phe-Ile-Ala-Trp-
Leu-Val-Arg(Pbf)-Gly-Arg(Pbf)-Gly-O-CTC resin
[SEQ ID NO: 67]
Cbz-Gln(Trt)-Ala-Ala-Lys(Glu-OtBu)-Glu(OtBu)-Phe-Ile-Ala-Trp-Leu-
Val-Arg(Pbf)-Gly-Arg(Pbf)-Gly-OH
[SEQ ID NO: 68]
Cbz-Gln(Trt)-Ala-Ala-Lys(Pal-Glu-OtBu)-Glu(OtBu)-Phe-Ile-Ala-Trp-
Leu-Val-Arg(Pbf)-Gly-Arg(Pbf)-Gly-OH
[SEQ ID NO: 69]
Cbz-Gln(Trt)-Ala-Ala-Lys(Mmt)-Glu(OtBu)-Phe-Ile-Ala-Trp-Leu-Val-
Arg(Pbf)-Gly-Arg(Pbf)-Gly-O-CTC resin
[SEQ ID NO: 70]
Cbz-Gln(Trt)-Ala-Ala-Lys(Mtt)-Glu(OtBu)-Phe-Ile-Ala-Trp-Leu-Val-
Arg(Pbf)-Gly-Arg(Pbf)-Gly-O-CTC resin
[SEQ ID NO: 71]
Cbz-Gln(Trt)-Ala-Ala-Lys-Glu(OtBu)-Phe-Ile-Ala-Trp-Leu-Val-
Arg(Pbf)-Gly-Arg(Pbf)-Gly-OH
[SEQ ID NO: 72]
Fmoc-Gln(Trt)-Ala-Ala-Lys-Glu(OtBu)-Phe-Ile-Ala-Trp-Leu-Val-
Arg(Pbf)-Gly-Arg(Pbf)-Gly-O-Wang resin
[SEQ ID NO: 73]
Fmoc-Gln(Trt)-Ala-Ala-Lys(Glu-OtBu)-Glu(OtBu)-Phe-Ile-Ala-Trp-Leu-
Val-Arg(Pbf)-Gly-Arg(Pbf)-Gly-O-Wang resin
[SEQ ID NO: 74]
Gln(Trt)-Ala-Ala-Lys(Mmt)-Glu(OtBu)-Phe-Ile-Ala-Trp-Leu-Val-
Arg(Pbf)-Gly-Arg(Pbf)-Gly-OH
[SEQ ID NO: 75]
Gln(Trt)-Ala-Ala-Lys(Mmt)-Glu(OtBu)-Phe-Ile-Ala-Trp-Leu-Val-
Arg(Pbf)-Gly-Arg(Pbf)-Gly-O-Wang resin
[SEQ ID NO: 76]
Gln(Trt)-Ala-Ala-Lys(Mtt)-Glu(OtBu)-Phe-Ile-Ala-Trp-Leu-Val-
Arg(Pbf)-Gly-Arg(Pbf)-Gly-OH
[SEQ ID NO: 77]
Gln(Trt)-Ala-Ala-Lys(Mtt)-Glu(OtBu)-Phe-Ile-Ala-Trp-Leu-Val-
Arg(Pbf)-Gly-Arg(Pbf)-Gly-O-Wang resin

(iv) Peptide fragments containing amino acid sequence (5-12) of liraglutide, i.e. Thr-Phe-Thr-Ser-Asp-Val-Ser-Ser [SEQ ID NO: 78], including:

F.

[SEQ ID NO: 79]

P1-Thr(P)-Phe-Thr(P)-Ser(P)-Asp(P)-Val-Ser(P)-
Ser(P)-O-P2

wherein P1 represents a protecting group for the N-terminal of Thr (preferably Fmoc or Cbz and more preferably Fmoc), each P represents side chain protecting groups which may be the same or different, and P2 is H or a solid support, preferably a CTC resin.
Preferred (5-12) peptides are as follows:

[SEQ ID NO: 80]

Fmoc-Thr(tBu)-Phe-Thr(tBu)-Ser(tBu)-Asp(OtBu)-Val-
Ser(tBu)-Ser(Ψ^{Me,Me}pro)-O-CTC resin

[SEQ ID NO: 298]

Fmoc-Thr(tBu)-Phe-Thr(tBu)-Ser(tBu)-Asp(OtBu)-Val-
Ser(Trt)-Ser(Trt)-O-CTC resin

[SEQ ID NO: 81]

Cbz-Thr(tBu)-Phe-Thr(tBu)-Ser(tBu)-Asp(OtBu)-Val-
Ser(tBu)-Ser(Ψ^{Me,Me}pro)-O-CTC resin

[SEQ ID NO: 299]

Cbz-Thr(tBu)-Phe-Thr(tBu)-Ser(tBu)-Asp(OtBu)-Val-
Ser(Trt)-Ser(Trt)-O-CTC resin

[SEQ ID NO: 82]

Fmoc-Thr(tBu)-Phe-Thr(tBu)-Ser(tBu)-Asp(OtBu)-Val-
Ser(tBu)-Ser(Ψ^{Me,Me}pro)-OH

[SEQ ID NO: 300]

Fmoc-Thr(tBu)-Phe-Thr(tBu)-Ser(tBu)-Asp(OtBu)-Val-
Ser(Trt)-Ser(Trt)-OH

[SEQ ID NO: 83]

Cbz-Thr(tBu)-Phe-Thr(tBu)-Ser(tBu)-Asp(OtBu)-Val-
Ser(tBu)-Ser(Ψ^{Me,Me}pro)-OH

[SEQ ID NO: 301]

Cbz-Thr(tBu)-Phe-Thr(tBu)-Ser(tBu)-Asp(OtBu)-Val-
Ser(Trt)-Ser(Trt)-OH

(v) Peptide fragments containing amino acid sequence (13-16) of liraglutide, i.e. Tyr-Leu-Glu-Gly [SEQ ID NO: 84], including:

G.

[SEQ ID NO: 85]

P1-Tyr(P)-Leu-Glu(P)-Gly-O-P2

wherein P1 represents a protecting group for the N-terminal of Tyr (preferably Fmoc or Cbz and more preferably Fmoc), each P represents side chain protecting groups which may be the same or different, and P2 is a solid support, preferably a CTC resin.
Preferred (13-16) peptides are as follows:

[SEQ ID NO: 86]

Fmoc-Tyr(tBu)-Leu-Glu(OtBu)-Gly-O-CTC resin

[SEQ ID NO: 87]

Cbz-Tyr(tBu)-Leu-Glu(OtBu)-Gly-O-CTC resin

[SEQ ID NO: 88 or SEQ ID NO: 163]

Tyr(tBu)-Leu-Glu(OtBu)-Gly-O-CTC resin

[SEQ ID NO: 302]

Fmoc-Tyr(tBu)-Leu-Glu(OtBu)-Gly-OH

[SEQ ID NO: 303]

Cbz-Tyr(tBu)-Leu-Glu(OtBu)-Gly-OH

[SEQ ID NO: 304]

Tyr(tBu)-Leu-Glu(OtBu)-Gly-OH

(vi) Peptide fragments containing amino acid sequence (20-31) of liraglutide, i.e. Lys-Glu-Phe-Ile-Ala-Trp-Leu-Val-Arg-Gly-Arg-Gly [SEQ ID NO: 89], including:

H.

[SEQ ID NO: 90]

P1-Lys(P)-Glu(P)-Phe-Ile-Ala-Trp-Leu-Val-Arg(P)-
Gly-Arg(P)-Gly-O-P2

wherein P1 represents a protecting group for the N-terminal of Lys (preferably Fmoc or Cbz and more preferably Fmoc), each P represents side chain protecting groups which may be the same or different, and P2 is a solid support, preferably a Wang resin.
Preferred (20-31) peptides are:

[SEQ ID NO: 91]

Fmoc-Lys(Mtt)-Glu(OtBu)-Phe-Ile-Ala-Trp-Leu-Val-
Arg(Pbf)-Gly-Arg(Pbf)-Gly-O-Wang resin

[SEQ ID NO: 92]

Fmoc-Lys(Mmt)-Glu(OtBu)-Phe-Ile-Ala-Trp-Leu-Val-
Arg(Pbf)-Gly-Arg(Pbf)-Gly-O-Wang resin

[SEQ ID NO: 93]

Fmoc-Lys-Glu(OtBu)-Phe-Ile-Ala-Trp-Leu-Val-
Arg(Pbf)-Gly-Arg(Pbf)-Gly-O-Wang resin

[SEQ ID NO: 94 or SEQ ID NO: 106]

Fmoc-Lys(Pal-Glu-OtBu)-Glu(OtBu)-Phe-Ile-Ala-Trp-
Leu-Val-Arg(Pbf)-Gly-Arg(Pbf)-Gly-O-Wang resin

[SEQ ID NO: 95]

Cbz-Lys(Mtt)-Glu(OtBu)-Phe-Ile-Ala-Trp-Leu-Val-
Arg(Pbf)-Gly-Arg(Pbf)-Gly-O-Wang resin

[SEQ ID NO: 96]

Cbz-Lys(Mmt)-Glu(OtBu)-Phe-Ile-Ala-Trp-Leu-Val-
Arg(Pbf)-Gly-Arg(Pbf)-Gly-O-Wang resin

[SEQ ID NO: 97]

Cbz-Lys-Glu(OtBu)-Phe-Ile-Ala-Trp-Leu-Val-
Arg(Pbf)-Gly-Arg(Pbf)-Gly-O-Wang resin

[SEQ ID NO: 98]

Cbz-Lys(Pal-Glu-OtBu)-Glu(OtBu)-Phe-Ile-Ala-Trp-
Leu-Val-Arg(Pbf)-Gly-Arg(Pbf)-Gly-O-Wang resin

[SEQ ID NO: 99]

Fmoc-Lys(Trt-Glu-OtBu)-Glu(OtBu)-Phe-Ile-Ala-Trp-
Leu-Val-Arg(Pbf)-Gly-Arg(Pbf)-Gly-O-Wang resin

[SEQ ID NO: 100 or SEQ ID NO: 109]

Fmoc-Lys(Glu-OtBu)-Glu(OtBu)-Phe-Ile-Ala-Trp-Leu-
Val-Arg(Pbf)-Gly-Arg(Pbf)-Gly-O-Wang resin

(vii) Peptide fragments containing amino acid sequence (18-31) of liraglutide, i.e. Ala-Lys-Glu-Phe-Ile-Ala-Trp-Leu-Val-Arg-Gly-Arg-Gly [SEQ ID NO: 101], including:

I.

[SEQ ID NO: 102]

Ala-Lys(Pal-Glu-P)-Glu(P)-Phe-Ile-Ala-Trp-Leu-Val-
Arg(P)-Gly-Arg(P)-Gly-O-P2,

wherein each P represents side chain protecting groups which may be the same or different, and P2 is a solid support, preferably a Wang resin.
A preferred (19-31) peptide is:

[SEQ ID NO: 103 or SEQ ID NO: 134]

Ala-Lys(Pal-Glu-OtBu)-Glu(OtBu)-Phe-Ile-Ala-Trp-
Leu-Val-Arg(Pbf)-Gly-Arg(Pbf)-Gly-O-Wang resin,

(viii) Peptide fragments containing amino acid sequence (18-31) of liraglutide, i.e. Ala-Ala-Lys-Glu-Phe-Ile-Ala-Trp-Leu-Val-Arg-Gly-Arg-Gly [SEQ ID NO: 104], including:

J.

[SEQ ID NO: 105]

Ala-Ala-Lys(Pal-Glu-P)-Glu(P)-Phe-Ile-Ala-Trp-Leu-
Val-Arg(P)-Gly-Arg(P)-Gly-O-P2

(ix) Other peptide fragments include:

[SEQ ID NO: 106 or SEQ ID NO: 94]

Fmoc-Lys(Pal-Glu-OtBu)-Glu(OtBu)-Phe-Ile-Ala-Trp-
Leu-Val-Arg(Pbf)-Gly-Arg(Pbf)-Gly-O-Wang resin

[SEQ ID NO: 107]

Fmoc-Ala-Lys(Pal-Glu-OtBu)-Glu(OtBu)-Phe-Ile-Ala-
Trp-Leu-Val-Arg(Pbf)-Gly-Arg(Pbf)-Gly-O-Wang resin

[SEQ ID NO: 108]

Fmoc-Ala-Ala-Lys(Pal-Glu-OtBu)-Glu(OtBu)-Phe-Ile-
Ala-Trp-Leu-Val-Arg(Pbf)-Gly-Arg(Pbf)-Gly-O-Wang
resin

[SEQ ID NO: 109 or SEQ ID NO: 100]

Fmoc-Lys(Glu-OtBu)-Glu(OtBu)-Phe-Ile-Ala-Trp-Leu-
Val-Arg(Pbf)-Gly-Arg(Pbf)-Gly-O-Wang resin

[SEQ ID NO: 110]

Fmoc-Ala-Lys(Glu-OtBu)-Glu(OtBu)-Phe-Ile-Ala-Trp-
Leu-Val-Arg(Pbf)-Gly-Arg(Pbf)-Gly-O-Wang resin

[SEQ ID NO: 111]

Fmoc-Ala-Ala-Lys(Glu-OtBu)-Glu(OtBu)-Phe-Ile-Ala-
Trp-Leu-Val-Arg(Pbf)-Gly-Arg(Pbf)-Gly-O-Wang resin

wherein each P represents side chain protecting groups which may be the same or different, and P2 is a solid support, preferably a Wang resin.
A preferred (18-31) peptide is:

[SEQ ID NO: 112 or SEQ ID NO: 140]

Ala-Ala-Lys(Pal-Glu-OtBu)-Glu(OtBu)-Phe-Ile-Ala-
Trp-Leu-Val-Arg(Pbf)-Gly-Arg(Pbf)-Gly-O-Wang resin

A further aspect of the present invention provides liraglutide of high purity. In particular, the liraglutide may contain less than 5%, less than 2%, less than 1%, less than 0.5%, less than 0.2%, or less than 0.1% by weight of the D-His isomer of liraglutide, and/or less than 5%, less than 2%, less than 1%, less than 0.5%, less than 0.2%, or less than 0.1% by weight of the [+Gly¹⁶] derivative of liraglutide, and/or less than 5%, less than 2%, less than 1%, less than 0.5%, less than 0.2%, or less than 0.1% by weight of the [+Gly³¹] derivative of liraglutide, and/or less than 5%, less than 2%, less than 1%, less than 0.5%, less than 0.2%, or less than 0.1% by weight of the [+Gly⁴] derivative of liraglutide.

The present invention further provides processes for preparing Semaglutide as set out in detail below, as well as a process for purifying liraglutide.

### BRIEF DESCRIPTION OF THE DRAWINGS

FIG. 1: Synthesis of Liraglutide on Wang resin

FIG. 2: Synthesis of Liraglutide by three-fragment condensation in solution Applying Fmoc-Lys(Trt-Glu-OtBu)

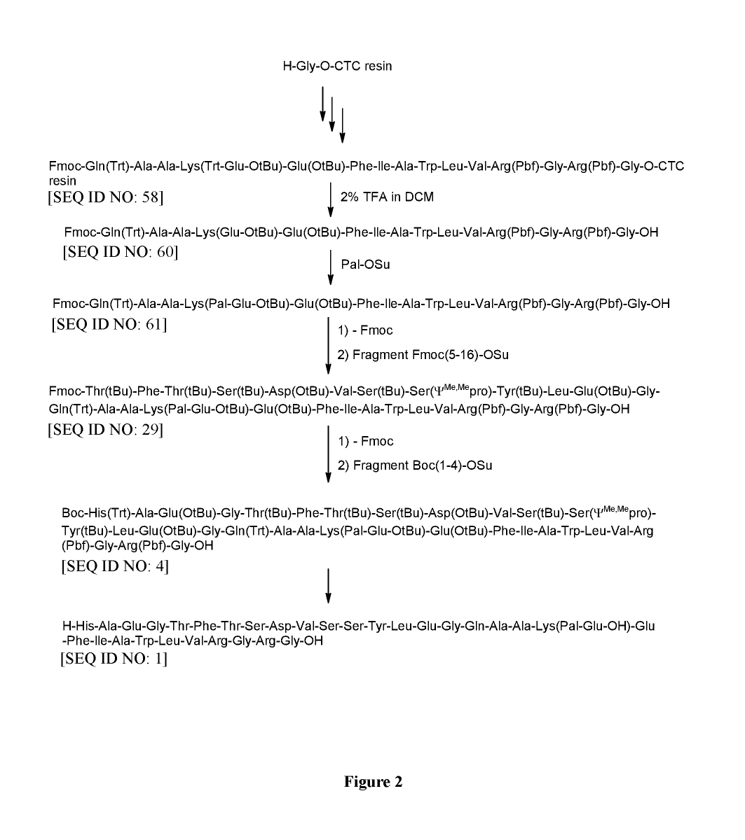

FIG. 3: Synthesis of Liraglutide by three-fragment condensation in solution applying Fmoc-Lys(Mmt)

FIG. 4: Synthesis of Liraglutide by two-fragment condensation on resin and one in solution applying Fmoc-Lys(Trt-Glu-OtBu)

FIG. 5: Synthesis of SEQ ID NO: 44 on CTC resin applying fragment condensation on solid support.

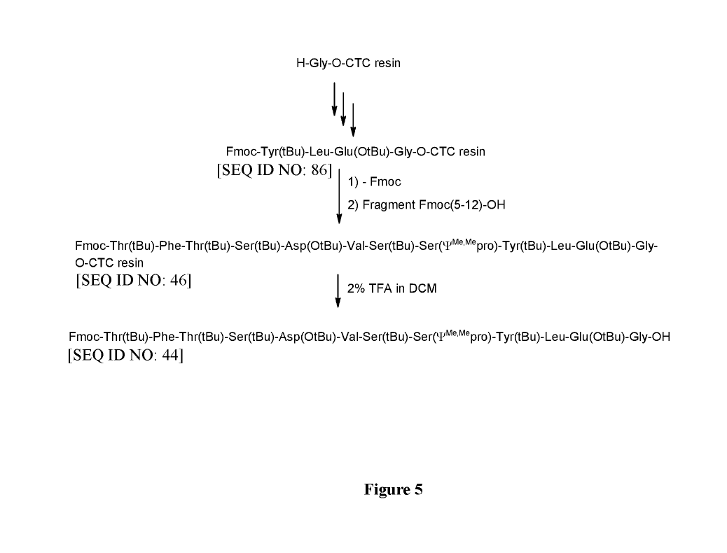

### DETAILED DESCRIPTION OF THE INVENTION

An objective of the present invention is to provide a method for the synthesis of GLP-1 peptides, such as Liraglutide or Semaglutide, with the advantages of a better quality (purity) and yield of the crude peptide at the end of the synthesis. Due to the reduced amounts of closely related impurities in the resulting peptide product such as liraglutide or semaglutide, the GLP-1 peptide such as liraglutide or semaglutide may be readily purified in order to achieve a high purity final product. For liraglutide, this synthetic approach makes possible the introduction of the Pal-Glu unit on the side chain of Lys at early stage of the synthesis, and for semaglutide, this synthetic approach enables facile introduction of the N-(17-carboxy-1-oxoheptadecyl)-L-γ-glutamyl-2-[2-(2-aminoethoxy)ethoxy]acetyl at Lys again at an early stage of the synthesis. Typically, for liraglutide, the prior art processes as discussed above involve the introduction of the Pal-Glu unit at the end of the synthesis of the liraglutide chain, for the likely reason that the introduction of such a bulky, hydrophobic group at an early stage in the synthesis would be expected to interfere in the synthesis of the peptide chain. However, the inventors of the present invention have surprisingly found that the introduction of the Pal-Glu-OtBu in an early stage of the synthesis of liraglutide does not interfere to the peptide chain elongation, and moreover, advantageously enables the production of Liraglutide in high yield and purity. Similarly, the introduction of the N-(17-carboxy-1-oxoheptadecyl)-L-γ-glutamyl-2-[2-(2-aminoethoxy)ethoxy]acetyl-2-[2-(2-aminoethoxy)ethoxy]acetyl at Lys also surpisingly does not interfere with the peptide chain elongation, and enables production of semaglutide in high yield and purity.

In certain embodiments, the present invention enables the coupling of the peptide fragments in solution, i.e. without the need for a hydrophobic solid support. Unexpectedly, the present inventors have found that the coupling of the peptide fragments in solution in the absence of a resin still enables production of GLP-1 proteins such as liraglutide and semaglutide in high yield and purity. The present inventors have further found that contrary to the regular fragment condensation approach in solution, where protection of the free carboxylic group of the C-terminus fragment as an ester is required, by employing fragments in a preactivated form (such as isolated OSu, OBt or OPfp esters, particularly isolated OSu or OPfp esters, and more particularly OPfp esters) in accordance with embodiments of the present invention, no such C-terminus protection is necessary. This avoids the need to use carboxyl protecting groups during the synthesis, and hence minimizes the associated loss of yield and purity resulting from the steps of introduction and removal of such protecting groups. A further advantage is that conducting the synthesis steps in solution enables lower quantities of reagents to be used, resulting in a more economical process, and also a lower likelihood of side reactions.

The present invention provides a convergent synthesis of GLP-1 peptides such as Liraglutide or Semaglutide. In particular the process involves the production of fragments of the Liraglutide or Semaglutide sequences (or other similar GLP-1 peptides) and condensing the fragments. A particular advantage of the present fragment condensation process is the ability to prepare high purity fragments without the need for complicated isolation and purification procedures. In this way, the process of the present invention further enables the production of GLP-1 peptides such as liraglutide or semaglutide with lower amounts of particular impurities, which facilitates subsequent purification of the GLP-1 peptides such as liraglutide or semaglutide following its synthesis. For example, the processes of the present invention enables production of GLP-1 peptides such as liraglutide or semaglutide having lower amounts of impurities, such as the D-His isomer of liraglutide or semaglutide (wherein the terminal His residue in liraglutide/semaglutide has D-configuration instead of L-configuration), and diglycine derivatives of liraglutide or semaglutide, wherein positions 4, 16 or 31 of liraglutide or semaglutide contain an extra Gly residue (referred to herein as [+Gly⁴], [+Gly¹⁶] and [+Gly³¹] impurities respectively).

In particular, since the synthesized sequence contains a His residue; partial racemization of this amino acid typically occurs during the coupling reaction, resulting in the formation of an undesirable D-His impurity. Typically, the D-His impurity can be present in an amount of several %. For example, in the prior art sequential syntheses of liraglutide, the separation of this impurity from the final peptide is extremely difficult and thus the purified peptide can contain varying amounts of D-His impurity. By using a convergent synthesis in accordance with the process of the present invention, and in particular, during the synthesis of a fragment containing the His residue (i.e. Peptide 1—for example, SEQ ID NOs: 5-18, such as SEQ ID NO: 7, or SEQ ID NOs: 177-189), it was found that although the D-His impurity (i.e. the D-His isomer of Peptide 1, which has the same formula as Peptide 1, but wherein the terminal His residue has D-configuration) is still formed (typically at amounts of up to 5 wt % the D-His impurity can be removed from the fragment to an amount of less than 0.5%, for example from about 0.2% to about 0.5%, e.g., not more than 0.2% by weight, or not more than 0.1% by weight. Advantageously, it has been found that Peptide 1 can be readily purified by simple procedures and does not require the use of preparative HPLC. Using this purified fragment enables the production of, for example, liraglutide and semaglutide with very low amounts of the D-His isomer of liraglutide/semaglutide. This is true also for other impurities that are typically obtained during the synthesis of liraglutide/semaglutide. For example, a further such impurity is the [+Gly⁴] impurity. This impurity can be easily detected by HPLC analysis of the fragment. It has been surprisingly found that the [+Gly⁴] derivative of the Peptide 1 fragment can be readily removed from the Peptide 1 product, so that during coupling to obtain liraglutide or semaglutide, the production of the [+Gly⁴] derivative of liraglutide or semaglutide is minimized or avoided.

Further, the process of the present invention, wherein liraglutide is prepared by coupling of a peptide containing amino acid sequence (1-4) with a peptide containing amino acid sequence (5-31) of liraglutide, prevents racemization reaction that is typically induced by coupling at other sites on the liraglutide sequence. Similarly, the process of the present invention wherein semaglutide is prepared by coupling of a peptide containing amino acid sequence (1-4) with a peptide containing amino acid sequence (5-31) of semaglutide, prevents racemization reaction that is typically induced by coupling at other sites on the semaglutide sequence. Therefore the present process minimizes or eliminates the production of difficult to separate side products resulting from racemization side reactions which inevitably occur during the coupling steps. The resulting product can be readily purified and thus can be obtained in high purity.

For the purpose of clarity and as an aid in the understanding of the invention, as disclosed and claimed herein, the following terms and abbreviations are defined below:
AOP (7-azabenzotriazol-1-yloxy)tris(dimethylamino)phosphonium hexafluorophosphate
BTFFH bis(tetramethylene)fluoroformamidinium hexafluorophosphate
Boc or t-Boc t-butyloxycarbonyl
BOP benzotriazol-1-yl-oxytris-(dimethylamino)-phosphonium hexafluorophosphate
BOP—Cl bis(2-oxo-3-oxazolidinyl)phosphonic chloride
BroP bromo-tris(dimethylamino)phosphonium hexafluorophosphate
Cbz carboxybenzyl
CDI 1,1′-Carbonyl-diimidazole
CIP 2-Chloro-1,3-dimethylimidazolidinium hexafluorophosphate
DCC N,N′-dicyclohexyl carbodiimide
DCM dichloromethane
DEPC diethylphosphorocyanidate
DIC N,N′-diisopropylcarbodiimide
DEE diethylether
DMF dimethylformamide
DPPA diphenylphosphorylazide
DTT dithiothreitol
EDC-HCl 1-ethyl-3-(3′-dimethyl-aminopropyl)carbodiimide hydrochloride
EEDQ (2-ethoxy-1-ethoxycarbonyl-1,2-dihydroquinoline)
Fmoc 9-fluorenylmethoxycarbonyl
HAPyU 1-(1-pyrrolidinyl-1H-1,2,3-triazolo[4,5-b]pyridin-1-yl-methylene)pyrrolidinmium hexafluorophosphate
HATU 2-[(dimethylamino)-1H-1,2,3-triazolo[4,5-b]pyridin-1-ylmethylene]-N-methylmethanaminium hexafluorophosphate
HBTU 2-(1H-benzotriazol-1-yl)-N,N,N′,N′-tetramethyluronium hexafluorophosphate
HCTU 2-(6-Chloro-1H-benzotriazol-1-yl)-N,N,N′,N′-tetramethyluronium hexafluorophosphate
HBPyU 1-[(1H-benzotryazole-1-yl-oxy)(pyrrolidine-1-yl)methylidene]pyrrolidinium hexafluorophosphate
HOBt N-hydroxybenzotriazole
HPLC High Performance Liquid Chromatography
IIDQ N-isobutoxycarbonyl-2-isobutoxy-1,2-dihydroquinoline
Mmt monomethoxytrityl [(4-methoxyphenyl)diphenylmethyl]
MTBE methyl-t-butyl ether
Mtt 4-methyltrityl
NMP N-methylpyrrolidone
OAt hydroxy-7-azabenzotriazole
OBt O-benzotriazole
OBzl O-benzyl
Oct ethyl 1-hydroxy-1H-1,2,3-triazole-4-carboxylate
ODhbt 1-oxo-2-hydroxydihydrobenzotriazine
ODNP dinitrophenol
Fm 9-fluorenylmethyl
ONB N-hydroxy-5-norbornene-endo-2,3-dicarboxyimide
OPfp O-pentafluorophenyl
OPht N-hydroxy-phthalimide
OPNP p-nitrophenol
OSu succinimide ester
Ot N-hydroxytetrazole
OtBu tert-butyl ester
OTCP trichlorophenol
TFFH 1,1,3,3-tetramethylfluoroformamidinium hexafluorophosphate
Pal palmitoyl
Pbf 2,2,4,6,7-pentamethyl-dihydrobenzofuran-5-sulfonyl
PfPU pentafluorophenol-tetramethyluronium hexafluorophosphate:

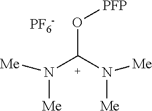

PfTU pentafluorophenyl-1,1,3,3-bis(tetramethylene)uronium hexafluorophosphate:

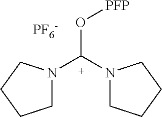

PyAOP (7-azabenzotriazol-1-yloxy)tris(pyrrolidino)phosphonium hexafluorophosphate
PyBOP benzotriazol-1-yl-oxy-tris-pyrrolidinophosphonium hexafluorophosphate
PyBrop bromo-tris-pyrrolidino-phosphonium hexafluorophosphate
SPPS solid phase peptide synthesis
LPPS liquid phase peptide synthesis
TBTU 2-(1H-benzotriazol-1-yl)-N,N,N′,N′-tetramethyluronium tetrafluoroborate
tBu tert-butyl
TFA trifluoroacetic acid
Trt trityl

As used herein, unless stated otherwise, percentages relate to weight percent.

The solid supports for the processes of the present invention are preferably resins that are cleavable using acid, preferably trifluoroacetic acid. Preferred resins for use in the processes of the present invention are Wang resins and hyper-acid labile resins, such as chlorotrityl based (CTC) resins, 4-methoxytrityl or 4-methyl-trityl resins. CTC resins are preferred. Hyper-acid labile resins such as CTC resins are cleavable under milder acidic conditions. For example, hyper-acid labile resins such as CTC resins can be removed using weak acid solutions, such as 2% trifluoroacetic acid. The term “Wang resin” typically refers to a polyethylene-based resin, preferably containing p-alkoxybenzyl alcohol or p-alkoxybenzyloxycarbonyhydrazide based resins, typically attached to a polyethylene glycol or polystyrene core (Wang, S., J. Am. Chem. Soc., 1973, 95(4), 1328-1333). Wang resins are typically removed under strong acid conditions, e.g. at least 50% trifluoroacetic acid solutions. Preferred Wang and CTC resins for the present invention are those on a polystyrene support. These resins are commercially available. For example, H-Gly-Wang resin or H-Gly-CTC resin, or the free resins themselves are commercially available and are suitable starting materials for use in the present invention.

As used herein, the term “sequential synthesis” or “linear synthesis” refers to a process whereby the final product or an intermediate thereof is prepared by sequential transformations of a single starting material. Typically, in a sequential or linear synthesis, the final product is prepared by sequential condensation of single amino acids to build the final peptide sequence. Typically the single amino acids are optionally side-chain protected as well as N-terminal protected with the usual protecting groups for peptide synthesis. Preferably, the N-terminal protecting groups are Fmoc, Boc, or Cbz, and more preferably Fmoc or Boc. The condensation(s) can be carried out as a solid phase synthesis (i.e. on a solid support, such as a resin) or in liquid phase (i.e. with the free peptide—i.e. a peptide that is not conjugated to a solid support/resin), or a combination of both.

As used herein, the term “convergent synthesis” refers to a process whereby subunits (peptide fragments) of the final product are prepared separately, and subsequently brought together or coupled together to form the final compound. Typically, in a convergent synthesis, the target peptide is prepared by the coupling of two or more subunits (peptide fragments) which together make up the final peptide sequence, and optionally deprotecting and removing any resin. The subunits (peptide fragments) may themselves be made by a convergent or by sequential synthesis. The peptide fragments may be protected or unprotected during the coupling step. Preferably, in all embodiments of the present invention, one or more amino acids in the peptide fragments are side chain protected during the coupling step. Moreover, one of the peptides may be present on a resin, such as a CTC resin or a Wang resin. In the synthesis of liraglutide or semaglutide, each of which contains 31 amino acids making up the liraglutide or semaglutide backbone, the convergent synthesis preferably involves condensing two, three or four peptide fragments to form the liraglutide or semaglutide sequence, and optionally deprotecting and removing any resin.

As used herein, the term “peptide” refers to a compound containing at least two amino acids in which the carboxyl group of one acid is linked to the amino group of the other (i.e. the two amino acids are linked by a peptide bond). The term “peptide” as used herein encompasses amino acid sequences in which carboxyl and/or amino groups are protected or unprotected. Suitable protecting groups for the carboxyl groups of the amino acids include OtBu, OBzl, OFm. Suitable protecting groups for the amino groups of the amino acids include Fmoc, Boc, Mmt, Mtt, Cbz, Trt.

Suitable protecting groups for the N-terminal amino acid include Fmoc, Boc and Cbz.

In the coupling reactions of any embodiment of the present invention, the amino acid or peptide fragment is coupled using Fmoc, Boc, or Cbz strategy which is well known in the art of peptide synthesis. Thus, the typically side-chain protected amino acid or peptide fragment to be coupled onto another amino acid or peptide fragment is generally also N-terminal protected with Fmoc, Boc or Cbz to form a peptide or peptide fragment containing an N-terminal Fmoc, Boc or Cbz group. Preferably the N-terminal protection is Fmoc or Boc, and more preferably Fmoc. In any subsequent coupling step, the N-terminal protection of the peptide formed in the preceding coupling step is removed, for example by reaction with, e.g. a base such as piperidine in the case of Fmoc, or an acid, such as TFA (trifluoroacetic acid) in the case of Boc, before the next amino acid or peptide is coupled. Preferably in the final step involving coupling of peptide fragment containing amino acids (1-4) with (5-31), to form the liraglutide or semaglutide sequence (1-31), the peptide fragment (1-4) is Boc-protected at the N-terminal His residue (i.e. Boc-His). Fmoc (or a combination of Fmoc and Cbz), is the preferred N-terminal protecting group used in the preparation of the other peptide fragments according to the present invention.

In the case of solid phase synthesis according to the present invention, the coupling is carried out with Fmoc strategy using peptide fragments containing amino acid side chain protecting groups which are only acid-cleavable (i.e. are stable to the basic conditions that are generally employed to remove the base-cleavable N-terminal protecting groups), and the removal of the N-terminal protection (e.g. Fmoc) is conducted with a base. The coupling of Peptide 1 with Peptide 2 to form liraglutide or semaglutide which typically contains protected amino acid residues, is preferably carried out using an acid-labile N-terminal protecting group in the His residue of Peptide 1, such as Boc, so that the N-terminal protecting group and the amino acid protecting groups in the protected liraglutide or semaglutide sequence can be removed (optionally along with any solid support, e.g. Wang resin) in one step. For example, the His N-terminal Boc group may be removed together with the acid-labile protecting groups and Wang resin by treatment with a cleavage cocktail (typically a cleavage cocktail comprises trifluoroacetic acid (TFA), and can be a mixture of TFA with dithiothreitol in dichloromethane), thereby producing liraglutide or semaglutide. In the steps preceding the coupling of Peptide 1 with Peptide 2, i.e. the preparation of Peptides 1, 2, 3, 4, 4A, 4B, 5 and 6, protection at the N-terminal amino acid with a base-cleavable protecting group, preferably Fmoc is preferred, with the exception of the coupling of the terminal His¹ residue in Peptide 1, in which the N-terminal protection is preferably an acid-cleavable protecting group, preferably Boc. In this way, the Fmoc can be readily removed using base (e.g. piperidine) without affecting the acid-cleavable protecting groups on other amino acids in the peptide fragments, and in the final coupling step of Peptide 1 with Peptide 2, the Boc-protected His residue can be readily cleaved under the acidic conditions used to cleave the side chain protecting groups and any resin. In one of the preferred embodiments of the present invention, liraglutide or semaglutide is prepared by liquid phase coupling, i.e. wherein a resin is not employed. In this embodiment, although the intermediate peptide fragments may be prepared on a resin (e.g. CTC resin), the final coupling reaction of Peptide 1 with Peptide 2 is conducted in the liquid phase. For example, in these embodiments, Peptide 4 may be prepared on a resin (i.e. a hyper-acid labile resin) such as a CTC resin (preferred), and the resin is cleaved under mild acid conditions (which removes the resin but does not affect the acid-cleavable protecting groups) before coupling with Peptide 3 to form Peptide 2. CTC resin is particularly suitable for such a process because this resin can be cleaved under mild conditions, such as dilute TFA solution (e.g. ≦10%, ≦5%, ≦2% vol/vol in a suitable organic solvent such as dichloromethane. These conditions leave most of the other acid-cleavable amino acid protecting groups intact. As another example, Peptide 2 may be prepared by the coupling of Peptide 3 with Peptide 4A on a resin (preferably a hyper-acid labile resin such as CTC resin), completing the sequence of Peptide 2, and then removing the Peptide 2 from the resin before coupling with Peptide 1 in the liquid phase.

As used herein, the term “segment” or “fragment” of liraglutide or semaglutide refer to a sequence of two or more amino acids present in liraglutide or semaglutide respectively. The amino acids in the segment or fragment may be protected or unprotected.

Preferably in any embodiment of the present invention, the amino acids in the fragments are protected, preferably with acid-cleavable protecting groups. In particular, the trifunctional amino acids, namely: Thr, Ser, Asp, Tyr, Glu, Gln, Lys and Arg residues are protected with acid-cleavable protecting groups. Suitable acid-cleavable protecting groups are selected from the group consisting of: tBu, OtBu, Ψ^{Me,Me}pro, Trt, and Pbf. In particular the residues are protected as follows: Thr(tBu), Ser⁸(tBu), Ser⁸(Trt), Ser¹¹(tBu), Ser¹¹(Trt), Lys(Mtt) or Lys(Mmt) or Lys(Trt-Glu-OtBu), Asp (OtBu), Ser¹²(Ψ^{Me,Me}pro), Ser¹²(Trt), Tyr(tBu), Glu(OtBu), Gln(Trt), and Arg(Pbf). In a preferred embodiment, according to any process of the present invention, the residues are protected as follows: Thr(tBu), Ser⁸(tBu), Ser¹¹(tBu), Lys(Mtt) or Lys(Mmt) or Lys(Trt-Glu-OtBu) [of which Lys(Mmt) is particularly preferred), Asp (OtBu), Ser¹²(Ψ^{Me,Me}pro), Tyr(tBu), Glu(OtBu), Gln(Trt), and Arg(Pbf). In another preferred embodiment, according to any process of the invention, the residues are protected as follows: Thr(tBu), Ser⁸(tBu), Ser¹¹(Trt), Lys(Mtt) or Lys(Mmt) or Lys(Trt-Glu-OtBu), Asp (OtBu), Ser¹²(Trt), Tyr(tBu), Glu(OtBu), Gln(Trt), and Arg(Pbf).

As used herein, the term “D-His impurity of liraglutide” refers to H-D-His-Ala-Glu-Gly-Thr-Phe-Thr-Ser-Asp-Val-Ser-Ser-Tyr-Leu-Glu-Gly-Gln-Ala-Ala-Lys(Pal-Glu-OH)-Glu-Phe-Ile-Ala-Trp-Leu-Val-Arg-Gly-Arg-Gly-OH [SEQ ID NO: 113]. The term “D-His impurity” in relation to Peptide 1 refers to a peptide having the same formula as Peptide 1 (e.g. [SEQ ID NOs: 5-18 and 268-270 or SEQ ID NOs: 177-189]), but wherein the terminal His residue has D-configuration. Similarly, the D-His impurity of semaglutide refers to H-D-His-Aib-Glu-Gly-Thr-Phe-Thr-Ser-Asp-Val-Ser-Ser-Tyr-Leu-Glu-Gly-Gln-Ala-Ala-Lys(W)-Glu-Phe-Ile-Ala-Trp-Leu-Val-Arg-Gly-Arg-Gly-OH {W=N-(17-carboxy-1-oxoheptadecyl)-L-γ-glutamyl-2-[2-(2-aminoethoxy)ethoxy]acetyl-2-[2-(2-aminoethoxy)ethoxy]acetyl} [SEQ ID NO: 203].

As used herein, the term “[+Gly⁴] impurity of liraglutide” refers to liraglutide which contains an extra Gly residue at position 4 (i.e. the Gly residue at position 4 is replaced by Gly-Gly), i.e.: H-His-Ala-Glu-Gly-Gly-Thr-Phe-Thr-Ser-Asp-Val-Ser-Ser-Tyr-Leu-Glu-Gly-Gln-Ala-Ala-Lys(Pal-Glu-OH)-Glu-Phe-Ile-Ala-Trp-Leu-Val-Arg-Gly-Arg-Gly-OH [SEQ ID NO: 114]. As used herein, the term [+Gly⁴] impurity” in relation to Peptide 1” refers to a peptide having the same amino acid sequence as Peptide 1 (e.g. [SEQ ID NOs: 5-18 and 268-270 or SEQ ID NOs: 177-189]) with the exception of an additional terminal Gly residue, i.e. His-Ala-Glu-Gly-Gly [SEQ ID NO: 115] in the case of liraglutide, or His-Aib-Glu-Gly-Gly [SEQ ID NO: 204], in the case of semaglutide. Similarly, the “[+Gly⁴] impurity of semaglutide” refers to semaglutide which contains an extra Gly residue at position 4 (i.e. the Gly residue at position 4 is replaced by Gly-Gly), i.e.: the H-His-Aib-Glu-Gly-Gly-Thr-Phe-Thr-Ser-Asp-Val-Ser-Ser-Tyr-Leu-Glu-Gly-Gln-Ala-Ala-Lys(W)-Glu-Phe-Ile-Ala-Trp-Leu-Val-Arg-Gly-Arg-Gly-OH {W=N-(17-carboxy-1-oxoheptadecyl)-L-γ-glutamyl-2-[2-(2-aminoethoxy)ethoxy]acetyl-2-[2-(2-aminoethoxy)ethoxy]acetyl} [SEQ ID NO: 205]

Unless otherwise indicated, the reference to the residue “H-His-” denotes that the terminal His residue (i.e. at amino acid position 1 of liraglutide or semaglutide) does not contain an N-terminal protecting group, whereas, for example, “Boc-His” refers to a His residue which is protected at the N-terminal group with Boc. Similarly, unless otherwise indicated, “H-AA” refers to a terminal amino acid (AA) residue that does not contain an N-terminal protecting group.

Unless otherwise indicated, the reference to the residue “Gly-OH” denotes that the carboxylic acid group of the Gly residue is unsubstituted, and thus contains a free —OH group, whereas, for example, “Gly-OtBu” refers to a Gly residue in which the carboxylic acid OH group is substituted to form OtBu, and Gly-O-resin refers to a terminal Gly residue which is attached to a solid support (e.g. Gly-O-Wang resin, or Gly-O-CTC resin). In some instances, the term “AA-OH” may also be specified to refer to a terminal amino acid residue that is either optionally conjugated to a resin via the carboxylic acid terminal group or optionally the amino acid contains a carboxylic acid terminal group in activated form such as e.g. OSu.

As used herein, the term “[+Gly¹⁶] impurity of liraglutide” refers to liraglutide which contains an extra Gly residue at position 16 (i.e. the Gly residue at position 16 is replaced by Gly-Gly), i.e.: H-His-Ala-Glu-Gly-Thr-Phe-Thr-Ser-Asp-Val-Ser-Ser-Tyr-Leu-Glu-Gly-Gly-Gln-Ala-Ala-Lys(Pal-Glu-OH)-Glu-Phe-Ile-Ala-Trp-Leu-Val-Arg-Gly-Arg-Gly-OH [SEQ ID NO: 116].

As used herein, the term “[+Gly³¹] impurity of liraglutide” refers to liraglutide which contains an extra terminal Gly residue, i.e. H-His-Ala-Glu-Gly-Thr-Phe-Thr-Ser-Asp-Val-Ser-Ser-Tyr-Leu-Glu-Gly-Gln-Ala-Ala-Lys(Pal-Glu-OH)-Glu-Phe-Ile-Ala-Trp-Leu-Val-Arg-Gly-Arg-Gly-Gly-OH [SEQ ID NO: 117].

As used herein, the term “[+Gly¹⁶] impurity of semaglutide” refers to semaglutide which contains an extra Gly residue at position 16 (i.e. the Gly residue at position 16 is replaced by Gly-Gly), i.e.: H-His-Aib-Glu-Gly-Thr-Phe-Thr-Ser-Asp-Val-Ser-Ser-Tyr-Leu-Glu-Gly-Gly-Gln-Ala-Ala-Lys(W)-Glu-Phe-Ile-Ala-Trp-Leu-Val-Arg-Gly-Arg-Gly-OH [SEQ ID NO: 206].

As used herein, the term “[+Gly³¹] impurity of semaglutide” refers to semaglutide which contains an extra terminal Gly residue, i.e. H-His-Aib-Glu-Gly-Thr-Phe-Thr-Ser-Asp-Val-Ser-Ser-Tyr-Leu-Glu-Gly-Gln-Ala-Ala-Lys(W)-Glu-Phe-Ile-Ala-Trp-Leu-Val-Arg-Gly-Arg-Gly-Gly-OH [SEQ ID NO: 207].

The purity of the GLP-1 peptide, such as Liraglutide or Semaglutide can be determined by any suitable analytical method for example HPLC, LC/MS or chiral amino acid analysis (chiral AAA).

The above processes proceed via novel synthetic intermediates, including intermediates (i)-(xxx) as set out below. The present invention encompasses these intermediates, as well as their use in a process for the manufacture of Liraglutide or Semaglutide as appropriate.

### Preparation of Liraglutide

In one aspect, the process involves preparing liraglutide [SEQ ID NO: 1] comprising:

(i) coupling a Peptide 1 having the sequence [SEQ ID NO: 5]

His-Ala-Glu-Gly

wherein:
   the N-terminal of His is optionally protected with a protecting group, preferably selected from the group consisting of Boc, Cbz or Fmoc, and
   the Gly carboxylic acid group in Peptide 1 may be in the form of an activated carboxylic acid derivative;
      with a Peptide 2 having the sequence:

[SEQ ID NO: 24]

Thr-Phe-Thr-Ser-Asp-Val-Ser-Ser-Tyr-Leu-Glu-Gly-
Gln-Ala-Ala-Lys(Pal-Glu-OX)-Glu-Phe-Ile-Ala-Trp-
Leu-Val-Arg-Gly-Arg-Gly-OH

wherein:
Peptide 2 is optionally conjugated to a solid support (preferably, the Peptide 2 is either conjugated to a Wang resin, or Peptide 2 is not present on a resin—i.e. the coupling of Peptide 1 with Peptide 2 is conducted in the liquid phase);
X represents H or a protecting group for the Glu carboxylic acid group,
   and wherein one or more of the amino acid residues in Peptide 1 and Peptide 2 may be protected or unprotected;
   (ii) optionally removing any protecting groups and/or cleaving the resin to form liraglutide; and
   (iii) optionally purifying the liraglutide.
   Preferably, in any embodiment of the present invention, Peptide 1 is SEQ ID NO: 6 as described above. More preferably, Peptide 1 is any of SEQ ID NOs: 7-15 or SEQ ID NOs: 268-270.

The coupling of Peptide 1 with Peptide 2, particularly in the case of a liquid phase coupling may be conducted on an activated form of Peptide 1, wherein the Gly carboxylic acid group in Peptide 1 is in the form of an activated carboxylic acid derivative, preferably wherein the activated carboxylic acid derivative is selected from the group consisting of:
an activated ester, preferably wherein the activated ester is selected from the group consisting of OSu, OPfp, OBt, OAt, ODhbt, ONB, OPht, ONP, ODNP, Ot, Oct, and more preferably OSu or OPfp;
a mixed anhydride; and
an acid halide, preferably OCl or OF.
   Preferred activated Peptide 1 are represented by SEQ ID NO: 10, SEQ ID NO: 11, SEQ ID NO: 12, SEQ ID NO: 13, SEQ ID NO: 14 and SEQ ID NO: 15 or SEQ ID NO: 268, SEQ ID NO: 269 and SEQ ID NO: 270. More preferred are SEQ ID NO: 10, SEQ ID NO: 13 or SEQ ID NO: 268, and particularly SEQ ID NO: 10 or SEQ ID NO: 268 and especially SEQ ID NO: 268.

Preferably in any embodiment of the present invention, during the coupling reaction of Peptide 1 with Peptide 2 as well as during the coupling reactions used to prepare Peptides 1 and 2, the amino acids are protected as necessary at the side chains with acid-cleavable protecting groups. In particular, the amino acid residues His, Thr, Ser, Asp, Tyr, Glu, Gln and Arg are preferably protected with acid-cleavable protecting groups. Suitable amino acid protecting groups are well known in the art of peptide synthesis. In the processes of the present invention, preferred protecting groups are tBu, OtBu, Ψ^{Me,Me}pro, Trt, and Pbf. In particular, the amino acid residues are protected as follows: His(Trt), Thr(tBu), Ser⁸(tBu), Ser⁸(Trt), Ser¹¹(tBu), Ser¹¹(Trt) (preferably the protecting groups for Ser⁸ and Ser¹¹ are tBu), Asp (OtBu), Ser¹²(Ψ^{Me,Me}pro), Ser¹²(Trt), Tyr(tBu), Glu(OtBu), Gln(Trt), and Arg(Pbf). In a preferred embodiment, the amino acid residues of Peptides 1 and 2 are protected as follows: Thr(tBu), Ser⁸(tBu), Ser¹¹(tBu), Asp (OtBu), Ser¹²(Ψ^{Me,Me}pro), Tyr(tBu), Glu(OtBu), Gln(Trt), and Arg(Pbf). In another preferred embodiment, the amino acid residues of Peptides 1 and 2 are protected as follows: Thr(tBu), Ser⁸(tBu), Ser¹¹(Trt), Asp (OtBu), Ser¹²(Trt), Tyr(tBu), Glu(OtBu), Gln(Trt), and Arg(Pbf). These side chain protecting groups preferably remain intact until after the coupling of Peptide 1 to Peptide 2 has been carried out, and are typically removed in a subsequent deprotection step.

In the coupling of Peptide 1 to Peptide 2, Peptide 1 is preferably protected at the N-terminal His with a protecting group which is preferably selected from the group consisting of Boc, Fmoc or Cbz, and more preferably Boc. The N-terminal His protection may be removed along with the side chain protecting groups, and the resin where present, to form Liraglutide.

The coupling of Peptide 1 with Peptide 2 according to any embodiment of the present invention may additionally or alternatively be conducted in the presence of a coupling agent. Coupling agents that are customarily used in peptide syntheses may be employed. These include BOP, AOP, PyBOP, PyAOP, HBTU, HATU, HCTU, HBPyU, HAPyU, TFFH, TBTU, BTFFH, EDC-HCl, PyBrop, DPPA, BOP—Cl, DCC, DIC, DEPC, EEDQ, IIDQ, CIP, PfTU, PfPU, BroP and CDI. TBTU and DIC are preferred coupling agents.

Preferably, in the process according to the invention, the Peptide 1 that is coupled to Peptide 2 has the sequence:

[SEQ ID NO: 6]

P1-His(P)-Ala-Glu(P)-Gly-O-P2

wherein P1 represents a protecting group for the N-terminal of His (preferably Boc, Fmoc or Cbz, and more particularly Boc), each P represents side chain protecting groups which may be the same or different, and P2 is selected from: H (i.e. the carboxylic acid of the Gly⁴ residue is unsubstituted, and thus contains a free —OH group), or a solid support (preferably a CTC resin), or P2 represents an activated carboxylic ester of the Gly⁴ residue (preferably Su, Bt of Pfp, and more preferably Pfp esters). Preferably P1 represents Boc, Fmoc or CBz (more preferably Boc) and P2 represents H, Su, Bt or a CTC resin.
   More preferably, Peptide 1 is selected from the following:

[SEQ ID NO: 7]

Boc-His(Trt)-Ala-Glu(OtBu)-Gly-OH

[SEQ ID NO: 8]

Fmoc-His(Trt)-Ala-Glu(OtBu)-Gly-OH

[SEQ ID NO: 9]

Cbz-His(Trt)-Ala-Glu(OtBu)-Gly-OH

[SEQ ID NO: 10]

Boc-His(Trt)-Ala-Glu(OtBu)-Gly-OSu

[SEQ ID NO: 11]

Fmoc-His(Trt)-Ala-Glu(OtBu)-Gly-OSu

[SEQ ID NO: 12]

Cbz-His(Trt)-Ala-Glu(OtBu)-Gly-OSu

[SEQ ID NO: 13]

Boc-His(Trt)-Ala-Glu(OtBu)-Gly-OBt

[SEQ ID NO: 14]

Fmoc-His(Trt)-Ala-Glu(OtBu)-Gly-OBt

[SEQ ID NO: 15]

Cbz-His(Trt)-Ala-Glu(OtBu)-Gly-OBt

or Peptide 1 can be selected from the following:

[SEQ ID NO: 268]

Boc-His(Trt)-Ala-Glu(OtBu)-Gly-OPfp

[SEQ ID NO: 269]

Fmoc-His(Trt)-Ala-Glu(OtBu)-Gly-OPfp

[SEQ ID NO: 270]

Cbz-His(Trt)-Ala-Glu(OtBu)-Gly-OPfp.

More preferably, Peptide 1 is selected from the following:

[SEQ ID NO: 7]

Boc-His(Trt)-Ala-Glu(OtBu)-Gly-OH

[SEQ ID NO: 8]

Fmoc-His(Trt)-Ala-Glu(OtBu)-Gly-OH

[SEQ ID NO: 10]

Boc-His(Trt)-Ala-Glu(OtBu)-Gly-OSu

[SEQ ID NO: 11]

Fmoc-His(Trt)-Ala-Glu(OtBu)-Gly-OSu

or particularly, Peptide 1 can be selected from the following:

[SEQ ID NO: 268]

Boc-His(Trt)-Ala-Glu(OtBu)-Gly-OPfp

[SEQ ID NO: 269]

Fmoc-His(Trt)-Ala-Glu(OtBu)-Gly-OPfp

especially

[SEQ ID NO: 268]

Boc-His(Trt)-Ala-Glu(OtBu)-Gly-OPfp.

The coupling of Peptide 1 with Peptide 2 may be conducted as a solid phase synthesis, whereby Peptide 2 is conjugated to a solid support, which can be an acid-cleavable resin, preferably a polystyrene-based resin, and more preferably a Wang resin. Thus, in a preferred embodiment wherein the coupling of Peptide 1 with Peptide 2 is conducted in the solid phase, Peptide 2 is conjugated to a Wang resin.

When the coupling of Peptide 1 with Peptide 2 is conducted in the solid phase, such as on a Wang resin, the Gly carboxylic acid in Peptide 1 need not be preactivated by derivatisation into an activated carboxylic acid group (i.e. in the form of an isolated activated ester). However, the coupling may be conducted in the presence of a coupling agent (i.e. in situ activation), such as those typically employed in peptide coupling reactions. Preferred coupling agents include BOP, AOP, PyBOP, PyAOP, HBTU, HATU, HCTU, HBPyU, HAPyU, TFFH, TBTU, BTFFH, EDC-HCl, PyBrop, DPPA, BOP—Cl, DCC, DIC, DEPC, EEDQ, IIDQ, CIP, PfTU, PfPU, BroP and CDI, with TBTU and DIC (e.g. DIC/HOBt) being particularly preferred.

For solid state synthesis on a resin, such as a Wang resin, Peptide 1 is preferably selected from:

[SEQ ID NO: 7]

Boc-His(Trt)-Ala-Glu(OtBu)-Gly-OH

[SEQ ID NO: 8]

Fmoc-His(Trt)-Ala-Glu(OtBu)-Gly-OH

with

[SEQ ID NO: 7]

Boc-His(Trt)-Ala-Glu(OtBu)-Gly-OH

being particularly preferred.

In another preferred embodiment, the coupling of Peptide 1 with Peptide 2 may also advantageously be conducted in the liquid phase, whereby no solid support is used. In the liquid phase synthesis according to this embodiment, Peptide 1 is preferably activated, i.e. the Gly carboxylic acid in Peptide 1 is reacted to form an activated carboxylic acid derivative in order to facilitate the coupling reaction. Preferably the activated carboxylic acid group can be an activated ester (preferably wherein the activated ester is selected from the group consisting of OSu, OPfp, OBt, OAt, ODhbt, ONB, OPht, ONP, ODNP, Ot, Oct, and more preferably OSu, OBt, or OPfp ester, and most preferably OPfp); a mixed anhydride; and an acid halide (preferably OCl or OF). These activated derivatives are typically isolated before the coupling reaction. Particularly preferred Peptide 1 fragments for liquid phase synthesis are the following activated esters:

[SEQ ID NO: 10]

Boc-His(Trt)-Ala-Glu(OtBu)-Gly-OSu

[SEQ ID NO: 11]

Fmoc-His(Trt)-Ala-Glu(OtBu)-Gly-OSu

[SEQ ID NO: 12]

Cbz-His(Trt)-Ala-Glu(OtBu)-Gly-OSu

[SEQ ID NO: 13]

Boc-His(Trt)-Ala-Glu(OtBu)-Gly-OBt

[SEQ ID NO: 14]

Fmoc-His(Trt)-Ala-Glu(OtBu)-Gly-OBt

[SEQ ID NO: 15]

Cbz-His(Trt)-Ala-Glu(OtBu)-Gly-OBt

or

[SEQ ID NO: 268]

Boc-His(Trt)-Ala-Glu(OtBu)-Gly-OPfp

[SEQ ID NO: 269]

Fmoc-His(Trt)-Ala-Glu(OtBu)-Gly-OPfp

[SEQ ID NO: 270]

Cbz-His(Trt)-Ala-Glu(OtBu)-Gly-OPfp,

more preferably:

[SEQ ID NO: 10]

Boc-His(Trt)-Ala-Glu(OtBu)-Gly-OSu

[SEQ ID NO: 11]

Fmoc-His(Trt)-Ala-Glu(OtBu)-Gly-OSu

[SEQ ID NO: 13]

Boc-His(Trt)-Ala-Glu(OtBu)-Gly-OBt

[SEQ ID NO: 14]

Fmoc-His(Trt)-Ala-Glu(OtBu)-Gly-OBt

or

[SEQ ID NO: 268]

Boc-His(Trt)-Ala-Glu(OtBu)-Gly-OPfp

[SEQ ID NO: 269]

Fmoc-His(Trt)-Ala-Glu(OtBu)-Gly-OPfp

with

[SEQ ID NO: 10]

Boc-His(Trt)-Ala-Glu(OtBu)-Gly-OSu

[SEQ ID NO: 13]

Boc-His(Trt)-Ala-Glu(OtBu)-Gly-OBt

or

[SEQ ID NO: 268]

Boc-His(Trt)-Ala-Glu(OtBu)-Gly-OPfp

being especially preferred. Boc-His(Trt)-Ala-Glu(OtBu)-Gly-OPfp—[SEQ ID NO: 268] is a particularly preferred Peptide 1 fragment.

The Peptide 2 fragment that is to be coupled to Peptide 1 according to any embodiment of the present invention is preferably represented by the amino acid sequence:

[SEQ ID NO: 19]

Thr-Phe-Thr-Ser-Asp-Val-Ser-Ser-Tyr-Leu-Glu-Gly-
Gln-Ala-Ala-Lys-Glu-Phe-Ile-Ala-Trp-Leu-Val-Arg-
Gly-Arg-Gly

which corresponds to the sequence (5-31) of the liraglutide backbone. In particular, Peptide 2 is preferably represented by:
(i) Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly [SEQ ID NO: 19], including:

C1.

[SEQ ID NO: 20]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(P)-Glu(P)-Phe-Ile-Ala-Trp-Leu-Val-Arg(P)-Gly-
Arg(P)-Gly-OP2

wherein P1 represents H, or a protecting group for the N-terminal of Thr (preferably Fmoc or Cbz and more preferably Fmoc), each P represents side chain protecting groups which may be the same or different, and P2 is selected from H (i.e. the carboxylic acid of the Gly³¹ residue is unsubstituted, and thus contains a free —OH group), or a solid support, preferably a CTC or Wang resin;
(ii) Thr-Phe-Thr-Ser-Asp-Val-Ser-Ser-Tyr-Leu-Glu-Gly-Gln-Ala-Ala-Lys(Glu)-Glu-Phe-Ile-Ala-Trp-Leu-Val-Arg-Gly-Arg-Gly [SEQ ID NO: 21], including:

C2.

[SEQ ID NO: 22]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(Glu-P)-Glu(P)-Phe-Ile-Ala-Trp-Leu-Val-Arg(P)-
Gly-Arg(P)-Gly-OP2

wherein P1 represents H, or a protecting group for the N-terminal of Thr (preferably Fmoc or Cbz and more preferably Fmoc), each P represents side chain protecting groups which may be the same or different, and P2 is selected from H (i.e. the carboxylic acid of the Gly³¹ residue is unsubstituted, and thus contains a free —OH group), or a solid support, preferably a CTC or Wang resin;
or

C3.

[SEQ ID NO: 23]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(P1-Glu-P)-Glu(P)-Phe-Ile-Ala-Trp-Leu-Val-
Arg(P)-Gly-Arg(P)-Gly-OP2

wherein P1 represents H, or N-terminal protecting groups for the N-Thr and/or Glu wherein P1 may be the same or different (preferably for Thr, P1 is Fmoc or Cbz and more preferably Fmoc, and preferably for Glu, P1 is Trt), each P represents side chain protecting groups which may be the same or different, and P2 is selected from H (i.e. the carboxylic acid of the Gly³¹ residue is unsubstituted, and thus contains a free —OH group), or a solid support, preferably a CTC or Wang resin;
and
(iii) Thr-Phe-Thr-Ser-Asp-Val-Ser-Ser-Tyr-Leu-Glu-Gly-Gln-Ala-Ala-Lys(Pal-Glu)-Glu-Phe-Ile-Ala-Trp-Leu-Val-Arg-Gly-Arg-Gly [SEQ ID NO: 24], including:

C4.

[SEQ ID NO: 25]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(Pal-Glu-P)-Glu(P)-Phe-Ile-Ala-Trp-Leu-Val-
Arg(P)-Gly-Arg(P)-Gly-OP2

wherein P1 represents H, or a protecting group for the N-terminal of Thr (preferably Fmoc or Cbz and more preferably Fmoc), each P represents side chain protecting groups which may be the same or different, and P2 is selected from H (i.e. the carboxylic acid of the Gly³¹ residue is unsubstituted, and thus contains a free —OH group), or a solid support, preferably a CTC or Wang resin.
   Especially preferred Peptide 2 fragments for the coupling reaction with Peptide 1 according to the present invention are:

[SEQ ID NO: 33]

Thr(tBu)-Phe-Thr(tBu)-Ser(tBu)-Asp(OtBu)-Val-
Ser(tBu)-Ser(Ψ^{Me,Me}pro)-Tyr(tBu)-
Leu-Glu(OtBu)-Gly-Gln(Trt)-Ala-Ala-Lys(Pal-Glu-
OtBu)-Glu(OtBu)-Phe-Ile-Ala-Trp-Leu-Val-
Arg(Pbf)-Gly-Arg(Pbf)-Gly-O-Wang resin

or

[SEQ ID NO: 278 or SEQ ID NO: 316]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-
Gly-O-Wang resin

for the solid state synthesis, and

[SEQ ID NO: 31]

Thr(tBu)-Phe-Thr(tBu)-Ser(tBu)-Asp(OtBu)-Val-
Ser(tBu)-Ser(Ψ^{Me,Me}pro)-Tyr(tBu)-
Leu-Glu(OtBu)-Gly-Gln(Trt)-Ala-Ala-Lys(Pal-Glu-
OtBu)-Glu(OtBu)-Phe-Ile-Ala-Trp-Leu-Val-
Arg(Pbf)-Gly-Arg(Pbf)-
Gly-OH

[SEQ ID NO: 276 or SEQ ID NO: 309]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-
Phe-Ile-Ala-Trp-Leu-Val-Arg(Pbf)-Gly-
Arg(Pbf)-Gly-OH

(i.e. wherein Peptide 2 is not present on a resin) for the liquid phase synthesis.

In a preferred embodiment, the present invention encompasses a process for preparing Liraglutide comprising:

(i) coupling a Peptide 1 having the formula selected from the group consisting of:

[SEQ ID NO: 7]

Boc-His(Trt)-Ala-Glu(OtBu)-Gly-OH

[SEQ ID NO: 8]

Fmoc-His(Trt)-Ala-Glu(OtBu)-Gly-OH

with a Peptide 2 having the formula:

[SEQ ID NO: 33]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-Gln(Trt)-Ala-Ala-Lys(Pal-Glu-OtBu)-
Glu(OtBu)-Phe-Ile-Ala-Trp-Leu-Val-Arg(Pbf)-Gly-
Arg(Pbf)-Gly-O-Wang resin,

or

[SEQ ID NO: 278 or SEQ ID NO: 316]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-
Gly-O-Wang resin,

and
(ii) removing the protecting groups and resin to form liraglutide, and optionally
(iii) purifying the liraglutide.
Preferably in this embodiment, Peptide 1 is:

[SEQ ID NO: 7]

Boc-His(Trt)-Ala-Glu(OtBu)-Gly-OH

In another preferred embodiment, the present invention encompasses a process for preparing liraglutide comprising:
(i) coupling a Peptide 1 having the formula selected from the group consisting of

[SEQ ID NO: 10]

Boc-His(Trt)-Ala-Glu(OtBu)-Gly-OSu,

[SEQ ID NO: 11]

Fmoc-His(Trt)-Ala-Glu(OtBu)-Gly-OSu,

[SEQ ID NO: 13]

Boc-His(Trt)-Ala-Glu(OtBu)-Gly-OBt,

and

[SEQ ID NO: 14]

Fmoc-His(Trt)-Ala-Glu(OtBu)-Gly-OBt,

or Peptide 1 has the formula:

[SEQ ID NO: 268]

Boc-His(Trt)-Ala-Glu(OtBu)-Gly-OPfp

[SEQ ID NO: 269]

Fmoc-His(Trt)-Ala-Glu(OtBu)-Gly-OPfp

with a Peptide 2 having the formula:

[SEQ ID NO: 31]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-Gln(Trt)-Ala-Ala-Lys(Pal-Glu-OtBu)-
Glu(OtBu)-Phe-Ile-Ala-Trp-Leu-Val-Arg(Pbf)-Gly-
Arg(Pbf)-Gly-OH,

or

[SEQ ID NO: 276 or SEQ ID NO: 309]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-OH,

wherein the coupling is carried out in liquid phase,
(ii) removing the protecting groups to form liraglutide, and optionally
(iii) purifying the liraglutide.

In this embodiment, Peptide 1 is preferably:

[SEQ ID NO: 10]

Boc-His(Trt)-Ala-Glu(OtBu)-Gly-OSu,

and

[SEQ ID NO: 13]

Boc-His(Trt)-Ala-Glu(OtBu)-Gly-OBt,

and more preferably:

[SEQ ID NO: 10]

Boc-His(Trt)-Ala-Glu(OtBu)-Gly-OSu.

Alternatively, in this embodiment, Peptide 1 is preferably:

[SEQ ID NO: 268]

Boc-His(Trt)-Ala-Glu(OtBu)-Gly-OPfp

or

[SEQ ID NO: 269]

Fmoc-His(Trt)-Ala-Glu(OtBu)-Gly-OPfp,

and most preferably Peptide 1 is:

[SEQ ID NO: 268]

Boc-His(Trt)-Ala-Glu(OtBu)-Gly-OPfp.

In the coupling of Peptide 1 with Peptide 2 in accordance with any embodiment of the present invention, Peptide 1 preferably contains less than 4%, less than 2%, less than 1%, less than 0.5%, less than 0.2% or less than 0.1% of the corresponding D-His analogue of Peptide 1, i.e. Peptide 1 wherein the terminal His group has D-configuration. Alternatively or additionally, Peptide 1 preferably contains less than 4%, less than 2%, less than 1%, less than 0.5%, less than 0.2% or less than 0.1% of the diglycine analogue of Peptide 1, i.e. a peptide corresponding to Peptide 1, but having the amino acid sequence His-Ala-Glu-Gly-Gly [SEQ ID NO: 115].

In any embodiment of the present invention, Peptide 1 is preferably prepared by:
(i) sequential coupling of amino acids on a resin, preferably CTC resin,
(ii) cleaving the Peptide 1 from the resin, and
(iii) optionally purifying the Peptide 1.

Preferably, step (ii) cleaving Peptide 1 from the resin using an acid in the presence of at least one organic solvent,
(ii) neutralizing the reaction mixture and/or washing with water and/or an aqueous base;
(iii) concentrating the mixture containing Peptide 1 to produce a concentrated solution of Peptide 1 in the organic solvent, and
(iv) precipitating Peptide 1 from the resulting solution by addition of an antisolvent.
   In any embodiment of the present invention, Peptide 1 is purified before the coupling reaction with Peptide 2 by precipitation from a solution in organic solvent with an antisolvent. Particularly, Peptide 1 is purified to remove the corresponding D-His impurity before the coupling reaction with Peptide 2. Preferably, the purification of Peptide 1 comprises precipitating Peptide 1 from a solution comprising at least one organic solvent, with an antisolvent. The precipitation can be repeated one or more times in order to further purify Peptide 1. Alternatively, Peptide 1 may be purified by other techniques including recrystallization, HPLC, or a combination thereof.

Preferred organic solvents from which Peptide 1 is precipitated from are halogenated hydrocarbons, preferably a bromo- or chloroalkane, and more preferably a brominated or chlorinated hydrocarbon, such as a brominated or chlorinated C₁-C₆ hydrocarbon, or brominated or chlorinated C₁-C₄ hydrocarbon, or mixtures thereof. More preferably, the organic solvent is selected from the group consisting of dichloromethane, dibromomethane, and ethylene dichloride or mixtures thereof. Dichloromethane is a preferred organic solvent.

Preferred antisolvents used to precipitate Peptide 1 comprise an ether and/or a hydrocarbon, or mixtures thereof.

Preferably, the antisolvent is a straight chain or branched C₄-C₈ dialkyl ether preferably a C₄-C₆ dialkyl ether, more preferably diethyl ether methyl tert-butyl ether (MTBE) or mixtures thereof. More preferably, the antisolvent is methyl tert-butyl ether (MTBE).

The antisolvent may also comprise a C₆-C₁₀ hydrocarbon either alone, or in a mixture with the ether. Preferably, the hydrocarbon is a C₆-C₈ hydrocarbon, more preferably hexane or petroleum ether, and most preferably is hexane.

The antisolvent is preferably MTBE alone or MTBE in combination with hexane or petroleum ether.

In a particularly preferred embodiment, Peptide 1 is purified by precipitation from a solution of Peptide 1 in a solvent comprising dichloromethane with an antisolvent comprising MTBE.

The Peptide 2 used in the coupling reaction with Peptide 1 in accordance with any embodiment of the present invention can be prepared by a convergent synthesis. In particular, the convergent synthesis of Peptide 2 preferably involves the condensation of peptide fragments containing amino acids (5-16) with amino acids (17-31) of liraglutide.

Peptide 2 can thus be prepared by coupling of Peptides 3 and 4, wherein one of Peptide 3 or Peptide 4 contains the residue:

-Lys(Pal-Glu-OX)-

wherein:
Peptide 3 and Peptide 4 together form the amino acid sequence of Peptide 2,
X represents H or a protecting group for the Glu carboxylic acid group,
one or more of the amino acid residues in Peptide 3 and Peptide 4 are optionally protected, and
Peptide 3 or Peptide 4, preferably Peptide 4, is optionally conjugated to a solid support.

Preferably, Peptide 2 is prepared by coupling of Peptide 3 with Peptide 4, wherein the amino acid sequence in Peptide 3 is:

[SEQ ID NO: 42]

Thr-Phe-Thr-Ser-Asp-Val-Ser-Ser-Tyr-Leu-Glu-Gly-OH

wherein the Gly carboxylic acid group is optionally activated, preferably as an OSu or OPfp (more preferably OPfp) ester,
and the amino acid sequence in Peptide 4 is:

[SEQ ID NO: 52]

Gln-Ala-Ala-Lys(PalA-Glu-OX)-Glu-Phe-Ile-Ala-Trp-
Leu-Val-Arg-Gly-Arg-Gly-OH

wherein Peptide 4 is optionally conjugated to a resin at the terminal Gly-OH, preferably wherein the resin is a Wang resin.

As mentioned above in respect of Peptide 2, one or more of the amino acid residues in Peptide 3 and Peptide 4 which are coupled to form Peptide 2, are protected with acid-cleavable protecting groups. Preferred acid-cleavable protecting groups for Peptides 3 and 4 are as discussed above, i.e. tBu, OtBu, Ψ^{Me,Me}pro, Trt, and Pbf. The protected amino acid residues in Peptides 3 and 4 are preferably as follows: Thr(tBu), Ser⁸ (tBu), Ser⁸(Trt), Ser¹¹(tBu), Ser¹¹(Trt), Asp (OtBu), Ser¹²(Ψ^{Me,Me}pro), Ser¹²(Trt), Tyr(tBu), Glu(OtBu), Gln(Trt), and Arg(Pbf). In a preferred embodiment, the amino acid residues of Peptides 3 and 4 are protected as follows: Thr(tBu), Ser⁸(tBu), Ser¹¹(tBu), Asp (OtBu), Ser¹²(Ψ^{Me,Me}pro), Tyr(tBu), Glu(OtBu), Gln(Trt), and Arg(Pbf). In another preferred embodiment, the amino acid residues of Peptides 3 and 4 are protected as follows: Thr(tBu), Ser⁸(tBu), Ser¹¹(Trt), Asp (OtBu), Ser¹²(Trt), Tyr(tBu), Glu(OtBu), Gln(Trt), and Arg(Pbf).

In an embodiment of the present invention, wherein the coupling of Peptides 1 and 2 are carried out in the solid phase, the preparation of Peptide 2 by coupling of Peptides 3 and 4 is also carried out in the solid phase. In this embodiment, Peptide 2 is prepared by a process comprising:

(i) coupling Peptide 3 of formula:

[SEQ ID NO: 44]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-OH,

or

[SEQ ID NO: 287]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

to Peptide 4 of formula:

[SEQ ID NO: 55 or SEQ ID NO: 128]

Gln(Trt)-Ala-Ala-Lys(Pal-Glu-OtBu)-Glu(OtBu)-Phe-
AIle-la-Trp-Leu-Val-Arg(Pbf)-Gly-Arg(Pbf)-Gly-O-
Wang resin

and removing the Fmoc protecting group to form Peptide 2 of formula:

[SEQ ID NO: 33]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-Gln(Trt)-Ala-Ala-Lys(Pal-Glu-OtBu)-
Glu(OtBu)-Phe-Ile-Ala-Trp-Leu-Val-Arg(Pbf)-Gly-
Arg(Pbf)-Gly-O-Wang resin,

or

[SEQ ID NO: 278 or SEQ ID NO: 316]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-O-
Wang resin,

(ii) coupling the Peptide 1 of formula:

[SEQ ID NO: 7]

Boc-His(Trt)-Ala-Glu(OtBu)-Gly-OH

to Peptide 2 to form an optionally protected liraglutide sequence,
(iii) deprotecting and removing the resin to form liraglutide, and optionally
(iv) purifying the liraglutide.
The Peptide 4 in this embodiment is preferably prepared by sequential synthesis on a resin, preferably a Wang resin, using Fmoc strategy, and wherein the -Lys(Pal-Glu-OX)-residue is formed by:
(i) sequential coupling of Fmoc-Lys(Mtt)-OH or Fmoc-Lys(Mmt)-OH
(ii) selectively removing the Mtt or Mmt protecting group with acid, and coupling a Pal-Glu-OX residue to the Lys residue,
wherein step (ii) can be carried out on the partial or completed sequence of Peptide 4, i.e. step (ii) can be carried out at any stage after coupling of the residue containing Lys in step (i), e.g. immediately after step (i) or at any stage after the addition of the Ala, Ala, Gln residues forming the sequence of Peptide 4.

In one embodiment of the present invention, wherein the coupling of Peptides 1 and 2 are carried out in the liquid phase, the preparation of Peptide 2 by coupling of Peptides 3 and 4 is also carried out in liquid phase. In this embodiment, Peptide 2 can be prepared by a process comprising:

(i) liquid phase coupling of Peptide 3 of formula:

[SEQ ID NO: 45]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-OSu

or

[SEQ ID NO: 288]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OSu

or Peptide 3 of formula:

[SEQ ID NO: 289]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OPfp

or

[SEQ ID NO: 290]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-OPfp

to Peptide 4 of formula:

[SEQ ID NO: 56 or SEQ ID NO: 148]

Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-OH

and removing the Fmoc to form Peptide 2 of formula:

[SEQ ID NO: 31]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-Gly-
OH,

or

[SEQ ID NO: 276 or SEQ ID NO: 309]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-OH

(ii) coupling the Peptide 1 of formula:

[SEQ ID NO: 10]

Boc-His(Trt)-Ala-Glu(OtBu)-Gly-OSu

[SEQ ID NO: 13]

Boc-His(Trt)-Ala-Glu(OtBu)-Gly-OBt,

or the Peptide 1 of formula:

[SEQ ID NO: 268]

Boc-His(Trt)-Ala-Glu(OtBu)-Gly-OPfp

to Peptide 2 to form an optionally protected liraglutide sequence, and
(iii) deprotecting to form liraglutide, and optionally
(iv) purifying the liraglutide.

According to this embodiment, Peptide 4 may be prepared by a process involving sequential synthesis on a resin, preferably a CTC resin, using Fmoc strategy, comprising:

(i) forming the Lys(Pal-Glu-OX) residue by sequential coupling of Fmoc-Lys(Trt-Glu-OtBu)-OH
(ii) coupling one or more amino acid residues sequentially to the Lys residue to form the amino acid sequence of Peptide 4,
(iii) simultaneously removing the Trt protecting group of the Lys(Trt-Glu-OtBu) residue and cleavage of the peptide from the resin,
(iv) coupling Pal to Glu by reaction with Pal-OSu, and
(v) removing the Fmoc group.
Alternatively, Peptide 4 may be prepared by a process involving: sequential synthesis on a resin, preferably a CTC, using Fmoc strategy, comprising:
(i) forming the -Lys(Pal-Glu-OX)- residue by sequential coupling of Fmoc-Lys(Mmt)-OH or Fmoc-Lys(Mtt)-OH
(ii) coupling one or more amino acid residues sequentially to the Lys residue to form the amino acid sequence of Peptide 4,
(iii) simultaneously removing the Mmt or Mtt protecting group and cleavage of the peptide from the resin,
(iv) coupling a Pal-Glu-OX residue to the Lys residue, wherein the side chain carboxylic acid group in Glu may be activated in the form of an OSu ester or an OPfp ester, and
(v) removing the Fmoc group.

As a further alternative, Peptide 4 may be prepared by a process comprising

(i) coupling of Peptides 3 and 4A, wherein one of Peptide 3 or Peptide 4A contains the residue:

-Lys(Y-Glu-OX)-

wherein:
Peptide 3 and Peptide 4A together form the amino acid sequence of Peptide 2,
X represents H or a protecting group for the Glu carboxylic acid group,
Y represents a protecting group for the Glu amino group, and
one or more of the amino acid residues in Peptide 3 and Peptide 4A are optionally protected, and
Peptide 3 or Peptide 4A is optionally conjugated to a solid support,
   (ii)) removal of the protecting group Y and if present, cleaving the peptide from the support, and
   (iii) coupling Pal to the Glu residue to form Peptide 2, and
   (iv) removing the Fmoc group.

Peptide 2 may also be prepared by a process comprising coupling Peptide 3 with Peptide 4A, wherein the amino acid sequence in Peptide 3 is:

[SEQ ID NO: 42]

Thr-Phe-Thr-Ser-Asp-Val-Ser-Ser-Tyr-Leu-Glu-Gly

wherein the Gly carboxylic acid group is optionally activated with an OSu ester or OPfp ester,
and wherein the amino acid sequence in Peptide 4A is:

[SEQ ID NO: 51]

Gln-Ala-Ala-Lys(Y-Glu-OX)-Glu-Phe-Ile-Ala-Trp-Leu-
Val-Arg-Gly-Arg-Gly

wherein Peptide 4A is optionally conjugated to a resin at the terminal Gly residue, preferably wherein the resin is selected from a Wang resin or a CTC resin, and more preferably a CTC resin, and wherein the amino acid residues in Peptide 3 and Peptide 4A are optionally protected.

In the preferred embodiment whereby Peptide 2 is prepared by coupling Peptide 3 to Peptide 4A, the process comprises:

(i) coupling Peptide 3 of formula:

[SEQ ID NO: 44]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-OH

or

[SEQ ID NO: 287]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

to Peptide 4A of formula:

[SEQ ID NO: 59 or SEQ ID NO: 143]

Gln(Trt)-Ala-Ala-Lys(Trt-Glu-OtBu)-Glu(OtBu)-Phe-
Ile-Ala-Trp-Leu-Val-Arg(Pbf)-Gly-Arg(Pbf)-Gly-O-
CTC resin

(ii) simultaneously removing the Glu-Trt protecting group and resin
(iii) coupling the peptide product from step (ii) with Pal-OSu and removing Fmoc to form Peptide 2 of formula:

[SEQ ID NO: 31]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-Gly-
OH

or

[SEQ ID NO: 276 or SEQ ID NO: 309]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-OH

(iv) coupling the Peptide 1 of formula:

[SEQ ID NO: 10]

Boc-His(Trt)-Ala-Glu(OtBu)-Gly-OSu,

or

[SEQ ID NO: 13]

Boc-His(Trt)-Ala-Glu(OtBu)-Gly-OBt,

or the Peptide 1 of formula:

[SEQ ID NO: 268]

Boc-His(Trt)-Ala-Glu(OtBu)-Gly-OPfp

to Peptide 2 to form an optionally protected liraglutide sequence, and
(v) deprotecting to form liraglutide, and
(vi) optionally purifying the liraglutide.

The Peptide 4A is preferably prepared by sequential synthesis on a resin, preferably a CTC resin, using Fmoc strategy, and wherein the -Lys(Pal-Glu-OX)-residue is formed by sequential coupling of Fmoc-Lys(Trt-Glu-OtBu).

Peptides 3 and 4A according to these embodiments preferably contain amino acid residues which are protected as necessary, using acid-cleavable protecting groups, preferably selected from the group consisting of: tBu, OtBu, Ψ^{Me,Me}pro, Trt, and Pbf More preferably, the protected amino acid residues in Peptides 3 and 4A are: Thr(tBu), Ser⁸ (tBu), Ser⁸(Trt), Ser¹¹(tBu), Ser¹¹(Trt), Asp(OtBu), Ser¹²(Ψ^{Me,Me}pro), Ser¹²(Trt), Tyr(tBu), Glu(OtBu), Gln(Trt), and Arg(Pbf). In a preferred embodiment, according to any process of the present invention, the amino acid residues of Peptides 3 and 4A are protected as follows: Thr(tBu), Ser⁸(tBu), Ser¹¹(tBu), Lys(Trt-Glu-OtBu), Asp (OtBu), Ser¹²(Ψ^{Me,Me}pro), Tyr(tBu), Glu(OtBu), Gln(Trt), and Arg(Pbf). In another preferred embodiment, the amino acid residues of Peptides 3 and 4A are protected as follows: Thr(tBu), Ser⁸(tBu), Ser¹¹(Trt), Lys(Trt-Glu-OtBu), Asp (OtBu), Ser¹²(Trt), Tyr(tBu), Glu(OtBu), Gln(Trt), and Arg(Pbf).

Peptide 4A according to these embodiments is preferably conjugated to a resin, preferably a CTC resin.

In any embodiment of the present invention the N-terminal of Thr in Peptide 3 which is to be coupled with Peptide 4 or 4A, is protected with Fmoc or CBz, and preferably with Fmoc.

As to Peptide 4, which is to be coupled with Peptide 3 as described in any of the above embodiments, this peptide preferably contains the residue -Lys(Pal-Glu-OX)-, and is prepared by sequential synthesis on a resin, preferably a Wang resin, using Fmoc strategy (i.e. using Fmoc-protected N-terminal amino acids, following by removal of Fmoc before coupling of the next Fmoc-protected N-terminal amino acid), and wherein the -Lys(Pal-Glu-OX)- residue is formed by:

(i) sequential coupling of Fmoc-Lys(Mtt)-OH or Fmoc-Lys(Mmt)-OH,
(ii) selectively removing the Mtt or Mmt protecting group with acid, and coupling a Pal-Glu-OX residue to the Lys residue,
wherein step (ii) can be carried out on the partial or complete sequence of Peptide 4, i.e. step (ii) can be carried out at any stage after coupling of the residue containing Lys in step (i), e.g. immediately after step (i) or at any stage after the addition of the Ala, Ala, Gln residues forming the sequence of Peptide 4.
In this embodiment, step (ii) preferably comprises coupling with Pal-Glu-OtBu wherein the side chain carboxylic acid group in Glu is optionally in the form of an activated carboxylic acid derivative, wherein the activated carboxylic acid derivative is preferably in the form of an activated ester. More preferably, the reaction is carried out with Pal-Glu-OtBu, Pal-Glu(OSu)-OtBu, Pal-Glu(OPfp)-OtBu or Pal-Glu(OBt)-OtBu, preferably Pal-Glu-OtBu.

The Peptide 4 which is to be coupled with Peptide 3 as described in any of the above embodiments, containing the residue -Lys(Pal-Glu-OX)-, can alternatively be prepared by sequential synthesis on a resin, preferably a CTC resin, using Fmoc strategy, comprising:

(i) sequential coupling of Fmoc-Lys(Trt-Glu-OtBu)-OH,
(ii) coupling amino acid residues sequentially to the Lys residue to form the amino acid sequence of Peptide 4,
(iii) simultaneously removing the Trt protecting group of the Lys(Trt-Glu-OtBu) residue and cleavage of the peptide from the resin, and
(iv) coupling Pal to Glu to form Peptide 4.
In this embodiment, step (iv) can comprise reaction with palmitic acid, preferably wherein the carboxylic acid group in the palmitic acid (Pal-OH) is in the form of an activated carboxylic acid derivative, preferably in the form of an activated ester. More preferably, step (iv) involves reaction with Pal-OSu, Pal-OPfp or Pal-OBt, preferably Pal-OSu or Pal-OPfp.

The Peptide 4 containing the residue -Lys(Pal-Glu-OX)-, which is to be coupled with Peptide 3 as described in any of the above embodiments, can alternatively be prepared by sequential synthesis on a resin, preferably a CTC, using Fmoc strategy, comprising:

(i) sequential coupling of Fmoc-Lys(Mmt)-OH or Fmoc-Lys(Mtt)-OH [preferably Fmoc-Lys(Mmt)-OH],
(ii) coupling one or more amino acid residues sequentially to the Lys residue to form the amino acid sequence of Peptide 4,
(iii) simultaneously removing the Mmt or Mtt protecting group (preferably Mmt protecting group) and cleavage of the peptide from the resin, and
(iv) coupling a Pal-Glu-OX residue to the Lys residue, wherein the side chain carboxylic acid group in the Glu may be in the form of an activated carboxylic acid derivative.
In accordance with this embodiment, step (iv) preferably comprises reaction with Pal-Glu-OtBu wherein the side chain carboxylic acid group in Glu is preferably in the form of an activated carboxylic acid derivative, wherein the activated carboxylic acid derivative is preferably an activated ester. More preferably, step (iv) is carried out by reaction with Pal-Glu(OSu)-OtBu, Pal-Glu(OPfp)-OtBu or Pal-Glu(OBt)-OtBu, preferably Pal-Glu(OSu)-OtBu.

In any of the above described alternative embodiments for preparing Peptide 4, the amino acid sequence of Peptide 4 is preferably:

[SEQ ID NO: 51]

Gln-Ala-Ala-Lys(Pal-Glu-OX)-Glu-Phe-Ile-Ala-Trp-
Leu-Val-Arg-Gly-Arg-Gly-OH

wherein one or more amino acid residues are optionally protected, and preferably wherein Peptide 4 is:

[SEQ ID NO: 56 or SEQ ID NO: 148]

Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-OH

wherein Peptide 4 may be conjugated to a resin, preferably a Wang resin.

In any of the above-described embodiments Peptide 4A which is to be coupled to Peptide 3 to prepare Peptide 2, Peptide 4A preferably contains the residue -Lys(Y-Glu-OX)-, wherein Peptide 4A is prepared by sequential synthesis on a resin, preferably a CTC resin, using Fmoc strategy, and wherein the -Lys(Y-Glu-OX)-residue is formed by:

(i) sequential coupling of Fmoc-Lys(Trt-Glu-OtBu)-OH,
(ii) coupling amino acid residues sequentially to the Lys residue to form the amino acid sequence of Peptide 4A.
In this embodiment, a preferred Peptide 4A has the amino acid sequence:

[SEQ ID NO: 51]

Gln-Ala-Ala-Lys(Y-Glu-OX)-Glu-Phe-Ile-Ala-Trp-Leu-
Val-Arg-Gly-Arg-Gly-OH

wherein one or more amino acids are optionally protected. More preferably, Peptide 4A is:
Gln(Trt)-Ala-Ala-Lys(Trt-Glu-OtBu)-Glu(OtBu)-Phe-Ile-Ala-Trp-Leu-Val-Arg(Pbf)-Gly-Arg(Pbf)-Gly-OH
which is preferably conjugated to a resin, preferably a CTC resin [SEQ ID NO: 59 or SEQ ID NO: 143].

The Peptide 3 which is coupled to Peptide 4 or 4A to prepare Peptide 2, in accordance with any embodiment disclosed herein, preferably has the amino acid sequence:

[SEQ ID NO: 42]

Thr-Phe-Thr-Ser-Asp-Val-Ser-Ser-Tyr-Leu-Glu-Gly

wherein
the Gly carboxylic acid group may be in the form of an activated carboxylic acid derivative, and
   one or more amino acids are optionally protected, and preferably wherein Peptide 3 is:

[SEQ ID NO: 44]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-OH

or

[SEQ ID NO: 287]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

wherein the Gly carboxylic acid group may be in the form of an activated carboxylic acid derivative. More preferably, Peptide 3 is:

[SEQ ID NO: 141]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-OH

or

[SEQ ID NO: 305]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

Preferably, the N-terminal of Thr(tBu) in Peptide 3 is protected with Boc or Fmoc (more preferably Fmoc), and optionally the Gly carboxylic acid group is reacted to form an activated carboxylic acid derivative, preferably an activated ester. Preferably, the Gly carboxylic acid group is reacted to form an activated carboxylic acid derivative when the coupling of Peptide 3 with Peptide 4 is to be conducted in the liquid phase (i.e. in the absence of a resin). Preferably, when Peptide 3 is to be coupled with Peptide 4 in the liquid phase, the Gly carboxylic acid group in Peptide 3 is activated as an ester, preferably as the OSu ester or as the OPfp ester, i.e.:

[SEQ ID NO: 45]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-OSu

or

[SEQ ID NO: 288]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OSu

or

[SEQ ID NO: 289]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OPfp

or

[SEQ ID NO: 290]

Fmoc-Thr(tBu)-Phe-Thr(tBu)-Ser(tBu)-Asp(OtBu)-
Val-Ser(tBu)-Ser(Ψ^{Me,Me}pro)-Tyr(tBu)-
Leu-Glu(OtBu)-Gly-OPfp.

In a further embodiment, the Peptide 2 which is to be coupled with Peptide 1 in accordance with any embodiments of the invention as described above, may be prepared by a process comprising:
(i) coupling of Peptides 3 and 4B, wherein one of Peptide 3 or Peptide 4B contains the residue:

-Lys²⁰(Y)

wherein:
   Peptide 3 and Peptide 4B together form the amino acid sequence of Peptide 2 wherein the residue at position 20 is Lys(Y),
   Y represents a protecting group for the Glu amino group selected from Mtt or Mmt,
   one or more of the amino acid residues in Peptide 3 and 4B are optionally protected, and
   Peptide 3 or Peptide 4B is optionally conjugated to a solid support,
(ii) optionally cleaving the peptide from the support,
(iii) removing the protecting group Y,
(iv) coupling a Pal-Glu-OX residue to the Lys, wherein X is H or a protecting group for the Glu carboxylic acid group and wherein the side chain carboxylic acid group in Glu is optionally in the form of an activated carboxylic acid derivative, and optionally,
(v) deprotecting and removing the peptide from the support.

In this process, the Pal-Glu group is attached to the Lys residue after the sequence of amino acids forming the Peptide 2 backbone is completed. The coupling of Peptide 3 with 4B may be carried out as a solid phase synthesis, or as a liquid phase synthesis.

In this process, the amino acid sequence in Peptide 3 is preferably:
Thr-Phe-Thr-Ser-Asp-Val-Ser-Ser-Tyr-Leu-Glu-Gly-OH [SEQ ID NO: 42]
   wherein the Gly carboxylic acid group is optionally in the form of an activated ester derivative, preferably an OSu ester or an OPfp ester,
   and the amino acid sequence in Peptide 4B is:

[SEQ ID NO: 50]

Gln-Ala-Ala-Lys(Y)-Glu-Phe-Ile-Ala-Trp-
Leu-Val-Arg-Gly-Arg-Gly-OH

wherein Y is Mtt or Mmt, preferably Mmt,
and wherein Peptide 4B is optionally conjugated to a resin at the terminal Gly residue, preferably wherein the resin is a Wang resin and wherein the amino acid residues in Peptide 3 and Peptide 4B are optionally protected. Preferably, the amino acid residues in Peptides 3 and 4B are protected with acid-cleavable protecting groups. Preferred acid-cleavable protecting groups are selected from the group consisting of: tBu, OtBu, Ψ^{Me,Me}pro, Trt, Mmt, Mtt and Pbf More preferably, the protected amino acid residues in Peptides 3 and 4B are as follows: Thr(tBu), Ser(tBu) or Ser (Trt), Asp (OtBu), Ser(Ψ^{Me,Me}pro), Lys(Mmt) or Lys(Mtt), Tyr(tBu), Glu(OtBu), Gln(Trt), and Arg(Pbf). In a preferred embodiment, according to any process of the present invention, the amino acid residues of Peptides 3 and 4B are protected as follows: Thr(tBu), Ser⁸(tBu), Ser¹¹(tBu), Lys(Mmt) or Lys(Mtt) [preferably Lys(Mmt)], Asp (OtBu), Ser¹²(Ψ^{Me,Me}pro), Tyr(tBu), Glu(OtBu), Gln(Trt), and Arg(Pbf). In another preferred embodiment, the amino acid residues of Peptides 3 and 4B are protected as follows: Thr(tBu), Ser⁸(tBu), Ser¹¹(Trt), Lys(Mtt) or Lys(Mmt) [preferably Lys(Mmt)], Asp (OtBu), Ser¹²(Trt), Tyr(tBu), Glu(OtBu), Gln(Trt), and Arg(Pbf).

A preferred process according to this embodiment, comprises:
(i) coupling Peptide 3 of formula:

[SEQ ID NO: 44]

Fmoc-Thr(tBu)-Phe-Thr(tBu)-Ser(tBu)-Asp(OtBu)-
Val-Ser(tBu)-Ser(Ψ^{Me,Me}pro)-Tyr(tBu)-
Leu-Glu(OtBu)-Gly-OH,

or

[SEQ ID NO: 287]
Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

with Peptide 4B of formula:

[SEQ ID NO: 75 or SEQ ID NO: 77]

Gln(Trt)-Ala-Ala-Lys(Y)-Glu(OtBu)-Phe-Ile-Ala-
Trp-Leu-Val-Arg(Pbf)-Gly-Arg(Pbf)-Gly-O-Wang resin

wherein Y is Mmt or Mtt (preferably Mmt),
(ii) removing the protecting group Y,
(iii) coupling a Pal-Glu-OX residue to the Lys, and
(iv) removing Fmoc and cleaving the peptide from the support to form Peptide 2.
   The Peptide 4B may be prepared by sequential synthesis on a resin, preferably a Wang resin, using Fmoc strategy, wherein the Lys(Y) residue is formed by sequential coupling of Fmoc-Lys(Y)-OH. Preferably, step (iii) comprises reaction with Pal-Glu-OX, wherein X represents a protecting group for the Glu carboxylic acid group, and preferably wherein X represents OtBu.

Alternatively, Peptide 2 may be prepared by coupling Peptides 3 and 4B as defined above in liquid phase, i.e. wherein Peptide 3 and 4B are not conjugated to a solid support. In this embodiment, the process comprises:
(i) liquid phase coupling of Peptide 3 of formula:

[SEQ ID NO: 44]

Fmoc-Thr(tBu)-Phe-Thr(tBu)-Ser(tBu)-Asp(OtBu)-
Val-Ser(tBu)-Ser(Ψ^{Me,Me}pro)-Tyr(tBu)-Leu-
Glu(OtBu)-Gly-OH

or

[SEQ ID NO: 287]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

wherein the Gly carboxylic acid group is preferably in the form of an activated derivative, preferably an OSu ester or OPfp ester,
with Peptide 4B of formula:

[SEQ ID NO: 74 or SEQ ID NO: 76]

Gln(Trt)-Ala-Ala-Lys(Y)-Glu(OtBu)-Phe-Ile-Ala-
Trp-Leu-Val-Arg(Pbf)-Gly-Arg(Pbf)-Gly-OH

wherein Y is Mmt or Mtt (preferably Mmt),
(ii) removing the protecting group Y,
(iii) coupling a Pal-Glu-OX residue to the Lys residue by reaction with Pal-Glu-OX, wherein the side chain carboxylic acid group in Glu is in the form of an activated carboxylic acid derivative.

The coupling step (iii) is preferably conducted using a Pal-Glu-OX residue in which the side chain of the Glu carboxylic acid is in the form of an activated derivative, preferably an activated ester. Preferably, step (iii) comprises reaction with Pal-Glu(OSu)-OtBu, Pal-Glu(OPfp)-OtBu, Pal-Glu(OBt)-OtBu, preferably Pal-Glu(OSu)-OtBu. Preferably, Peptide 4B is prepared by sequential synthesis on a resin, preferably a CTC resin, using Fmoc strategy, wherein the Lys(Y) residue is formed by sequential coupling of Fmoc-Lys(Y)-OH, and removing the Fmoc group and cleaving the peptide from the resin to form Peptide 4B.

In any embodiment of the present invention, the Peptide 3 which is to be coupled to Peptide 4, Peptide 4A or Peptide 4B, may be prepared by a two fragment coupling on a resin, followed by cleavage of the peptide from the resin. Preferably, Peptide 3 is prepared by coupling a peptide containing amino acids (5-12) of liraglutide with a peptide containing amino acids (13-16) of liraglutide. Thus, Peptide 3 for use in accordance with preferred embodiments of the present invention is prepared by a process comprising:

(i) coupling a Peptide 5 containing the optionally protected amino acid sequence:

[SEQ ID NO: 78]

Thr-Phe-Thr-Ser-Asp-Val-Ser-Ser

with a Peptide 6 containing the optionally protected amino acid sequence:

[SEQ ID NO: 84]

Tyr-Leu-Glu-Gly-OH

which is conjugated to a resin, and
(ii) cleaving the Peptide 3 from the resin.

More preferably, Peptide 3 may be prepared by:

(i) coupling of Peptide 5 having the formula:

[SEQ ID NO: 82]

Fmoc-Thr(tBu)-Phe-Thr(tBu)-Ser(tBu)-Asp(OtBu)-
Val-Ser(tBu)-Ser(Ψ^{Me,Me}pro)

or

[SEQ ID NO: 300]

Fmoc-Thr(tBu)-Phe-Thr(tBu)-Ser(tBu)-Asp(OtBu)-
Val-Ser(Trt)-Ser(Trt)

with a Peptide 6:

Tyr(tBu)-Leu-Glu(OtBu)-Gly-OH

which is conjugated to a CTC resin (SEQ ID NO: 88 or SEQ ID NO: 163) [e.g. Fmoc-Tyr(tBu)-Leu-Glu(OtBu)-Gly-O-CTC resin (SEQ ID NO: 86) or Cbz-Tyr(tBu)-Leu-Glu(OtBu)-Gly-O-CTC resin (SEQ ID NO: 87) or (preferably) H-Tyr(tBu)-Leu-Glu(OtBu)-Gly-O-CTC resin (SEQ ID NO: 88) and
   (ii) cleaving the peptide from the resin.
   The coupling of Peptide 5 sub-fragment with Peptide 6 sub-fragment to prepare the Peptide 3 fragment is advantageous since couplings to prepare Peptide 3 by condensation at different amino acid residues may lead to racemization. For example, racemization during fragment coupling to form Peptide 3 can be avoided by using pseudoproline at the C-terminus of Ser in Peptide 5. In another aspect, the present invention provides fragmental peptides of Liraglutide, selected from the group consisting of:

(i)
[SEQ ID NO: 118]
His(Trt)-Ala-Glu(OtBu)-Gly-OH,
(ii)
[SEQ ID NO: 119]
His(Trt)-Ala-Glu(OtBu)-Gly-Osu,
(iii)
[SEQ ID NO: 120]
His(Trt)-Ala-Glu(OtBu)-Gly-OBt,
(iv)
[SEQ ID NO: 121]
His(Trt)-Ala-Glu(OtBu)-Gly-O,
(v)
[SEQ ID NO: 122]
His(Trt)-Ala-Glu(OtBu)-Gly-O-CTC resin,
(vi)
[SEQ ID NO: 123]
Lys(Mtt)-Glu(OtBu)-Phe-Ile-Ala-Trp-Leu-Val-Arg(Pbf)-Gly-
Arg(Pbf)-Gly-O-Wang resin,
(vii)
[SEQ ID NO: 124]
Lys(Mmt)-Glu(OtBu)-Phe-Ile-Ala-Trp-Leu-Val-Arg(Pbf)-Gly-
Arg(Pbf)-Gly-O-Wang resin,
(viii)
[SEQ ID NO: 125]
Lys-Glu(OtBu)-Phe-Ile-Ala-Trp-Leu-Val-Arg(Pbf)-Gly-Arg(Pbf)-
Gly-O-Wang resin,
(ix)
[SEQ ID NO: 126 or SEQ ID NO: 167]
Lys(Glu-OtBu)-Glu(OtBu)-Phe-Ile-Ala-Trp-Leu-Val-Arg(Pbf)-Gly-
Arg(Pbf)-Gly-O-Wang resin,
(x)
[SEQ ID NO: 127]
Lys(Pal-Glu-OtBu)-Glu(OtBu)-Phe-Ile-Ala-Trp-Leu-Val-Arg(Pbf)-
Gly-Arg(Pbf)-Gly-O-Wang resin,
(xi)
[SEQ ID NO: 128 or SEQ ID NO: 55]
Gln(Trt)-Ala-Ala-Lys(Pal-Glu-OtBu)-Glu(OtBu)-Phe-Ile-Ala-Trp-
Leu-Val-Arg(Pbf)-Gly-Arg(Pbf)-Gly-O-Wang resin,
(xii)
[SEQ ID NO: 129]
Ala-Lys(Mtt)-Glu(OtBu)-Phe-Ile-Ala-Trp-Leu-Val-Arg(Pbf)-Gly-
Arg(Pbf)-Gly-O-Wang re,
(xiii)
[SEQ ID NO: 130]
Ala-Lys(Mmt)-Glu(OtBu)-Phe-Ile-Ala-Trp-Leu-Val-Arg(Pbf)-Gly-
Arg(Pbf)-Gly-O-Wang re,
(xiv)
[SEQ ID NO: 131]
Ala-Lys(Trt-Glu-OtBu)-Glu(OtBu)-Phe-Ile-Ala-Trp-Leu-Val-
Arg(Pbf)-Gly-Arg(Pbf)-Gly-O-Wang re,
(xv)
[SEQ ID NO: 132]
Ala-Lys(Glu-OtBu)-Glu(OtBu)-Phe-Ile-Ala-Trp-Leu-Val-Arg(Pbf)-
Gly-Arg(Pbf)-Gly-O-Wang re,
(xvi)
[SEQ ID NO: 133]
Ala-Lys-Glu(OtBu)-Phe-Ile-Ala-Trp-Leu-Val-Arg(Pbf)-Gly-
Arg(Pbf)-Gly-O-Wang re,
(xvii)
[SEQ ID NO: 134 or SEQ ID NO: 103]
Ala-Lys(Pal-Glu-OtBu)-Glu(OtBu)-Phe-Ile-Ala-Trp-Leu-Val-
Arg(Pbf)-Gly-Arg(Pbf)-Gly-O-Wang resin,
(xviii)
[SEQ ID NO: 135]
Ala-Ala-Lys(Mtt)-Glu(OtBu)-Phe-Ile-Ala-Trp-Leu-Val-Arg(Pbf)-
Gly-Arg(Pbf)-Gly-O-Wang re,
(xix)
[SEQ ID NO: 136]
Ala-Ala-Lys(Mmt)-Glu(OtBu)-Phe-Ile-Ala-Trp-Leu-Val-Arg(Pbf)-
Gly-Arg(Pbf)-Gly-O-Wang re,
(xx)
[SEQ ID NO: 137]
Ala-Ala-Lys(Trt-Glu-OtBu)-Glu(OtBu)-Phe-Ile-Ala-Trp-Leu-Val-
Arg(Pbf)-Gly-Arg(Pbf)-Gly-O-Wang re,
(xxi)
[SEQ ID NO: 138]
Ala-Ala-Lys(Glu-OtBu)-Glu(OtBu)-Phe-Ile-Ala-Trp-Leu-Val-
Arg(Pbf)-Gly-Arg(Pbf)-Gly-O-Wang re,
(xxii)
[SEQ ID NO: 139]
Ala-Ala-Lys-Glu(OtBu)-Phe-Ile-Ala-Trp-Leu-Val-Arg(Pbf)-Gly-
Arg(Pbf)-Gly-O-Wang re,
(xxiii)
[SEQ ID NO: 140 or SEQ ID NO: 112]
Ala-Ala-Lys(Pal-Glu-OtBu)-Glu(OtBu)-Phe-Ile-Ala-Trp-Leu-Val-
Arg(Pbf)-Gly-Arg(Pbf)-Gly-O-Wang resin,
(xxiv)
[SEQ ID NO: 141]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-OH,
(xxv)
[SEQ ID NO: 305]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OH,
(xxvi)
[SEQ ID NO: 142]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-OSu,
(xxvii)
[SEQ ID NO: 306]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OSu,
(xxviii)
[SEQ ID NO: 307]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Opfp
(xxix)
[SEQ ID NO: 308]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OPfp
(xxx)
[SEQ ID NO: 143 or SEQ ID NO: 59]
Gln(Trt)-Ala-Ala-Lys(Trt-Glu-OtBu)-Glu(OtBu)-Phe-Ile-Ala-Trp-
Leu-Val-Arg(Pbf)-Gly-Arg(Pbf)-Gly-O-CTC resin,
(xxxi)
[SEQ ID NO: 144 or SEQ ID NO: 154]
Gln(Trt)-Ala-Ala-Lys(Mmt)-Glu(OtBu)-Phe-Ile-Ala-Trp-Leu-Val-
Arg(Pbf)-Gly-Arg(Pbf)-Gly-O-CTC resin,
(xxxii)
[SEQ ID NO: 145 or SEQ ID NO: 153]
Gln(Trt)-Ala-Ala-Lys(Mtt)-Glu(OtBu)-Phe-Ile-Ala-Trp-Leu-Val-
Arg(Pbf)-Gly-Arg(Pbf)-Gly-O-CTC resin,
(xxxiii)
[SEQ ID NO: 146 or SEQ ID NO: 153]
Gln(Trt)-Ala-Ala-Lys-Glu(OtBu)-Phe-Ile-Ala-Trp-Leu-Val-
Arg(Pbf)-Gly-Arg(Pbf)-Gly-OH,
(xxxiv)
[SEQ ID NO: 147]
Gln(Trt)-Ala-Ala-Lys(Glu-OtBu)-Glu(OtBu)-Phe-Ile-Ala-Trp-Leu-
Val-Arg(Pbf)-Gly-Arg(Pbf)-Gly-OH,
(xxxv)
[SEQ ID NO: 148 or SEQ ID NO: 56]
Gln(Trt)-Ala-Ala-Lys(Pal-Glu-OtBu)-Glu(OtBu)-Phe-Ile-Ala-Trp-
Leu-Val-Arg(Pbf)-Gly-Arg(Pbf)-Gly-OH,
(xxxvi)
[SEQ ID NO: 149]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Pal-Glu-OtBu)-Glu(OtBu)-Phe-Ile-Ala-Trp-Leu-Val-Arg(Pbf)-
Gly-Arg(Pbf)-Gly-OH,
(xxxvii)
[SEQ ID NO: 309 or SEQ 276]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Pal-Glu-
OtBu)-Glu(OtBu)-Phe-Ile-Ala-Trp-Leu-Val-Arg(Pbf)-Gly-
Arg(Pbf)-Gly-OH,
(xxxviii)
[SEQ ID NO: 150]
Gln(Trt)-Ala-Ala-Lys(Trt-Glu-OtBu)-Glu(OtBu)-Phe-Ile-Ala-Trp-
Leu-Val-Arg(Pbf)-Gly-Arg(Pbf)-Gly-O-Wang resin,
(xxxix)
[SEQ ID NO: 151]
Gln(Trt)-Ala-Ala-Lys(Glu-OtBu)-Glu(OtBu)-Phe-Ile-Ala-Trp-Leu-
Val-Arg(Pbf)-Gly-Arg(Pbf)-Gly-O-Wang re,
(xl)
[SEQ ID NO: 152 or SEQ ID NO: 145]
Gln(Trt)-Ala-Ala-Lys(Mtt)-Glu(OtBu)-Phe-Ile-Ala-Trp-Leu-Val-
Arg(Pbf)-Gly-Arg(Pbf)-Gly-O-CTC resin,
(xli)
[SEQ ID NO: 153 or SEQ ID NO: 146]
Gln(Trt)-Ala-Ala-Lys-Glu(OtBu)-Phe-Ile-Ala-Trp-Leu-Val-
Arg(Pbf)-Gly-Arg(Pbf)-Gly-OH,
(xlii)
[SEQ ID NO: 154 or SEQ ID NO: 144]
Gln(Trt)-Ala-Ala-Lys(Mmt)-Glu(OtBu)-Phe-Ile-Ala-Trp-Leu-Val-
Arg(Pbf)-Gly-Arg(Pbf)-Gly-O-CTC resin,
(xliii)
[SEQ ID NO: 155]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mtt)-Glu(OtBu)-Phe-Ile-Ala-Trp-Leu-Val-Arg(Pbf)-Gly-
Arg(Pbf)-Gly-O-CTC resin,
(xliv)
[SEQ ID NO: 310]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mtt)-
Glu(OtBu)-Phe-Ile-Ala-Trp-Leu-Val-Arg(Pbf)-Gly-Arg(Pbf)-Gly-
O-CTC resin,
(xlv)
[SEQ ID NO: 156]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys-Glu(OtBu)-Phe-Ile-Ala-Trp-Leu-Val-Arg(Pbf)-Gly-Arg(Pbf)-
Gly-OH,
(xlvi)
[SEQ ID NO: 311]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys-Glu(OtBu)-
Phe-Ile-Ala-Trp-Leu-Val-Arg(Pbf)-Gly-Arg(Pbf)-Gly-OH,
(xlvii)
[SEQ ID NO: 157]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mtt)-Glu(OtBu)-Phe-Ile-Ala-Trp-Leu-Val-Arg(Pbf)-Gly-
Arg(Pbf)-Gly-O-Wang resin,
(xlviii)
[SEQ ID NO: 312]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mtt)-
Glu(OtBu)-Phe-Ile-Ala-Trp-Leu-Val-Arg(Pbf)-Gly-Arg(Pbf)-Gly-
O-Wang resin,
(xlix)
[SEQ ID NO: 158]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mmt)-Glu(OtBu)-Phe-Ile-Ala-Trp-Leu-Val-Arg(Pbf)-Gly-
Arg(Pbf)-Gly-O-Wang resin,
(l)
[SEQ ID NO: 313]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mmt)-
Glu(OtBu)-Phe-Ile-Ala-Trp-Leu-Val-Arg(Pbf)-Gly-Arg(Pbf)-Gly-
O-Wang resin,
(li)
[SEQ ID NO: 159]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys-Glu(OtBu)-Phe-Ile-Ala-Trp-Leu-Val-Arg(Pbf)-Gly-Arg(Pbf)-
Gly-O-Wang resin,
(lii)
[SEQ ID NO: 314]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys-Glu(OtBu)-
Phe-Ile-Ala-Trp-Leu-Val-Arg(Pbf)-Gly-Arg(Pbf)-Gly-O-Wang
resin,
(liii)
[SEQ ID NO: 160]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Trt-Gly-OtBu)-Glu(OtBu)-Phe-Ile-Ala-Trp-Leu-Val-Arg(Pbf)-
Gly-Arg(Pbf)-Gly-O-Wang resin,
(liv)
[SEQ ID NO: 315]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Trt-Gly-
OtBu)-Glu(OtBu)-Phe-Ile-Ala-Trp-Leu-Val-Arg(Pbf)-Gly-
Arg(Pbf)-Gly-O-Wang resin,
(lv)
[SEQ ID NO: 161]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Pal-Glu-OtBu)-Glu(OtBu)-Phe-Ile-Ala-Trp-Leu-Val-Arg(Pbf)-
Gly-Arg(Pbf)-Gly-O-Wang resin,
(lvi)
[SEQ ID NO: 316 or SEQ ID NO: 278]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Pal-Glu-
OtBu)-Glu(OtBu)-Phe-Ile-Ala-Trp-Leu-Val-Arg(Pbf)-Gly-
Arg(Pbf)-Gly-O-Wang resin,
(lvii)
[SEQ ID NO: 162]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Glu-OtBu)-Phe-Ile-Ala-Trp-Leu-Val-Arg(Pbf)-Gly-Arg(Pbf)-
Gly-OH,
(lviii)
[SEQ ID NO: 317]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Glu-OtBu)-
Phe-Ile-Ala-Trp-Leu-Val-Arg(Pbf)-Gly-Arg(Pbf)-Gly-OH,
(lix)
[SEQ ID NO: 163 or SEQ ID NO: 88]
Tyr(tBu)-Leu-Glu(OtBu)-Gly-O-CTC resin,
(lx)
[SEQ ID NO: 164]
Thr(tBu)-Phe-Thr(tBu)-Ser(tBu)-Asp(OtBu)-Val-Ser(tBu)-
Ser(Ψ^{Me,Me}pro)-O-CTC resin,
(lxi)
[SEQ ID NO: 318]
Thr(tBu)-Phe-Thr(tBu)-Ser(tBu)-Asp(OtBu)-Val-Ser(Trt)-Ser(Trt)-
O-CTC resin,
(lxii)
[SEQ ID NO: 165]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-O-CTC resin,
(lxiii)
[SEQ ID NO: 319]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-O-CTC resin,
(lxiv)
[SEQ ID NO: 166]
Lys(Trt-Glu-OtBu)-Glu(OtBu)-Phe-Ile-Ala-Trp-Leu-Val-Arg(Pbf)-
Gly-Arg(Pbf)-Gly-O-Wang resin,
(lxv)
[SEQ ID NO: 167 or SEQ ID NO: 126]
Lys(Glu-OtBu)-Glu(OtBu)-Phe-Ile-Ala-Trp-Leu-Val-Arg(Pbf)-Gly-
Arg(Pbf)-Gly-O-Wang resin,
(lxvi)
[SEQ ID NO: 168]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mmt)-Glu(OtBu)-Phe-Ile-Ala-Trp-Leu-Val-Arg(Pbf)-Gly-
Arg(Pbf)-Gly-O-CTC resin,
(lxvii)
[SEQ ID NO: 320]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mmt)-
Glu(OtBu)-Phe-Ile-Ala-Trp-Leu-Val-Arg(Pbf)-Gly-Arg(Pbf)-Gly-
O-CTC resin,
(lxviii)
[SEQ ID NO: 169]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Trt-Glu-OtBu)-Phe-Ile-Ala-Trp-Leu-Val-Arg(Pbf)-Gly-
Arg(Pbf)-Gly-O-CTC resin,
(lxix)
[SEQ ID NO: 321]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Trt-Glu-
OtBu)-Phe-Ile-Ala-Trp-Leu-Val-Arg(Pbf)-Gly-Arg(Pbf)-Gly-O-
CTC resin,
(lxx)
[SEQ ID NO: 170]
His(Trt)-Ala-Glu(OtBu)-Gly-Thr(tBu)-Phe-Thr(tBu)-Ser(tBu)-
Asp(OtBu)-Val-Ser(tBu)-Ser(Ψ^{Me,Me}pro)-Tyr(tBu)-Leu-Glu(OtBu)-
Gly-Gln(Trt)-Ala-Ala-Lys(Glu-OtBu)-Phe-Ile-Ala-Trp-Leu-Val-
Arg(Pbf)-Gly-Arg(Pbf)-Gly-O-Wang resin,

and

(lxxi)

[SEQ ID NO: 322]

His(Trt)-Ala-Glu(OtBu)-Gly-Thr(tBu)-Phe-Thr(tBu)-
Ser(tBu)-Asp(OtBu)-Val-Ser(Trt)-Ser(Trt)-Tyr(tBu)-
Leu-Glu(OtBu)-Gly-Gln(Trt)-Ala-Ala-Lys(Glu-OtBu)-
Phe-Ile-Ala-Trp-Leu-Val-Arg(Pbf)-Gly-Arg(Pbf)-
Gly-O-Wang resin

or selected from the group consisting of:

(lxxii)

[SEQ ID NO: 307]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-OPfp
(lxxiii)

[SEQ ID NO: 308]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OPfp

wherein the N-terminal amino acids in (i)-(v) are optionally protected with Fmoc, CBz or Boc, and wherein the N-terminal amino acids in (vi)-(lxxi), or (lxxii)-(lxxiii) are optionally protected with Fmoc or Cbz.

Preferably, for peptides (i)-(v), the N-terminal amino acid with Fmoc, Boc or CBz. More preferably, peptides (i)-(v) are protected at the N-terminal amino acid with Boc.

Preferably for peptides (vi)-(lxxi), or (lxxii)-(lxxiii), the N-terminal amino acid is protected with Fmoc or Cbz, and more preferably with Fmoc.

Particularly preferred peptide fragments in accordance with the present invention are:

(i)

[SEQ ID NO: 118]

His(Trt)-Ala-Glu(OtBu)-Gly-OH,
(ii)

[SEQ ID NO: 119]

His(Trt)-Ala-Glu(OtBu)-Gly-OSu,
(iii)

[SEQ ID NO: 120]

His(Trt)-Ala-Glu(OtBu)-Gly-OBt,
(iv)

[SEQ ID NO: 121]

His(Trt)-Ala-Glu(OtBu)-Gly-OPfp,
(v)

[SEQ ID NO: 122]

His(Trt)-Ala-Glu(OtBu)-Gly-O-CTC resin,

or

[SEQ ID NO: 7]

Boc-His(Trt)-Ala-Glu(OtBu)-Gly-OH,

[SEQ ID NO: 10]

Boc-His(Trt)-Ala-Glu(OtBu)-Gly-OSu,

and

[SEQ ID NO: 13]

Boc-His(Trt)-Ala-Glu(OtBu)-Gly-OBt

or

[SEQ ID NO: 268]

Boc-His(Trt)-Ala-Glu(OtBu)-Gly-OPfp.

Preferably, these peptide fragments contain minimal (e.g. <0.5%, <0.2%, <0.1% by weight of the D-His impurity. More preferably, these peptide fragments contain minimal (e.g. <0.5%, <0.2%, <0.1% by weight of the [+Gly⁴] impurity. The above peptide fragments are useful as intermediates in the synthesis of liraglutide.

In a further aspect, the present invention provides liraglutide of high purity. The Liraglutide of the present invention preferably contains less than 5 wt %, less than 2 wt %, less than 1%, less than 0.5%, less than 0.2%, or less than 0.1% of the D-His isomer of liraglutide. The liraglutide of the present invention may also contain less than 5 wt %, less than 2 wt %, less than 1%, less than 0.5%, less than 0.2%, or less than 0.1% of the [+Gly¹⁶] derivative of liraglutide [SEQ ID NO: 116]. Moreover, the liraglutide of the present invention may further contain less than 5 wt %, less than 2 wt %, less than 1%, less than 0.5%, less than 0.2%, or less than 0.1% of the [+Gly³¹] derivative of liraglutide [SEQ ID NO: 117]. The liraglutide of the present invention may further contain less than 5 wt %, less than 2 wt %, less than 1%, less than 0.5%, less than 0.2%, or less than 0.1% of the [+Gly⁴] derivative of liraglutide [SEQ ID NO: 114].

The liraglutide of the present invention may also contain:
(a) less than 5 wt %, less than 2 wt %, less than 1%, less than 0.5%, less than 0.2%, or less than 0.1% of the D-His isomer of liraglutide and/or
(b) less than 5 wt %, less than 2 wt %, less than 1%, less than 0.5%, less than 0.2%, or less than 0.1% of the [+Gly¹⁶] derivative of liraglutide, and/or
(c) less than 5 wt %, less than 2 wt %, less than 1%, less than 0.5%, less than 0.2%, or less than 0.1% of the [+Gly³¹] derivative of liraglutide and/or
(d) less than 5 wt %, less than 2 wt %, less than 1%, less than 0.5%, less than 0.2%, or less than 0.1% of the [+Gly⁴] derivative of liraglutide.

Preferably, liraglutide according to the present invention contains less than 0.5% of the D-His isomer of liraglutide, and less than 0.5% of the [+Gly⁴] derivative of liraglutide. More preferably, liraglutide according to the present invention contains less than 0.2% of the D-His isomer of liraglutide, and less than 0.2% of the [+Gly⁴]derivative of liraglutide. Most preferably, liraglutide according to the present invention contains less than 0.1% of the D-His isomer of liraglutide, and less than 0.1% of the [+Gly⁴] derivative of liraglutide.

### Preparation of Other GLP-1 Proteins Including Semaglutide

The above-described process for preparing liraglutide may be employed for the synthesis of other GLP-1 proteins, particularly those sharing a similar backbone to liraglutide. For example, the above-described process can be used to prepare semaglutide, which has a similar backbone to liraglutide, and also contains a long side chain at Lys²⁰.

Thus, in another aspect of the present invention, there is provided a process for preparing a GLP-1 peptide comprising liquid or solid phase peptide synthesis or a combination thereof, wherein the process comprises a final coupling step in which at least two fragments are coupled at a terminal Gly residue, and wherein at least one of the fragments is prepared by coupling of at least two sub-fragments.

Preferably, the process comprises a final coupling step in which two fragments are coupled at a terminal Gly residue.

Preferably, the GLP-1 peptide comprises at least one non-terminal Gly residue, more preferably, the GLP-1 peptide contains at least two non-terminal Gly residues, and most preferably, the GLP-1 peptide contains two, three or four non-terminal Gly residues, and especially, the GLP-1 peptide contains three non-terminal Gly residues. In preferred embodiments, the GLP-1 peptide contains at least one-non-terminal Gly residue, preferably wherein the non-terminal Gly residue is at least the third (i.e. Gly^{>3}) preferably at least the fourth (i.e. Gly^{>4}) amino acid from the N-terminus.

In the above process, it is preferred that at least one least one of the fragments is prepared by coupling of at least two (and preferably two) sub-fragments at a terminal Gly residue.

This strategy is employed in the process for preparing liraglutide as discussed in detail above. However, the process is generally applicable to other GLP-1 peptides particularly those containing at least two non-terminal Gly residues, especially two, three or four non-terminal Gly residues. Semaglutide is a particularly preferred GLP-1 peptide.

Thus, in further aspect of the present invention, there is provided a process for preparing semaglutide of formula:

```
  1 2 3 4 5 6 7 8 9 10 11 12
His-Aib-Glu-Gly-Thr-Phe-Thr-Ser-Asp-Val-Ser-Ser-
  13 14 15 16 17 18 19 20 21
Tyr-Leu-Glu-Gly-Gln-Ala-Ala-Lys(W)-Glu-Phe-
  22 23 24 25 26 27 28 29 30 31
Ile-Ala-Trp-Leu-Val-Arg-Gly-Arg-Gly-OH
```

wherein W=N-(17-carboxy-1-oxoheptadecyl)-L-γ-glutamyl-2-[2-(2-aminoethoxy)ethoxy]acetyl-2-[2-(2-aminoethoxy)ethoxy]acetyl [SEQ ID NO: 174]
   by a similar synthetic strategy as discussed above for liraglutide, namely a process comprising:
(i) coupling a Peptide 1 having the sequence:

[SEQ ID NO: 175]

His-Aib-Glu-Gly

wherein:
   the N-terminal of His is optionally protected with a protecting group, preferably selected from the group consisting of Boc, Cbz or Fmoc, and
   the Gly carboxylic acid group in Peptide 1 may be in the form of an activated carboxylic acid derivative;
with a Peptide 2 having the sequence:
Thr-Phe-Thr-Ser-Asp-Val-Ser-Ser-Tyr-Leu-Glu-Gly-Gln-Ala-Ala-Lys(W1)-Glu-Phe-Ile-Ala-Trp-Leu-Val-Arg-Gly-Arg-Gly-OH wherein W1=N-(17-carboxy-1-oxoheptadecyl)-L-γ-glutamyl-2-[2-(2-aminoethoxy)ethoxy]acetyl-2-[2-(2-aminoethoxy)ethoxy]acetyl [SEQ ID NO: 176]
wherein:
   Peptide 2 is optionally conjugated to a solid support;
and wherein one or more of the amino acid residues in Peptide 1 and Peptide 2 and W1 may be protected or unprotected, and are preferably protected, more preferably with acid-cleavable protecting groups,
(ii) optionally removing any protecting groups and/or cleaving the resin to form semaglutide; and
(iii) optionally purifying the semaglutide
wherein the Peptide 2 is preferably prepared by a two or three fragment convergent synthesis, preferably a two fragment convergent synthesis.

The Peptide 2 is either conjugated to a Wang resin, or the Peptide 2 is not conjugated to a solid support on a resin.

The Peptide 1 preferably has the formula:

B.

[SEQ ID NO: 177]

P1-His(P)-Aib-Glu(P)-Gly-O-P2

wherein P1 represents a protecting group for the N-terminal of His (preferably Boc, Fmoc or Cbz), each P represents side chain protecting groups which may be the same or different, and P2 is selected from: H, or a solid support (preferably a CTC resin), or P2 represents an activated carboxylic ester of the Gly⁴ residue (preferably Su or Bt or Pfp)

Particularly, Peptide 1 is selected from the group consisting of:

[SEQ ID NO: 178]

Boc-His(Trt)-Aib-Glu(OtBu)-Gly-OH,

[SEQ ID NO: 179]

Fmoc-His(Trt)-Aib-Glu(OtBu)-Gly-OH,

[SEQ ID NO: 180]

Cbz-His(Trt)-Aib-Glu(OtBu)-Gly-OH,

[SEQ ID NO: 181]

Boc-His(Trt)-Aib-Glu(OtBu)-Gly-OSu,

[SEQ ID NO: 182,]

Fmoc-His(Trt)-Aib-Glu(OtBu)-Gly-OSu

[SEQ ID NO: 183]

Cbz-His(Trt)-Aib-Glu(OtBu)-Gly-OSu,

[SEQ ID NO: 184]

Boc-His(Trt)-Aib-Glu(OtBu)-Gly-OBt,

[SEQ ID NO: 185,]

Fmoc-His(Trt)-Aib-Glu(OtBu)-Gly-OBt

and

[SEQ ID NO: 186]

Cbz-His(Trt)-Aib-Glu(OtBu)-Gly-OBt.

More particularly, Peptide 1 is selected from the group consisting of:

[SEQ ID NO: 187]

Boc-His(Trt)-Aib-Glu(OtBu)-Gly-OPfp,

[SEQ ID NO: 188]

Fmoc-His(Trt)-Aib-Glu(OtBu)-Gly-OPfp,

and

[SEQ ID NO: 189]

Cbz-His(Trt)-Aib-Glu(OtBu)-Gly-OPfp.

Especially preferred Peptide 1 compounds are selected from the group consisting of:

[SEQ ID NO: 178]

Boc-His(Trt)-Aib-Glu(OtBu)-Gly-OH

[SEQ ID NO: 181]

Boc-His(Trt)-Aib-Glu(OtBu)-Gly-OSu,

[SEQ ID NO: 184]

Boc-His(Trt)-Aib-Glu(OtBu)-Gly-OBt,

and

[SEQ ID NO: 187]

Boc-His(Trt)-Aib-Glu(OtBu)-Gly-OPfp

A most preferred Peptide 1 is Boc-His(Trt)-Aib-Glu(OtBu)-Gly-OPfp—[SEQ ID NO: 187]

In the above process for preparing semaglutide, Peptide 2 preferably has the formula:

P1-Thr(P)-Phe-Thr(P)-Ser(P)-Asp(P)-Val-Ser(P)-Ser(P)-Tyr(P)-Leu-Glu(P)-Gly-Gln(P)-Ala-Ala-Lys(W1)-Glu(P)-Phe-Ile-Ala-Trp-Leu-Val-Arg(P)-Gly-Arg(P)-Gly-OP2,
wherein W1=N-(17-carboxy(P)-1-oxoheptadecyl)-L-γ-glutamyl(P)-2-[2-(2-aminoethoxy)ethoxy]acetyl-2-[2-(2-aminoethoxy)ethoxy]acetyl [SEQ ID NO: 190],
wherein P1 represents H, or a protecting group for the N-terminal of Thr (preferably Fmoc or Cbz and more preferably Fmoc), each P represents side chain protecting groups which may be the same or different, and P2 is selected from H, or a solid support, preferably a CTC or Wang resin.

Thus, in a preferred embodiment, the present invention provides a process for preparing semaglutide, comprising:
(i) coupling a Peptide 1 having the formula selected from the group consisting of:

[SEQ ID NO: 178]

Boc-His(Trt)-Aib-Glu(OtBu)-Gly-OH,

and
with a Peptide 2 having the formula:

Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(W1)-Glu(OtBu)-Phe-Ile-Ala-Trp-Leu-Val-Arg(Pbf)-Gly-Arg(Pbf)-Gly-O-Wang resin
wherein W1=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl [SEQ ID NO: 191],
or
Thr(tBu)-Phe-Thr(tBu)-Ser(tBu)-Asp(OtBu)-Val-Ser(Trt)-Ser(Trt)-Tyr(tBu)-Leu-Glu(OtBu)-Gly-Gln(Trt)-Ala-Ala-Lys(W1)-Glu(OtBu)-Phe-Ile-Ala-Trp-Leu-Val-Arg(Pbf)-Gly-Arg(Pbf)-Gly-O-Wang resin wherein W1=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl [SEQ ID NO: 192],
(ii) removing the protecting groups and resin to form semaglutide, and optionally
(iii) purifying the semaglutide.

In another embodiment, the present invention provides a process for preparing semaglutide, comprising the steps of:
(i) coupling a Peptide 1 having the formula selected from the group consisting of

[SEQ ID NO: 181]

Boc-His(Trt)-Aib-Glu(OtBu)-Gly-OSu,

[SEQ ID NO: 182]

Fmoc-His(Trt)-Aib-Glu(OtBu)-Gly-OSu,

[SEQ ID NO: 184]

Boc-His(Trt)-Aib-Glu(OtBu)-Gly-OBt,

and

[SEQ ID NO: 185]

Fmoc-His(Trt)-Aib-Glu(OtBu)-Gly-OBt,

or a Peptide 1 having the formula selected from the group consisting of:

[SEQ ID NO: 187]

Boc-His(Trt)-Aib-Glu(OtBu)-Gly-OPfp

[SEQ ID NO: 188]

Fmoc-His(Trt)-Aib-Glu(OtBu)-Gly-OPfp

with a Peptide 2 having the formula:

Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(W1)-Glu(OtBu)-Phe-Ile-Ala-Trp-Leu-Val-Arg(Pbf)-Gly-Arg(Pbf)-Gly-OH
wherein W1=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl [SEQ ID NO: 193],
or

Thr(tBu)-Phe-Thr(tBu)-Ser(tBu)-Asp(OtBu)-Val-Ser(Trt)-Ser(Trt)-Tyr(tBu)-Leu-Glu(OtBu)-Gly-Gln(Trt)-Ala-Ala-Lys(W1)-Glu(OtBu)-Phe-Ile-Ala-Trp-Leu-Val-Arg(Pbf)-Gly-Arg(Pbf)-Gly-OH
wherein W1=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl [SEQ ID NO: 194], and
wherein the coupling is carried out in liquid phase,
(ii) removing the protecting groups to form semaglutide, and optionally
(iii) purifying the semaglutide.

In any of the above-described processes for preparing semaglutide, Peptide 2 is preferably prepared by coupling of Peptide 3 with Peptide 4, wherein the amino acid sequence in Peptide 3 is:

[SEQ ID NO: 42]

Thr-Phe-Thr-Ser-Asp-Val-Ser-Ser-Tyr-Leu-Glu-Gly-OH

wherein the Gly carboxylic acid group is optionally activated, preferably as an OSu ester or as an OPfp ester,
and the amino acid sequence in Peptide 4 is:

[SEQ ID NO: 195]

Gln-Ala-Ala-Lys(W1)-Glu-Phe-Ile-Ala-Trp-Leu-Val-
Arg-Gly-Arg-Gly-OH

wherein W1=N-(17-carboxy-1-oxoheptadecyl)-L-γ-glutamyl-2-[2-(2-aminoethoxy)ethoxy]acetyl-2-[2-(2-aminoethoxy)ethoxy]acetyl
wherein Peptide 4 is optionally conjugated to a resin at the terminal Gly-OH, preferably wherein the resin is a Wang resin,
and wherein one or more of the amino acid residues in Peptide 1, Peptide 2 and W1 may be unprotected or protected, and are preferably protected, more preferably with acid cleavable protecting groups.
   The Peptide 3 in the above process can preferably have the formula:

[SEQ ID NO: 43]

P1-Thr(P)-Phe-Thr(P)-Ser(P)-Asp(P)-Val-Ser(P)-
Ser(P)-Tyr(P)-Leu-Glu(P)-Gly-OP2

wherein P1 represents a protecting group for the N-terminal of Thr (preferably Fmoc or Cbz and more preferably Fmoc), each P represents side chain protecting groups which may be the same or different, and P2 is selected from H, or P2 represents an activated carboxylic ester of the Gly¹⁶ residue (preferably Su), or P2 represents a solid support, preferably a CTC resin.
   The Peptide 4 may preferably have the formula:

P1-Gln(P)-Ala-Ala-Lys(W1)-Glu(P)-Phe-Ile-Ala-Trp-Leu-Val-Arg(P)-Gly-Arg(P)-Gly-O-P2,
wherein W1=N-(17-carboxy(P)-1-oxoheptadecyl)-L-γ-glutamyl(P)-2-[2-(2-aminoethoxy)ethoxy]acetyl-2-[2-(2-aminoethoxy)ethoxy]acetyl [SEQ ID NO: 196]
wherein:
P1 represents H or a protecting group for the N-terminal of Gin (preferably Fmoc or Cbz, and more preferably Cbz), each P represents side chain protecting groups which may be the same or different, P2 is selected from H, or P2 represents a solid support, preferably a CTC or Wang resin, more preferably a Wang resin.
   In preferred embodiments, Peptide 2 can be prepared by a process comprising:

Process A, which comprises:
(i) coupling Peptide 3 of formula:

[SEQ ID NO: 44]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-OH,

or

[SEQ ID NO: 287]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

to Peptide 4 of formula:

Gln(Trt)-Ala-Ala-Lys(W1)-Glu(OtBu)-Phe-Ile-Ala-Trp-Leu-Val-Arg(Pbf)-Gly-Arg(Pbf)-Gly-O-Wang resin
wherein W1=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl [SEQ ID NO: 197],
and removing the Fmoc protecting group to form Peptide 2 of formula:

Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(W1)-Glu(OtBu)-Phe-Ile-Ala-Trp-Leu-Val-Arg(Pbf)-Gly-Arg(Pbf)-Gly-O-Wang resin
wherein W1=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl [SEQ ID NO: 198]
or

Thr(tBu)-Phe-Thr(tBu)-Ser(tBu)-Asp(OtBu)-Val-Ser(Trt)-Ser(Trt)-Tyr(tBu)-Leu-Glu(OtBu)-Gly-Gln(Trt)-Ala-Ala-Lys(W1)-Glu(OtBu)-Phe-Ile-Ala-Trp-Leu-Val-Arg(Pbf)-Gly-Arg(Pbf)-Gly-O-Wang resin
wherein W1=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl [SEQ ID NO: 199];
or

Process B, which comprises:
(i) liquid phase coupling of Peptide 3 of formula:

[SEQ ID NO: 45]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-OSu

or

[SEQ ID NO: 288]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OSu

or a Peptide 3 of formula:

[SEQ ID NO: 289]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OPfp

[SEQ ID NO: 290]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-OPfp

to Peptide 4 of formula:

Gln(Trt)-Ala-Ala-Lys(W1)-Glu(OtBu)-Phe-Ile-Ala-Trp-Leu-Val-Arg(Pbf)-Gly-Arg(Pbf)-Gly-OH,
wherein W1=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl [SEQ ID NO: 200]
and removing the Fmoc to form Peptide 2 of formula:

Thr(tBu)-Phe-Thr(tBu)-Ser(tBu)-Asp(OtBu)-Val-Ser(tBu)-Ser (Ψ^{Me,Me}pro)-Tyr(tBu)-Leu-Glu(OtBu)-Gly-Gln(Trt)-Ala-Ala-Lys(W1)-Glu(OtBu)-Phe-Ile-Ala-Trp-Leu-Val-Arg(Pbf)-Gly-Arg(Pbf)-Gly-OH
wherein W1=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl [SEQ ID NO: 201],
or

Thr(tBu)-Phe-Thr(tBu)-Ser(tBu)-Asp(OtBu)-Val-Ser(Trt)-Ser(Trt)-Tyr(tBu)-Leu-Glu(OtBu)-Gly-Gln(Trt)-Ala-Ala-Lys(W1)-Glu(OtBu)-Phe-Ile-Ala-Trp-Leu-Val-Arg(Pbf)-Gly-Arg(Pbf)-Gly-OH
wherein W1=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl [SEQ ID NO: 202].

In a preferred embodiment of Process A, the Peptide 4 is prepared by sequential synthesis on a resin, preferably a Wang resin, using Fmoc strategy, and wherein the -Lys(W1)- residue is formed by a process comprising the steps of:
(i) sequential coupling of Fmoc-Lys(Mtt)-OH or Fmoc-Lys(Mmt)-OH
(ii) coupling one or more amino acid residues sequentially to the Lys residue to form the amino acid sequence of Peptide 4 wherein P1 is Cbz,
(iii) removing the Mmt or Mtt protecting group,
(iv) coupling 2-{[2-(Fmoc-amino)ethoxy]ethoxy}acetic acid to the Lys residue and removing the Fmoc group
(v) coupling 2-{[2-(Fmoc-amino)ethoxy]ethoxy}acetic acid to the free amine and removing the Fmoc group,
(vi) coupling with Fmoc-Glu-OtBu and removing the Fmoc group, and
(vii) coupling with octadecanedioic acid mono-tert-butyl ester.

The coupling agents are preferably selected to provide the OtBu protecting groups on W1, wherein W1 represents=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl.

In a preferred embodiment of Process B, the Peptide 4 is prepared by sequential synthesis on a resin, preferably a Wang resin, using Fmoc strategy, and wherein the -Lys(W1)- residue is formed by a process comprising the steps of:
(i) sequential coupling of Fmoc-Lys(Mtt)-OH or Fmoc-Lys(Mmt)-OH
(ii) coupling one or more amino acid residues sequentially to the Lys residue to form the amino acid sequence of Peptide 4,
(iii) simultaneously removing the Mmt or Mtt protecting group and cleaving the peptide from the resin, and
   coupling 17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetic acid to the Lys residue.

The N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyc acid is preferably prepared by a process comprising:
a) loading 2-{[2-(Fmoc-amino)ethoxy]ethoxy}acetic acid to a CTC resin, and removing Fmoc,
b) coupling 2-{[2-(Fmoc-amino)ethoxy]ethoxy}acetic acid unit to the free amine group, and removing Fmoc,
c) coupling Fmoc-Glu-OtBu to the free amine, and removing Fmoc,
d) coupling octadecanedioic acid mono-tert-butyl ester, and
e) cleaving the 17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetic acid from the resin.

The coupling agents are preferably selected to provide the OtBu protecting groups on W1, wherein W1 represents=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl.

In another preferred embodiment of Process A, Peptide 4 is prepared by sequential synthesis on a resin, preferably a Wang resin, using Fmoc strategy, and wherein the -Lys(W1)- residue is formed by a process comprising the steps of:
(i) sequential coupling of Fmoc-Lys(W1)-OH wherein W1=N-(17-carboxy-1-oxoheptadecyl)-L-γ-glutamyl-2-[2-(2-aminoethoxy)ethoxy]acetyl-2-[2-(2-aminoethoxy)ethoxy]acetyl,
(ii) coupling one or more amino acid residues sequentially to the Lys residue to form the amino acid sequence of Peptide 4, and
(iii) cleaving the peptide from the resin.
   The Fmoc-Lys(W1)-OH may preferably be prepared by a process comprising the steps of:
a) coupling Cbz-Lys(Fmoc)-OH to a CTC resin, and removing Fmoc,
b) coupling 2-{[2-(Fmoc-amino)ethoxy]ethoxy}acetic acid to the free amine group of Lys, and removing Fmoc,
c) coupling 2-{[2-(Fmoc-amino)ethoxy]ethoxy}acetic acid unit to the free amine group, and removing Fmoc,
d) coupling Fmoc-Glu-OtBu to the free amine group, and removing Fmoc
e) coupling octadecanedioic acid mono-tert-butyl ester to Glu to form Cbz-Lys{N-(17-carboxy-1-oxoheptadecyl)-L-γ-glutamyl[2-(2-aminoethoxy) ethoxy]acetyl[2-(2-aminoethoxy)ethoxy]acetic acid}, and removing the Cbz group,
f) protecting the N-terminus with Fmoc group, and
g) cleaving Fmoc-Lys{N-(17-carboxy-1-oxoheptadecyl)-L-γ-glutamyl[2-(2-aminoethoxy) ethoxy]acetyl[2-(2-aminoethoxy)ethoxy]acetic acid} from the resin.

The coupling agents are preferably selected to provide the OtBu protecting groups on W1, wherein W1 represents=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl.

Fmoc-Lys(W1)-OH may alternatively be prepared by a process comprising coupling 17-carboxy(OtBu)-1-oxoheptadecyl)-L-γ-glutamyl(OtBu)-[2-(2-aminoethoxy)ethoxy]acetyl[2-(2-aminoethoxy)ethoxy]acetic acid to Fmoc-Lys-OH in solution or attached to the CTC resin.

As for the synthesis of liraglutide, the Peptide 3 is preferably prepared by a convergent process. In any embodiment of the process for preparing semaglutide, the Peptide 3 is preferably prepared by a two fragment coupling on a resin, cleaving the peptide from the resin, and optionally activating the Gly carboxylic group.

In a preferred embodiment, Peptide 3 may advantageously be prepared by:
(i) coupling a Peptide 5 containing the optionally protected amino acid sequence:

[SEQ ID NO: 78]

Thr-Phe-Thr-Ser-Asp-Val-Ser-Ser

with a Peptide 6 containing the optionally protected amino acid sequence:

[SEQ ID NO: 84]

Tyr-Leu-Glu-Gly-OH

which is conjugated to a resin,
(ii) cleaving the Peptide 3 from the resin, and optionally
(iii) activating the Gly carboxylic group in Peptide 3.

More preferably, the Peptide 3 is prepared by:
(i) coupling of Peptide 5 having the formula:

[SEQ ID NO: 323]

Fmoc-Thr(tBu)-Phe-Thr(tBu)-Ser(tBu)-Asp(OtBu)-Val-
Ser(tBu)-Ser (Ψ^{Me,Me}pro),

or

[SEQ ID NO: 297]

Fmoc-Thr(tBu)-Phe-Thr(tBu)-Ser(tBu)-Asp(OtBu)-Val-
Ser(Trt)-Ser(Trt),

and preferably wherein Peptide 5 has the formula [SEQ ID NO: 323]
with a Peptide 6 of formula:

[SEQ ID NO: 83]

Tyr(tBu)-Leu-Glu(OtBu)-Gly-OH

which is conjugated to a CTC resin,
(ii) cleaving the peptide from the resin, and optionally
(iii) activating the Gly carboxylic group in Peptide 3.

In the above process, Peptide 5 of [SEQ ID NO: 78] has the formula:

[SEQ ID NO: 79]

P1-Thr(P)-Phe-Thr(P)-Ser(P)-Asp(P)-Val-Ser(P)-
Ser(P)-O-P2

wherein P1 represents a protecting group for the N-terminal of Thr (preferably Fmoc or Cbz and more preferably Fmoc), each P represents side chain protecting groups which may be the same or different, and P2 is H or a solid support, preferably a CTC resin.
   Peptide 6 of the above [SEQ ID NO: 84] preferably has the formula:

[SEQ ID NO: 85]

P1-Tyr(P)-Leu-Glu(P)-Gly-O-P2

wherein P1 represents a protecting group for the N-terminal of Tyr (preferably Fmoc or Cbz and more preferably Fmoc), each P represents side chain protecting groups which may be the same or different, and P2 is a solid support, preferably a CTC resin.

As an alternative to the above processes for preparing semaglutide, the present invention further provides a process for preparing semaglutide:

```
  1 2 3 4 5 6 7 8 9 10 11 12 13 14 15 16 17 18 19
His-Aib-Glu-Gly-Thr-Phe-Thr-Ser-Asp-Val-Ser-Ser-Tyr-Leu-Glu-Gly-Gln-Ala-Ala-
  20 21 22 23 24 25 26 27 28 29 30 31
Lys(W)-Glu-Phe-Ile-Ala-Trp-Leu-Val-Arg-Gly-Arg-Gly-OH
```

wherein W=N-(17-carboxy-1-oxoheptadecyl)-L-γ-glutamyl-2-[2-(2-aminoethoxy)ethoxy]acetyl-2-[2-(2-aminoethoxy)ethoxy]acetyl [SEQ ID NO: 174]
wherein the process comprises:
a) coupling P1-Gly-OH to a resin, preferably a Wang or CTC resin, and removing P1;
b) sequentially coupling N-terminal P1-protected amino acids Arg, Gly, Arg, Val, Leu, Trp, Ala, Ile, Phe and Glu which may be optionally side-chain protected, and removing P1 to form a peptide-resin comprising amino acid sequence 21-31 of semaglutide;
c) coupling of Fmoc-Lys(W1)-OH wherein W1=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl to the Glu²¹ residue, and removing Fmoc
d) sequentially coupling N-terminal P1-protected amino acids: Ala, Ala, Gln, Gly, Glu, Leu, Tyr, Ser, Ser, Val, Asp, Ser, Thr, Phe and Thr which may be optionally side-chain protected, and removing P1 to form a peptide-resin comprising amino acid sequence 5-31 of semaglutide; and
e) coupling with a Peptide 1 containing the sequence His-Aib-Glu-Gly [SEQ ID NO: 175].
   Alternatively, the synthesis of semaglutide:

```
  1 2 3 4 5 6 7 8 9 10 11 12 13 14 15 16 17 18 19
His-Aib-Glu-Gly-Thr-Phe-Thr-Ser-Asp-Val-Ser-Ser-Tyr-Leu-Glu-Gly-Gln-Ala-Ala-
  20 21 22 23 24 25 26 27 28 29 30 31
Lys(W)-Glu-Phe-Ile-Ala-Trp-Leu-Val-Arg-Gly-Arg-Gly-OH
```

wherein W=N-(17-carboxy-1-oxoheptadecyl)-L-γ-glutamyl-2-[2-(2-aminoethoxy)ethoxy]acetyl-2-[2-(2-aminoethoxy)ethoxy]acetyl[SEQ ID NO: 174]
can comprise:
a) coupling P1-Gly-OH to a resin, preferably a Wang or CTC resin, and removing P1;
b) sequentially coupling N-terminal P1-protected amino acids Arg, Gly, Arg, Val, Leu, Trp, Ala, Ile, Phe and Glu which may be optionally side-chain protected, and removing P1 to form a peptide-resin comprising amino acid sequence 21-31 of semaglutide;
c) coupling Cbz-Lys(Fmoc)-OH to Glu²¹, and removing Fmoc,
d) coupling 2-{[2-(Fmoc-amino)ethoxy]ethoxy}acetic acid to the free amine group of Lys, and removing Fmoc,
e) coupling 2-{[2-(Fmoc-amino)ethoxy]ethoxy}acetic acid unit to the free amine group and removing Fmoc,
f) coupling Fmoc-Glu-OtBu to the free amine group and removing Fmoc
g) coupling octadecanedioic acid mono-tert-butyl ester to Glu to form Cbz-Lys{N-(17-carboxy(OtBu)-1-oxoheptadecyl)-L-γ-glutamyl(OtBu)[2-(2-aminoethoxy) ethoxy]acetyl[2-(2-aminoethoxy)ethoxy]acetic acid}, and removing the Cbz group,
h) sequentially coupling P1-protected amino acids: Ala, Ala, Gln, Gly, Glu, Leu, Tyr, Ser, Ser, Val, Asp, Ser, Thr, Phe and Thr, and removing P1 to form a peptide-resin comprising amino acid sequence 5-31 of semaglutide, and
i) coupling with a Peptide 1 containing the sequence His-Aib-Glu-Gly [SEQ ID NO: 175].

In the above described processes for preparing semaglutide, the W side chain on Lys²⁰ of semaglutide is preferably prepared with side-chain protecting groups, (i.e. W1), i.e. wherein W1=N-(17-carboxy(P)-1-oxoheptadecyl)-L-γ-glutamyl(P)-2-[2-(2-aminoethoxy)ethoxy]acetyl-2-[2-(2-aminoethoxy)ethoxy]acetyl [SEQ ID NO: 198]. The preferred side chain protecting group in W1 is OtBu. The side chain protection is preferably cleaved after completion of the semaglutide synthesis (e.g. along with the other side chain protecting groups in the semaglutide backbone.

The coupling agents employed in the above process that form the Lys²⁰ side chain selected to provide the OtBu protecting groups on W1, wherein W1 represents=N-(17-carboxy(OtBu)-1-oxoheptadecyl)-L-γ-glutamyl(OtBu)-2-[2-(2-aminoethoxy)ethoxy]acetyl-2-[2-(2-aminoethoxy)ethoxy]acetyl.

In the above two processes, the 5-31 amino acid backbone of semaglutide is prepared by linear, sequential synthesis, wherein the Lys²⁰ side chain in protected form (i.e. W1) is installed after addition of the Lys²⁰ residue. The completed 5-31 peptide can then be condensed with the 1-4 amino acid backbone (i.e. Peptide 1) in a convergent manner. Preferably, in the above synthesis processes for preparing semaglutide, each P1 independently represents Fmoc, Cbz or Boc, or a combination thereof. Preferably, the Thr, Ser, Asp, Tyr, Glu, Gln, Lys and Arg residues employed in the above processes are side chain protected with acid-cleavable protecting groups. Particularly preferred acid cleavable protecting groups are selected from the group consisting of: tBu, OtBu, Ψ^{Me,Me}pro, Trt, and Pbf. Thus, for example, the amino acids are side chain protected as: Thr(tBu), Ser⁸(tBu), Ser⁸(Trt), Ser¹¹(tBu), Ser¹¹(Trt), Lys(Mtt) or Lys(Mmt), Asp (OtBu), Ser¹²(Ψ^{Me,Me}pro), Ser¹²(Trt), Tyr(tBu), Glu(OtBu), Gln(Trt), and Arg(Pbf). The Peptide 1, containing the amino acids 1-4 of semaglutide, which is condensed onto the completed 5-31 fragment is preferably selected from the group consisting of:

[SEQ ID NO: 178]

Boc-His(Trt)-Aib-Glu(OtBu)-Gly-OH,

[SEQ ID NO: 179,]

Fmoc-His(Trt)-Aib-Glu(OtBu)-Gly-OH

[SEQ ID NO: 180]

Cbz-His(Trt)-Aib-Glu(OtBu)-Gly-OH,

[SEQ ID NO: 181]

Boc-His(Trt)-Aib-Glu(OtBu)-Gly-OSu,

[SEQ ID NO: 182]

Fmoc-His(Trt)-Aib-Glu(OtBu)-Gly-OSu,

[SEQ ID NO: 183]

Cbz-His(Trt)-Aib-Glu(OtBu)-Gly-OSu,

[SEQ ID NO: 184]

Boc-His(Trt)-Aib-Glu(OtBu)-Gly-OBt,

[SEQ ID NO: 185]

Fmoc-His(Trt)-Aib-Glu(OtBu)-Gly-OBt,

and

[SEQ ID NO: 186]

Cbz-His(Trt)-Aib-Glu(OtBu)-Gly-OBt.

More preferably, Peptide 1 is selected from the group consisting of:

[SEQ ID NO: 187]

Boc-His(Trt)-Aib-Glu(OtBu)-Gly-OPfp,

[SEQ ID NO: 188]

Fmoc-His(Trt)-Aib-Glu(OtBu)-Gly-OPfp,

and

[SEQ ID NO: 189]

Cbz-His(Trt)-Aib-Glu(OtBu)-Gly-OPfp.

Particularly preferred are Peptide 1 compounds selected from the group consisting of:

[SEQ ID NO: 178]

Boc-His(Trt)-Aib-Glu(OtBu)-Gly-OH,

[SEQ ID NO: 181]

Boc-His(Trt)-Aib-Glu(OtBu)-Gly-OSu,

[SEQ ID NO: 184]

Boc-His(Trt)-Aib-Glu(OtBu)-Gly-OBt,

and

[SEQ ID NO: 187]

Boc-His(Trt)-Aib-Glu(OtBu)-Gly-OPfp.

A most preferred Peptide 1 compound is:

[SEQ ID NO: 187]

Boc-His(Trt)-Aib-Glu(OtBu)-Gly-OPfp

The present invention further provides fragmental peptide of Semaglutide, wherein the fragmental peptide is selected from the group consisting of:

[SEQ ID NO: 178]

Boc-His(Trt)-Aib-Glu(OtBu)-Gly-OH,

[SEQ ID NO: 179]

Fmoc-His(Trt)-Aib-Glu(OtBu)-Gly-OH,

[SEQ ID NO: 180]

Cbz-His(Trt)-Aib-Glu(OtBu)-Gly-OH,

[SEQ ID NO: 181]

Boc-His(Trt)-Aib-Glu(OtBu)-Gly-OSu,

[SEQ ID NO: 182]

Fmoc-His(Trt)-Aib-Glu(OtBu)-Gly-OSu,

[SEQ ID NO: 183]

Cbz-His(Trt)-Aib-Glu(OtBu)-Gly-OSu,

[SEQ ID NO: 184]

Boc-His(Trt)-Aib-Glu(OtBu)-Gly-OBt,

[SEQ ID NO: 185]

Fmoc-His(Trt)-Aib-Glu(OtBu)-Gly-OBt,

[SEQ ID NO: 186]

Cbz-His(Trt)-Aib-Glu(OtBu)-Gly-OBt,

[SEQ ID NO: 187]

Boc-His(Trt)-Aib-Glu(OtBu)-Gly-OPfp,

[SEQ ID NO: 188]

Fmoc-His(Trt)-Aib-Glu(OtBu)-Gly-OPfp,

and

[SEQ ID NO: 189]

Cbz-His(Trt)-Aib-Glu(OtBu)-Gly-OPfp,

preferably wherein the fragmental peptide is selected from the group consisting of:

[SEQ ID NO 178]
Boc-His(Trt)-Aib-Glu(OtBu)-Gly-OH,
[SEQ ID NO: 181]
Boc-His(Trt)-Aib-Glu(OtBu)-Gly-OSu,
[SEQ ID NO: 184]
Boc-His(Trt)-Aib-Glu(OtBu)-Gly-OBt,

and

[SEQ ID NO: 187]
Boc-His(Trt)-Aib-Glu(OtBu)-Gly-OPfp

and more preferably wherein the fragmental peptide is:

[SEQ ID NO: 187]
Boc-His(Trt)-Aib-Glu(OtBu)-Gly-OPfp.

A further aspect of the present invention provides the use of any of the above fragmental peptides as an intermediate in the synthesis of semaglutide.

Also provided is semaglutide containing less than 5 wt %, less than 2 wt %, less than 1%, less than 0.5%, less than 0.2%, or less than 0.1% of the D-His isomer of semaglutide; or semaglutide containing less than 5 wt %, less than 2 wt %, less than 1%, less than 0.5%, less than 0.2%, or less than 0.1% of the [+Gly¹⁶] derivative of semaglutide; or semaglutide containing less than 5 wt %, less than 2 wt %, less than 1%, less than 0.5%, less than 0.2%, or less than 0.1% of the [+Gly³¹] derivative of semaglutide; or semaglutide containing less than 5 wt %, less than 2 wt %, less than 1%, less than 0.5%, less than 0.2%, or less than 0.1% of the [+Gly⁴] derivative of semaglutide.

The present invention further provides semaglutide containing:
(a) less than 5 wt %, less than 2 wt %, less than 1%, less than 0.5%, less than 0.2%, or less than 0.1% of the D-His isomer of semaglutide, and/or
(b) less than 5 wt %, less than 2 wt %, less than 1%, less than 0.5%, less than 0.2%, or less than 0.1% of the [+Gly¹⁶] derivative of semaglutide, and/or
(c) less than 5 wt %, less than 2 wt %, less than 1%, less than 0.5%, less than 0.2%, or less than 0.1% of the [+Gly³¹] derivative of semaglutide, and/or.
(d) less than 5 wt %, less than 2 wt %, less than 1%, less than 0.5%, less than 0.2%, or less than 0.1% of the [+Gly⁴] derivative of semaglutide.

### Liraglutide Purification

In a further aspect of the present invention, there is provided a facile method of purifying liraglutide which can achieve a high purity product suitable for use in pharmaceutical formulations. The process employs a two stage HPLC process using two different mobile phase systems. The process comprises:
(a) dissolving crude Liraglutide in a solution comprising acetonitrile and water,
(b) subjecting the solution in step (a) to reversed-phase HPLC on a C8 or C18 silica column using a mobile phase A, comprising water, and a mobile phase B, comprising acetonitrile and at least one C₁-4 alcohol, and collecting the liraglutide fractions,
(c) optionally repeating step (b),
(d) subjecting the fractions to reversed-phase HPLC on a C8 or C18 silica column using a mobile phase C, comprising water, and a mobile phase D, comprising acetonitrile, and collecting the purified Liraglutide fractions,
(e) optionally concentrating the purified liraglutide fractions to form a purified liraglutide concentrate,
(f) optionally repeating step (d), or optionally repeating steps (d) and (e), and
(g) drying the purified liraglutide fractions or purified liraglutide concentrate,
wherein the purified liraglutide fractions or purified liraglutide concentrate before drying has a pH of 6.0-8.5.

Preferably, the purified liraglutide concentrate before drying has a pH of 6.0-8.0, preferably 6.0-7.5, more preferably 6.5-7.5, particularly 6.5-7.4, or 6.8-7.3 or 7.0-7.3.

In the first HPLC stage, two mobile phases, A and B are employed, preferably as a gradient elution. Mobile phase A preferably comprises an aqueous solution of a chemical modifier. The chemical modifier is preferably an ammonium salt or a sodium salt or a combination thereof. Particularly, the chemical modifier is selected from the group consisting of: ammonium chloride, ammonium bicarbonate, ammonium phosphate, ammonium sulphate, ammonium hydroxide, sodium chloride, sodium bicarbonate, sodium phosphate and sodium sulphate, or a combination thereof. More preferably, the chemical modifier is an ammonium salt, especially ammonium chloride, ammonium bicarbonate, ammonium phosphate, ammonium sulphate and ammonium hydroxide or a combination thereof, and more preferably ammonium chloride.

Preferably, the chemical modifier is present in mobile phase A in a concentration of 0.001-1.0 M, preferably 0.002M-0.5 M, more preferably 0.005 M-0.1 M, most preferably 0.02M-0.05 M or especially about 0.01M.

The pH of the mobile phase A is preferably from 5.5-11.5, more preferably 6.0-11.0, most preferably 6.5-10.5 or 7.0-9.5, or particularly, the pH of the mobile phase A is about 8.5.

As mentioned above, mobile phase A comprises acetonitrile and at least one C₁₋₄ alcohol. Preferably the ratio (vol:vol) of acetonitrile to the least one C₁₋₄ alcohol (vol:vol) in mobile phase B is from 60:40 to 95:5, more preferably 65:35 to 80:20, and most preferably 70:30 to 75:25 or about 70:30. A particularly preferred C₁₋₄ alcohol in mobile phase B is ethanol.

According to preferred embodiments of the purification process of the present invention, step (b) is carried out by gradient elution, preferably from 75:25 (vol mobile phase A:vol mobile phase B) to 35:65 (vol mobile phase A:vol mobile phase B) over a period of 30 minutes to 1 hour, preferably over about 30 minutes.

In the second HPLC stage, two mobile phases, C and D are employed, preferably as a gradient elution.

Mobile phase C preferably comprises an aqueous solution of a chemical modifier. The chemical modifier is preferably an ammonium salt or a sodium salt or a combination thereof. Particularly, the chemical modifier is selected from the group consisting of: ammonium chloride, ammonium bicarbonate, ammonium phosphate, ammonium sulphate, ammonium hydroxide, sodium chloride, sodium bicarbonate, sodium phosphate and sodium sulphate, or a combination thereof. More preferably, the chemical modifier is an ammonium salt, especially ammonium chloride, ammonium bicarbonate, ammonium phosphate, ammonium sulphate and ammonium hydroxide or a combination thereof, and more preferably ammonium chloride.

Preferably, the aqueous solution of the chemical modifier in mobile phase C has a pH of 7.5-10.0, more preferably 7.5-9.5, and particular 7.8-9.0 or about 8.0.

In preferred embodiments, mobile phase C may further comprise an organic solvent selected from the group consisting of: acetonitrile, IPA, ethanol, THF, or a combination thereof. Preferably, mobile phase C further comprises acetonitrile. When mobile phase C includes an organic solvent (e.g. acetonitrile), the ratio (vol:vol) of the water or the aqueous solution of a chemical modifier to organic solvent is preferably from 98:2 to 70:30, more preferably 95:5 to 80:20, most preferably 95:5 to 85:15, or about 90:10.

Step (d) of the purification process is preferably carried out by gradient elution, preferably from 10:90 (vol mobile phase C:vol mobile phase D) to 50:50 (vol mobile phase C:vol mobile phase D) over a period of 30 minutes to 1 hour, preferably over about 30 minutes.

In the above purification process, the fractions from steps (b), (c), (d) and/or (e) are concentrated by evaporation before the subsequent steps.

Following the final HPLC run, the liraglutide fractions are concentrated in order to produce a purified liraglutide concentrate. This purified liraglutide concentrate can be directly used to prepare a dried liraglutide product which is suitable for preparing a pharmaceutical composition. Preferably, the concentrate employed in step (g) has a liraglutide concentration of 2-40 mg/ml, more preferably 5-30 mg/ml or 5-25 mg/ml, and most preferably 10-25 mg/ml or 15-25 mg/ml.

The purified liraglutide concentrate can be dried by any suitable process, especially processes which enable a rapid removal of water at low temperature, such as by spray drying, or lyophilization. Preferably, the drying step (g) comprises lyophilisation.

The above described purification process for liraglutide is especially useful for purifying liraglutide obtained by chemical peptide synthesis techniques. More preferably, the crude liraglutide is obtained from a solid-phase or liquid phase peptide synthesis.

The crude liraglutide from such a synthesis is preferably treated before the HPLC steps, wherein the treatment comprises stirring the crude liraglutide with an aqueous alkaline buffer solution at a pH of 8-12, preferably a pH of 9-11.5, more preferably a pH of 9.5-11, and most preferably a pH of 10-11 or 10.5-11.

A preferred aqueous alkaline buffer solution comprises aqueous glycine. A preferred buffer concentration is 1.0 M-0.001 M, more preferably 0.5 M-0.01 M and most preferably 0.3 M-0.05 M.

The stirring is preferably for a period of 0.5-6 hours, 0.5-5 hours, 1-4 hours or 2-4 hours.

The stirring may be done at temperature ranging from 10° C. to 50° C. preferably, 15° C. to 40° C. and most preferably 20° C. to 30° C., or a room or ambient temperature.

The solution may optionally contain an organic solvent in an amount of 0-70 vol %, 5%-50%, or 10%-30%. The organic solvent may preferably be selected from the group consisting of: acetonitrile, THF and IPA, or a combination thereof. Acetonitrile is a particularly preferred organic solvent.

After the treatment step, the pH of the mixture is preferably adjusted to 7.5-11, 8-10.5, preferably 8.5-10 and more preferably 8.5-9.5 or about 9 with an acid, preferably an organic acid. Suitable organic acids may preferably be selected from acetic acid and trifluoroacetic acid, and more preferably trifluoroacetic acid.

The above-described purification process can produce liraglutide of high purity suitable for the preparation of a pharmaceutical composition. Preferably, the dried liraglutide product has a purity of 98.5% or more, 99.0% or more, 99.5% or more, 99.8% or more, 99.9% or more, or 99.95% or more. The dried liraglutide product may be combined with at least one pharmaceutically acceptable excipient to form a pharmaceutical composition.

Further aspects and embodiments of the present invention are set out in the following numbered paragraphs:

1. A process for preparing liraglutide [SEQ ID NO: 1] of formula:

```
  1 2 3 4 5 6 7 8 9 10 11 12
His-Ala-Glu-Gly-Thr-Phe-Thr-Ser-Asp-Val-Ser-Ser-
  13 14 15 16 17 18 19 20 21 22
Tyr-Leu-Glu-Gly-Gln-Ala-Ala-Lys(Pal-Glu)-Glu-Phe-
  23 24 25 26 27 28 29 30 31
Ile-Ala-Trp-Leu-Val-Arg-Gly-Arg-Gly-OH
```

comprising:
(i) coupling a Peptide 1 having the sequence:

[SEQ ID NO: 5]
His-Ala-Glu-Gly

wherein:
   the N-terminal of His is optionally protected with a protecting group, preferably selected from the group consisting of Boc, Cbz or Fmoc, and
   the Gly carboxylic acid group in Peptide 1 may be in the form of an activated carboxylic acid derivative;
with a Peptide 2 having the sequence:

[SEQ ID NO: 24]
Thr-Phe-Thr-Ser-Asp-Val-Ser-Ser-Tyr-Leu-Glu-Gly-
Gln-Ala-Ala-Lys(Pal-Glu-OX)-Glu-Phe-Ile-Ala-Trp-
Leu-Val-Arg-Gly-Arg-Gly-OH-

wherein:
   Peptide 2 is optionally conjugated to a solid support (preferably, in this process, the Peptide 2 is either conjugated to a Wang resin, or Peptide 2 is not present on a resin. When the Peptide 2 is not present on a resin, the coupling of Peptide 1 with Peptide 2 is conducted as a liquid phase synthesis);
   X represents H or a protecting group for the Glu carboxylic acid group, and wherein one or more of the amino acid residues in Peptide 1 and Peptide 2 may be protected or unprotected;
(ii) optionally removing any protecting groups and/or cleaving the resin to form liraglutide; and
(iii) optionally purifying the liraglutide.
   2. A process according to Paragraph 1 wherein the Gly carboxylic acid group in Peptide 1 may be in the form of activated carboxylic acid derivative, preferably wherein the activated carboxylic acid derivative is selected from the group consisting of:
an activated ester, preferably wherein the activated ester is selected from the group consisting of OSu, OPfp, OBt, OAt, ODhbt, ONB, OPht, ONP, ODNP, Ot, Oct, and more preferably OSu or OPfp;
a mixed anhydride; and
an acid halide, preferably OCl or OF.
   3. A process according to Paragraph 1 or Paragraph 2 wherein the coupling is conducted in the presence of a coupling agent, preferably wherein the coupling agent is selected from the group consisting of BOP, AOP, PyBOP, PyAOP, HBTU, HATU, HCTU, HBPyU, HAPyU, TFFH, TBTU, BTFFH, EDC-HCl, PyBrop, DPPA, BOP—Cl, DCC, DIC, DEPC, EEDQ, IIDQ, CIP, PfTU, PfPU, BroP and CDI, more preferably TBTU and DIC.
   4. A process according to any of Paragraphs 1-3 wherein one or more of the amino acid residues in Peptide 1 and Peptide 2 are protected with acid-cleavable protecting groups.
   5. A process according to Paragraph 4 wherein the acid-cleavable protecting groups are selected from the group consisting of: tBu, OtBu, Ψ^{Me,Me}pro, Trt, and Pbf
   6. A process according to any of Paragraphs 1-5 wherein the protected amino acid residues in Peptides 1 and 2 are as follows: His(Trt), Thr(tBu), Ser(tBu) or Ser(Trt), Asp (OtBu), Ser(Ψ^{Me,Me}pro), Tyr(tBu), Glu(OtBu), Gln(Trt), and Arg(Pbf), and preferably wherein the amino acid residues of Peptides 1 and 2 are protected as follows: Thr(tBu), Ser⁸(tBu), Ser¹¹(tBu), Asp (OtBu), Ser¹²(Ψ^{Me,Me}pro), Tyr(tBu), Glu(OtBu), Gln(Trt), and Arg(Pbf), or wherein the amino acid residues of Peptides 1 and 2 are protected as follows: Thr(tBu), Ser⁸(tBu), Ser¹¹(Trt), Asp (OtBu), Ser¹²(Trt), Tyr(tBu), Glu(OtBu), Gln(Trt), and Arg(Pbf).
   7. A process according to any of Paragraphs 1-6, wherein Peptide 1 is selected from the group consisting of:

[SEQ ID NO: 7]
Boc-His(Trt)-Ala-Glu(OtBu)-Gly-OH,
[SEQ ID NO: 8]
Fmoc-His(Trt)-Ala-Glu(OtBu)-Gly-OH,
[SEQ ID NO: 9]
Cbz-His(Trt)-Ala-Glu(OtBu)-Gly-OH,
[SEQ ID NO: 10]
Boc-His(Trt)-Ala-Glu(OtBu)-Gly-OSu,
[SEQ ID NO: 11]
Fmoc-His(Trt)-Ala-Glu(OtBu)-Gly-OSu,
[SEQ ID NO: 12]
Cbz-His(Trt)-Ala-Glu(OtBu)-Gly-OSu,
[SEQ ID NO: 13]
Boc-His(Trt)-Ala-Glu(OtBu)-Gly-OBt,
[SEQ ID NO: 14]
Fmoc-His(Trt)-Ala-Glu(OtBu)-Gly-OBt,

and

[SEQ ID NO: 15]
Cbz-His(Trt)-Ala-Glu(OtBu)-Gly-OBt

or wherein Peptide 1 is selected from the group consisting of:

[SEQ ID NO: 268]
Boc-His(Trt)-Ala-Glu(OtBu)-Gly-OPfp
[SEQ ID NO: 269]
Fmoc-His(Trt)-Ala-Glu(OtBu)-Gly-OPfp.

and

[SEQ ID NO: 270]
Cbz-His(Trt)-Ala-Glu(OtBu)-Gly-OPfp.

8. A process according to any of Paragraphs 1-7 wherein Peptide 1 is selected from the group consisting of:

[SEQ ID NO: 7]
Boc-His(Trt)-Ala-Glu(OtBu)-Gly-OH,
[SEQ ID NO: 8]
Fmoc-His(Trt)-Ala-Glu(OtBu)-Gly-OH,
[SEQ ID NO: 10]
Boc-His(Trt)-Ala-Glu(OtBu)-Gly-OSu,

and

[SEQ ID NO: 11]
Fmoc-His(Trt)-Ala-Glu(OtBu)-Gly-OSu

or wherein Peptide 1 is selected from the group consisting of:

[SEQ ID NO: 268]
Boc-His(Trt)-Ala-Glu(OtBu)-Gly-OPfp.

and

[SEQ ID NO: 269]
Fmoc-His(Trt)-Ala-Glu(OtBu)-Gly-OPfp.

9. A process according to any of Paragraphs 1-8, which is carried out as a solid state peptide synthesis wherein Peptide 2 is conjugated to a solid support, preferably a Wang resin.
10. A process according to any of Paragraphs 1-9 wherein the Gly carboxylic acid group in Peptide 1 is not activated, and preferably wherein Peptide 1 is selected from the group consisting of:

[SEQ ID NO: 7]
Boc-His(Trt)-Ala-Glu(OtBu)-Gly-OH,

and

[SEQ ID NO: 8]

Fmoc-His(Trt)-Ala-Glu(OtBu)-Gly-OH,

and more preferably:

[SEQ ID NO: 7]

Boc-His(Trt)-Ala-Glu(OtBu)-Gly-OH,

11. A process according to any of Paragraphs 1-8, which is conducted in liquid phase.
12. A process according to Paragraph 11 wherein the Gly carboxylic acid in Peptide 1 is reacted to form an activated carboxylic acid derivative, and preferably wherein the activated carboxylic acid is selected from the group consisting of:

[SEQ ID NO: 10]

Boc-His(Trt)-Ala-Glu(OtBu)-Gly-OSu,

[SEQ ID NO: 11]

Fmoc-His(Trt)-Ala-Glu(OtBu)-Gly-OSu,

[SEQ ID NO: 13]

Boc-His(Trt)-Ala-Glu(OtBu)-Gly-OBt,

and

[SEQ ID NO: 14]

Fmoc-His(Trt)-Ala-Glu(OtBu)-Gly-OBt,

or
preferably wherein the activated carboxylic acid is selected from the group consisting of:

[SEQ ID NO: 268]

Boc-His(Trt)-Ala-Glu(OtBu)-Gly-OPfp,

and

[SEQ ID NO: 269]

Fmoc-His(Trt)-Ala-Glu(OtBu)-Gly-OPfp.

and more preferably:

[SEQ ID NO: 10]

Boc-His(Trt)-Ala-Glu(OtBu)-Gly-OSu,

or

[SEQ ID NO: 13]

Boc-His(Trt)-Ala-Glu(OtBu)-Gly-OBt

or

[SEQ ID NO: 268]

Boc-His(Trt)-Ala-Glu(OtBu)-Gly-OPfp.

13. A process according to any of Paragraphs 1-12 wherein Peptide 2 is selected from the group consisting of:

[SEQ ID NO: 33]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-Gly-
O-Wang resin,

[SEQ ID NO: 278 or SEQ ID NO: 316]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-O-
Wang resin,

[SEQ ID NO: 31]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-Gly-
OH,

and

[SEQ ID NO: 276 or SEQ ID NO: 309]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-OH,

14. A process for preparing Liraglutide according to Paragraph 1 comprising:
(i) coupling a Peptide 1 having the formula selected from the group consisting of:

[SEQ ID NO: 7]

Boc-His(Trt)-Ala-Glu(OtBu)-Gly-OH,

and

[SEQ ID NO: 8]

Fmoc-His(Trt)-Ala-Glu(OtBu)-Gly-OH,

with a Peptide 2 having the formula:

[SEQ ID NO: 33]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-Gly-
O-Wang resin

or

[SEQ ID NO: 278 or SEQ ID NO: 316]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-O-
Wang resin,

and
(ii) removing the protecting groups and resin to form liraglutide, and optionally
(iii) purifying the liraglutide.
   15. A process according to Paragraph 14 wherein Peptide 1 is:

[SEQ ID NO: 7]

Boc-His(Trt)-Ala-Glu(OtBu)-Gly-OH

16. A process for preparing liraglutide according to Paragraph 1 comprising:
(i) coupling a Peptide 1 having the formula selected from the group consisting of

[SEQ ID NO: 10]

Boc-His(Trt)-Ala-Glu(OtBu)-Gly-OSu,

[SEQ ID NO: 11]

Fmoc-His(Trt)-Ala-Glu(OtBu)-Gly-OSu,

[SEQ ID NO: 13]

Boc-His(Trt)-Ala-Glu(OtBu)-Gly-OBt,

and

[SEQ ID NO: 14]

Fmoc-His(Trt)-Ala-Glu(OtBu)-Gly-OBt,

or a Peptide 1 having the formula selected from the group consisting of:

[SEQ ID NO: 268]

Boc-His(Trt)-Ala-Glu(OtBu)-Gly-OPfp,

and

[SEQ ID NO: 269]

Fmoc-His(Trt)-Ala-Glu(OtBu)-Gly-OPfp

with a Peptide 2 having the formula:

[SEQ ID NO: 31]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-
Gly-OH

or

[SEQ ID NO: 276 or SEQ ID NO: 309]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-Gly-
OH

wherein the coupling is carried out in liquid phase,
(ii) removing the protecting groups to form liraglutide, and optionally
(iii) purifying the liraglutide.
   17. A process according to Paragraph 16 wherein Peptide 1 is selected from the group consisting of:

[SEQ ID NO: 10]

Boc-His(Trt)-Ala-Glu(OtBu)-Gly-OSu,

and

[SEQ ID NO: 13]

Boc-His(Trt)-Ala-Glu(OtBu)-Gly-OBt,

or wherein Peptide 1 is selected from the group consisting of:

[SEQ ID NO: 268]

Boc-His(Trt)-Ala-Glu(OtBu)-Gly-OPfp.

and preferably

[SEQ ID NO: 10]

Boc-His(Trt)-Ala-Glu(OtBu)-Gly-OSu

or

[SEQ ID NO: 268]

Boc-His(Trt)-Ala-Glu(OtBu)-Gly-OPfp..

18. A process according to any of Paragraphs 1-17 wherein Peptide 1 is purified to remove the D-His impurity of Peptide 1, having the amino acid sequence:

[SEQ ID NO: 5]

His-Ala-Glu-Gly

wherein the His has D configuration, and wherein the amino acid residues are optionally protected with acid-cleavable protecting groups corresponding to Peptide 1.
19. A process according to any preceding paragraph wherein Peptide 1 is prepared by:
(i) sequential coupling of amino acids on a resin, preferably CTC resin,
(ii) cleaving the Peptide 1 from the resin, and
(iii) optionally purifying the Peptide 1.
   20. A process according to Paragraph 18 or Paragraph 19 wherein Peptide 1 is purified by precipitating from a solution comprising at least one organic solvent, using an antisolvent.
   21. A process according to Paragraph 19 or Paragraph 20 wherein:
(i) Peptide 1 is cleaved from the resin using an acid in the presence of at least one organic solvent,
(ii) the reaction mixture is neutralised and/or washed with water and/or an aqueous base;
(iii) the mixture containing Peptide 1 is concentrated to produce a solution of Peptide 1 in the organic solvent, and
(iv) Peptide 1 is precipitated from the resulting mixture by addition of an antisolvent.
   22. A process according to Paragraph 20 or Paragraph 21 wherein the organic solvent is a halogenated hydrocarbon, preferably a bromo- or chloroalkane, and more preferably a brominated or chlorinated hydrocarbon, such as a brominated or chlorinated C₁-C₆ hydrocarbon, or brominated or chlorinated C₁-C₄ hydrocarbon, or mixtures thereof.
   23. A process according to Paragraph 22 wherein the organic solvent is dichloromethane, dibromomethane, and ethylene dichloride or mixtures thereof.
   24. A process according to any of Paragraphs 20-23 wherein the antisolvent comprises at least one of an ether and a hydrocarbon or mixtures thereof.
   25. A process according to Paragraph 24 wherein the antisolvent comprises a straight chain or branched C₄-C₈ dialkyl ether preferably a C₄-C₆ dialkyl ether, more preferably diethyl ether, methyl tert-butyl ether (MTBE) or mixtures thereof.
   26. A process according to any of Paragraphs 20-25 wherein the antisolvent is methyl tert-butyl ether (MTBE).
   27. A process according to any of Paragraphs 24-26 wherein the antisolvent comprises a C₆-C₁₀ hydrocarbon, preferably a C₆-C₈ hydrocarbon, more preferably hexane or petroleum ether, or mixtures thereof, and most preferably is hexane.
   28. A process according to any of Paragraphs 20-27 wherein the antisolvent comprises MTBE and optionally hexane or petroleum ether.
   29. A process according to any of Paragraphs 18-28 wherein Peptide 1 is purified by precipitation from a solution of Peptide 1 in a solvent comprising dichloromethane with an antisolvent comprising MTBE.
   30. A process according to any of Paragraphs 1-29 wherein the Peptide 1 contains less than 4%, less than 2%, less than 1%, less than 0.5%, less than 0.2% or less than 0.1% of the D-His analogue of Peptide 1.
   31. A process according to any of Paragraphs 1-30 wherein Peptide 1 contains less than 4%, less than 2%, less than 1%, less than 0.5%, less than 0.2% or less than 0.1% of the diglycine analogue of Peptide 1.
   32. A process according to any of Paragraphs 1-31 wherein the Peptide 2 is made by a two or three fragment convergent synthesis, preferably a two fragment convergent synthesis.
   33. A process according to any of Paragraphs 1-32 wherein Peptide 2 is prepared by coupling of Peptides 3 and 4, wherein one of Peptide 3 or Peptide 4 contains the residue:

-Lys(Pal-Glu-OX)-

wherein:
Peptide 3 and Peptide 4 together form the amino acid sequence of Peptide 2,
X represents H or a protecting group for the Glu carboxylic acid group,
one or more of the amino acid residues in Peptide 3 and Peptide 4 are optionally protected, and
Peptide 3 or Peptide 4, preferably Peptide 4, is optionally conjugated to a solid support.
   34. A process according to Paragraph 33 wherein Peptide 2 is prepared by coupling of Peptide 3 with Peptide 4, wherein the amino acid sequence in Peptide 3 is:

[SEQ ID NO: 42]

Thr-Phe-Thr-Ser-Asp-Val-Ser-Ser-Tyr-Leu-Glu-Gly-OH

wherein the Gly carboxylic acid group is optionally activated, preferably as an OSu ester or OPfp ester,
and the amino acid sequence in Peptide 4 is:

[SEQ ID NO: 50]

Gln-Ala-Ala-Lys(Pal-Glu-OX)-Glu-Phe-Ile-Ala-Trp-
Leu-Val-Arg-Gly-Arg-Gly-OH

wherein Peptide 4 is optionally conjugated to a resin at the terminal Gly-OH, preferably wherein the resin is a Wang resin.
35. A process according to any of Paragraphs 33-34 wherein one or more of the amino acid residues in Peptide 3 and Peptide 4 are protected with acid-cleavable protecting groups.
36. A process according to Paragraph 35 wherein the acid-cleavable protecting groups are selected from the group consisting of: tBu, OtBu, Ψ^{Me,Me}pro, Trt, and Pbf.
37. A process according to Paragraph 36 wherein the protected amino acid residues in Peptides 3 and 4 are as follows: Thr(tBu), Ser(tBu) or Ser(Trt), Asp (OtBu), Ser(Ψ^{Me,Me}pro), Tyr(tBu), Glu(OtBu), Gln(Trt), and Arg(Pbf), preferably wherein the amino acid residues of Peptides 3 and 4 are protected as follows: Thr(tBu), Ser⁸(tBu), Ser¹¹(tBu), Asp (OtBu), Ser¹²(Ψ^{Me,Me}pro), Tyr(tBu), Glu(OtBu), Gln(Trt), and Arg(Pbf), or wherein the amino acid residues of Peptides 3 and 4 are protected as follows: Thr(tBu), Ser⁸(tBu), Ser¹¹(Trt), Asp (OtBu), Ser¹²(Trt), Tyr(tBu), Glu(OtBu), Gln(Trt), and Arg(Pbf).
38. A process according to any of Paragraphs 33-37, wherein the coupling of Peptides 3 and 4 is carried out in liquid phase.
39. A process according to any of Paragraphs 32-37 comprising:
(i) coupling Peptide 3 of formula:

[SEQ ID NO: 44]

Fmoc-Thr(tBu)-Phe-Thr(tBu)-Ser(tBu)-Asp(OtBu)-
Val-Ser(tBu)-Ser(Ψ^{Me,Me}pro)-Tyr(tBu)-
Leu-Glu(OtBu)-Gly-OH

or

[SEQ ID NO: 287]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH,

to Peptide 4 of formula:

[SEQ ID NO: 55 or SEQ ID NO: 128]

Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-O-
Wang resin

and removing the Fmoc protecting group to form Peptide 2 of formula:

[SEQ ID NO: 33]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-Gln(Trt)-Ala-Ala-Lys(Pal-Glu-OtBu)-
Glu(OtBu)-Phe-Ile-Ala-Trp-Leu-Val-Arg(Pbf)-Gly-
Arg(Pbf)-Gly-O-Wang resin

or

[SEQ ID NO: 278 or SEQ ID NO: 316]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-
Gly-O-Wang resin,

(ii) coupling the Peptide 1 of formula:

[SEQ ID NO: 7]

Boc-His(Trt)-Ala-Glu(OtBu)-Gly-OH

to Peptide 2 to form an optionally protected liraglutide sequence,
(iii) deprotecting and removing the resin to form liraglutide, and optionally
(iv) purifying the liraglutide.
   40. A process according to Paragraph 39 wherein Peptide 4 is prepared by sequential synthesis on a resin, preferably a Wang resin, using Fmoc strategy, and wherein the -Lys(Pal-Glu-OX)- residue is formed by:
(i) sequential coupling of Fmoc-Lys(Mtt)-OH or Fmoc-Lys(Mmt)-OH
(ii) selectively removing the Mtt or Mmt protecting group with acid, and coupling a Pal-Glu-OX residue to the Lys residue,
   wherein step (ii) can be carried out on the partial or complete sequence of Peptide 4.
      41. A process according to Paragraph 38 comprising:
(i) liquid phase coupling of Peptide 3 of formula:

[SEQ ID NO: 45]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-OSu

or

[SEQ ID NO: 288]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OSu

or

[SEQ ID NO: 289]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OPfp

or

[SEQ ID NO: 290]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-OPfp

to Peptide 4 of formula:

[SEQ ID NO: 56 or SEQ ID NO: 148]

Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-OH

and removing the Fmoc to form Peptide 2 of formula:

[SEQ ID NO: 31]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-
Glu(OtBu)-Gly-Gln(Trt)-Ala-Ala-Lys(Pal-Glu-OtBu)-
Glu(OtBu)-Phe-Ile-Ala-Trp-Leu-Val-Arg(Pbf)-
Gly-Arg(Pbf)-Gly-OH

or

[SEQ ID NO: 276 or SEQ ID NO: 309

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-
Gly-OH,

(ii) coupling the Peptide 1 of formula:

[SEQ ID NO: 10]

Boc-His(Trt)-Ala-Glu(OtBu)-Gly-OSu

[SEQ ID NO: 13]

Boc-His(Trt)-Ala-Glu(OtBu)-Gly-OBt

or the Peptide 1 of formula:

[SEQ ID NO: 268]

Boc-His(Trt)-Ala-Glu(OtBu)-Gly-OPfp,

to Peptide 2 to form an optionally protected liraglutide sequence, and
(iii) deprotecting to form liraglutide, and optionally
(iv) purifying the liraglutide.
   42. A process according to Paragraph 41 wherein Peptide 4 is prepared by sequential synthesis on a resin, preferably a CTC resin, using Fmoc strategy, comprising:
(i) forming the Lys(Pal-Glu-OX) residue by sequential coupling of Fmoc-Lys(Trt-Glu-OtBu)-OH
(ii) coupling one or more amino acid residues sequentially to the Lys residue to form the amino acid sequence of Peptide 4,
(iii) simultaneously removing the Trt protecting group of the Lys(Trt-Glu-OtBu) residue and cleavage of the peptide from the resin,
(iv) coupling Pal to Glu by reaction with Pal-OSu or Pal-OPfp, and
(v) removing the Fmoc group
   43. A process according to Paragraph 41 wherein Peptide 4 is prepared by sequential synthesis on a resin, preferably a CTC, using Fmoc strategy, comprising:
(i) forming the -Lys(Pal-Glu-OX)- residue by sequential coupling of Fmoc-Lys(Mmt)-OH or Fmoc-Lys(Mtt)-OH
(ii) coupling one or more amino acid residues sequentially to the Lys residue to form the amino acid sequence of Peptide 4,
(iii) simultaneously removing the Mmt or Mtt protecting group and cleavage of the peptide from the resin,
(iv) coupling a Pal-Glu-OX residue to the Lys residue, wherein the side chain carboxylic acid group in Glu may be activated in the form of an OSu ester or OPfp ester, and
(v) removing the Fmoc group.
   44. A process according to any of Paragraphs 1-32 wherein Peptide 2 is prepared by a process comprising:
(i) coupling of Peptides 3 and 4A, wherein one of Peptide 3 or Peptide 4A contains the residue:

-Lys(Y-Glu-OX)-

wherein:
   Peptide 3 and Peptide 4A together form the amino acid sequence of Peptide 2,
   X represents H or a protecting group for the Glu carboxylic acid group,
   Y represents a protecting group for the Glu amino group, and
   one or more of the amino acid residues in Peptide 3 and Peptide 4A are optionally protected, and
   Peptide 3 or Peptide 4A is optionally conjugated to a solid support,
(ii)) removal of the protecting group Y and if present, cleaving the peptide from the support, and
(iii) coupling Pal to the Glu residue to form Peptide 2, and
(iv) removing the Fmoc group.
   45. A process according to Paragraph 44 wherein Peptide 2 is prepared by coupling of Peptide 3 with Peptide 4A, wherein the amino acid sequence in Peptide 3 is:

[SEQ ID NO: 42]

Thr-Phe-Thr-Ser-Asp-Val-Ser-Ser-Tyr-Leu-Glu-Gly-OH

wherein the Gly-OH is optionally activated with an OSu ester or OPfp ester, and wherein the amino acid sequence in Peptide 4A is:

[SEQ ID NO: 51]

Gln-Ala-Ala-Lys(Y-Glu-OX)-Glu-Phe-Ile-Ala-Trp-Leu-
Val-Arg-Gly-Arg-Gly-OH

wherein Peptide 4A is optionally conjugated to a resin at the terminal Gly residue, preferably wherein the resin is selected from a Wang resin or a CTC resin, and more preferably a CTC resin, and wherein the amino acid residues in Peptide 3 and Peptide 4A are optionally protected.
46. A process according to Paragraph 44 or Paragraph 45, comprising:
(i) coupling Peptide 3 of formula:

[SEQ ID NO: 44]

Fmoc-Thr(tBu)-Phe-Thr(tBu)-Ser(tBu)-Asp(OtBu)-Val-
Ser(tBu)-Ser (Ψ^{Me,Me}pro)-Tyr(tBu)-Leu-
Glu(OtBu)-Gly-OH

or

[SEQ ID NO: 287]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

to Peptide 4A of formula:

[SEQ ID NO: 59 or SEQ ID NO: 143]

Gln(Trt)-Ala-Ala-Lys(Trt-Glu-OtBu)-Glu(OtBu)-Phe-
Ile-Ala-Trp-Leu-Val-Arg(Pbf)-Gly-Arg(Pbf)-Gly-O-
CTC resin,

(ii) simultaneously removing the Glu-Trt protecting group and resin,
(iii) coupling the peptide product from step (ii) with Pal-OSu or Pal-OPfp and removing Fmoc to form Peptide 2 of formula:

[SEQ ID NO: 31]

Thr(tBu)-Phe-Thr(tBu)-Ser(tBu)-Asp(OtBu)-Val-
Ser(tBu)-Ser(Ψ^{Me,Me}pro)-Tyr(tBu)-
Leu-Glu(OtBu)-Gly-Gln(Trt)-Ala-Ala-
Lys(Pal-Glu-OtBu)-Glu(OtBu)-Phe-Ile-Ala-Trp-
Leu-Val-Arg(Pbf)-Gly-Arg(Pbf)-Gly-OH

or

[SEQ ID NO: 276 or SEQ ID NO: 309]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-
Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-OH

(iv) coupling the Peptide 1 of formula:

[SEQ ID NO: 10]

Boc-His(Trt)-Ala-Glu(OtBu)-Gly-OSu,

or

[SEQ ID NO: 13]

Boc-His(Trt)-Ala-Glu(OtBu)-Gly-OBt,

or

[SEQ ID NO: 268]

Boc-His(Trt)-Ala-Glu(OtBu)-Gly-OPfp.

to Peptide 2 to form an optionally protected liraglutide sequence, and
(v) deprotecting to form liraglutide, and
(vi) optionally purifying the liraglutide.
   47. A process according to any of Paragraphs 44-46 wherein Peptide 4A is prepared by sequential synthesis on a resin, preferably a CTC resin, using Fmoc strategy, and wherein the -Lys(Pal-Glu-OX)- residue is formed by sequential coupling of Fmoc-Lys(Trt-Glu-OtBu).
   48. A process according to any of Paragraphs 44-47 wherein one or more of the amino acid residues in Peptide 3 and Peptide 4A are protected with acid-cleavable protecting groups.
   49. A process according to Paragraph 48 wherein the acid-cleavable protecting groups are selected from the group consisting of: tBu, OtBu, Ψ^{Me,Me}pro, Trt, and Pbf.
   50. A process according to Paragraph 49 wherein the protected amino acid residues in Peptides 3 and 4A are as follows: Thr(tBu), Ser(tBu) or Ser(Trt), Asp(OtBu), Ser(Ψ^{Me,Me}pro), Tyr(tBu), Glu(OtBu), Gln(Trt), and Arg(Pbf), preferably wherein the amino acid residues of Peptides 3 and 4A are protected as follows: Thr(tBu), Ser⁸(tBu), Ser¹¹(tBu), Lys(Trt-Glu-OtBu), Asp (OtBu), Ser¹²(Ψ^{Me,Me}pro), Tyr(tBu), Glu(OtBu), Gln(Trt), and Arg(Pbf), or wherein the amino acid residues of Peptides 3 and 4A are protected as follows: Thr(tBu), Ser⁸(tBu), Ser¹¹(Trt), Lys(Trt-Glu-OtBu), Asp (OtBu), Ser¹²(Trt), Tyr(tBu), Glu(OtBu), Gln(Trt), and Arg(Pbf).
   51. A process according to any of Paragraphs 44-50 wherein Peptide 4A is conjugated to a resin, preferably a CTC resin.
   52. A process according to any of Paragraphs 33-51 wherein the N-terminal of Thr in Peptide 3 is protected with Fmoc or CBz, and preferably with Fmoc.
   53. A process according to any of Paragraphs 33-37 wherein Peptide 4 contains the residue -Lys(Pal-Glu-OX)-, wherein Peptide 4 is prepared by sequential synthesis on a resin, preferably a Wang resin, using Fmoc strategy, and wherein the -Lys(Pal-Glu-OX)-residue is formed by:
   (i) sequential coupling of Fmoc-Lys(Mtt)-OH or Fmoc-Lys(Mmt)-OH,
   (ii) selectively removing the Mtt or Mmt protecting group with acid, and coupling a Pal-Glu-OX residue to the Lys residue, wherein step (ii) can be carried out on the partial or complete sequence of Peptide 4.
      54. A process according to Paragraph 53 wherein step (ii) comprises coupling with Pal-Glu-OtBu wherein the side chain carboxylic acid group in Glu is optionally in the form of an activated carboxylic acid derivative, wherein the activated carboxylic acid derivative is preferably in the form of an activated ester.
      55. A process according to Paragraph 54 wherein the reaction is with Pal-Glu-OtBu, Pal-Glu(OSu)-OtBu, Pal-Glu(OPfp)-OtBu or Pal-Glu(OBt)-OtBu, preferably Pal-Glu-OtBu.
      56. A process according to any of Paragraphs 33-37 wherein Peptide 4 contains the residue -Lys(Pal-Glu-OX)-, wherein Peptide 4 is prepared by sequential synthesis on a resin, preferably a CTC resin, using Fmoc strategy, comprising:
   (i) sequential coupling of Fmoc-Lys(Trt-Glu-OtBu)-OH,
   (ii) coupling amino acid residues sequentially to the Lys residue to form the amino acid sequence of Peptide 4,
   (iii) simultaneously removing the Trt protecting group of the Lys(Trt-Glu-OtBu) residue and cleavage of the peptide from the resin, and
   (iv) coupling Pal to Glu to form Peptide 4.
      57. A process according to Paragraph 56 wherein step (iv) comprises reaction with palmitic acid, preferably wherein the carboxylic acid group in the palmitic acid is in the form of an activated carboxylic acid derivative, preferably in the form of an activated ester.
      58. A process according to Paragraph 57 wherein the reaction is with Pal-OSu, Pal-OPfp or Pal-OBt, preferably Pal-OSu or Pal-OPfp.
      59. A process according to any of Paragraphs 33-37 wherein Peptide 4 contains the residue -Lys(Pal-Glu-OX)-, wherein Peptide 4 is prepared by sequential synthesis on a resin, preferably a CTC, using Fmoc strategy, comprising:
   (i) sequential coupling of Fmoc-Lys(Mmt)-OH or Fmoc-Lys(Mtt)-OH,
   (ii) coupling one or more amino acid residues sequentially to the Lys residue to form the amino acid sequence of Peptide 4,
   (iii) simultaneously removing the Mmt or Mtt protecting group and cleavage of the peptide from the resin, and
   (iv) coupling a Pal-Glu-OX residue to the Lys residue, wherein the side chain carboxylic acid group in the Glu may be in the form of an activated carboxylic acid derivative.
      60. A process according to Paragraph 59 wherein step (iv) comprises reaction with Pal-Glu-OtBu wherein the side chain carboxylic acid group in Glu is preferably in the form of an activated carboxylic acid derivative, wherein the activated carboxylic acid derivative is preferably an activated ester.
      61. A process according to Paragraph 60 wherein the reaction is with Pal-Glu(OSu)-OtBu, Pal-Glu(OPfp)-OtBu or Pal-Glu(OBt)-OtBu, preferably Pal-Glu(OSu)-OtBu or Pal-Glu(OPfp)-OtBu.
      62. A process according to any of Paragraphs 53-55 wherein the amino acid sequence in Peptide 4 is:

[SEQ ID NO: 51]

Gln-Ala-Ala-Lys(Pal-Glu-OX)-Glu-Phe-Ile-Ala-Trp-
Leu-Val-Arg-Gly-Arg-Gly-OH

wherein one or more amino acid residues are optionally protected, and preferably wherein Peptide 4 is:

[SEQ ID NO: 56 or SEQ ID NO: 148]

Gln(Trt)-Ala-Ala-Lys(Pal-Glu-OtBu)-Glu(OtBu)-Phe-
Ile-Ala-Trp-Leu-Val-Arg(Pbf)-Gly-Arg(Pbf)-Gly-OH

wherein Peptide 4 may be conjugated to a resin, preferably a Wang resin [SEQ ID NO: 57].
   63. A process according to any of Paragraphs 44-52 wherein Peptide 4A contains the residue -Lys(Y-Glu-OX)-, wherein Peptide 4A is prepared by sequential synthesis on a resin, preferably a CTC resin, using Fmoc strategy, and wherein the -Lys(Y-Glu-OX)-residue is formed by:
(i) sequential coupling of Fmoc-Lys(Trt-Glu-OtBu)-OH,
(ii) coupling amino acid residues sequentially to the Lys residue to form the amino acid sequence of Peptide 4A.
   64. A process according to Paragraph 63 wherein the amino acid sequence in Peptide 4A is

[SEQ ID NO: 51]

Gln-Ala-Ala-Lys(Y-Glu-OX)-Glu-Phe-Ile-Ala-Trp-Leu-
Val-Arg-Gly-Arg-Gly-OH

wherein one or more amino acids are optionally protected, and wherein Peptide 4A is preferably:

Gln(Trt)-Ala-Ala-Lys(Trt-Glu-OtBu)-Glu(OtBu)-Phe-Ile-Ala-Trp-Leu-Val-Arg(Pbf)-Gly-Arg(Pbf)-Gly-OH
which is preferably conjugated to a resin, preferably a CTC resin [SEQ ID NO: 58].
   65. A process according to any of Paragraphs 33-64 wherein the amino acid sequence in Peptide 3 is:

[SEQ ID NO: 42]

Thr-Phe-Thr-Ser-Asp-Val-Ser-Ser-Tyr-Leu-Glu-Gly-OH

wherein
   the Gly carboxylic acid group may be in the form of an activated carboxylic acid derivative, and
one or more amino acids are optionally protected, and preferably wherein Peptide 3 is:

[SEQ ID NO: 44]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-OH

or

[SEQ ID NO: 287]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

wherein the Gly carboxylic acid group may be in the form of an activated carboxylic acid derivative.
   66. A process according to Paragraph 65 wherein Peptide 3 is:

[SEQ ID NO: 141]

Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-OH

or

[SEQ ID NO: 305]

Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

67. A process according to Paragraph 66 wherein the N-terminal of Thr(tBu) is protected with Boc or Fmoc (preferably Fmoc), and optionally the Gly carboxylic acid group is reacted to form an activated carboxylic acid derivative, preferably an activated ester.
68. A process according to Paragraph 67 wherein Peptide 3 is:

[SEQ ID NO: 45]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-OSu

or

[SEQ ID NO: 288]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OSu

or wherein Peptide 3 is:

[SEQ ID NO: 289]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OPfp

or

[SEQ ID NO: 290]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-OPfp

69. A process according to any of Paragraphs 1-32 wherein Peptide 2 is prepared by:
(i) coupling of Peptides 3 and 4B, wherein one of Peptide 3 or Peptide 4B contains the residue:

-Lys²⁰(Y)

wherein:
   Peptide 3 and Peptide 4B together form the amino acid sequence of Peptide 2 wherein the residue at position 20 is Lys(Y),
   Y represents a protecting group for the Glu amino group selected from Mtt or Mmt,
   one or more of the amino acid residues in Peptide 3 and 4B are optionally protected, and
   Peptide 3 or Peptide 4B is optionally conjugated to a solid support,
(ii) optionally cleaving the peptide from the support,
(iii) removing the protecting group Y,
(iv) coupling a Pal-Glu-OX residue to the Lys, wherein X is H or a protecting group for the Glu carboxylic acid group and wherein the side chain carboxylic acid group in Glu is optionally in the form of an activated carboxylic acid derivative, and optionally,
(v) deprotecting and removing the peptide from the support.
   70. A process according to Paragraph 69 wherein the amino acid sequence in Peptide 3 is:

[SEQ ID NO: 42]

Thr-Phe-Thr-Ser-Asp-Val-Ser-Ser-Tyr-Leu-Glu-Gly-OH

wherein the Gly carboxylic acid group is optionally in the form of an activated ester derivative, preferably an OSu ester or an OPfp ester,
and wherein the amino acid sequence in Peptide 4B is:

[SEQ ID NO: 50]

Gln-Ala-Ala-Lys(Y)-Glu-Phe-Ile-Ala-Trp-Leu-Val-
Arg-Gly-Arg-Gly-OH

### and Y is Mtt or Mmt,

wherein Peptide 4B is optionally conjugated to a resin at the terminal Gly residue, preferably wherein the resin is a Wang resin and wherein the amino acid residues in Peptide 3 and Peptide 4B are optionally protected.
71. A process according to any of Paragraphs 69-70 wherein one or more of the amino acid residues in Peptide 3 and Peptide 4B are protected with acid-cleavable protecting groups.
72. A process according to Paragraph 71 wherein the acid-cleavable protecting groups are selected from the group consisting of: tBu, OtBu, Ψ^{Me,Me}pro, Trt, Mmt, Mtt and Pbf.
73. A process according to Paragraph 72 wherein the protected amino acid residues in Peptides 3 and 4B are as follows: Thr(tBu), Ser(tBu) or Ser(Trt), Asp(OtBu), Ser(Ψ^{Me,Me}pro), Lys(Mmt) or Lys(Mtt), Tyr(tBu), Glu(OtBu), Gln(Trt), and Arg(Pbf), preferably wherein the amino acid residues of Peptides 3 and 4B are protected as follows: Thr(tBu), Ser⁸(tBu), Ser¹¹(tBu), Lys(Mmt) or Lys(Mtt), Asp (OtBu), Ser¹²(Ψ^{Me,Me}pro), Tyr(tBu), Glu(OtBu), Gln(Trt), and Arg(Pbf), or wherein the amino acid residues of Peptides 3 and 4B are protected as follows: Thr(tBu), Ser⁸(tBu), Ser¹¹(Trt), Lys(Mtt) or Lys(Mmt), Asp (OtBu), Ser¹²(Trt), Tyr(tBu), Glu(OtBu), Gln(Trt), and Arg(Pbf).
74. A process according to any of Paragraphs 69-73 comprising:
(i) coupling Peptide 3 of formula:

[SEQ ID NO: 44]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-OH,

or

[SEQ ID NO: 287]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

with Peptide 4B of formula:

[SEQ ID NO: 75 or SEQ ID NO: 77]

Gln(Trt)-Ala-Ala-Lys(Y)-Glu(OtBu)-Phe-Ile-Ala-Trp-
Leu-Val-Arg(Pbf)-Gly-Arg(Pbf)-Gly-O-Wang resin

wherein Y is Mmt or Mtt,
(ii) removing the protecting group Y,
(iii) coupling a Pal-Glu-OX residue to the Lys, and
(iv) removing Fmoc and cleaving the peptide from the support to form Peptide 2.
   75. A process according to any of Paragraphs 69-74 wherein Peptide 4B is prepared by sequential synthesis on a resin, preferably a Wang resin, using Fmoc strategy, wherein the Lys(Y) residue is formed by sequential coupling of Fmoc-Lys(Y)-OH
   76. A process according to Paragraph 74 or Paragraph 75 wherein step (iii) comprises reaction with Pal-Glu-OX, wherein X represents a protecting group for the Glu carboxylic acid group, and preferably wherein X represents OtBu.
   77. A process according to any of Paragraphs 69-73, wherein the coupling of Peptides 3 and 4B is carried out in liquid phase.
   78. A process according to Paragraph 77 comprising:
(i) liquid phase coupling of Peptide 3 of formula:

[SEQ ID NO: 44]

Fmoc-Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-
Gly-OH,

or

[SEQ ID NO: 287]

Fmoc-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-OH

wherein the Gly carboxylic acid group is preferably in the form of an activated derivative, preferably an OSu ester or an OPfp ester, with Peptide 4B of formula:

[SEQ ID NO: 74 or SEQ ID NO: 76]

Gln(Trt)-Ala-Ala-Lys(Y)-Glu(OtBu)-Phe-Ile-Ala-Trp-
Leu-Val-Arg(Pbf)-Gly-Arg(Pbf)-Gly-OH

wherein Y is Mmt or Mtt,
(ii) removing the protecting group Y,
(iii) coupling a Pal-Glu-OX residue to the Lys residue by reaction with Pal-Glu-OX, wherein the side chain carboxylic acid group in Glu is in the form of an activated carboxylic acid derivative.
   79. A process according to Paragraph 78, preferably wherein step (iii) comprises reaction with Pal-Glu(OSu)-OtBu, Pal-Glu(OPfp)-OtBu, Pal-Glu(OBt)-OtBu, preferably Pal-Glu(OSu)-OtBu or Pal-Glu(OPfp)-OtBu.
   80. A process according to any of Paragraphs 77-79 wherein Peptide 4B is prepared by sequential synthesis on a resin, preferably a CTC resin, using Fmoc strategy, wherein the Lys(Y) residue is formed by sequential coupling of Fmoc-Lys(Y)-OH, and removing the Fmoc group and cleaving the peptide from the resin to form Peptide 4B.
   81. A process according to any of Paragraphs 33-80 wherein Peptide 3 is prepared by a two fragment coupling on a resin, and cleaving the peptide from the resin.
   82. A process according to Paragraph 81 wherein Peptide 3 is prepared by:
(i) coupling a Peptide 5 containing the optionally protected amino acid sequence:

[SEQ ID NO: 78]

Thr-Phe-Thr-Ser-Asp-Val-Ser-Ser

with a Peptide 6 containing the optionally protected amino acid sequence:

[SEQ ID NO: 84]

Tyr-Leu-Glu-Gly-OH

which is conjugated to a resin, and
(ii) cleaving the Peptide 3 from the resin.
   83. A process according to Paragraph 82 wherein Peptide 3 is prepared by:
(i) coupling of Peptide 5 having the formula:

[SEQ ID NO: 82]

Fmoc-Thr(tBu)-Phe-Thr(tBu)-Ser(tBu)-Asp(OtBu)-Val-
Ser(tBu)-Ser(Ψ^{Me,Me}pro)

or

[SEQ ID NO: 300]

Fmoc-Thr(tBu)-Phe-Thr(tBu)-Ser(tBu)-Asp(OtBu)-Val-
Ser(Trt)-Ser(Trt)

with a Peptide 6 of formula:

Tyr(tBu)-Leu-Glu(OtBu)-Gly-OH

which is conjugated to a CTC resin—[SEQ ID NO: 88 or SEQ ID NO: 163], and
(ii) cleaving the peptide from the resin.
   84. A peptide selected from the group consisting of:

A.

[SEQ ID NO: 2]

P1-His(P)-Ala-Glu(P)-Gly-Thr(P)-Phe-Thr(P)-Ser(P)-
Asp(P)-Val-Ser(P)-Ser(P)-Tyr(P)-Leu-Glu(P)-Gly-
Gln(P)-Ala-Ala-Lys(Pal-Glu-P)-Glu(P)-Phe-Ile-Ala-
Trp-Leu-Val-Arg(P)-Gly-Arg(P)-Gly-O-P2

wherein P1 represents a protecting group for the N-terminal of His (preferably an acid-cleavable protecting group, more preferably Boc or Cbz, and most preferably Boc), each P represents side chain protecting groups (preferably acid-cleavable protecting groups) which may be the same or different, and P2 is H or P2 represents a solid support, preferably a Wang resin;

B.

[SEQ ID NO: 6]

P1-His(P)-Ala-Glu(P)-Gly-O-P2

wherein P1 represents a protecting group for the N-terminal of His (preferably Fmoc, Boc or CBZ, preferably an acid-cleavable protecting group, and most preferably Boc), each P represents side chain protecting groups (preferably acid-cleavable protecting groups) which may be the same or different, and P2 is selected from: H, or a solid support (preferably a CTC resin), or P2 represents an activated carboxylic ester of the Gly⁴ residue (preferably Su or Bt or Pfp);

C1.

[SEQ ID NO: 20]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(P)-Glu(P)-Phe-Ile-Ala-Trp-Leu-Val-Arg(P)-Gly-
Arg(P)-Gly-OP2

wherein P1 represents H, or a protecting group for the N-terminal of Thr (preferably a base-cleavable protecting group, more preferably Fmoc or Cbz, and most preferably Fmoc), each P represents side chain protecting groups which may be the same or different (preferably acid-cleavable protecting groups), and P2 is selected from H, or a solid support, preferably a CTC or Wang resin;

C2.

[SEQ ID NO: 22]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(Glu-P)-Glu(P)-Phe-Ile-Ala-Trp-Leu-Val-Arg(P)-
Gly-Arg(P)-Gly-OP2

wherein P1 represents H, or a protecting group for the N-terminal of Thr (preferably Fmoc or Cbz and more preferably Fmoc), each P represents side chain protecting groups which may be the same or different, and P2 is selected from H (i.e. the carboxylic acid of the Gly³¹ residue is unsubstituted, and thus contains a free —OH group), or a solid support, preferably a CTC or Wang resin;

C3.

[SEQ ID NO: 23]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(P1-Glu-P)-Glu(P)-Phe-Ile-Ala-Trp-Leu-Val-
Arg(P)-Gly-Arg(P)-Gly-OP2

wherein P1 represents H, or N-terminal protecting groups for the N-Thr and/or Glu wherein P1 may be the same or different (preferably for Thr, P1 is Fmoc or Cbz and more preferably Fmoc, and preferably for Glu, P1 is Trt), each P represents side chain protecting groups which may be the same or different, and P2 is selected from H (i.e. the carboxylic acid of the Gly³¹ residue is unsubstituted, and thus contains a free —OH group), or a solid support, preferably a CTC or Wang resin;

C4.

[SEQ ID NO: 25]

P1-Thr(P)-Phe-Thr(P)-Ser(P)-Asp(P)-Val-Ser(P)-
Ser(P)-Tyr(P)-Leu-Glu(P)-Gly-Gln(P)-Ala-Ala-
Lys(Pal-Glu-P)-Glu(P)-Phe-Ile-Ala-Trp-Leu-Val-
Arg(P)-Gly-Arg(P)-Gly-OP2

wherein P1 represents H, or a protecting group for the N-terminal of Thr (preferably Fmoc or Cbz and more preferably Fmoc), each P represents side chain protecting groups which may be the same or different, and P2 is selected from H (i.e. the carboxylic acid of the Gly³¹ residue is unsubstituted, and thus contains a free —OH group), or a solid support, preferably a CTC or Wang resin.

D.

[SEQ ID NO: 43]

P1-Thr(P)-Phe-Thr(P)-Ser(P)-Asp(P)-Val-Ser(P)-
Ser(P)-Tyr(P)-Leu-Glu(P)-Gly-OP2

wherein P1 represents a protecting group for the N-terminal of Thr (preferably a base-cleavable protecting group, more preferably Fmoc or Cbz, and most preferably Fmoc), each P represents side chain protecting groups which may be the same or different (preferably acid-cleavable protecting groups), and P2 is selected from H (i.e. the carboxylic acid of the Gly¹⁶ residue is unsubstituted, and thus contains a free —OH group), or P2 represents an activated carboxylic ester of the Gly¹⁶ residue (preferably Su or OPfp), or P2 represents a solid support, preferably a CTC resin;

E1.

[SEQ ID NO: 53]

P1-Gln(P)-Ala-Ala-Lys(P3-Glu-P)-Glu(P)-Phe-Ile-
Ala-Trp-Leu-Val-Arg(P)-Gly-Arg(P)-Gly-O-P2

wherein P1 represents H or a protecting group for the N-terminal of Gin (preferably Fmoc or Cbz and more preferably Fmoc), each P represents side chain protecting groups which may be the same or different, P2 is selected from H (i.e. the carboxylic acid of the Gly³¹ residue is unsubstituted, and thus contains a free —OH group), or P2 represents a solid support, preferably a CTC or Wang resin, and P3 represents a protecting group for the Glu nitrogen atom or Pal

E2.

[SEQ ID NO: 54]

P1-Gln(P)-Ala-Ala-Lys(Y)-Glu(P)-Phe-Ile-Ala-Trp-
Leu-Val-Arg(P)-Gly- Arg(P)-Gly-O-P2

wherein P1 represents H or a protecting group for the N-terminal of Gln (preferably Fmoc or Cbz and more preferably Fmoc), each P represents side chain protecting groups which may be the same or different, P2 is selected from H (i.e. the carboxylic acid of the Gly³¹ residue is unsubstituted, and thus contains a free —OH group), or P2 represents a solid support, preferably a CTC or Wang resin, and Y represents Mmt or Mtt.

F.

[SEQ ID NO: 79]

P1-Thr(P)-Phe-Thr(P)-Ser(P)-Asp(P)-Val-Ser(P)-
Ser(P)-O-P2

wherein P1 represents a protecting group for the N-terminal of Thr (preferably a base-cleavable protecting group, more preferably Fmoc or Cbz and most preferably Fmoc), each P represents side chain protecting groups which may be the same or different (preferably acid-cleavable protecting groups), and P2 is H or a solid support, preferably a CTC resin;

G.

[SEQ ID NO: 85]

P1-Tyr(P)-Leu-Glu(P)-Gly-O-P2

wherein P1 represents a protecting group for the N-terminal of Tyr (preferably a base-cleavable protecting group, more preferably Fmoc or Cbz, and most preferably Fmoc), each P represents side chain protecting groups which may be the same or different (preferably acid-cleavable protecting groups), and P2 is a solid support, preferably a CTC resin;

H.

[SEQ ID NO: 90]

P1-Lys(P)-Glu(P)-Phe-Ile-Ala-Trp-Leu-Val-Arg(P)-
Gly-Arg(P)-Gly-O-P2

wherein P1 represents a protecting group for the N-terminal of Lys (preferably a base-cleavable protecting group, more preferably Fmoc or Cbz and most preferably Fmoc), each P represents side chain protecting groups which may be the same or different (preferably acid-cleavable protecting groups), and P2 is a solid support, preferably a Wang resin;

I.

[SEQ ID NO: 102]

Ala-Lys(Pal-Glu-P)-Glu(P)-Phe-Ile-Ala-Trp-Leu-
Val-Arg(P)-Gly-Arg(P)-Gly-O-P2,

wherein each P represents side chain protecting groups which may be the same or different (preferably acid-cleavable protecting groups), and P2 is a solid support, preferably a Wang resin; and

J.

[SEQ ID NO: 105]

Ala-Ala-Lys(Pal-Glu-P)-Glu(P)-Phe-Ile-Ala-Trp-Leu-
Val-Arg(P)-Gly-Arg(P)-Gly-O-P2

wherein each P represents side chain protecting groups which may be the same or different (preferably acid-cleavable protecting groups), and P2 is a solid support, preferably a Wang resin.
   85. A peptide according to Paragraph 84 wherein the side chain protecting groups P are: Thr(tBu), Ser⁸ (tBu), Ser⁸(Trt), Ser¹¹(tBu), Ser¹¹(Trt), Lys(Mtt) or Lys(Mmt), Asp (OtBu), Ser¹²(Ψ^{Me,Me}pro), Ser¹²(Trt), Tyr(tBu), Glu(OtBu), Gln(Trt), and Arg(Pbf) (wherein the amino acid numbering is based on the liraglutide backbone sequence).
   86. A fragmental peptide of Liraglutide, wherein the fragmental peptide is selected from the group consisting of:

(i)
[SEQ ID NO: 118]
His(Trt)-Ala-Glu(OtBu)-Gly-OH,
(ii)
[SEQ ID NO: 119]
His(Trt)-Ala-Glu(OtBu)-Gly-OSu,
(iii)
[SEQ ID NO: 120]
His(Trt)-Ala-Glu(OtBu)-Gly-OBt,
(iv)
[SEQ ID NO: 121]
His(Trt)-Ala-Glu(OtBu)-Gly-OPfp,
(v)
[SEQ ID NO: 122]
His(Trt)-Ala-Glu(OtBu)-Gly-O-CTC resin,
(vi)
[SEQ ID NO: 123]
Lys(Mtt)-Glu(OtBu)-Phe-Ile-Ala-Trp-Leu-Val-Arg(Pbf)-Gly-
Arg(Pbf)-Gly-O-Wang resin,
(vii)
[SEQ ID NO: 124]
Lys(Mmt)-Glu(OtBu)-Phe-Ile-Ala-Trp-Leu-Val-Arg(Pbf)-Gly-
Arg(Pbf)-Gly-O-Wang resin,
(viii)
[SEQ ID NO: 125]
Lys-Glu(OtBu)-Phe-Ile-Ala-Trp-Leu-Val-Arg(Pbf)-Gly-Arg(Pbf)-
Gly-O-Wang resin,
(ix)
[SEQ ID NO: 126 or SEQ ID NO: 167]
Lys(Glu-OtBu)-Glu(OtBu)-Phe-Ile-Ala-Trp-Leu-Val-Arg(Pbf)-Gly-
Arg(Pbf)-Gly-O-Wang resin.
(x)
[SEQ ID NO: 127]
Lys(Pal-Glu-OtBu)-Glu(OtBu)-Phe-Ile-Ala-Trp-Leu-Val-Arg(Pbf)-
Gly-Arg(Pbf)-Gly-O-Wang resin,
(xi)
[SEQ ID NO: 128 or SEQ ID NO: 55]
Gln(Trt)-Ala-Ala-Lys(Pal-Glu-OtBu)-Glu(OtBu)-Phe-Ile-Ala-Trp-
Leu-Val-Arg(Pbf)-Gly-Arg(Pbf)-Gly-O-Wang resin,
(xii)
[SEQ ID NO: 129]
Ala-Lys(Mtt)-Glu(OtBu)-Phe-Ile-Ala-Trp-Leu-Val-Arg(Pbf)-Gly-
Arg(Pbf)-Gly-O-Wang resin,
(xiii)
[SEQ ID NO: 130]
Ala-Lys(Mmt)-Glu(OtBu)-Phe-Ile-Ala-Trp-Leu-Val-Arg(Pbf)-Gly-
Arg(Pbf)-Gly-O-Wang resin,
(xiv)
[SEQ ID NO: 131]
Ala-Lys(Trt-Glu-OtBu)-Glu(OtBu)-Phe-Ile-Ala-Trp-Leu-Val-
Arg(Pbf)-Gly-Arg(Pbf)-Gly-O-Wang resin,
(xv)
[SEQ ID NO: 132]
Ala-Lys(Glu-OtBu)-Glu(OtBu)-Phe-Ile-Ala-Trp-Leu-Val-Arg(Pbf)-
Gly-Arg(Pbf)-Gly-O-Wang resin,
(xvi)
[SEQ ID NO: 133]
Ala-Lys-Glu(OtBu)-Phe-Ile-Ala-Trp-Leu-Val-Arg(Pbf)-Gly-
Arg(Pbf)-Gly-O-Wang resin,
(xvii)
[SEQ ID NO: 134 or SEQ ID NO: 103]
Ala-Lys(Pal-Glu-OtBu)-Glu(OtBu)-Phe-Ile-Ala-Trp-Leu-Val-
Arg(Pbf)-Gly-Arg(Pbf)-Gly-O-Wang resin,
(xviii)
[SEQ ID NO: 135]
Ala-Ala-Lys(Mtt)-Glu(OtBu)-Phe-Ile-Ala-Trp-Leu-Val-Arg(Pbf)-
Gly-Arg(Pbf)-Gly-O-Wang resin,
(xix)
[SEQ ID NO: 136]
Ala-Ala-Lys(Mmt)-Glu(OtBu)-Phe-Ile-Ala-Trp-Leu-Val-Arg(Pbf)-
Gly-Arg(Pbf)-Gly-O-Wang resin,
(xx)
[SEQ ID NO: 137]
Ala-Ala-Lys(Trt-Glu-OtBu)-Glu(OtBu)-Phe-Ile-Ala-Trp-Leu-Val-
Arg(Pbf)-Gly-Arg(Pbf)-Gly-O-Wang resin,
(xxi)
[SEQ ID NO: 138]
Ala-Ala-Lys(Glu-OtBu)-Glu(OtBu)-Phe-Ile-Ala-Trp-Leu-Val-
Arg(Pbf)-Gly-Arg(Pbf)-Gly-O-Wang resin,
(xxii)
[SEQ ID NO: 139]
Ala-Ala-Lys-Glu(OtBu)-Phe-Ile-Ala-Trp-Leu-Val-Arg(Pbf)-Gly-
Arg(Pbf)-Gly-O-Wang resin,
(xxiii)
[SEQ ID NO: 140 or SEQ ID NO: 112]
Ala-Ala-Lys(Pal-Glu-OtBu)-Glu(OtBu)-Phe-Ile-Ala-Trp-Leu-Val-
Arg(Pbf)-Gly-Arg(Pbf)-Gly-O-Wang resin,
(xxiv)
[SEQ ID NO: 141]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-OH,
(xxv)
[SEQ ID NO: 305]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OH,
(xxvi)
[SEQ ID NO: 142]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-OSu,
(xxvii)
[SEQ ID NO: 306]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OSu,
(xxviii)
[SEQ ID NO: 307]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-OPfp
(xxix)
[SEQ ID NO: 308]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-OPfp
(xxx)
[SEQ ID NO: 143 or SEQ ID NO: 59]
Gln(Trt)-Ala-Ala-Lys(Trt-Glu-OtBu)-Glu(OtBu)-Phe-Ile-Ala-Trp-
Leu-Val-Arg(Pbf)-Gly-Arg(Pbf)-Gly-O-CTC resin,
(xxxi)
[SEQ ID NO: 144 or SEQ ID NO: 154]
Gln(Trt)-Ala-Ala-Lys(Mmt)-Glu(OtBu)-Phe-Ile-Ala-Trp-Leu-Val-
Arg(Pbf)-Gly-Arg(Pbf)-Gly-O-CTC resin,
(xxxii)
[SEQ ID NO: 145 or SEQ ID NO: 152]
Gln(Trt)-Ala-Ala-Lys(Mtt)-Glu(OtBu)-Phe-Ile-Ala-Trp-Leu-Val-
Arg(Pbf)-Gly-Arg(Pbf)-Gly-O-CTC resin,
(xxxiii)
[SEQ ID NO: 146 or SEQ ID NO: 153]
Gln(Trt)-Ala-Ala-Lys-Glu(OtBu)-Phe-Ile-Ala-Trp-Leu-Val-
Arg(Pbf)-Gly-Arg(Pbf)-Gly-OH,
(xxxiv)
[SEQ ID NO: 147]
Gln(Trt)-Ala-Ala-Lys(Glu-OtBu)-Glu(OtBu)-Phe-Ile-Ala-Trp-Leu-
Val-Arg(Pbf)-Gly-Arg(Pbf)-Gly-OH,
(xxxv)
[SEQ ID NO: 148 or SEQ ID NO: 56]
Gln(Trt)-Ala-Ala-Lys(Pal-Glu-OtBu)-Glu(OtBu)-Phe-Ile-Ala-Trp-
Leu-Val-Arg(Pbf)-Gly-Arg(Pbf)-Gly-OH,
(xxxvi)
[SEQ ID NO: 149]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Pal-Glu-OtBu)-Glu(OtBu)-Phe-Ile-Ala-Trp-Leu-Val-Arg(Pbf)-
Gly-Arg(Pbf)-Gly-OH,
(xxxvii)
[SEQ ID NO: 309 or SEQ ID NO: 276]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Pal-Glu-
OtBu)-Glu(OtBu)-Phe-Ile-Ala-Trp-Leu-Val-Arg(Pbf)-Gly-
Arg(Pbf)-Gly-OH,
(xxxviii)
[SEQ ID NO: 150]
Gln(Trt)-Ala-Ala-Lys(Trt-Glu-OtBu)-Glu(OtBu)-Phe-Ile-Ala-Trp-
Leu-Val-Arg(Pbf)-Gly-Arg(Pbf)-Gly-O-Wang resin,
(xxxix)
[SEQ ID NO: 151]
Gln(Trt)-Ala-Ala-Lys(Glu-OtBu)-Glu(OtBu)-Phe-Ile-Ala-Trp-Leu-
Val-Arg(Pbf)-Gly-Arg(Pbf)-Gly-O-Wang resin,
(xl)
[SEQ ID NO: 152 or SEQ ID NO: 145]
Gln(Trt)-Ala-Ala-Lys(Mtt)-Glu(OtBu)-Phe-Ile-Ala-Trp-Leu-Val-
Arg(Pbf)-Gly-Arg(Pbf)-Gly-O-CTC resin,
(xli)
[SEQ ID NO: 153 or SEQ ID NO: 146]
Gln(Trt)-Ala-Ala-Lys-Glu(OtBu)-Phe-Ile-Ala-Trp-Leu-Val-
Arg(Pbf)-Gly-Arg(Pbf)-Gly-OH,
(xlii)
[SEQ ID NO: 154 or SEQ ID NO: 144]
Gln(Trt)-Ala-Ala-Lys(Mmt)-Glu(OtBu)-Phe-Ile-Ala-Trp-Leu-Val-
Arg(Pbf)-Gly-Arg(Pbf)-Gly-O-CTC resin,
(xliii)
[SEQ ID NO: 155]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mtt)-Glu(OtBu)-Phe-Ile-Ala-Trp-Leu-Val-Arg(Pbf)-Gly-
Arg(Pbf)-Gly-O-CTC resin,
(xliv)
[SEQ ID NO: 310]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mtt)-
Glu(OtBu)-Phe-Ile-Ala-Trp-Leu-Val-Arg(Pbf)-Gly-Arg(Pbf)-Gly-
O-CTC resin,
(xlv)
[SEQ ID NO: 156]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys-Glu(OtBu)-Phe-Ile-Ala-Trp-Leu-Val-Arg(Pbf)-Gly-Arg(Pbf)-
Gly-OH,
(xlvi)
[SEQ ID NO: 311]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys-Glu(OtBu)-
Phe-Ile-Ala-Trp-Leu-Val-Arg(Pbf)-Gly-Arg(Pbf)-Gly-OH,
(xlvii)
[SEQ ID NO: 157]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mtt)-Glu(OtBu)-Phe-Ile-Ala-Trp-Leu-Val-Arg(Pbf)-Gly-
Arg(Pbf)-Gly-O-Wang resin,
(xlviii)
[SEQ ID NO: 312]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mtt)-
Glu(OtBu)-Phe-Ile-Ala-Trp-Leu-Val-Arg(Pbf)-Gly-Arg(Pbf)-Gly-
O-Wang resin,
(xlix)
[SEQ ID NO: 158]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mmt)-Glu(OtBu)-Phe-Ile-Ala-Trp-Leu-Val-Arg(Pbf)-Gly-
Arg(Pbf)-Gly-O-Wang resin,
(l)
[SEQ ID NO: 313]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mmt)-
Glu(OtBu)-Phe-Ile-Ala-Trp-Leu-Val-Arg(Pbf)-Gly-Arg(Pbf)-Gly-
O-Wang resin,
(li)
[SEQ ID NO: 159]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys-Glu(OtBu)-Phe-Ile-Ala-Trp-Leu-Val-Arg(Pbf)-Gly-Arg(Pbf)-
Gly-O-Wang resin,
(lii)
[SEQ ID NO: 314]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys-Glu(OtBu)-
Phe-Ile-Ala-Trp-Leu-Val-Arg(Pbf)-Gly-Arg(Pbf)-Gly-O-Wang
resin,
(liii)
[SEQ ID NO: 160]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Trt-Gly-OtBu)-Glu(OtBu)-Phe-Ile-Ala-Trp-Leu-Val-Arg(Pbf)-
Gly-Arg(Pbf)-Gly-O-Wang resin,
(liv)
[SEQ ID NO: 315]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Trt-Gly-
OtBu)-Glu(OtBu)-Phe-Ile-Ala-Trp-Leu-Val-Arg(Pbf)-Gly-
Arg(Pbf)-Gly-O-Wang resin,
(lv)
[SEQ ID NO: 161]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Pal-Glu-OtBu)-Glu(OtBu)-Phe-Ile-Ala-Trp-Leu-Val-Arg(Pbf)-
Gly-Arg(Pbf)-Gly-O-Wang resin,
(lvi)
[SEQ ID NO: 316 or SEQ ID NO: 278]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Pal-Glu-
OtBu)-Glu(OtBu)-Phe-Ile-Ala-Trp-Leu-Val-Arg(Pbf)-Gly-
Arg(Pbf)-Gly-O-Wang resin,
(lvii)
[SEQ ID NO: 162]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Glu-OtBu)-Phe-Ile-Ala-Trp-Leu-Val-Arg(Pbf)-Gly-Arg(Pbf)-
Gly-OH,
(lviii)
[SEQ ID NO: 317]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Glu-OtBu)-
Phe-Ile-Ala-Trp-Leu-Val-Arg(Pbf)-Gly-Arg(Pbf)-Gly-OH,
(lix)
[SEQ ID NO: 163 or SEQ ID NO: 88]
Tyr(tBu)-Leu-Glu(OtBu)-Gly-O-CTC resin,
(lx)
[SEQ ID NO: 164]
Thr(tBu)-Phe-Thr(tBu)-Ser(tBu)-Asp(OtBu)-Val-Ser(tBu)-
Ser(Ψ^{Me,Me}pro)-O-CTC resin,
(lxi)
[SEQ ID NO: 318]
Thr(tBu)-Phe-Thr(tBu)-Ser(tBu)-Asp(OtBu)-Val-Ser(Trt)-Ser(Trt)-
O-CTC resin,
(lxii)
[SEQ ID NO: 165]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-O-CTC resin,
(lxiii)
[SEQ ID NO: 319]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-O-CTC resin,
(lxiv)
[SEQ ID NO: 166]
Lys(Trt-Glu-OtBu)-Glu(OtBu)-Phe-Ile-Ala-Trp-Leu-Val-Arg(Pbf)-
Gly-Arg(Pbf)-Gly-O-Wang resin,
(lxv)
[SEQ ID NO: 167 or SEQ ID NO: 126]
Lys(Glu-OtBu)-Glu(OtBu)-Phe-Ile-Ala-Trp-Leu-Val-Arg(Pbf)-Gly-
Arg(Pbf)-Gly-O-Wang resin,
(lxvi)
[SEQ ID NO: 168]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Mmt)-Glu(OtBu)-Phe-Ile-Ala-Trp-Leu-Val-Arg(Pbf)-Gly-
Arg(Pbf)-Gly-O-CTC resin,
(lxvii)
[SEQ ID NO: 320]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Mmt)-
Glu(OtBu)-Phe-Ile-Ala-Trp-Leu-Val-Arg(Pbf)-Gly-Arg(Pbf)-Gly-
O-CTC resin,
(lxviii)
[SEQ ID NO: 169]
Thr(tBu)-Phe-Thr(tBu)-Ser(tBu)-Asp(OtBu)-Val-Ser(tBu)-
Ser(Ψ^{Me,Me}pro)-Tyr(tBu)-Leu-Glu(OtBu)-Gly-Gln(Trt)-Ala-Ala-
Lys(Trt-Glu-OtBu)-Phe-Ile-Ala-Trp-Leu-Val-Arg(Pbf)-Gly-
Arg(Pbf)-Gly-O-CTC resin,
(lxix)
[SEQ ID NO: 321]
Thr(tBu)-Phe-Thr(tBu)-Ser(tBu)-Asp(OtBu)-Val-Ser(Trt)-Ser(Trt)-
Tyr(tBu)-Leu-Glu(OtBu)-Gly-Gln(Trt)-Ala-Ala-Lys(Trt-Glu-
OtBu)-Phe-Ile-Ala-Trp-Leu-Val-Arg(Pbf)-Gly-Arg(Pbf)-Gly-O-
CTC resin,
(lxx)
[SEQ ID NO: 170]
His(Trt)-Ala-Glu(OtBu)-Gly-Thr(tBu)-Phe-Thr(tBu)-Ser(tBu)-
Asp(OtBu)-Val-Ser(tBu)-Ser(Ψ^{Me,Me}pro)-Tyr(tBu)-Leu-Glu(OtBu)-
Gly-Gln(Trt)-Ala-Ala-Lys(Glu-OtBu)-Phe-Ile-Ala-Trp-Leu-Val-
Arg(Pbf)-Gly-Arg(Pbf)-Gly-O-Wang resin,
and
(lxxi)
[SEQ ID NO: 322]
His(Trt)-Ala-Glu(OtBu)-Gly-Thr(tBu)-Phe-Thr(tBu)-Ser(tBu)-
Asp(OtBu)-Val-Ser(Trt)-Ser(Trt)-Tyr(tBu)-Leu-Glu(OtBu)-Gly-
Gln(Trt)-Ala-Ala-Lys(Glu-OtBu)-Phe-Ile-Ala-Trp-Leu-Val-
Arg(Pbf)-Gly-Arg(Pbf)-Gly-O-Wang resin

wherein the N-terminal amino acids in (i)-(v) are optionally protected with Fmoc, CBz or Boc, and wherein the N-terminal amino acids in (vi)-(lxix) or (lxx) and (lxxi) are optionally protected with Fmoc or Cbz.
   87. A peptide (i)-(v) according to Paragraph 86 which is protected at the N-terminal amino acid with Fmoc, Boc or CBz.
   88. A peptide (i)-(v) according to Paragraph 87 which is protected at the N-terminal amino acid with Boc.
   89. A peptide (vi)-(lxix) or (lxx) and (lxxi) according to Paragraph 86 which is protected at the N-terminal amino acid with Fmoc or Cbz.
   90. A peptide according to Paragraph 89 wherein (vi)-(lxix) are protected at the N-terminal amino acid with Fmoc.
   91. Use of a peptide of any of Paragraphs 84-90 as an intermediate in a synthesis of liraglutide.

92.

[SEQ ID NO: 7]

Boc-L-His(Trt)-Ala-Glu(OtBu)-Gly-OH
93.

[SEQ ID NO: 10]

Boc-L-His(Trt)-Ala-Glu(OtBu)-Gly-OSu
94.

[SEQ ID NO: 13]

Boc-L-His(Trt)-Ala-Glu(OtBu)-Gly-OBt
95.

[SEQ ID NO: 268]

Boc-His(Trt)-Ala-Glu(OtBu)-Gly-OPfp

96. Liraglutide containing less than 5 wt %, less than 2 wt %, less than 1%, less than 0.5%, less than 0.2%, or less than 0.1% of the D-His isomer of liraglutide.
97. Liraglutide containing less than 5 wt %, less than 2 wt %, less than 1%, less than 0.5%, less than 0.2%, or less than 0.1% of the [+Gly¹⁶] derivative of liraglutide.
98. Liraglutide containing less than 5 wt %, less than 2 wt %, less than 1%, less than 0.5%, less than 0.2%, or less than 0.1% of the [+Gly³¹] derivative of liraglutide.
99. Liraglutide containing less than 5 wt %, less than 2 wt %, less than 1%, less than 0.5%, less than 0.2%, or less than 0.1% of the [+Gly⁴] derivative of liraglutide.
100. Liraglutide containing:
(a) less than 5 wt %, less than 2 wt %, less than 1%, less than 0.5%, less than 0.2%, or less than 0.1% of the D-His isomer of liraglutide, and/or
(b) less than 5 wt %, less than 2 wt %, less than 1%, less than 0.5%, less than 0.2%, or less than 0.1% of the [+Gly¹⁶] derivative of liraglutide, and/or
(c) less than 5 wt %, less than 2 wt %, less than 1%, less than 0.5%, less than 0.2%, or less than 0.1% of the [+Gly³¹] derivative of liraglutide, and/or.
(d) less than 5 wt %, less than 2 wt %, less than 1%, less than 0.5%, less than 0.2%, or less than 0.1% of the [+Gly⁴] derivative of liraglutide.

The following examples are provided to illustrate various aspects and embodiments of the present invention.

### EXAMPLES

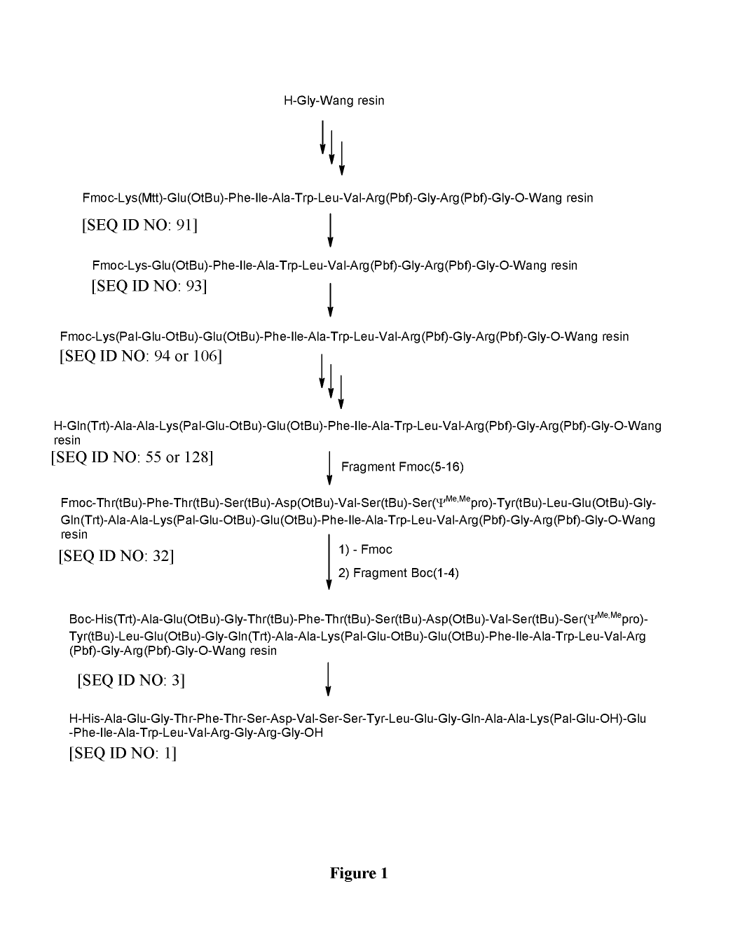

### Example 1: Synthesis of Liraglutide on Wang Resin (FIG. 1)

### 1.1. Synthesis of Fmoc-Gln(Trt)-Ala-Ala-Lys(Pal-Glu-OtBu)-Glu(OtBu)-Phe-Ile-Ala-Trp-Leu-Val-Arg(Pbf)-Gly-Arg(Pbf)-Gly-O-Wang Resin [SEQ ID NO: 57]

Synthesis of the peptide sequence is carried out by a stepwise Fmoc SPPS (solid phase peptide synthesis) procedure starting with H-Gly-Wang resin. The resin is washed by several washings with DMF and after the washing the second amino acid (Fmoc-Arg(Pbf)-OH) is introduced to start the first coupling step. The Fmoc protected amino acid is pre-activated using DIC/HOBt (N-hydroxybenzotriazole) and subsequently coupled to the resin for about 50 minutes. Completion of the coupling is indicated by a Ninhydrine test. After washing of the resin, the Fmoc protecting group on the α-amine is removed by washing with 20% piperidine in DMF for 20 min. These steps are repeated each time with another amino acid according to peptide sequence. All amino acids used are Fmoc-N^{α} protected. Trifunctional amino acids are side chain protected as follows: Gln(Trt), Arg(Pbf), Lys(Mtt), and Glu(OtBu). Up to three equivalents of the activated amino acids are used in the coupling reactions. After addition of Fmoc-Lys(Mtt)-OH the resin is washed with 1% TFA in DCM to remove Mtt group. Pal-Glu(OSu)-OtBu (or Pal-Glu-OtBu in the presence of a coupling agent, such as TBTU or DIC) is reacted with the free amino residue on Lys side chain. Completion of the reaction is monitored by ninhydrine test. After washing with DMF Fmoc group is removed by washing with piperidine/DMF solution and the synthesis is continued to obtain sequence of the peptide fragment.

### 1.2 Synthesis of Fmoc-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-OH [SEQ ID NO: 44]

Synthesis of the peptide sequence was carried out by a stepwise Fmoc SPPS (solid phase peptide synthesis) procedure starting with CTC resin 200 gr. The first amino acid (Fmoc-Gly-OH, 52.3 gram 1.1 eq) was loaded on the resin by DIPEA in NMP to obtain Fmoc-Gly-CTC resin (0.8 mmol/gram). The resin was washed with NMP followed by washing three times with DMF. Fmoc protecting group was removed with 20% piperidine in DMF solution. The resin was washed by several washings with DMF and after the washing the second amino acid (Fmoc-Glu(OtBu)-OH) was introduced. The Fmoc protected amino acid was pre-activated using DIC/HOBt/collidine and subsequently coupled to the resin for 60 minutes. After washing of the resin, the Fmoc protecting group on the α-amine was removed by washing with 20% piperidine in DMF for 40 min. These steps were repeated each time with another amino acid according to peptide sequence. All amino acids used were Fmoc-N^{α} protected. Trifunctional amino acids were side chain protected as follows: Thr(tBu), Ser(tBu), Asp(OtBu), Tyr(tBu) and Glu(OtBu). In addition Ser(tBu)-Ser(Ψ^{Me,Me}pro) unit was used at stage 4 of the synthesis. Up to three equivalents of the activated amino acids were used in the coupling reactions. At the end of the synthesis the peptide-resin was washed with DMF, followed by DCM, and dried under vacuum to obtain 383.9 g dry peptide-resin (58.6% yield due to weight added).

The peptide, prepared as described above, was cleaved from the resin using a 2% TFA solution in DCM by three repeated washings (15 min each). The acidic peptide solution was neutralized with DIPEA. The solvent was evaporated under reduced pressure and the protected peptide was precipitated by Hexane, filtered and dried in vacuum to obtain 141.9 g powder (45.2% yield). It was identified as Fmoc-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-OH [SEQ ID NO: 44] by MS.

### 1.3 Synthesis of Boc-His(Trt)-Ala-Glu(OtBu)-Gly-OH [SEQ ID NO: 7]

Synthesis of the peptide sequence was carried out by a stepwise Fmoc SPPS (solid phase peptide synthesis) procedure starting with CTC resin 200 gr). The first amino acid (Fmoc-Gly-OH, 52.3 gram 1.1 eq) was loaded on the resin by DIPEA in NMP to obtain Fmoc-Gly-CTC resin (0.8 mmol/gram). The resin was washed with NMP followed by washing three times with DMF. Fmoc protecting group was removed with 20% piperidine in DMF solution. The resin was washed by several washings with DMF and after the washing the second amino acid (Fmoc-Glu(OtBu)-OH) was introduced to start the first coupling step. The Fmoc protected amino acid was pre-activated using DIC/HOBt/collidine and subsequently coupled to the resin for 30 minutes. Completion of the coupling was indicated by a Ninhydrine test. After washing of the resin, the Fmoc protecting group on the α-amine was removed by washing with 20% piperidine in DMF for 40 min. These steps were repeated for Fmoc-Glu(OtBu)-OH and Boc-His(Trt)-OH according to peptide sequence. Up to three equivalents of the activated amino acids were used in the coupling reactions. At the end of the synthesis the peptide-resin was washed with DMF, followed by DCM, and dried under vacuum to obtain 330 g dry peptide-resin(100% yield due to weight added).

Peptide was cleaved from the peptide-resin (170 gram), using a 2% TFA solution by three repeated washings (15 min each total 2000 ml) and DCM (dichloromethane) as solvent. The acidic peptide solution was washed with H₂O two times (500 ml each) and additional wash with 1% NH₄OH pH=8 solution (1000 ml). After phase separation the organic phase was evaporated under reduced pressure to obtain 500 ml of a solution containing the product peptide in dichloromethane (the purity of the peptide was 71.9%). 1000 ml of cold MTBE were added and the solution was cooled ˜4° C., 1 h. The precipitated peptide was filtered and dried in vacuum to obtain 30 g powder (46.2% yield). After precipitation, the purity of the peptide was 94.0%. It was identified as Boc-His(Trt)-Ala-Glu(OtBu)-Gly-OH [SEQ ID NO: 7] by MS. The content of [D-His] impurity, i.e. Boc-D-His(Trt)-Ala-Glu(OtBu)-Gly-OH [SEQ ID NO: 173] before precipitation was 4.3% determined by HPLC, after precipitation the content of [D-His] impurity in the fragment was reduced to only 0.4%. The content of the [+Gly⁴] impurity, namely [Boc-His(Trt)-Ala-Glu(OtBu)-Gly-Gly-OH [SEQ ID NO: 115], before the precipitation was 16.2% determined by HPLC, and after precipitation the content of [+Gly⁴] impurity was under the level of detection.

### 1.4 Preparation of Fmoc-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(Pal-Glu-OtBu)-Glu(OtBu)-Phe-Ile-Ala-Trp-Leu-Val-Arg(Pbf)-Gly-Arg(Pbf)-Gly-O-Wang resin [SEQ ID NO: 32]

After removal of the Fmoc group from SEQ ID NO: 50 (i.e. Fmoc-Gln(Trt)-Ala-Ala-Lys(Pal-Glu-OtBu)-Glu(OtBu)-Phe-Ile-Ala-Trp-Leu-Val-Arg(Pbf)-Gly-Arg(Pbf)-Gly-O-Wang resin (Example 1.1, SEQ ID NO: 57), it is coupled to the peptide fragment Fmoc-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-OH] (Example 1.2, SEQ ID NO: 44) on solid support (Wang resin). Thus the peptide fragment of Example 1.2 is dissolved in DMF and activated by reaction with DIC/HOBt. This solution is added to the reactor containing SEQ ID NO: 50 (i.e. the Fmoc-deprotected peptide fragment of Example 1.1) on solid support (Wang resin). The reaction is continued until completion as monitored by HPLC. At the end of the reaction the resin is washed with DMF. The peptide on the resin is identified by MS analysis.

### 1.5 Preparation of Boc-His(Trt)-Ala-Glu(OtBu)-Gly-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(Pal-Glu-OtBu)-Glu(OtBu)-Phe-Ile-Ala-Trp-Leu-Val-Arg(Pbf)-Gly-Arg(Pbf)-Gly-O-Wang resin [SEQ ID NO: 3]

After removal of the Fmoc group from Fmoc-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Aa-Ala-Lys(Pal-Glu-OtBu)-Glu(OtBu)-Phe-Ile-Ala-Trp-Leu-Val-Arg(Pbf)-Gly-Arg(Pbf)-Gly-O-Wang resin (Example 1.4, SEQ ID NO: 32), it is coupled to Boc-His(Trt)-Ala-Glu(OtBu)-Gly-OH (SEQ ID NO: 7) on solid support (Wang resin). Thus Boc-His(Trt)-Ala-Glu(OtBu)-Gly-OH (SEQ ID NO: 7) is dissolved in DMF and preactivated by reaction with DIC/HOBt. This solution is added to the reactor containing previously prepared H-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(Pal-Glu-OtBu)-Glu(OtBu)-Phe-Ile-Ala-Trp-Leu-Val-Arg(Pbf)-Gly-Arg(Pbf)-Gly-O-Wang resin (SEQ ID NO: 33) on solid support (Wang resin). The reaction is continued until completion as monitored by HPLC analysis. At the end of the reaction the resin is washed with DMF. The peptide on the resin is identified by MS analysis.

### 1.6 Preparation of Liraglutide [SEQ ID NO: 1]

The cleavage of the peptide from the resin with simultaneous deprotection of the protecting groups is performed as following: a. peptide resin obtained as described above is added to the reactor containing a cold solution of cleavage cocktail; b the mixture is mixed for about 2 hours at room temperature; c. the product is precipitated by the addition of 10 volumes of ether (MTBE), filtered and dried in vacuum to obtain crude product.

The crude peptide obtained above, is dissolved and loaded on a C₁₈ RP-HPLC column and purified to obtain fractions containing Liraglutide at a purity of >97.5%. The pure fractions are collected and lyophilized to obtain a final dry peptide.

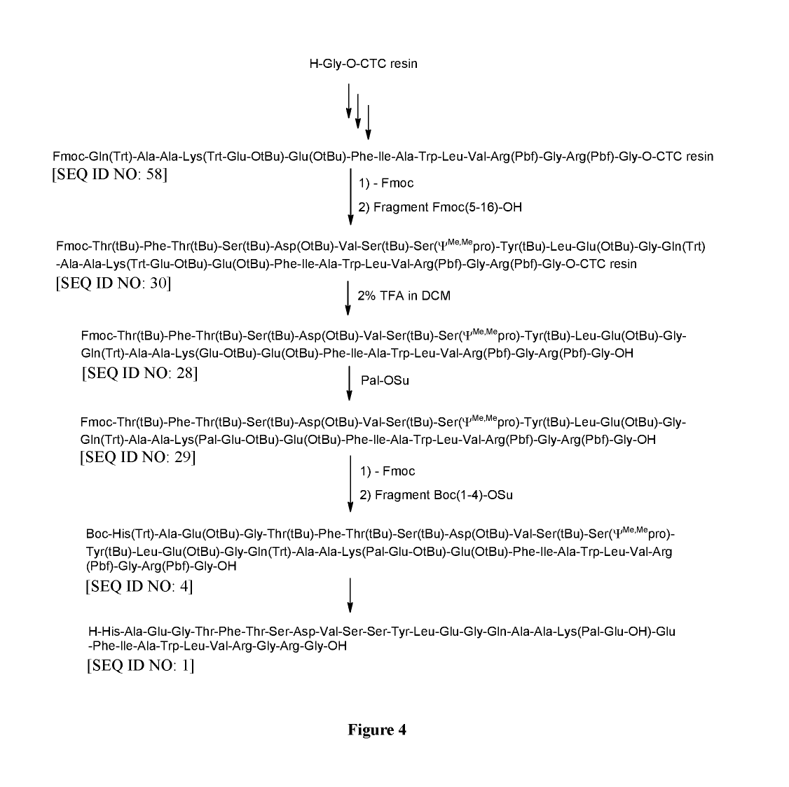

### Example 2: Synthesis of Liraglutide using Fmoc-Lys(Trt-Glu-OtBu) and Coupling of Peptide 3 and 4a on Resin (FIG. 4)

### 2.1 Synthesis of Fmoc-Gln(Trt)-Ala-Ala-Lys(Trt-Glu-OtBu)-Glu(OtBu)-Phe-Ile-Ala-Trp-Leu-Val-Arg(Pbf)-Gly-Arg(Pbf)-Gly-O-CTC resin [SEQ ID NO: 58]

Synthesis of the peptide sequence was carried out by a stepwise Fmoc SPPS (solid phase peptide synthesis) procedure starting with H-Gly-CTC resin (0.5 g, 0.2 mmol/g). The resin was washed by several washings with DMF and after the washing the second amino acid (Fmoc-Arg(Pbf)-OH) was introduced to start the first coupling step. The Fmoc protected amino acids were pre-activated using DIC/HOBt (N-hydroxybenzotriazole) and subsequently coupled to the resin for 50 minutes. Completion of the coupling was indicated by a Ninhydrine test. After washing of the resin, the Fmoc protecting group on the α-amine was removed by washing with 20% piperidine in DMF for 20 min. These steps were repeated each time with another amino acid according to peptide sequence. All amino acids used are Fmoc-N^{a}protected. Trifunctional amino acids are side chain protected as follows: Gln(Trt), Arg(Pbf), Lys(Trt-Glu-OtBu), and Glu(OtBu). Up to three equivalents of the activated amino acids were used in the coupling reactions. At the end of the synthesis the peptide-resin was washed with DMF, followed by MeOH, and dried under vacuum to obtain dry peptide-resin.

### 2.2 Preparation of Fmoc-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(Trt-Glu-OtBu)-Glu(OtBu)-Phe-Ile-Ala-Trp-Leu-Val-Arg(Pbf)-Gly-Arg(Pbf)-Gly-O-CTC resin [SEQ ID NO: 30]

After removal of the Fmoc group from SEQ ID NO: 51 (i.e. Fmoc-Gln(Trt)-Ala-Ala-Lys(Trt-Glu-OtBu)-Glu(OtBu)-Phe-Ile-Ala-Trp-Leu-Val-Arg(Pbf)-Gly-Arg(Pbf)-Gly-O-CTC resin (Example 2.1, SEQ ID NO: 58) on resin to form H-Gln(Trt)-Ala-Ala-Lys(Trt-Glu-OtBu)-Glu(OtBu)-Phe-Ile-Ala-Trp-Leu-Val-Arg(Pbf)-Gly-Arg(Pbf)-Gly-O-CTC resin (SEQ ID NO: 59/SEQ ID NO: 143), it was coupled to (Fmoc-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-OH) (SEQ ID NO: 44) on solid support. Thus SEQ ID NO: 44 (390 mg) was dissolved in DMF and preactivated by reaction with DIC/HOBt. This solution is added to the reactor containing H-Gln(Trt)-Ala-Ala-Lys(Trt-Glu-OtBu)-Glu(OtBu)-Phe-Ile-Ala-Trp-Leu-Val-Arg(Pbf)-Gly-Arg(Pbf)-Gly-O-CTC resin (SEQ ID NO: 59/SEQ ID NO: 143) on solid support. The reaction continued until completion. The completion of the reaction is monitored by HPLC analysis. At the end of the reaction the resin was washed with DMF. The peptide on the resin is identified by MS analysis.

### 2.3 Preparation of Fmoc-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(Pal-Glu-OtBu)-Glu(OtBu)-Phe-Ile-Ala-Trp-Leu-Val-Arg(Pbf)-Gly-Arg(Pbf)-Gly-OH [SEQ ID NO: 29]

The peptide Fmoc-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(Trt-Glu-OtBu)-Glu(OtBu)-Phe-Ile-Ala-Trp-Leu-Val-Arg(Pbf)-Gly-Arg(Pbf)-Gly-O-CTC resin (SEQ ID NO: 30), prepared as described above, was cleaved from the resin using a 1.5% TFA solution in DCM by four repeated washings (15 min each). The acidic peptide solution was extracted with water to remove TFA. Organic phase was concentrated and the peptide precipitated in ether. It was dissolved in DCM and Pal-OSu was added to react with free amino group on the Lys residue. The completion of the reaction was monitored by HPLC.

### 2.4 Preparation of Boc-His(Trt)-Ala-Glu(OtBu)-Gly-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(Pal-Glu-OtBu)-Glu(OtBu)-Phe-Ile-Ala-Trp-Leu-Val-Arg(Pbf)-Gly-Arg(Pbf)-Gly-OH [SEQ ID NO: 4] and liraglutide

To the previously prepared solution of the peptide fragment piperidine was added to remove Fmoc group. After completion of the Fmoc deprotection, the solution was extracted using 0.1N HCl to remove excess piperidine. The organic phase was concentrated and the peptide precipitated in ether, filtrated and dried to obtain H-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(Pal-Glu-OtBu)-Glu(OtBu)-Phe-Ile-Ala-Trp-Leu-Val-Arg(Pbf)-Gly-Arg(Pbf)-Gly-OH [SEQ ID NO: 31].

The peptide SEQ ID NO: 31 was dissolved in DMF (3 ml). It was then condensed with Boc-His(Trt)-Ala-Glu(OtBu)-Gly-OH [SEQ ID NO: 7] (79.5 mg) using DIC/HOBt. The completion of the reaction was monitored by HPLC and the Ninhydrine test. After completion of the condensation the protected peptide was precipitated by the addition of water and deprotected according to the standard procedure using TFA based cleavage cocktail. It was precipitated in ether, filtered and dried to obtain crude Liraglutide (330 mg) with purity of 64.5%.

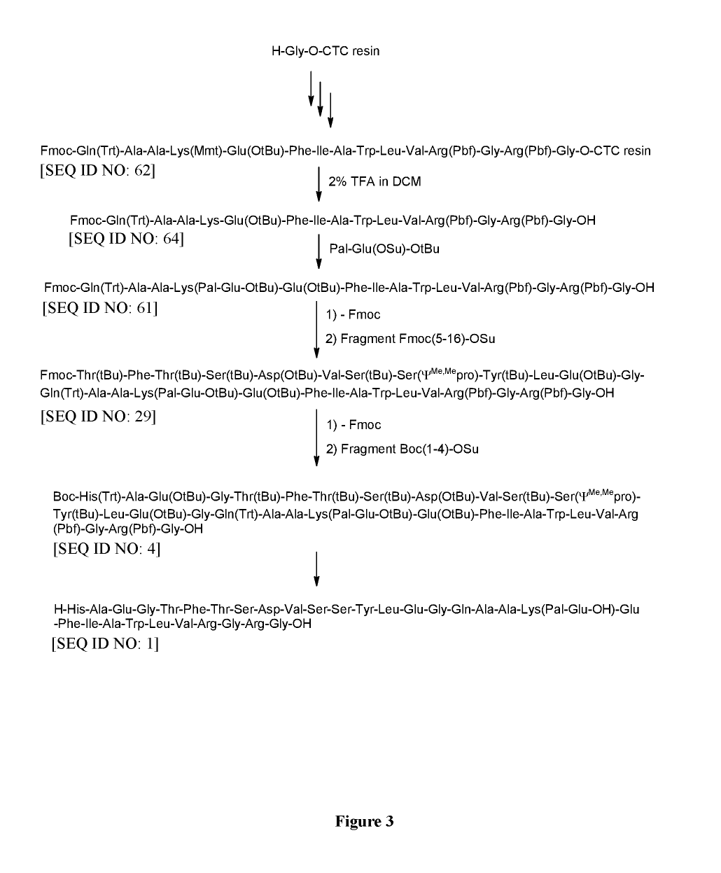

### Example 3: Synthesis of H-Gln(Trt)-Ala-Ala-Lys(Pal-Glu-OtBu)-Glu(OtBu)-Phe-Ile-Ala-Trp-Leu-Val-Arg(Pbf)-Gly-Arg (Pbf)-Gly-OH [SEQ ID NO: 56/SEQ ID NO: 148] on CTC resin using Fmoc-Lys(Mmt) (FIG. 3)

### Synthesis of H-Gln(Trt)-Ala-Ala-Lys(Pal-Glu-OtBu)-Glu(OtBu)-Phe-Ile-Ala-Trp-Leu-Val-Arg(Pbf)-Gly-Arg(Pbf)-Gly-OH [SEQ ID NO: 56/SEQ ID NO: 148]

Synthesis of the peptide sequence was carried out by a stepwise Fmoc SPPS (solid phase peptide synthesis) procedure starting with H-Gly-CTC resin. The resin was washed by several washings with DMF and after the washing the second amino acid (Fmoc-Arg(Pbf)-OH) was introduced to start the first coupling step. The Fmoc protected amino acid was pre-activated using DIC/HOBt (N-hydroxybenzotriazole) and subsequently coupled to the resin for 50 minutes. Completion of the coupling was indicated by a Ninhydrine test. After washing of the resin, the Fmoc protecting group on the α-amine was removed by washing with 20% piperidine in DMF for 20 min. These steps were repeated each time with another amino acid according to peptide sequence. All amino acids used were Fmoc-N^{α} protected. Trifunctional amino acids were side chain protected as follows: Gln(Trt), Arg(Pbf), Lys(Mmt), and Glu(OtBu). Up to three equivalents of the activated amino acids were used in the coupling reactions. At the end of the synthesis the peptide-resin was washed with DMF, followed by MeOH, and dried under vacuum to obtain dry peptide-resin.

The peptide-resin (7.75 g, 1 mmol), prepared as described above, was cleaved from the resin using a 1.5% TFA solution in DCM by four repeated washings (2 min each). TFA was removed from the solution by extraction with water till pH 4-5. The obtained organic phase was concentrated in vacuum and protected Fmoc-17-31-OH (deprotected at Lys²⁰ side chain) was precipitated by addition of diethyl ether, washed by DEE on filter and dried for 30 min at RT. The obtained protected peptide was dissolved in DCM and 593.6 mg (1.1 mmol) 1-tert-butyl 5-(2,5-dioxopyrrolidin-1-yl) 2-palmitamidopentanedioate [Pal-Glu(OSu)-OtBu] were added and the mixture was stirred for 12 h at RT. Then 425.5 mg (5.0 mmol) piperidine was added and the obtained mixture was stirred for additional 3 h at RT. The mixture was then extracted with 0.1N HCl followed by extraction with water till pH 4-5 in order to remove piperidine. The obtained organic phase was then concentrated in vacuum and H-Gln(Trt)-Ala-Ala-Lys(Pal-Glu-OtBu)-Glu(OtBu)-Phe-Ile-Ala-Trp-Leu-Val-Arg(Pbf)-Gly-Arg(Pbf)-Gly-OH [SEQ ID NO: 56/SEQ ID NO: 148] was precipitated by the addition of DEE (diethylether), washed with DEE (×3) and dried in vacuum to constant weight. Yield 3.12 g (100%).

### Example 4: Synthesis of Fmoc-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-OSu—[SEQ ID NO: 45]

Fmoc-(5-16)-OH [SEQ ID NO: 44] (979.7 mg, 0.5 mmol), prepared as described above (Example 1.2), was dissolved in 3 mL DCM. N-hydroxysuccinimide (HOSu) (74.8 mg, 0.65 mmol) was added in 0.2 ml THF followed by addition of DIC (63.0 mg, 0.5 mmol). The mixture was then stirred for 3 h at RT, concentrated in vacuum, precipitated by the addition of MTBE, washed with MTBE and dried in vacuum to constant weight. Yield: 1.01 g.

### Example 5: Synthesis of Boc-His(Trt)-Ala-Glu(OtBu)-Gly-OSu—[SEQ ID NO: 10]

Boc-His(Trt)-Ala-Glu(OtBu)-Gly-OSu is prepared similar to the procedure above starting with Boc-His(Trt)-Ala-Glu(OtBu)-Gly-OH [SEQ ID NO: 7].

### Example 6: Synthesis of Liraglutide by Fragment Condensation in Solution

### 6.1 Preparation of H-Thr(tBu)-Phe-Thr(tBu)-Ser(tBu)-Asp(OtBu)-Val-Ser(tBu)-Ser(Ψ^{Me,Me}pro)-Tyr(tBu)-Leu-Glu(OtBu)-Gly-Gln(Trt)-Ala-Ala-Lys(Pal-Glu-OtBu)-Glu(OtBu)-Phe-Ile-Ala-Trp-Leu-Val-Arg(Pbf)-Gly-Arg(Pbf)-Gly-OH [SEQ ID NO: 31]

[Fmoc-(5-16)-OSu] [SEQ ID NO: 45] (514 mg, 0.25 mmol), prepared as described above (Example 4), was dissolved in NMP (10 ml). [H-(17-31)-OH] [SEQ ID NO: 56/SEQ ID NO: 148] (Example 3) (606 mg, 0.2 mmol) was added and the resulting mixture was stirred for 8 h at RT. DIPEA (0.02 ml, 0.12 mmol) was added and the reaction mixture was stirred for additional 4 h at RT. Then piperidine (176.2 mg, 2.0 mmol) was added and the mixture was stirred for additional 3 h at RT. The reaction mixture was diluted with DCM (40 ml) and extracted by aq. washings. The organic phase was concentrated in vacuum to obtain protected H-(5-31)-OH [SEQ ID NO: 31] as oily residue.

### 6.2 Preparation of Liraglutide [SEQ ID NO: 1]

The obtained as described above protected H-(5-31)-OH [SEQ ID NO: 31] (Example 6.1) without further purification was dissolved in DCM (10 ml). To the resulting solution protected [Boc-(1-4)-OSu] [SEQ ID NO: 10] (Example 5) (204.2 mg, 0.22 mmol) was added and the mixture was stirred for 1 h at RT. Then DIPEA (0.04 ml, 0.24 mmol) was added and the mixture was stirred for additional 3 h. The resulting mixture was concentrated in vacuum to obtain protected Liraglutide. Side chain deprotection is carried by addition of a mixture of TFA/DCM/DTT (94:3:3) for 3 h at RT (cleavage cocktail) The reaction mixture is then concentrated on a rotary evaporator and the deprotected peptide was precipitated by the addition of pre chilled diethyl ether, collected by filtration washed with Et₂O (×3) and dried to a constant weight in air and then in vacuum. The Liraglutide purified on preparative HPLC column. Yield: 330.1 mg (44%) and 99.4% purity.

### Example 7: Purification and Isolation of Liraglutide

The Liraglutide crude (10 gram, 56.5% purity) was dissolved and loaded on a HPLC RP preparative column with, 15 am. It was purified using linear gradient of aqueous buffer and organic solvent comprising acetonitrile. Fractions containing Liraglutide >97.0% were combined and transferred to ion exchange.

Fractions containing Liraglutide at a purity of >97% (0.1 g) were loaded to RP HPLC column. After the loading the column was washed with 0.5M Ammonium acetate solution (pH=8.4) until the pH of the eluent was >8. Then, the column was washed with 2% (w/w) AcOH, 2% ACN water solution until the pH of the eluent was <4. The Liraglutide was eluted with linear gradient of MPA: 0.2% (w/w) AcOH solution, MPB: ACN. Pure fractions were collected and lyophilized to obtain a final dry peptide (36 mg)>98.0% pure (HPLC).

### Example 8: Purification of Liraglutide

10 gram of crude Liraglutide were dissolved in 1 litre of Glycine buffer (80% 0.1 M glycine and 20% ACN, pH=10.8±0.2 adjusted with NH₄OH). The mixture was stirred at room temperature (RT) for about 1 hr. After 1 hr, the pH adjusted to about 9 with trifluoroacetic acid (TFA). The obtained solution was loaded to a preparative 2 inch column containing C8, 15 μm silica.

A purification cycle was performed with gradient of:

Mobile phase A: 0.01M Ammonium Chloride, pH=8.5 and

Mobile phase B: 7:3 ACN:EtOH solution

1.54 gram of Liraglutide with a purity of more than 97% were obtained after several purification cycles (40% yield).

Fractions containing Liraglutide with >97% purity (10 gram of Liraglutide) were further purified using preparative HPLC, on C8, 15 μm silica with the following gradient:

Mobile phase C: water pH=8 (adjusted with NH₄OH)

Mobile phase D: acetonitrile

4.5 gram of Liraglutide with a purity more than 98.5% were obtained after several purification cycles) 45% yield).

The obtained solution is evaporated (up to 20% of the volume is evaporated).

The pH of the solution in the end of the evaporation is 6.5-7.5. The solution is lyophilized to obtain pure Liraglutide powder (>98.5% purity, each impurity <0.5%).

### Synthesis of GLP-1 Peptides

Disclosed are processes for the synthesis of GLP-1 peptides, such as liraglutide and semaglutide, and a process for purifying liraglutide.

1. A process for preparing a GLP-1 peptide containing a lysine having a derivatized side chain, wherein the process comprises liquid or solid phase peptide synthesis or a combination thereof, wherein the process comprises a final coupling step in which at least two fragments are coupled at a terminal Gly residue, and wherein at least one of the fragments is prepared by coupling of at least two sub-fragments.

2.-8. (canceled)

9. The process according to claim 1, wherein the GLP-1 peptide is liraglutide [SEQ ID NO: 1] consisting of the following formula:
1 2 3 4 5 6 7 8 9 10
His-Ala-Glu-Gly-Thr-Phe-Thr-Ser-Asp-Val-
11 12 13 14 15 16 17 18 19 20
Ser-Ser-Tyr-Leu-Glu-Gly-Gln-Ala-Ala-Lys(Pal-Glu)-
21 22 23 24 25 26 27 28 29 30 31
Glu-Phe-Ile-Ala-Trp-Leu-Val-Arg-Gly-Arg-Gly-OH
wherein the process comprises:
(iv) coupling a Peptide 1 consisting of the sequence:
[SEQ ID NO: 5]
His-Ala-Glu-Gly
wherein:
the N-terminal of His is optionally protected with a protecting group, and
the Gly carboxylic acid group in Peptide 1 may be in the form of an activated carboxylic acid derivative;
with a Peptide 2 having the sequence:
[SEQ ID NO: 24]
Thr-Phe-Thr-Ser-Asp-Val-Ser-Ser-Tyr-Leu-Glu-Gly-
Gln-Ala-Ala-Lys(Pal-Glu-OX)-Glu-Phe-Ile-Ala-Trp-
Leu-Val-Arg-Gly-Arg-Gly-OH
wherein:
Peptide 2 is optionally conjugated to a solid support;
X represents H or a protecting group for the Glu carboxylic acid group, and wherein one or more of the amino acid residues in Peptide 1 and Peptide 2 may be protected or unprotected,
(v) cleaving from the resin to form liraglutide; and
(vi) optionally purifying the liraglutide
wherein the Peptide 2 is prepared by a two or three fragment convergent synthesis.

10.-31. (canceled)

32. A fragmental peptide of Liraglutide, selected from the group consisting of:
(i)
[SEQ ID NO: 119]
His(Trt)-Ala-Glu(OtBu)-Gly-OSu,
(ii)
[SEQ ID NO: 120]
His(Trt)-Ala-Glu(OtBu)-Gly-OBt,
(iii)
[SEQ ID NO: 121]
His(Trt)-Ala-Glu(OtBu)-Gly-OPfp,
(iv)
[SEQ ID NO: 123]
Lys(Mtt)-Glu(OtBu)-Phe-Ile-Ala-Trp-Leu-Val-Arg(Pbf)-Gly-Arg(Pbf)-Gly-O-
Wang resin,
(v)
[SEQ ID NO: 124]
Lys(Mmt)-Glu(OtBu)-Phe-Ile-Ala-Trp-Leu-Val-Arg(Pbf)-Gly-Arg(Pbf)-Gly-O-
Wang resin,
(iv)
[SEQ ID NO: 125]
Lys-Glu(OtBu)-Phe-Ile-Ala-Trp-Leu-Val-Arg(Pbf)-Gly-Arg(Pbf)-Gly-O-Wang
resin,
(vii)
[SEQ ID NO: 126 or SEQ ID NO: 167]
Lys(Glu-OtBu)-Glu(OtBu)-Phe-Ile-Ala-Trp-Leu-Val-Arg(Pbf)-Gly-Arg(Pbf)-Gly-
O-Wang resin,
(viii)
[SEQ ID NO: 127]
Lys(Pal-Glu-OtBu)-Glu(OtBu)-Phe-Ile-Ala-Trp-Leu-Val-Arg(Pbf)-Gly-Arg(Pbf)-
Gly-O-Wang resin,
(ix)
[SEQ ID NO: 128 or SEQ ID NO: 55]
Gln(Trt)-Ala-Ala-Lys(Pal-Glu-OtBu)-Glu(OtBu)-Phe-Ile-Ala-Trp-Leu-Val-
Arg(Pbf)-Gly-Arg(Pbf)-Gly-O-Wang resin,
(x)
[SEQ ID NO: 129]
Ala-Lys(Mtt)-Glu(OtBu)-Phe-Ile-Ala-Trp-Leu-Val-Arg(Pbf)-Gly-Arg(Pbf)-Gly-O-
Wang resin,
(xi)
[SEQ ID NO: 130]
Ala-Lys(Mmt)-Glu(OtBu)-Phe-Ile-Ala-Trp-Leu-Val-Arg(Pbf)-Gly-Arg(Pbf)-Gly-
O-Wang resin,
(xii)
[SEQ ID NO: 131]
Ala-Lys(Trt-Glu-OtBu)-Glu(OtBu)-Phe-Ile-Ala-Trp-Leu-Val-Arg(Pbf)-Gly-
Arg(Pbf)-Gly-O-Wang resin,
(xiii)
[SEQ ID NO: 132]
Ala-Lys(Glu-OtBu)-Glu(OtBu)-Phe-Ile-Ala-Trp-Leu-Val-Arg(Pbf)-Gly-Arg(Pbf)-
Gly-O-Wang resin,
(xiv)
[SEQ ID NO: 133]
Ala-Lys-Glu(OtBu)-Phe-Ile-Ala-Trp-Leu-Val-Arg(Pbf)-Gly-Arg(Pbf)-Gly-O-
Wang resin,
(xv)
[SEQ ID NO: 134 or SEQ ID NO: 103]
Ala-Lys(Pal-Glu-OtBu)-Glu(OtBu)-Phe-Ile-Ala-Trp-Leu-Val-Arg(Pbf)-Gly-
Arg(Pbf)-Gly-O-Wang resin,
(xvi)
[SEQ ID NO: 135]
Ala-Ala-Lys(Mtt)-Glu(OtBu)-Phe-Ile-Ala-Trp-Leu-Val-Arg(Pbf)-Gly-Arg(Pbf)-
Gly-O-Wang resin,
(xvii)
[SEQ ID NO: 136]
Ala-Ala-Lys(Mmt)-Glu(OtBu)-Phe-Ile-Ala-Trp-Leu-Val-Arg(Pbf)-Gly-Arg(Pbf)-
Gly-O-Wang resin,
(xviii)
[SEQ ID NO: 137]
Ala-Ala-Lys(Trt-Glu-OtBu)-Glu(OtBu)-Phe-Ile-Ala-Trp-Leu-Val-Arg(Pbf)-Gly-
Arg(Pbf)-Gly-O-Wang resin,
(xix)
[SEQ ID NO: 138]
Ala-Ala-Lys(Glu-OtBu)-Glu(OtBu)-Phe-Ile-Ala-Trp-Leu-Val-Arg(Pbf)-Gly-
Arg(Pbf)-Gly-O-Wang resin,
(xx)
[SEQ ID NO: 139]
Ala-Ala-Lys-Glu(OtBu)-Phe-Ile-Ala-Trp-Leu-Val-Arg(Pbf)-Gly-Arg(Pbf)-Gly-O-
Wang resin,
(xxi)
[SEQ ID NO: 140 or SEQ ID NO: 112]
Ala-Ala-Lys(Pal-Glu-OtBu)-Glu(OtBu)-Phe-Ile-Ala-Trp-Leu-Val-Arg(Pbf)-Gly-
Arg(Pbf)-Gly-O-Wang resin,
(xxii)
[SEQ ID NO: 141]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-OH,
(xxiii)
[SEQ ID NO: 305]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-OH,
(xxiv)
[SEQ ID NO: 142]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-OSu
(xxv)
[SEQ ID NO: 306]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-OSu,
(xxvi)
[SEQ ID NO: 143 or SEQ ID NO: 59]
Gln(Trt)-Ala-Ala-Lys(Trt-Glu-OtBu)-Glu(OtBu)-Phe-Ile-Ala-Trp-Leu-Val-
Arg(Pbf)-Gly-Arg(Pbf)-Gly-O-CTC resin,
(xxvii)
[SEQ ID NO: 144 or SEQ ID NO: 154]
Gln(Trt)-Ala-Ala-Lys(Mmt)-Glu(OtBu)-Phe-Ile-Ala-Trp-Leu-Val-Arg(Pbf)-Gly-
Arg(Pbf)-Gly-O-CTC resin ,
(xxviii)
[SEQ ID NO: 145 or SEQ ID NO: 152]
Gln(Trt)-Ala-Ala-Lys(Mtt)-Glu(OtBu)-Phe-Ile-Ala-Trp-Leu-Val-Arg(Pbf)-Gly-
Arg(Pbf)-Gly-O-CTC resin,
(xxix)
[SEQ ID NO: 146 or SEQ ID NO: 153]
Gln(Trt)-Ala-Ala-Lys-Glu(OtBu)-Phe-Ile-Ala-Trp-Leu-Val-Arg(Pbf)-Gly-
Arg(Pbf)-Gly-OH,
(xxx)
[SEQ ID NO: 147]
Gln(Trt)-Ala-Ala-Lys(Glu-OtBu)-Glu(OtBu)-Phe-Ile-Ala-Trp-Leu-Val-Arg(Pbf)-
Gly-Arg(Pbf)-Gly-OH,
(xxxi)
[SEQ ID NO: 148 or SEQ ID NO: 56]
Gln(Trt)-Ala-Ala-Lys(Pal-Glu-OtBu)-Glu(OtBu)-Phe-Ile-Ala-Trp-Leu-Val-
Arg(Pbf)-Gly-Arg(Pbf)-Gly-OH,
(xxxii)
[SEQ ID NO: 149]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-Gly-OH,
(xxxiii)
[SEQ ID NO: 309 or SEQ ID NO: 276]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-Gln(Trt)-Ala-Ala-Lys(Pal-Glu-OtBu)-Glu(OtBu)-Phe-Ile-Ala-Trp-
Leu-Val-Arg(Pbf)-Gly-Arg(Pbf)-Gly-OH,
(xxxiv)
[SEQ ID NO: 150]
Gln(Trt)-Ala-Ala-Lys(Trt-Glu-OtBu)-Glu(OtBu)-Phe-Ile-Ala-Trp-Leu-Val-
Arg(Pbf)-Gly-Arg(Pbf)-Gly-O-Wang resin,
(xxxv)
[SEQ ID NO: 151]
Gln(Trt)-Ala-Ala-Lys(Glu-OtBu)-Glu(OtBu)-Phe-Ile-Ala-Trp-Leu-Val-Arg(Pbf)-
Gly-Arg(Pbf)-Gly-O-Wang resin,
(xxxvi)
[SEQ ID NO: 152 or SEQ ID NO: 145]
Gln(Trt)-Ala-Ala-Lys(Mtt)-Glu(OtBu)-Phe-Ile-Ala-Trp-Leu-Val-Arg(Pbf)-Gly-
Arg(Pbf)-Gly-O-CTC resin,
(xxxvii)
[SEQ ID NO: 153 or SEQ ID NO: 146]
Gln(Trt)-Ala-Ala-Lys-Glu(OtBu)-Phe-Ile-Ala-Trp-Leu-Val-Arg(Pbf)-Gly-
Arg(Pbf)-Gly-OH,
(xxxviii)
[SEQ ID NO: 154 or SEQ ID NO: 144]
Gln(Trt)-Ala-Ala-Lys(Mmt)-Glu(OtBu)-Phe-Ile-Ala-Trp-Leu-Val-Arg(Pbf)-Gly-
Arg(Pbf)-Gly-O-CTC resin,
(xxxix)
[SEQ ID NO: 155]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-Gln(Trt)-Ala-Ala-Lys(Mtt)-Glu(OtBu)-Phe-Ile-Ala-
Trp-Leu-Val-Arg(Pbf)-Gly-Arg(Pbf)-Gly-O-CTC resin,
(xl)
[SEQ ID NO: 310]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-Gln(Trt)-Ala-Ala-Lys(Mtt)-Glu(OtBu)-Phe-Ile-Ala-Trp-Leu-Val-
Arg(Pbf)-Gly-Arg(Pbf)-Gly-0-CTC resin,
(xli)
[SEQ ID NO: 156]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-Gln(Trt)-Ala-Ala-Lys-Glu(OtBu)-Phe-Ile-Ala-Trp-
Leu-Val-Arg(Pbf)-Gly-Arg(Pbf)-Gly-OH,
(xlii)
[SEQ ID NO: 311]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-Gln(Trt)-Ala-Ala-Lys-Glu(OtBu)-Phe-Ile-Ala-Trp-Leu-Val-
Arg(Pbf)-Gly-Arg(Pbf)-Gly-OH,
(xliii)
[SEQ ID NO: 157]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-Gln(Trt)-Ala-Ala-Lys(Mtt)-Glu(OtBu)-Phe-Ile-Ala-
Trp-Leu-Val-Arg(Pbf)-Gly-Arg(Pbf)-Gly-O-Wang resin,
(xliv)
[SEQ ID NO: 312]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-Gln(Trt)-Ala-Ala-Lys(Mtt)-Glu(OtBu)-Phe-Ile-Ala-Trp-Leu-Val-
Arg(Pbf)-Gly-Arg(Pbf)-Gly-O-Wang resin,
(xlv)
[SEQ ID NO: 158]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-Gln(Ttr)-Ala-Ala-Lys(Mmt)-Glu(OtBu)-Phe-Ile-
Ala-Trp-Leu-Val-Arg(Pbf)-Gly-Arg(Pbf)-Gly-O-Wang resin,
(xlvi)
[SEQ ID NO: 313]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-Gln(Trt)-Ala-Ala-Lys(Mmt)-Glu(OtBu)-Phe-Ile-Ala-Trp-Leu-Val-
Arg(Pbf)-Gly-Arg(Pbf)-Gly-O-Wang resin,
(xlvii)
[SEQ ID NO: 159]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-Gln(Trt)-Ala-Ala-Lys-Glu(OtBu)-Phe-Ile-Ala-Trp-
Leu-Val-Arg(Pbf)-Gly-Arg(Pbf)-Gly-O-Wang resin,
(xlviii)
[SEQ ID NO: 314]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-Gln(Trt)-Ala-Ala-Lys-Glu(OtBu)-Phe-Ile-Ala-Trp-Leu-Val-
Arg(Pbf)-Gly-Arg(Pbf)-Gly-O-Wang resin,
(xlix)
[SEQ ID NO: 160]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-Gln(Trt)-Ala-Ala-Lys(Trt-Gly-OtBu)-Glu(OtBu)-
Phe-Ile-Ala-Trp-Leu-Val-Arg(Pbf)-Gly-Arg(Pbf)-Gly-O-Wang resin,
(l)
[SEQ ID NO: 315]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-Gln(Trt)-Ala-Ala-Lys(Trt-Gly-OtBu)-Glu(OtBu)-Phe-Ile-Ala-Trp-
Leu-Val-Arg(Pbf)-Gly-Arg(Pbf)-Gly-O-Wang resin,
(li)
[SEQ ID NO: 161]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-Gln(Trt)-Ala-Ala-Lys(Pal-Glu-OtBu)-Glu(OtBu)-
Phe-Ile-Ala-Trp-Leu-Val-Arg(Pbf)-Gly-Arg(Pbf)-Gly-O-Wang resin,
(lii)
[SEQ ID NO: 316 or SEQ ID NO: 278]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-Gln(Trt)-Ala-Ala-Lys(Pal-Glu-OtBu)-Glu(OtBu)-Phe-Ile-Ala-Trp-
Leu-Val-Arg(Pbf)-Gly-Arg(Pbf)-Gly-O-Wang resin,
(liii)
[SEQ ID NO: 162]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-Gln(Ttr)-Ala-Ala-Lys(Glu-OtBu)-Phe-Ile-Ala-Trp-
Leu-Val-Arg(Pbf)-Gly-Arg(Pbf)-Gly-OH,
(liv)
[SEQ ID NO: 317]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-Gln(Trt)-Ala-Ala-Lys(Glu-OtBu)-Phe-Ile-Ala-Trp-Leu-Val-
Arg(Pbf)-Gly-Arg(Pbf)-Gly-OH,
(lv)
[SEQ ID NO: 163 or SEQ ID NO: 88]
Tyr(tBu)-Leu-Glu(OtBu)-Gly-O-CTC resin,
(lvi)
[SEQ ID NO: 164]
Thr(tBu)-Phe-Thr(tBu)-Ser(tBu)-Asp(OtBu)-Val-Ser(tBu)-Ser(Ψ^{Me,Me}pro)-O-CTC
resin,
(lvii)
[SEQ ID NO: 318]
Thr(tBu)-Phe-Thr(tBu)-Ser(tBu)-Asp(OtBu)-Val-Ser(Trt)-Ser(Trt)-O-CTC resin,
(lviii)
[SEQ ID NO: 165]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-O-CTC resin,
(lix)
[SEQ ID NO: 319]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-O-CTC resin,
(lx)
[SEQ ID NO: 166]
Lys(Trt-Glu-OtBu)-Glu(OtBu)-Phe-Ile-Ala-Trp-Leu-Val-Arg(Pbf)-Gly-Arg(Pbf)-
Gly-O-Wang resin,
(lxi)
[SEQ ID NO: 167 or SEQ ID NO: 126]
Lys(Glu-OtBu)-Glu(OtBu)-Phe-Ile-Ala-Trp-Leu-Val-Arg(Pbf)-Gly-Arg(Pbf)-Gly-
O-Wang resin,
(lxii)
[SEQ ID NO: 168]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OTBu)-Gly-Gln(Trt)-Ala-Ala-Lys(Mmt)-Glu(OtBu)-Phe-Ile-
Ala-Trp-Leu-Val-Arg(Pbf)-Gly-Arg(Pbf)-Gly-O-CTC resin,
(lxiii)
[SEQ ID NO: 320]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-Gln(Trt)-Ala-Ala-Lys(Mmt)-Glu(OtBu)-Phe-Ile-Ala-Trp-Leu-Val-
Arg(Pbf)-Gly-Arg(Pbf)-Gly-O-CTC resin,
(lxiv)
[SEQ ID NO: 169]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-Gln(Trt)-Ala-Ala-Lys(Trt-Glu-OtBu)-Phe-Ile-Ala-
Trp-Leu-Val-Arg(Pbf)-Gly-Arg(Pbf)-Gly-O-CTC resin,
(lxv)
[SEQ ID NO: 321]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-Gln(Trt)-Ala-Ala-Lys(Trt-Glu-OtBu)-Phe-Ile-Ala-Trp-Leu-Val-
Arg(Pbf)-Gly-Arg(Pbf)-Gly-O-CTC resin,
(lxvi)
[SEQ ID NO: 170]
His(Trt)-Ala-Glu(OtBu)-Gly-Thr(tBu)-Phe-Thr(tBu)-Ser(tBu)-Asp(OtBu)-Val-
Ser(tBu)-Ser(Ψ^{Me,Me}pro)-Tyr(tBu)-Leu-Glu(OtBu)-Gly-Gln(Trt)-Ala-Ala-Lys(Glu-
OtBu)-Phe-Ile-Ala-Trp-Leu-Val-Arg(Pbf)-Gly-Arg(Pbf)-Gly-O-Wang resin,
[[and]]
(lxvii)
[SEQ ID NO: 322]
His(Trt)-Ala-Glu(OtBu)-Gly-Thr(tBu)-Phe-Thr(tBu)-Ser(tBu)-Asp(OtBu)-Val-
Ser(Trt)-Ser(Trt)-Tyr(tBu)-Leu-Glu(OtBu)-Gly-Gln(Trt)-Ala-Ala-Lys(Glu-OtBu)-
Phe-Ile-Ala-Trp-Leu-Val-Arg(Pbf)-Gly-Arg(Pbf)-Gly-O-Wang resin,
(lxviii)
[SEQ ID NO: 307]
Thr(tBu)-Phe-Thr(tBu)-Ser(tBu)-Asp(OtBu)-Val-Ser(tBu)-Ser(Ψ^{Me,Me}pro)-
Tyr(tBu)-Leu-Glu(OtBu)-Gly-OPfp,
and
(lxix)
[SEQ ID NO: 308]
Thr(tBu)-Phe-Thr(tBu)-Ser(tBu)-Asp(OtBu)-Val-Ser(Trt)-Ser(Trt)-Tyr(tBu)-Leu-
Glu(OtBu)-Gly-OPfp
wherein the N-terminal amino acids in (i)-(iii) are optionally protected with Fmoc, CBz or Boc, and wherein the N-terminal amino acids in (iv)-(lxvii) or (lxviii)-(lxix) are optionally protected with Fmoc or Cbz.

33.-34. (canceled)

35. A process of synthesizing liraglutide comprising coupling one or more peptides of claim 32 with at least one other sub-peptide to form liraglutide.

36. A composition comprising Liraglutide and
(a) less than 1% of the D-His isomer of liraglutide, and/or
(b) less than 1% of the [+Gly¹⁶] derivative of liraglutide, and/or
(c) less than 1% of the [+Gly³¹] derivative of liraglutide, and/or
(d) less than 1% of the [+Gly⁴] derivative of liraglutide.

37. The process according to claim 1, wherein the GLP-1 peptide is semaglutide consisting of the following formula:
1 2 3 4 5 6 7 8 9 10 11 12
His-Aib-Glu-Gly-Thr-Phe-Thr-Ser-Asp-Val-Ser-Ser-
13 14 15 16 17 18 19 20 21 22
Tyr-Leu-Glu-Gly-Gln-Ala-Ala-Lys(W)-Glu-Phe-Ile-
23 24 25 26 27 28 29 30 31
Ala-Trp-Leu-Val-Arg-Gly-Arg-Gly-OH
wherein W=N-(17-carboxy-1-oxoheptadecyl)-L-γ-glutamyl-2-[2-(2-aminoethoxy)ethoxy]acetyl-2-[2-(2-aminoethoxy)ethoxy]acetyl [SEQ ID NO: 174].

38.-67. (canceled)

68. A composition comprising semaglutide and:
(a) less than 1% of the D-His isomer of semaglutide, and/or
(b) less than 1 wt % of the [+Gly¹⁶] derivative of semaglutide, and/or
(c) less than 1 wt % of the [+Gly³¹] derivative of semaglutide, and/or
(d) less than 1 wt % of the [+Gly⁴] derivative of semaglutide.

69.-72. (canceled)

73. A process for purifying crude liraglutide comprising:
(a) dissolving crude Liraglutide in a solution comprising acetonitrile and water,
(b) subjecting the solution in step (a) to reversed-phase HPLC on a C8 or C18 silica column using a mobile phase A, comprising water, and a mobile phase B, comprising acetonitrile and at least one C₁₋₄ alcohol, and collecting the liraglutide fractions,
(c) optionally repeating step (b),
(d) subjecting the fractions to reversed-phase HPLC on a C8 or C18 silica column using a mobile phase C, comprising water, and a mobile phase D, comprising acetonitrile, and collecting the purified Liraglutide fractions,
(e) optionally concentrating the purified liraglutide fractions to form a purified liraglutide concentrate,
(f) optionally repeating step (d), or optionally repeating steps (d) and (e), and
(g) drying the purified liraglutide fractions or purified liraglutide concentrate,
wherein the purified liraglutide fractions or purified liraglutide concentrate before drying has a pH of 6.0-8.0.

74.-102. (canceled)

103. The process according to claim 73, wherein the liraglutide obtained after step (g) has a purity of 98.5% or more.

104. The process according to claim 73, further comprising combining the dried liraglutide with at least one pharmaceutically acceptable excipient to form a pharmaceutical composition.

105. A process for preparing semaglutide, wherein the process comprises conjugating a peptide selected from the group selected from one or more of Peptide 1, P1-His(P)-Aib-Glu(P)-Gly-O-P2 (SEQ ID NO:177), Peptide 3, P1-Thr(P)-Phe-Thr(P)-Ser(P)-Asp(P)-Val-Ser(P)-Ser(P)-Tyr(P)-Leu-Glu(P)-Gly-OP2 (SEQ ID NO: 43), and Peptide 4, P1-Gln(P)-Ala-Ala-Lys(W1)-Glu(P)-Phe-Ile-Ala-Trp-Leu-Val-Arg(P)-Gly-Arg(P)-Gly-O-P2 (SEQ ID NO: 196) with at least one additional peptide fragment, wherein P1 for Peptide 1 is a protecting group, each P in Peptide 1 represents a side chain protecting group which may be the same or different, and P2 for Peptide 1 is H, a solid support, or an activated carboxylic ester of the Gly⁴ residue, wherein P1 for Peptide 3 is a protecting group, each P in Peptide 3 represents a side chain protecting group which may be the same or difference and P2 for Peptide 3 is H, is an activated carboxylic ester of the Gly¹⁶ residue, or is a solid support, and wherein P1 in Peptide 4 is H or a protecting group for the N-terminal of Gln, each P of Peptide 4 is a side chain protecting group which may be the same or different, P2 in Peptide 4 is H or a solid support, and W1 in Peptide 4 is N-(17-carboxy-1-oxoheptadecyl)-L-γ-glutamyl[2-(2-aminoethoxy)ethoxy]acetyl[2-(2-aminoethoxy)ethoxy]acetic acid.

106. A peptide consisting of Gln-Ala-Ala-Lys(W)-Glu-Phe-Ile-Ala-Trp-Leu-Val-Arg-Gly-Arg-Gly-OH, wherein W1 is N-(17-carboxy-1-oxoheptadecyl)-L-γ-glutamyl[2-(2-aminoethoxy)ethoxy]acetyl[2-(2-aminoethoxy)ethoxy]acetic acid, wherein Peptide 4 is optionally conjugated to a solid support at the terminal Gly-OH, and wherein one or more of the amino acid residues in Peptide 4 is optionally protected.

107. A compound that is Fmoc-Lys(W1)-OH, wherein W1 is N-(17-carboxy-1-oxoheptadecyl)-L-γ-glutamyl[2-(2-aminoethoxy)ethoxy]acetyl[2-(2-aminoethoxy)ethoxy]acetic acid.

108. A process for preparing the compound of claim 107 comprising
a) loading 2-{[2-(Fmoc-amino)ethoxy]ethoxy}acetic acid to a CTC resin, and removing Fmoc,
b) coupling 2-{[2-(Fmoc-amino)ethoxy]ethoxy}acetic acid unit to the free amine group, and removing Fmoc,
c) coupling Fmoc-Glu-OtBu to the free amine, and removing Fmoc,
d) coupling octadecanedioic acid mono-tert-butyl ester, and
e) cleaving the 17-carboxy(OtBu)-1-oxoheptadecyl)-L-Y-glutamyl(OtBu)-2-[2-(2-aminoethoxy)ethoxy]acetyl-2-[2-(2-aminoethoxy)ethoxy]acetic acid from the resin.

109. A process for preparing the compound of claim 107 comprising
a) loading Cbz-Lys(Fmoc)-OH to a CTC resin, and removing Fmoc,
b) coupling 2-{[2-(Fmoc-amino)ethoxy]ethoxy}acetic acid unit to the free amine group, and removing Fmoc,
c) coupling 2-{[2-(Fmoc-amino)ethoxy]ethoxy}acetic acid unit to the free amine group, and removing Fmoc,
d) coupling Fmoc-Glu-OtBu to the free amine group, and removing Fmoc
e) coupling octadecanedioic acid mono-tert-butyl ester to Glu to form Cbz-Lys{N-(17-carboxy-1-oxoheptadecyl)-L-γ-glutamyl[2-(2-aminoethoxy) ethoxy]acetyl[2-(2-aminoethoxy)ethoxy] acetic acid}.

110. A process for preparing semaglutide or fragment (17-31) of semaglutide, wherein the process comprises a sequential synthesis on a resin in which the compound of claim 107 is incorporated.

111. A composition prepared by the process of claim 9, wherein the composition comprises liraglutide and
(a) less than 1% of the D-His isomer of liraglutide, and/or
(b) less than 1% of the [+Gly¹⁶] derivative of liraglutide, and/or
(c) less than 1 of the [+Gly³¹] derivative of liraglutide, and/or
(d) less than 1 of the [+Gly⁴] derivative of liraglutide.

112. A composition prepared by the process of claim 105, wherein the composition comprises semaglutide and
(a) less than 1% of the D-His isomer of semaglutide, and/or
(b) less than 1 wt % of the [+Gly¹⁶] derivative of semaglutide, and/or
(c) less than 1 wt % of the [+Gly³¹] derivative of semaglutide, and/or
(d) less than 1 wt % of the [+Gly⁴] derivative of semaglutide.
